(19) United States
(12) Patent Application Publication
(10) Pub. No.: US 2006/0134801 A1
(43) Pub. Date: Jun. 22, 2006

(54) Methods and compositions involving MDA-7

(75) Inventors: Sunil Chada, Missouri City, TX (US); John B. Mumm, Sugar Land, TX (US); Rajagopal Ramesh, Sugar Land, TX (US); Abner M. Mhashilkar, Birmingham, AL (US); Raymond E. Meyn, Houston, TX (US); Elizabeth Grimm, Houston, TX (US)

Correspondence Address: FULBRIGHT & JAWORSKI L.L.P., 600 CONGRESS AVE., SUITE 2400, AUSTIN, TX, 78701, US

(73) Assignees: Board of Regents, The University of Texas System; Introgen Therapeutics, Inc.

(21) Appl. No.: 10/791,692

(22) Filed: Mar. 2, 2004

Related U.S. Application Data
(60) Provisional application No. 60/452,257, filed on Mar. 3, 2003.
(60) Provisional application No. 60/474,529, filed on May 30, 2003.
(60) Provisional application No. 60/476,159, filed on Jun. 4, 2003.
(60) Provisional application No. 60/486,862, filed on Jul. 11, 2003.
(60) Provisional application No. 60/515,285, filed on Oct. 29, 2003.
(60) Provisional application No. 60/528,506, filed on Dec. 10, 2003.

(51) Int. Cl. G01N 1/10

(52) U.S. Cl. 436/177

(57) ABSTRACT

The present invention relates to compositions and methods involving MDA-7. More specifically, the present invention is directed to diagnostic, prognostic, and therapeutic treatment compositions and methods for treatment of cancer and other angiogenesis-related disorders (anti-angiogenesis therapy). The present invention is also directed to methods of purification of MDA-7.

This application claims the benefit of priority to U.S. application Ser. No. 60/452,257 filed Mar. 3, 2003, U.S. application Ser. No. 60/474,529 filed on May 30, 2003, U.S. application Ser. No. 60/476,159 filed Jun. 4, 2003, U.S. application Ser. No. 60/486,862 filed on Jul. 11,.2003, U.S. application Ser. No. 60/515,285 filed on Oct. 29, 2003, and U.S. application Ser. No. 60/528,506 filed on Dec. 10, 2003, which all have the same title and inventors as the present application. Each of these applications is hereby incorporated by reference in its entirety.

The United States Government may own rights in the invention pursuant to grant numbers CA86587, R41 CA88421, P01 CA78778, P01 CA06294, CA70907, and P30 CA16672 from the National Cancer Institute.

### BACKGROUND OF THE INVENTION

A. Field of the Invention

The present invention relates generally to the fields of molecular biology and gene therapy. More specifically, the present invention is directed to diagnostic, prognostic, and therapeutic treatment compositions and methods for treatment of cancer and other angiogenesis-related disorders (anti-angiogenesis therapy). The present invention is also directed to methods of purification of MDA-7 and compositions including purified MDA-7.

B. Description of Related Art

1. Angiogenesis

Blood vessels are constructed by two processes: vasculogenesis, whereby a primitive vascular network is established during embryogenesis from multipotential mesenchymal progenitors; and angiogenesis, in which preexisting vessels send out capillary sprouts to produce new vessels. Endothelial cells are centrally involved in each process. They migrate, proliferate and then assemble into tubes with tight cell-cell connections to contain the blood (Hanahan, 1997). Angiogenesis occurs when enzymes, released by endothelial cells, and leukocytes begin to erode the basement membrane, which surrounds the endothelial cells, allowing the endothelial cells to protrude through the membrane. These endothelial cells then begin to migrate in response to angiogenic stimuli, forming offshoots of the blood vessels, and continue to proliferate until the off-shoots merge with each other to form the new vessels.

Normally, angiogenesis occurs in humans and animals in a very limited set of circumstances, such as embryonic development, wound healing, and formation of the corpus luteum, endometrium and placenta. However, aberrant angiogenesis is associated with a number of disorders, including, tumor metastasis. In fact, it is commonly believed that tumor growth is dependent upon angiogenic processes. Thus, the ability to increase or decrease angiogenesis has significant implications for clinical situations, such as wound healing (e.g., graft survival) or cancer therapy, respectively.

Several lines of direct evidence now suggest that angiogenesis is essential for the growth and persistence of solid tumors and their metastases (Folkman, 1989; Hon et al., 1991; Kim et al., 1993; Millauer et al., 1994). To stimulate angiogenesis, tumors up-regulate their production of a variety of angiogenic factors, including the fibroblast growth factors (FGF and DTCF) (Kandel et al., 1991) and vascular endothelial cell growth factor/vascular permeability factor (VEGF/VPP). However, many malignant tumors also generate inhibitors of angiogenesis, including angiostatin and thrombospondin (Chen et al., 1995; Good et al., 1990; O'Reilly et al., 1994). It is postulated that the angiogenic phenotype is the result of a net balance between these positive and negative regulators of neovascularization (Good et al., 1990; O'Reilly et al., 1994; Parangi et al., 1996; Rastineiad et al., 1989). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced, by interleukin-12 and/or interferon-gamma (Voest et al., 1995), gro-beta (Cao et al., 1995), and the 16 kDa N-terminal fragment of prolactin (Clapp et al., 1993).

2. Angiogenesis-Related Disease

The methods of the present invention are useful for treating endothelial cell-related diseases and disorders. A particularly important endothelial cell process is angiogenesis, the formation of blood vessels, as described above. Angiogenesis-related diseases may be treated using the methods described in present invention to inhibit endothelial cell proliferation. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, Rubeosis; Osler-Webber Syndrome; myocardial angiogenesis; plaque neovascularization; telangiectasia; hemophiliac joints; angiofibroma; and wound granulation. The endothelial cell proliferation inhibiting methods of the present invention are useful in the treatment of disease of excessive or abnormal stimulation of endothelial cells. These diseases include, but are not limited to, intestinal adhesions, atherosclerosis. scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

3. Cancer

Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Mougin et al., 1998). In fact, the occurrence of cancer is so high that over 500,000 deaths per year are attributed to cancer in the United States alone.

The maintenance of cell proliferation and cell death is at least partially regulated by proto-oncogenes. A proto-oncogene can encode proteins that induce cellular proliferation (e.g., sis, erbB, src, ras and myc), proteins that inhibit cellular proliferation (e.g., Rb, p16, p19, p21, p53, NF1 and WT1) or proteins that regulate programmed cell death (e.g., bc1-2) (Ochi et al., 1998; Johnson and Hamdy, 1998; Liebermann et al., 1998). However, genetic rearrangements or mutations to these proto-oncogenes, results in the conversion of a proto-oncogene into a potent cancer causing oncogene. Often, a single point mutation is enough to transform a proto-oncogene into an oncogene. For example, a point mutation in the p53 tumor suppressor protein results in the complete loss of wild-type p53 function (Vogelstein and Kinzler, 1992; Fulchi et al., 1998) and acquisition of “dominant” tumor promoting function.

Currently, there are few effective options for the treatment of many common cancer types. The course of treatment for a given individual depends on the diagnosis, the stage to which the disease has developed and factors such as age, sex and general health of the patient. The most conventional options of cancer treatment are surgery, radiation therapy and chemotherapy. Surgery plays a central role in the diagnosis and treatment of cancer. Typically, a surgical approach is required for biopsy and to remove cancerous growth. However, if the cancer has metastasized and is widespread, surgery is unlikely to result in a cure and an alternate approach must be taken. Radiation therapy, chemotherapy and immunotherapy are alternatives to surgical treatment of cancer (Mayer, 1998; Ohara, 1998; Ho et al., 1998). Radiation therapy involves a precise aiming of high energy radiation to destroy cancer cells and much like surgery, is mainly effective in the treatment of non-metastasized, localized cancer cells. Side effects of radiation therapy include skin irritation, difficulty swallowing, dry mouth, nausea, diarrhea, hair loss and loss of energy (Curran, 1998; Brizel, 1998).

Chemotherapy, the treatment of cancer with anti-cancer drugs, is another mode of cancer therapy. The effectiveness of a given anti-cancer drug therapy often is limited by the difficulty of achieving drug delivery throughout solid tumors (e1-Kareh and Secomb, 1997). Chemotherapeutic strategies are based on tumor tissue growth, wherein the anti-cancer drug is targeted to the rapidly dividing cancer cells. Most chemotherapy approaches include the combination of more than one anti-cancer drug, which has proven to increase the response rate of a wide variety of cancers (U.S. Pat. No. 5,824,348; U.S. Pat. No. 5,633,016 and U.S. Pat. No. 5,798,339, incorporated herein by reference). A major side effect of chemotherapy drugs is that they also affect normal tissue cells, with the cells most likely to be affected being those that divide rapidly (e.g., bone marrow, gastrointestinal tract, reproductive system and hair follicles). Other toxic side effects of chemotherapy drugs are sores in the mouth, difficulty swallowing, dry mouth, nausea, diarrhea, vomiting, fatigue, bleeding, hair loss and infection.

Immunotherapy, a rapidly evolving area in cancer research, is yet another option for the treatment of certain types of cancers. For example, the immune system identifies tumor cells as being foreign and thus are targeted for destruction by the immune system. Unfortunately, the -response typically is not sufficient to prevent most tumor growths. However, recently there has been a focus in the area of immunotherapy to develop methods that augment or supplement the natural defense mechanism of the immune system. Examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons), and (IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311).

4. Gene Therapy

Gene therapy is an emerging field in biomedical research with a focus on the treatment of disease by the introduction of therapeutic recombinant nucleic acids into somatic cells of patients. Various clinical trials using gene therapies have been initiated and include the treatment of various cancers, AIDS, cystic fibrosis, adenosine deaminase deficiency, cardiovascular disease, Gaucher's disease, rheumatoid arthritis, and others. Currently, adenovirus is the preferred vehicle for the delivery of gene therapy agents. Advantages in using adenovirus as a gene therapy agent are high transduction efficiency, infection of non-dividing cells, easy manipulation of its genome, and low probability of non-homologous recombination with the host genome.

5. Cytokines

IL-10 is a pleiotropic homodimeric cytokine produced by immune system cells, as well as some tumor cells (Howard et al., 1992; Ekmekcioglu et al., 1999). Its immunosuppressive function includes potent inhibition of proinflammatory cytokine synthesis, including that of IFNγ, TNFα, and IL-6 (De Waal Malefyt et al., 1991). The family of IL-10-like cytokines is encoded in a small 195 kb gene cluster on chromosome 1q32, and consists of a number of cellular proteins (IL-10, IL-19, L-20, MDA-7) with structural and sequence homology to EL-10 (Moore et al., 1990; Kotenko et al., 2000; Gallagher et al., 2000; Blumberg et al., 2001; Dumoutier et al., 2000; Knapp et al., 2000; Jiang et al., 1995a; Jiang et al., 1996). MDA-7 has been characterized as an IL-10 family member and is also known as IL-24.

Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3′UTR targeting mRNA for rapid degradation, MDA-7 has three AREs in its 3′UTR (Wang et al., 2002). Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine (Wang et al., 2002). The rat MDA-7 homolog C49a (Mob-5) is 78% homologous to the mda-7 gene and has been linked to wound healing (Soo et al. 1999; Zhang et al., 2000). Mob-5 was also shown to be a secreted protein and a putative cell surface receptor was identified on ras transformed cells (Zhang et al., 2000). Therefore, homologues of the mda-7 gene and the secreted MDA-7 protein are expressed and secreted in various species. However, no data has emerged to show MDA-7 has cytokine activity. Such activity has ramifications for the treatment of a wide variety of diseases and infections by promoting therapeutic immune responses or enhancing immunogenicity of an antigen.

### SUMMARY OF THE INVENTION

The present invention concerns methods of purifying MDA-7 and purified MDA-7, as well as methods and compositions involving MDA-7 protein, or nucleic acids encoding MDA-7, in therapeutic and preventative therapies, as well as in diagnostic assays.

A variety of embodiments for purifying MDA-7 are provided herein. The purified MDA-7 is human MDA-7 in some embodiments, and it may be full-length, or it may be truncated or fragments thereof. In other embodiments, the MDA-7 is from another species or source, such as another mammalian animal, including mice, rats, and monkeys. In some embodiments, the MDA-7 is glycosylated, whereas in other embodiments the MDA-7 is non-glycosylated. In some cases, the MDA-7 lacks its signal sequence, and in some cases, it has a heterologous signal sequence. All these MDA-7 polypeptides can be purified by the methods of the invention.

Purification methods described herein yield MDA-7 protein that has been purified to at least about 20%, 25%, 30%, 35%, 40%, 45%, 50%, 55%, 60%, 65%, 70%, 75%, 80%, 85%, 90%, 95% and up to about 100% homogeneity. Alternatively, MDA-7 protein is purified to at least or at most about 20%-95%, 30%-90%, 40%-80%, 50%-75%, 20%-50%, 50%-70%, 50%-90%, 70%-90% and ranges in between. The term “homogeneity” is used according to its plain and ordinary meaning to those skilled in the art of protein purification and is understood to refer to the level of purity of a particular protein. When used in the context of a percentage, it refers to the percent of MDA-7 protein as compared to the total amount of protein (by molecule). The term “homogeneous” is an adejective that refers to the level of homogeneity. The term “about” refers to the imprecision of determining protein amounts, and is intended to include at least one standard deviation of error for any particular assay or calibration for measuring protein concentration. For example, if protein concentration and homogeneity is measured by gel electrophoresis with coomassie gel staining, MDA-7 that has been purified to at least about 25% homogeneity means that the sample placed on the gel is at least 25% MDA-7, as compared to total protein concentration by molecule, plus or minus the standard deviation for a protein gel stained with coomassie dye.

Furthermore, it is contemplated that a composition of purified MDA-7 may have MDA-7 protein of which 20%, 25%, 30%, 35%, 40%, 45%, 50%, 55%, 60%, 65%, 70%, 75%, 80%, 85%, 90%, 95% and up to about 100% of it is active.

In the context of a pharmacologically or pharmaceutically acceptable solution or composition, the reference to MDA-7 purity in terms of homogeneity refers to how much of the solution or composition is MDA-7, as compared to the protein concentration of any contaminating proteins, where “contaminating proteins” refers to unwanted or undesired proteins. The distinction is meant to exclude protein concentration due to proteins intentionally placed in the solution or composition such as an immunogenic polypeptide against which the MDA-7 is being used to elicit an immune response.

In many embodiments of the invention, purified MDA-7 protein is active. The term “active” generally means the purified MDA-7 protein has some activity of MDA-7. This may be qualified by percentage, and in some embodiments, the purified MDA-7 protein is at least about 10%, 15%, 20%, 25%, 30%, 35%, 40%, 45%, 50%, 55%, 60%, 65%, 70%, 75%, 80%, 85%, 90%, 95% and up to about 100% as active as a control MDA-7 polypeptide, by any specified assay for MDA-7 activity. Such activity may be defined as including, but not limited to, any of the following: binding activity, functional activity (including, but not limited to, ability to induce apoptosis, to inhibit angiogenesis or induce anti-angiogenesis, to bind L-22, to activate STAT3, to modulate PKR, and to induce an immune response), ability to be post-translationally modified (glycosylated), ability to form proper tertiary structure, ability to be localized properly.

Methods of purifying MDA-7 protein involve a number of steps, which may be used separately or in combination with one another. MDA-7 can be purified from cells that express recombinant MDA-7 or non-recombinant MDA-7 (for example, from an endogenous MDA-7-expressing genomic gene). With cells expressing recombinant MDA-7, the host cell type will play a role in whether the MDA-7 is post-translationally modified. In specific embodiments, eukaryotic cells that allow the MDA-7 to be glycosylated are employed as host cells or cell sources for MDA-7 protein. Thus, the cell may be eukaryotic or prokaryotic, and it specifically is contemplated to be a mammalian, insect, bacterial, human, or fungal cell. In some embodiments, a cell extract or supernatant comprising MDA-7 protein is prepared and subjected to different purification steps, including chromatography.

The MDA-7 that is purified may be the secreted form of the protein, which corresponds to amino acids 49-206 of the full-length protein, identified as SEQ ID NO:2. Alternatively, the purified MDA-7 may be full length, or it may have one or more heterologous amino acid regions, such as a heterologous N-terminal region or a heterologous signal sequence. Moreover, the protein may be glycosylated. Glycosylation of MDA-7 may occur at different positions and to different extents. It is contemplated that the purified MDA-7 may not be uniformly glycosylated. Furthermore, it is contemplated that the MDA-7 may be purified as part of a complex, such as a dimer.

In specific embodiments, affinity chromatography is employed. In methods of the invention, affinity chromatography involves an anti-MDA-7 antibody. The use of monoclonal and polyclonal antibodies against MDA-7 are specifically contemplated. More than one monoclonal or polyclonal antibody may be employed, and the use of both polyclonal and monoclonal antibodies at the same time is contemplated. Affinity chromatography, in other embodiments, involves an affinity between MDA-7 and other molecules that are not protein-sequenced-based. Lectin, which binds glycosylated molecules, is employed in some aspects of the invention. The molecule that is the basis for the affinity with MDA-7 is complexed, in some cases, to a resin, which can be a non-reacting material, such as sepharose. A column of the affinity resin can be made, according to some embodiments of the invention. A cell extract or supernatant, can be passed over the resin as part of methods of the invention. In some embodiments, after affinity chromatography involving antibodies, the enriched or purified protein is exposed to Protein A, which binds any contaminating antibodies. The Protein A may be complexed or attached to a non-reacting structure such as a column or beads, so that it can be separated from the enriched or purified MDA-7 thereafter.

Other types of chromatography that may be employed are ion-exchange, particularly anion exchange. Furthermore, chromatography includes non-reacting purification processes, such as size exclusion chromatography. Size exclusion is contemplated to include, but not be limited to, gel electrophoresis, use of beads in a column for size exclusion, or any other type of non-reacting physical structure to distinguish molecule size. In some cases, at least one, two, three, four, five, or more different types of purification steps are employed. It is specifically contemplated that affinity chromatography be combined with anion exchange chromatography to purify MDA-7. In further embodiments, size exclusion chromatography is additionally employed. In one embodiment, a sample is subjected to affinity chromatography, size exclusion chromatography, then anion exchange chromatography. After each chromatographic procedure, protein carrier may be added to the sample, and/or the sample may be subjected to dialysis or size exclusion procedures. Depending on the type of purification process used, in some embodiments, the process is chosen to include or exclude polypeptides that bind MDA-7, such as the IL-22 or IL-20 receptors or PKR. Thus, it is specifically contemplated that the purification methods of the invention can be used to purify MDA-7 monomers, MDA-7 complexes-with or without glycosylation, and proteins that directly or indirectly bind MDA-7 (monomers or as a complex).

In specific embodiments, a protein carrier is added before, during or after chromatography is performed. The protein carrier may be added to a cell extract or supernatant prior to any chromatography or other enrichment step. In some cases, the carrier is added after MDA-7 is eluted from a column to stabilize it. In certain embodiments, the protein carrier is albumin. Albumin may be from one of a variety of sources, including humans. In some embodiments, the albumin is BSA. Furthermore, the protein carrier may be removed during subsequent steps of a purification process.

During chromatographic procedures, including anion exchange and affinity chromatography, salt gradients may be employed. Salt solutions may be employed, in some embodiments of the invention, at concentrations of 0.05, 0.1, 0.15, 0.20, 0.25, 0.30. 0.35, 0.40, 0.45, 0.50, 0.55, 0.60, 0.65, 0.70, 0.75, 0.80, 0.85, 0.90, 0.95, 1.00, 1.05, 1.10, 1.15, 1.20. 1.25 M or more and in increments of up to about 0.005, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.100, 0.200, 0.300, 0.400, 0.500 M or more between fractions. In some embodiments, anion exchange chromatography involves a step gradient of salt up to a concentration of 1.0 M. In further embodiments, the MDA-7 protein is eluted from a column or other physical structure in a solution with a salt concentration of about 0.9 M to 1.0 M. The salt used is NaCl is specific embodiments of the invention.

During chromatography procedures, there may be one or more washing steps. In some cases, a resin is washed after it is contacted with a cell extract or supernatant that has MDA-7 protein. Wash solution may comprise a buffer and have a salt concentration of at most about 0.05, 0.1, 0.15, 0.20, 0.25, 0.30. 0.35, 0.40, 0.45, 0.50, 0.55, 0.60, 0.65, 0.70, 0.75, 0.80, 0.85, 0.90, 0.95, 1.00 M or lower. After the wash steps, an elution step may follow. In some embodiments, a solution having 1 M salt or more and a pH below 5.0 is employed. The elution solution may have a pH of at most about 5.0, 4.5, 4.0, 3.5, 3.0 or below.

After elution, a neutralization step can follow, according to some embodiments of the invention. Neutralization involves, in specific embodiments, a buffer.

The invention includes compositions that contain MDA-7 protein that has been purified according to any method of the invention. Purified MDA-7 protein, as described above, is considered part of the invention.

Also included as part of the invention is the use of purified MDA-7. In some embodiments, there are methods for inhibiting angiogenesis in a patient comprising administering to the patient an effective amount of a pharmaceutically acceptable composition comprising purified MDA-7 protein, wherein the protein is active and at least about 80% homogeneous.

In other embodiments, there are methods for treating a cancer patient comprising administering to the patient an effective amount of a pharmaceutically acceptable composition comprising purified MDA-7 protein, wherein the protein is active and at least about 80% homogeneous. In additional embodiments, methods also include subjecting the patient to radiotherapy or chemotherapy. It is specifically contemplated that cancer patients who have cancers involving epithelial cancer cells or who have melanomas can benefit from methods of the invention. Endothelial cells express a receptor to which MDA-7 binds. Combining radiotherapy and MDA-7 administration results in apoptosis of tumor-associated endothelium. Thus the treatment of human tumors with MDA-7 is not limited to those tumors whose cells express a MDA-7 receptor. Furthermore, patients with leukemias or lymphomas whose cancer cells express a receptor for MDA-7 can also benefit from MDA-7 administration.

Moreover, the present invention concerns methods for inducing an immune response against an immunogenic molecule in a patient comprising administering to the patient the immunogenic molecule and a pharmaceutically acceptable composition comprising purified MDA-7 protein, wherein the protein is active and at least about 80% homogeneous. The term “homogeneous” refers to the extent to which MDA-7 protein has been purified to homogeneity. As discussed above, the composition may contain other proteins that are desired and not considered contaminants, such that they do not affect any reference to the extent of MDA-7 purity. In further embodiments of the invention, the immunogenic molecule against which an immune response in the patient is desired is a viral, microbial, fungal, or tumor antigen. In additional embodiments, interferon is administered to the patient. The interferon may be IFN-α, IFN-β, IFN-γ, or the lambda IFNs. In further embodiments, a cytokine or other immune stimulating molecule is administered to the patient.

Another method of the invention concerns the use of MDA-7 protein to induce anti-angiogenesis of a tumor. Tumors become vascularized and angiogenesis is induced around the tumor. The present invention uses MDA-7 polypeptide to inhibit or reverse that process by inducing anti-angiogenesis. The phrase “inducing anti-angiogenesis” refers to a reversal or inhibition of vascularization or to inhibition of angiogenesis that has already begun. In some embodiments, a patient with a tumor is administered an effective amount of an MDA-7 polypeptide to bind the IL-22 receptor on IL-22-receptor positive cells and induce anti-angiogenesis of the tumor. IL-22 receptor-positive cells are cells that express IL-22 receptor, which binds MDA-7, on their surface. Thus, in some embodiments, the IL-22 receptor-positive cells of the patient are given an effective amount of MDA-7. In further embodiments, the IL-22 receptor-positive cells are endothelial cells. Therefore, it is contemplated that endothelial cells in the patient may be given MDA-7 polypeptides. Furthermore, these cells do not need to be adjacent (“abutting” or “next to”) to the tumor or to tumor cells. It is contemplated that they may be remote (not adjacent) with respect to the tumor. Moreover, in some embodiments, the MDA-7 polypeptide is the secreted form MDA-7 and is glycosylated.

The present invention also includes methods and compositions related to the multiple administration, temporally and/or spatially, of MDA-7 to a cancer patient, including a patient with a tumor. Therefore, in some embodiments of the invention, there are methods of treating a patient with a tumor comprising injecting into a first site in the tumor a pharmaceutically acceptable composition comprising either i) an MDA-7 polypeptide or ii) an adenovirus vector comprising a nucleic acid, under the control of a promoter, encoding MDA-7; and injecting the composition into a second site in the tumor. It is contemplated that the patient may be given a composition comprising MDA-7 protein or a nucleic acid encoding MDA-7 at least, at most, or the following number: 1, 2, 3, 4, 5, 6, 7, 8, 9, 10, 11, 12, 13, 14, 15, 16, 17, 18, 19, 20, or more times. Thus, there may be, be at least, or be at most, 1, 2, 3, 4, 5, 6, 7, 8, 9, 10, 11, 12, 13, 14, 15, 16, 17, 18, 19, 20 injection sites within the tumor at which point an MDA-7 composition (referring to a composition comprising MDA-7 protein or a nucleic acid encoding MDA-7 protein) may be injected. An “injection site” refers to the point at which a needle or other puncturing device makes contact with the patient. The sites of injection may be, be at least, or be at most, within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 200 millimeters or more of each other, either along a surface of a tumor or along any plane of the tumor. It is specifically contemplated that when more than one injection is given, that two or more or all of the injections may be evenly spaced with respect to one another.

It is contemplated that the MDA-7 composition may also be injected outside the tumor, such as in the periphery. In some embodiments, the composition is injected within 24, 20, 16, 12, 8, 4, or 2 millimeters of the tumor.

In some embodiments of the invention, an injection is given at one time, and a subsequent injection is given as soon as the first one has been done. Alternatively, some time may elapse between injection. Therefore, it is contemplated that a subsequent injection may be given within, be given within at least, or be given within at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes or 1, 2, 3, 4, 5, 6, 7, 8, 9, 10, 11, 2, 13, 14, 15, 16, 17, 18, 19, 20, 21, 22, 23, 24 hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days or 1, 2, 3, 4, 5 weeks, or 1, 2, 3, 4, 5, 6, 7, 8, 9, 10, 11, 12 or more months of the previous injection.

Furthermore, the invention includes combination therapy strategies, including the use of one or more chemotherapeutics, radiotherapy, gene therapy, and/or immunotherapy.

It will be understood that purified MDA-7 may be purified from eukaryotic cells or prokaryotic cells, unless otherwise specified. In some cases, the MDA-7 is purified from a prokaryotic cell transfected with an MDA-7 encoding nucleic acid. In those circumstances, the MDA-7 will not be glycosylated but can still be utilized in some methods of the invention. In other embodiments, it is specifically contemplated that the MDA-7 is purified from a eukaryotic, and in some cases, a mammalian cell. In particular embodiments, the MDA-7 is purified from a mouse, rat, monkey, hamster, or human cell. The MDA-7 may be endogenously produced or exogenously produced in those cells.

Other methods of the invention include using purified MDA-7 as a treatment for a hyperproliferative disease, particularly cancer. Thus, in some embodiments of the invention, there are methods of treating cancer in a patient involving administering to the patient an effective amount of a pharmaceutically acceptable composition comprising purified MDA-7 protein that has been purified to a certain percentage homogeneity and is active. The percentage of homogeneity that can be used as part of the method include any of the percentages described herein.

In further embodiments of the invention, the patient is also exposed to radiotherapy. The invention specifically includes methods involving the radiosensitization of a cell. The term “radiosensitization” refers to rendering a cell more sensitive to radiation. Thus, radiosensitization of a cell prior to radiation treatment increases its susceptibility to radiation than a cell that has not been radiosensitized prior to radiation treatment. Thus, in some embodiments of the invention there are methods for radiosensitizing a cell using MDA-7.

Radiotherapy, which is a well known cancer treatment, can be given to the patient before or after administration of purified MDA-7 protein. It is contemplated that the patient may be exposed to at least one course of radiotherapy within, within at least, or within at most 5, 10, 15, 20, 25, 30, 35, 40, 45, 50, 55 minutes or 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 86, 84, 90, 96, 102, 108, 114, 120, 126, 130, 136, 142 hours, or 1, 2, 3, 4, 5, 6,7 days, or 1, 2, 3, 4, 5, 6, 7, 8, 9, 10, 11, 12 weeks or more, or a combination thereof, of the time that the patient is administered a dose of purified MDA-7 protein. In specific embodiments, the patient is exposed to radiotherapy within 96 hours of receiving a dose of purified MDA-7 protein therapy. It is contemplated that multiple administrations or courses of therapy with respect to radiotherapy, MDA-7 protein, or both may be given to the patient. It is contemplated that any cancer or cancer cell discussed herein may be treated with purified MDA-7 protein and/or radiotherapy. In specific embodiments, the cancer being treated is of pancreatic origin or the cancer is a melanoma. It is specifically contemplated that purified MDA-7 protein may be given to noncancerous cells that are adjacent or located near cancer or tumor cells.

In particular embodiments, methods include radiosensitizing a cancer cell comprising administering to the cell an effective amount of an adenovirus vector comprising a nucleic acid encoding MDA-7, wherein the nucleic acid is under the control of a promoter operable in the cell. Other vectors may be used as well. Furthermore, as with any other method of the invention, purified MDA-7 may be administered instead of an expression construct that encodes an MDA-7 polypeptide, or vice versa. It is specifically contemplated that the cancer cell may be an epithelial cell, or any other cancer cell described herein.

The invention further concerns methods involving an MDA-7-encoding polynucleotide, expression construct or vector that is complexed with protamine. Protamine is a charged molecule that can be in a composition with an MDA-7 nucleic acid molecule or it can be complexed with it.

Other methods of the invention include methods for treating a cancer cell in a subject or a cancer patient by administering both tamoxifen and either purified MDA-7 protein or an adenovirus vector comprising a nucleic acid encoding MDA-7 under the control of a promoter. Tamoxifen may be given at the same time as the MDA-7 protein or adenovirus vector is administered, or it may be given before or after then. It is contemplated that tamoxifen may be given to the patient or subject within, within at least, or within at most 5, 10, 15, 20, 25, 30, 35, 40, 45, 50, 55 minutes or 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 78, 86, 84, 90, 96, 102, 108, 114, 120, 126, 130, 136, 142 hours, or 1, 2, 3, 4, 5, 6,7 days, or 1, 2, 3, 4, 5, 6, 7, 8, 9, 10, 11, 12 weeks or more, or a combination thereof, of the time that the patient is administered a dose of purified MDA-7 protein or the adenovirus vector.

Additional embodiments of the invention include methods for inducing STAT3 activation in a cell comprising administering to the cell an effective amount of purified MDA-7 protein that has been purified to a certain percentage homogeneity and is active. The percentage of homogeneity that can be used as part of the method include any of the percentages described herein. The phrase “STAT3 activation” refers to provoking the activity of a STAT3 polypeptide. It is contemplated that STAT3 activation can be detected using methods described in the Examples section herein.

Other methods of the invention concern the use of MDA-7 to inhibit smooth muscle cells. Therefore, in some embodiments, there are methods of inhibiting a smooth muscle cell comprising administering to the cell an effective amount of a composition comprising either a purified MDA-7 protein or an adenovirus vector comprising a nucleic acid encoding MDA-7 under the control of a promoter. Inhibiting a smooth muscle cell includes inducing the cell to undergo apoptosis or inhibiting its migration.

The present invention further concerns methods of treating cancer or a cancer cell in a patient comprising administering an NF-κB inhibitor and a composition comprising either a purified MDA-7 protein or an adenovirus vector comprising a nucleic acid encoding MDA-7 under the control of a promoter. An NF-κB inhibitor refers to a substance that inhibits the expression or activity of NF-κB. In some embodiments, the NF-κB inhibitor is Sulindac. In other embodiments the NF-κB inhibitor is I-κB protein or a vector comprising a nucleic acid encoding I-κB. It is specifically contemplated that the patient may be administered a single vector encoding both MDA-7 and the NF-κB inhibitor or they may be provided for by separate vectors. Similarly, the patient may be administered a single composition comprising one or more vectors and/or one or more proteins.

Methods of the invention also include treatment of cancer involving the charged molecule protamine. In some embodiments, the invention concerns methods of treating cancer comprising administering to a cancer patient an effective amount of a viral composition comprising: (a) a protamine molecule; and (b) an expression construct comprising a nucleic acid encoding a human MDA-7 polypeptide under the control of a promoter. It is contemplated that the protamine molecule is complexed to the expression construct in some embodiments of the invention. Viral compositions may comprises a ratio of about 10⁸, 10⁹, 10¹⁰, 10¹¹, 10¹², 10¹³, 10¹⁴, 10¹⁵ or more viral particles to about 10, 20, 30, 40, 50, 60, 70, 80, 90, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000 or more μg protamine. In specific embodiments, the viral composition comprises a ratio of about 10¹⁰ or 10¹¹ viral particles to about 100 μg protamine, about 200 μg protamine, or about 300 μg protamine.

It is contemplated that the MDA-7 peptide or polypeptide employed in methods and compositions of the invention may comprise at least 10, 20, 30, 40, 50, 60, 70, 80, 90, 100, 110, 120, 130, 140, 150, 156, 157, 160, 170, 180, 190, 200 or 206 contiguous amino acids of SEQ ID NO:2 or comprise all of SEQ ID NO:2. The recombinant MDA-7 polypeptide may be modified, or it may be truncated at either end. In some embodiments of the invention, the MDA-7 polypeptide comprises amino acids 49 to 206, 75 to 206, or 100 to 206 of SEQ ID NO:2. The secreted form of MDA-7 has amino acids 49 to 206 of SEQ ID NO:2, but the first 48 amino acids are absent, and it is specifically contemplated that the secreted form qualifies as “the MDA-7 polypeptide” and may be used in any composition or method of the invention. Alternatively, an MDA-7 amino acid sequence may include a heterologous amino acid sequence, such as a secretory signal. In some embodiments, the secretory signal is a positively charged N-terminal region that has a hydrophobic core. In other embodiments, the secretory signal targets MDA-7, or a truncation thereof, to the endoplasmic reticulum or mitochondria.

Expression constructs may be viral or nonviral vectors. Viral vectors that are considered part of the invention include, but are not limited to, adenovirus, adeno-associated virus, herpesvirus, retrovirus (including lentiviruses), polyoma virus, or vaccinia virus.

Cancer cells that may be treated by methods and compositions of the invention include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Compositions may be administered to a cell or a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, by inhalation (e.g., aerosol inhalation), by injection, by infusion, by continuous infusion, by localized perfusion bathing target cells directly, via a catheter, via a lavage, in a creme, or in a lipid composition.

Other aspects of the invention serve a diagnostic or prognostic purpose. The invention includes methods for evaluating progression of cancer in a subject diagnosed with cancer or suspected of having cancer comprising: (a) obtaining a sample from the subject; (b) measuring MDA-7 expression in the sample of hyperproliferative tissue; (c) measuring iNOS expression in the sample of hyperproliferative tissue; and (d) comparing the level of MDA-7 expression to the level of iNOS expression. As is shown in the Examples, iNOS expression is higher when MDA-7 expression levels are lower or absent compared to the expression level of iNOS when MDA-7 expression levels are raised or are present at levels generally seen in a normal cell, such as a non-melanoma skin cell. A relative ratio of the level of MDA-7 expression and the level of iNOS expression in a particular sample can be calculated as part of evaluating or diagnosing cancer. Expression of MDA-7 or iNOS can be determined based on protein or transcript levels, according to techniques well known to those of skill in the art. The ratio can be compared to standards or controls obtained from noncancerous cells.

Furthermore, the ratio or levels of MDA-7 versus iNOS can be used to evaluate response to a cancer treatment. A reduction in iNOS expression serves as a positive indicator of therapy. In some embodiments, therapy with MDA-7 is monitored. Therefore, there are methods for measuring response to treatment of cancer in a subject, comprising: (a) obtaining a sample from the subject before and after treatment with MDA-7; and (b) comparing levels of iNOS expression in the samples. As discussed above, treatment with MDA-7 can comprise administering to the subject an effective amount of either purified and active MDA-7 protein or an expression cassette comprising a nucleic acid sequence encoding a human MDA-7 polypeptide under the control of a promoter.

This method is contemplated for use with a variety of cancers, but in specific embodiments, it applies to melanoma cancer, including metastatic melanoma cancer. A patient may be suspected of having cancer based on a patient interview or medical history, preliminary test results, or other indications/factors suggesting the patient may have cancer or be at risk for cancer.

The present invention also pertain to methods of treating a patient with ovarian cancer. Certain embodiments of the present invention pertain to methods for treating a patient with ovarian cancer that involve administering to the patient an effective amount of a pharmaceutically acceptable composition that includes MDA-7 protein. For example, the MDA-7 protein may be active and substantially purified MDA-7 protein that is at least about 80% homogeneous. Other embodiments pertain to methods of treating a patient with an ovarian tumor that involve injecting into a first site in the tumor an effective amount of a pharmaceutically acceptable composition that includes either i) an MDA-7 polypeptide or ii) an adenovirus vector comprising a nucleic acid, under the control of a promoter, encoding MDA-7; and injecting the composition into a second site in the tumor. Methods pertaining to use of adenovirus vectors that are discussed throughout this specification. Whenever discussed, these methods apply to compositions of MDA-7 for use in the treatment of ovarian cancer.

Other embodiments of the present invention involve methods of treating a patient with a tumor that involve administering to the patient an effective amount of pharmaceutically acceptable composition that includes an agent that induces APC expression in the tumor. Any agent that induces APC expression in the tumor is contemplated by the present invention. For example, the agent can be a small molecule, a nucleic acid, or a proteinaceous composition. In certain embodiments, the agent is MDA-7, an MDA-7 polypeptide, or an expression construct that includes a nucleic acid encoding an MDA-7 polypeptide. Methods pertaining to the use of expression constructs is discussed throughout this specification. One of skill in the art would be familiar with use of expression constructs, and the range of methodologies that are available in this regard.

Any method of administration of the agent that induces APC expression is contemplated for use by the present invention. One of ordinary skill in the art would be familiar with the range of means available for delivery of the agent to a patient. For example, the agent can be administered intravenously, intratumorally, or orally. In some embodiments of the present invention, the composition is further defined as a composition that decreases β-catenin expression in the tumor. Methods to measure β-catenin expression in a tumor include any method known to those of skill in the art. Examples of these methods are discussed elsewhere in the specification.

In some embodiments of the present invention, the composition decreases β-catenin expression in a subject. For example, the decrease in β-catenin expression may be present within a tumor of a subject.

Other embodiments of the present invention involve methods of screening for anticancer compounds that involve: (1) contacting a candidate substance with a first cancer cell; (2) measuring APC expression in the first cancer cell; and (3) comparing APC expression in the first cancer cell with a second cancer cell not contacted with the candidate substance, wherein an increase in APC expression in the first cancer cell identifies the candidate substance as a candidate anticancer compound. These methods may or may not involve measuring β-catenin in the first and second cancer cells and determining whether β-catenin expression is decreased in the first cancer cell compared to the second cancer cell. The steps involving measurement of β-catenin expression may or may not be independent of the steps involving measurement of APC. Any candidate substance is contemplated by the present invention, and one of ordinary skill in the art would be familiar with the wide range of types of candidate substances that are available. For example, the candidate substance may be a small molecule, a nucleic acid, or a proteinaceous composition, and may include a β-catenin ribozyme, siRNA, or an antisense molecule.

The present invention also includes polypeptides comprising amino acids 175 to 206 of SEQ ID NO:2 and an endoplasmic reticulum targeting sequence. The amino acid sequence of SEQ ID NO:2 is -provided elsewhere in this specification. Specifically contemplated are polypeptides that include amino acid residue 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, and 206 of SEQ ID NO:2. It is contemplated that amino acids 175 to 206 of SEQ ID NO:2 are contiguous amino acid residues.

In other embodiments of the present invention, the polypeptide includes amino acids 150 to 206 of SEQ ID NO:2 and an endoplasmic reticulum (ER) targeting sequence. In further embodiments, the polypeptide includes amino acids 100 to 206 of SEQ ID NO:2 and an endoplasmic reticulum targeting sequence. In still further embodiments, the polypeptide includes amino acids 49 to 206 of SEQ ID NO:2 and an endoplasmic reticulum targeting sequence. In each of these embodiments, it is contemplated that the amino acids of SEQ ID NO:2 are contiguous amino acid residues. The ER targeting sequence is operably linked to the N-terminal portion of a truncated MDA-7 polypeptide in some embodiments of the invention. ER-targeting sequences described herein and those known to those of skill in the art are contemplated as aspects of the invention in the context of MDA-7 polypeptides.

The embodiments of the present invention may or may not include an ER retention signal. One of ordinary skill in the art would be familiar with ER targeting sequences and endoplasmic reticulum retention signals.

The present invention also contemplates expression cassette that include a nucleic acids encoding MDA-7 sequences discussed above and an ER targeting sequence. Expression cassettes are discussed throughout this specification, and sections of the specification that discuss expression cassettes also apply to expression cassettes that include a nucleic acid encoding amino acids of SEQ ID NO:2.

In addition, the present invention concerns methods for treating a patient with cancer that include administering to the patient an effective amount of a pharmaceutically acceptable composition comprising MDA-7 protein and one or more interleukins selected from the group consisting of IL-2, IL-7, and IL-15. Furthermore, it is contemplated that MDA-7 can be used in combination with other interleukins in any of the methods of the present invention.

Certain interleukins have been demonstrated to have cytokine activity. Examples of such interleukins include IL-19, IL-20, IL-22, and IL-26. It is contemplated that these interleukins with cytokine activity can replace MDA-7 in the methods of the present invention that pertain to methods of inhibiting angiogenesis and methods of stimulating an immune response.

The present invention also concerns methods for inhibiting or preventing local invasiveness and/or metastasis of cancer in a patient, involving administering to the patient an effective amount of a pharmaceutically acceptable composition comprising MDA-7 protein, wherein the MDA-7 inhibits or prevents the local invasiveness and/or metastasis of the cancer. Any method of administration known to those of ordinary skill in the art is contemplated by the present invention. One of ordinary skill in the art would be able to determine whether local invasiveness and/or metastasis of the cancer has been prevented or inhibited.

The present invention contemplates methods for inhibiting or preventing local invasiveness and/or metastasis of any type of primary cancer. For example, the primary cancer may be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments of the present invention, the primary cancer is lung cancer. For example, the lung cancer may be non-small cell lung carcinoma.

Moreover, the present invention can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like. The progression to cancer or to a more severe form of cancer may be halted, disrupted, or delayed by methods of the invention involving MDA-7 polypeptides and expression constructs containing an MDA-7 encoding nucleic acid, as discussed herein.

Any method to prepare or formulate MDA-7 is contemplated for inclusion in the methods of the present invention. In certain embodiments, the MDA-7 is purified according to any of the methods discussed above in this summary of the invention. For example, the MDA-7 may be purified from a cell to at least 20% homogeneity by the method discussed above, wherein a cell extract or supernatant that includes MDA-7 protein is subjected to affinity chromatography, wherein the MDA-7 is purified to at least 20% homogeneity and is active.

Other aspects of the present invention involve methods for inhibiting or preventing local invasiveness and/or metastasis of cancer in a patient, involving administering to the patient an effective amount of a pharmaceutically acceptable composition that includes a polynucleotide encoding an MDA-7 polypeptide, wherein the MDA-7 polypeptide inhibits or prevents the local invasiveness and/or metastasis of the cancer. In certain embodiments, the polynucleotide encoding an MDA-7 polypeptide is included in an expression construct. For example, the expression construct may include an adenovirus vector comprising a nucleic acid, under the control of a promoter, encoding the MDA-7 polypeptide.

The present invention also encompasses other methods such as a method for treating microscopic residual cancer including the steps of identifying a patient having a resectable tumor, resecting that tumor, and contacting the tumor bed with an MDA-7 protein or an expression vector that involves a promoter functional in eukaryotic cells and a polynucleotide encoding an MDA-7 polypeptide, wherein the polynucleotide is under the transcriptional control of a promoter.

Further methods of the present invention are methods for treating a subject having a tumor including the steps of surgically revealing the tumor and contacting the tumor with an MDA-7 polypeptide or an expression vector containing a promoter functional in eukaryotic cells and a polynucleotide encoding an MDA-7 polypeptide, wherein the polynucleotide is under the transcriptional control of a promoter. Alternatively, after administration of MDA-7 polypeptide or an MDA-7 encoding nucleic acid, all or part of a tumor may be resected. This form of adjunct therapy is specifically contemplated as part of the invention.

The present invention contains yet other methods for treating a subject having a tumor including the step of perfusing the tumor, over an extended period of time, with an MDA-7 polypeptide or an expression vector comprising a promoter functional in eukaryotic cells and a polynucleotide encoding an MDA-7 polypeptide, wherein the polynucleotide is under the transcriptional control of a promoter.

The use of MDA-7 as an adjunct therapy is also contemplated as part of the invention. This adjunct therapy may be used in combination with one or more other cancer therapies, including, but not limited to, surgery, chemotherapy, radiotherapy, immunotherapy, or gene therapy. Examples include surgery and chemotherapy; surgery and radiation; surgery and immunotherapy; radiation and chemotherapy; radiation and immunotherapy; chemotherapy and immunotherapy; surgery, radiation, and chemotherapy; surgery, chemotherapy and immunotherapy, etc. Furthermore, it is contemplated that the chemotherapy treatment may involve more than one chemotherapeutic. In some embodiments of the invention, it is specifically contemplated that MDA-7 (polypeptide or encoding nucleic acid) may be used with taxotere, Herceptin, and Ad-mda7. This can be used quite effectively to treat breast cancer, for instance. As discussed above, Ad-mda7 is also contemplated for use with tamoxifen.

There is also provided a method of treating a subject with recurrent cancer comprising (a) selecting a patient based on (i) prior treatment of cancer with surgery, or a radio- or chemotherapy or immunotherapy; and (ii) recurrence of cancer subsequent to the treatment, and (b) administering to the patient an MDA-7 polypeptide or an expression construct comprising a nucleic acid segment encoding MDA-7, the segment under the control of a promoter active in a cancer cell of the patient, the expression construct expressing MDA-7 in the cancer cell. A subsequent step (c) that follows step (b) of administering to the patient a second radio- or chemotherapy or immunotherapy session, whereby MDA-7 sensitizes the cancer cell to said second radio- or chemotherapy or immunotherapy, thereby treating the cancer may also be provided.

The first cancer therapy and the second second cancer therapy may be the same or different. The patient may be a non-human animal, or a human patient. The first and/or second radio- or chemotherapy may be chemotherapy, such as busulfan, chlorambucil, cisplatinum, cyclophosphamide, dacarbazine, ifosfamide, mechlorethamine, melphalan, 5-FU, Ara-C, fludarabine, gemcitabine, methotrexate, doxorubicin, bleomycin, dactinomycin, daunorubicin, idarubicin, mitomycin C, docetaxel, taxol, etoposide, paclitaxel, vinblastine, vincristine, vinorelbine, camptothecin, carmustine, or lomustine. The first and/or second radio- or chemotherapy may be radiotherapy, such as x-rays, gamma rays, or microwaves. The first and/or second radio- or chemotherapy may be characterized as a DNA damaging therapy. Immunotherapy may involve treatment with a monoclonal antibody that targets a particular protein, such as herceptin (trastuzumab), rituxan (rituximab), Erbitux (cetuximab), ABX-EGF, bexxar, zevalin, oncolym, Mylotarg, LymphoCide, or Alemtuzumab.

The treated cancer may be brain cancer, head & neck cancer, esophageal cancer, tracheal cancer, lung cancer, liver cancer stomach cancer, colon cancer, pancreatic cancer, breast cancer, cervical cancer, uterine cancer, bladder cancer, prostate cancer, testicular cancer, skin cancer, rectal cancer lymphoma or leukemia.

The expression construct may be a viral expression construct, such as a retroviral construct, a herpesviral construct, an adenoviral construct, an adeno-associated viral construct, or a vaccinia viral construct. The viral expression construct may be a replication-competent virus or adenovirus, or a replication-defective virus or adenovirus. Alternatively, the expression construct may be a non-viral expression construct, such as one that is comprised within a lipid vehicle. The promoter may be CMV IE, RSV LTR, β-actin, Ad-E1, Ad-E2 or Ad-MLP. Other gene therapy vectors and promoters known to those skilled in the art may also be utilized.

The time period between steps (b) and (c) may be about 24 hours, about 2 days, about 3 days, about 7 days, about 14 days, about 1 month, about 2 months, about 3 months, or about 6 months. Recurrence may be recurrence at a primary tumor site or a metastatic site. The subject may have had surgical resection prior to step (b), and/or the method may further comprise surgical resection following step (c). Administering in step (b) may be intratumoral, to a tumor vasculature, local to a tumor, regional to a tumor, or systemic. Administering in step (c) may be intratumoral, to a tumor vasculature, local to a tumor, regional to a tumor, or systemic.

The present invention further includes methods and compositions for eliciting an immune response against MDA-7. Therefore, in some embodiments of the invention, all or part of an MDA-7 polypeptide or a nucleic acid encoding the same is provided to a subject as a vaccine. This vaccine may be used to prevent or treat any condition or disease involving MDA-7, including cancer.

Also included in methods and compositions of the present invention is the use of antibodies against MDA-7, particularly antibodies that neutralize MDA-7 activity, which includes those that inhibit the binding to its receptors (such as IL-20R and IL-22R). Monoclonal and polyclonal antibodies, as well as humanized version thereof, can be used to treat inflammatory diseases, autoimmune diseases and conditions, including psoriasis, inflammatory bowel disease (IBD), rheumatoid arthritis, and lupus. Methods of the invention include methods of treatment that are accomplished by administering to a patient an effective amount of an MDA-7 antibody (also referred to as anti-MDA-7 antibody) whereby a therapeutic benefit is conferred. The therapeutic benefit includes, but is not limited to, reduction of number of symptoms or reduction in severity of symptoms, induction of remission, reduction of inflammation or characteristics of inflammation, reduction in pain.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1A-D. sMDA-7 inhibits endothelial cell differentiation but not proliferation in vitro. HUVEC and HMVEC were serum starved for 24 h and plated in 2-well chamber slides containing 1 ng/ml bFGF and the indicated concentrations of sNDA-7 (A). Cells treated with PBS and angiostatin served as negative and positive controls, respectively. Proliferation was measured 72 h later as indicated in Example 1. (B), Lung tumor cells (H1299 and A549) plated in 2-well chamber slides were treated with the indicated concentrations of sDMA-7. Cells treated with PBS and Ad-mda7 served as negative and positive controls, respectively. Proliferation was measured 72 hours later as indicated in Example 1. HUVEC plated in Matrigel-coated 96-well plates were treated with PBS, sMDA-7 (50 ng/ml) or a preparation immunodepleted of sMDA-7 and observed for tube formation (C). All treatments were assayed in duplicate. sMDA-7 completely abrogated endothelial tube formation, while immunodepletion of sMDA-7 protein resulted in restoration of tube formation similar to those seen in PBS-treated control cells. Magnification, ×10. Semi-quantiative analysis of the number of endothelial tubes in sMDA-7/IL-24 treated HUVEC and HMVEC showed significant (P=0.001) inhibition of tube formation by sMDA-7 in both cell types (C). Bars, SE.

FIG. 2. sMDA-7 is more potent than endostatin in inhibiting endothelial cell differentiation in vitro. HUVEC were seeded in Matrigel-coated 96-well plates containing 1 ng/ml bFGF and the indicated equimolar concentrations of sMDA-7 and endostatin. Twenty-fours hours after treatment, plates were observed for tube formation under a microscope and the number of tubes counted. Cells treated with PBS served as negative control. All treatments were assayed in duplicate, and experiments were repeated 5-6 times. sMDA-7 but not endostatin significantly (P=0.001) inhibited endothelial tube formation in a dose-dependent manner with complete abrogation at concentrations higher than 10 ng/ml. Inhibition of tube formation by endostatin was observed only at the highest concentration (300 ng/ml). Values are presented as the average mean of 3 experiments. Bars, SE.

FIG. 3. sMDA-7 inhibits endothelial cell migration. HUVEC were starved overnight in 0.5% FBS and seeded in the upper chamber of 24-well Transwell insert and placed in a 24-well plate containing 100 ng/ml of VEGF and 10 ng/ml of sMDA-7. The number of cells that migrated to the lower chamber were counted under high-power magnification. SMDA-7 significantly (P=0.001) inhibited VEGF-induced HUVEC migration in a 24 h period compared to cells treated with VEGF alone. PBS treated cells served as negative control.

FIG. 4A-D. MDA-7 inhibitory activity is not due to IFN-γ and IP-10 production by HUVEC. HUVEC seeded in six-well plates were treated with sMDA-7/IL-24 (10 ng/ml). Cell culture supernatant was collected at the indicated time points and analyzed for IFN-γ (A) and IP-10 (B) by ELISA. Supernatant from HUVEC treated with IFN-γ or Ad-mda 7 served as positive controls for IP-10 and IFN-γ ELISA respectively. Supernatant from PBS treated cells served as negative control. All treatments were assayed in quadruplicate. (C), HUVEC seeded in Matrigel coated 96-well plates were treated with equimolar concentrations of sMDA-7, IFN-γ or IP-10 and analyzed for tube formation. The inhibitory activity was determined by counting the number of tubes. sMDA7 significantly (P=0.01) inhibited tube formation at low concentrations compared to IFN-γ or IP-10. Inhibitory activity for IFN-γ or IP-10 was observed only at high concentrations. (C). The inhibitory activity was determined by counting the number of tubes. sMDA-7 significantly (P=0.01) inhibited tube formation at low concentrations compared to IFN-γ or IP-10. Inhibitory activity for IFN-γ or P-10 was observed only at high concentrations. D, HUVEC pretreated with anti-IP-10 or anti-IFN-γ neutralizing antibody were seeded in Matrigen coated 96-well plates and treated with sMDA-7 and analyzed for tube formation. sMDA-7 significantly (P=0.001) inhibited tube formation. Bars, SE.

FIG. 5. sMDA-7 inhibits endothelial cell differentiation via the IL-22R1. HUVEC were either untreated or treated with two different concentrations of L-22R1 blocking antibody for 24 h prior to seeding in Matrigel-coated 96-well plates that contained PBS, or the indicated concentrations of sMDA-7, endostatin, or IP-10. The next day the cells were examined for tube formation under bright-field microscopy and quantitated. Tube formation was inhibited in sMDA-7/IL-24-treated HUVEC but not in PBS-treated control cells (A). However, in the presence of IL-22R1 blocking antibody the inhibitory effect of sMDA-7 on HUVEC tube formation was abrogated in a dose-dependent manner. Endostatin or IP-10 inhibited tube formation of HUVEC pretreated with IL-22R1 antibody (B). Bars, SE.

FIG. 6A-E. In vivo studies of angiogenesis and tumor growth. sMDA-7 (12.5 ng) encapsulated in Matrigel containing 60 ng of bFGF was implanted subcutaneously into athymic nude mice. Matrigel that contained bFGF only served as positive control, and Matrigel that contained PBS served as negative control. After 10 days, Matrigel was harvested and analyzed for hemoglobin levels as described in Example 1. A significant (P=0.0001) reduction in hemoglobin level was observed in Matrigel containing sMDA-7/IL-24 compared with controls (A). Measurement of subcutaneous tumors grown by implanting an equal mixture (1:1) of A549 tumor cells with parental 293 cells or 293-mda-7 cells in nude mice (B). Significant growth inhibition was observed in tumors that contained 293-mda-7 cells compared to tumors that contained parental 293 cells (P=0.001) (B). Each time point represents the mean tumor volume for each group. Bars represent standard error. MDA-7 protein expression was detected by western blot analysis in tumor tissues containing 293-mda-7 cells compared with tumors containing parental 293 cells. At the end of the experiment, tumors were harvested and analyzed. Hemoglobin level in tumor samples from animals that contained 293-mda-7 cells was lower than it was in tumor samples from animals that contained parental 293 cells (C). Subcutaneous tumors were established by injecting A549 tumors cells in the lower right flank (D). When the tumors were palpable, animals were implanted with Matrigel-encapsulated parental 293 cells or with Matrigel-encapsulated 293-mda-7 cells into the upper right flank of each mouse. Tumor growth was measured using calipers. Tumor growth was inhibited significantly more in tumors treated with 293-mda-7 cells than it was in tumors treated with 293 cells (P=0.001). Each time point represents the mean tumor volume for each group. Bars represent standard error. Semi-quantitative analysis of tumor tissues for CD31 staining showed significant reduction in microvessel density in tumors treated with 293-mda-7 compared to tumors treated with parental 293 cells (E). Bars, SE.

FIG. 7. Study design of Phase I dose-escalating clinical trial wherein mda-7 was administered via intratumoral injection to patients with advanced carcinoma using a non-replicating adenoviral construct (Ad-mda7). Study design demonstrates number of patients, viral dose, and biopsy time per cohort.

FIG. 8. Bar graph demonstrating number of DNA copies/μg DNA vs. hours after intratumoral injection. Within 24 hours of injection there is a dose-dependent increase in MDA-7 protein expression, which demonstrates a decrease by 96 hours

FIG. 9. Chart of patient results demonstrating that apoptosis by TUNEL staining was most intense in the center of the lesions. Sections in the periphery of the lesions demonstrated a heightened TUNEL reaction comparied with uninjected lesions.

FIG. 10. Graphic representation of kinetics of serum cytokine response to Ad-mda7, demonstrating % increase of serum cytokines vs. days post treatment. Results demonstrate a transient increase in serum cytokines following intratumoral injection of Ad-mda7.

FIG. 11. Serum cytokine response to intratumoral Ad-mda7 treatment per cohort. A majority of patients demonstrated transient increase in systemic cytokines (EL-6, IL-10, IFNγ, TNFα, GM-CSF).

FIG. 12. Level of increased CD8+ T cell frequency in patients who received intratumoral Ad-mda7. CD3+ CD8+ T cells were increased by 30±13% at day 15 following mda7 treatment.

FIG. 13. Increase in peripheral blood CD8+ cells following intratumoral Ad-mda7 injection in subjects.

FIG. 14. One step anion exchange purification of MDA-7. Each peak, (1, 2, 3, 4) from the anion exchange column contains MDA-7 detected by polyclonal anti-MDA-7 on western blot.

FIG. 15. Comparison of retention time to molecular weight. MDA-7 complex elutes between 85-95 kDa.

FIG. 16. MDA-7 overexpression inhibits cellular proliferation. Tumor cells (DU 145, LNCaP, and PC-3) and normal cells (PrEC) were treated with PBS, treated with Ad-luc or treated with Ad-mda7 and analyzed for MDA-7 expression or cell viability at various time points. Measurement of cellular proliferation of tumor and normal cells after treatment with PBS, Ad-luc and Ad-mda7. Values are represented as the mean of triplicates. Statistical significance was set as P=<0.05. Error bars denote standard error (SE).

FIG. 17. MDA-7 expression induces apoptosis in tumor cells but not in normal cells. Tumor cells (DU 145, LNCaP, and PC-3) and normal epithelial cells (PrEC) treated with PBS, Ad-luc or Ad-mda7 were harvested 72 h after treatment and analyzed for cells in sub-G0/G1 phase by flow cytometry. Twenty thousand events were captured for each treatment; and the data represented as histograms. Data are the average of triplicate values. Bars denote standard error (SE).

FIG. 18. Induction of G2 cell-cycle arrest by MDA-7. Tumor cells (DU 145, LNCaP, and PC-3) and normal cells (PrEC) were treated with PBS, Ad-luc, or Ad-mda7. Cells were harvested 72 h after treatment, and cell-cycle analysis was performed using flow cytometry. Twenty thousand events were captured for each treatment and the data shown as histograms. Y-axis represents the number of cells and X-axis represents the cell-cycle phase. Data are the average of duplicate experiments. Bars denote standard error (SE).

FIGS. 19A-D. Radiosensitization by Ad-mda7 determined on the basis of clonogenic survival assays. Vector concentrations used for Ad-mda7 and Ad-luc were 1000 vp/cell for the A549 cell line (A); 250 for the H1299 cell line (B) and 1500 for the CCD-16 (C) and MRC-9 (D) cell lines. Radiation was given 48 hours after transfection. Each data point represents the average of three independent experiments. Symbols represent mock infection, (closed diamond); Ad-mda7, (closed square); and Ad-luc, (closed triangle). Bar: SE.

FIGS. 20A-D. Apoptosis assessed by TUNEL assay for A549 (A), H1299 (B), CCD-16 (C), and MRC-9 (D) cells. Cells were irradiated 48 hours following transfection and harvested 2 days after irradiation or 4 days after transfection. Vector concentrations used were identical to those used for FIG. 19. Each data point represents the average of two independent experiments. Bar: SE.

FIG. 21. Cell cycle analysis of A549 and H1299 treated with either Ad-mda7 or Nocodazole (200 ng/ml). The dose and exposure time of Nocodazole to accumulate the same proportion of cells in G2/M phase as was present 48 hours after Ad-mda 7 transfection was determined in preliminary experiments. Data shown are representative of two independent experiments.

FIG. 22. Clonogenic survival assays to determine radiosensitization by G2/M arrest induced by Nocodazole (200 ng/ml). Radiation was given after 4 hours of Nocodazole exposure for the A549 cell line, and after 3.5 hours of Nocodazole exposure for the H1299 cell line. Symbols represent radiation alone, (closed diamond); Nocodazole, (open square). Bar: SE.

FIG. 23. Clonogenic survival assays to determine radiosensitization in A549 and H1299 cells treated with either curcumin or curcumin plus Ad-mda7. Radiation was given 2 days after transfection. Curcumin was added 1 day after transfection. The vector concentrations used were identical to that used for FIG. 19. Each data point represents the average of three independent experiments. Bar: SE.

FIG. 24. rhMDA-7 protein kills melanoma cells. MeWo cells were treated with 0-20 ng/ml rhMDA-7 and 4 days later, viability assessed using Trypan blue. Cells were also treated with 20 ng/ml rhMDA-7 in the presence of anti-MDA-7 antibodies (rabbit polyclonal:Pab or murine monoclonal:Mab) or control human IgG.

FIG. 25A-25B. Melanoma tumor MDA-7 expression negatively correlates with tumor iNOS expression. A negative association between mean iNOS count and MDA-7 count (A). The Kendall τ-b correlation coefficient is −0.209, and is significantly different from 0 with P<0.05. A negative association between mean iNOS intensity and MDA-7 intensity (B). The Kendall τ-b correlation coefficient is −0.201, and is significantly different from 0 with P<0.05; bars, ±SD.

FIG. 26. Immunoblotting analysis of IRF-1 and IRF-2 after 4 h of treatment of the human melanoma cell line MeWo with rhMDA-7. Treatments include medium only (Lane 1, negative control); supernatant from nontransfected HEK 293 cells (Lane 2, negative control); 5 ng/ml rhMDA-7 (Lane 3); and 20 ng/ml rhMDA-7 (Lane 4). The membrane was immunoblotted with anti-IRF1 and IRF-2 antibodies at 1:2000 dilutions. Shown is one representative experiment. Graphs indicate IRF-1 and IRF-2 expression after normalization to actin protein in the cell lysates, and represent the mean of two experiments; bars, ±SD.

FIG. 27. Ad-mda 7 augments anti-tumor efficacy of tamoxifen.

FIG. 28. Ad-mda7 and MDA-7 protein regulate cytokine secretion from melanoma cells.

FIG. 29. Effect of Ad-mda7 on A549 Lung Metastases.

FIG. 30. PAC1 cells are strongly transduced with adenoviral vectors. Human H1299 lung cancer or PAC1 cells were transduced with 50 or 100 pfu/cell of Ad-SM22-beta-gal (Ad-SM22) or Ad-RSV-beta-gal (Ad-RSV) at the indicated MOIs. 24 hours later, the cells were stained for beta-gal activity and X-gal positive cell enumerated. Data are shown as means of triplicate counts.

FIG. 31. MDA-7 suppresses PAC1 cell growth. PAC1 SMC were transduced with Ad-mda 7 or Ad-luc at indicated MOI. Viable cells were counted manually 3 days after transduction in triplicate. Data are shown as mean±SD. p<0.05 (*) compared with the control virus (Ad-luc).

FIGS. 32A-C. Induction of apoptosis in PAC1 by Ad-mda7. A. Ad-mda7 increases caspase-3 activity. PAC1 cells were transduced with Ad-mda7 or Ad-luc at 100 MOI. 48 hours after transduction, one set of cell lysates was used for caspase-3 activity assay and another set used for total protein quantification. Caspase-3 activities were normalized with the total protein and expressed as Units/10 μg total protein. p<0.05 (*) compared with the control virus (Ad-luc) and untreated control (A). Annexin V binding assay. PAC1 cells were transduced with Ad-mda7 or Ad-luc at 100 MOI and stained with FITC-labeled Annexin V 24 hours after transduction. The treated cells were analyzed by flow cytometry (B). The percentage of early apoptotic cells was calculated using Modfit software. p<0.05 (*) compared with the control virus (Ad-luc). DAPI staining assay (C). PAC1 cells were transduced with Ad-mda 7 or Ad-luc at 100 MOI and stained with DAPI 24, 48, 72 hours after transduction and apoptotic nuclei were scored positive if they exhibited evidence of chromatin decondensation. p<0.05 (*) compared with the control virus (Ad-luc).

FIG. 33. Inhibition of PAC1 cell migration by Ad-mda7. Confluent PAC1 cells were transduced with 100 MOI of Ad-mda7 or Ad-luc and treated as described in Example 22. Bar graph showing the quantified migration of cells into the wound by microscopy. p<0.01 (#) for +FBS vs −FBS; p<0.01 (*) for Ad-mda7 vs Untreated or Ad-luc+FBS; p<0.05 (A) for Ad-mda7 vs Untreated or Ad-luc +FBS.

FIG. 34. Time course and dose response for biological effects of INGN 241. Time course and dosages shown for 6 different cohorts in clinical trial of INGN 241.

FIG. 35. MDA-7 protein expression correlates with apoptosis induction. Protein levels of MDA-7 were determined and tunel assays were performed on sections from 10 different patients.

FIG. 36. RNA, DNA, and protein expression levels of MDA-7 in different tumor sections were evaluated in Patient 4, who had a melanoma.

FIG. 37. The spread of MDA-7 DNA and RNA from the site of injection was evaluated in 10 patients.

FIG. 38. MDA-7 protein levels and extent of apoptosis were evaluated in different sections from a number of patients.

FIG. 39. The spread of MDA-7 expression was also evaluated and correlated with apoptosis levels using TUNEL assays.

FIG. 40. A time course was done evaluating MDA-7 DNA from the point of injection.

FIG. 41. A time course was done evaluating MDA-7 protein and apotosis levels from point of injection.

FIG. 42. Phase II clinical trial initial results.

FIG. 43. AsPc1, Capan2 and MiaPaCa2 pancreatic cancer cells were treated with 2000 vp/cell Ad-mda7 and Ad-luc for 72 hours and analyzed for viability using trypan blue and apoptosis using Annexin V staining. Data shown as mean±SD.

FIG. 44. MiaPaCa2 cells were treated with Ad-mda7 or control, irradiated 40 hr later and plated for clonogenic assay.

FIG. 45. AsPc1 and MiaPaCa pancreatic cancer lines were treated with 2000 vp/cell Ad-luc or Ad-mda7 and 24 hours later treated with XRT (5 Gy). Cells were evaluated for cell cycle changes on day 3 by treatment with propidium iodide and analyzed by FACS. Note the G2/M block and sub-GO signal in Ad-mda7/XRT samples.

FIG. 46. Ad-mda 7 activates NF-κB-dependent reporter gene expression.

FIG. 47. Cytotoxic effect of Ad-mda7 in dominant negative I-κBα stable cells.

FIG. 48. Ad-mda7 significantly suppresses cell growth in dominant negative I-κBα cells.

FIG. 49A-C. Ad-mda7 synergizes with sulindac to induce apoptosis. A. Rate of apoptosis in cells treated with Ad-mda7 alone and in combination with sulindac. B. Tumor (A549 and H1299) and normal (CCD-16) cells were treated with PBS, Ad-luc, or Ad-mda7 for 3 h. After treatment, cells were incubated with sulindac at the indicated concentrations. After 72 h, cell viability was determined using the trypan-blue exclusion assay method. Percentage of cell growth was calculated as the average of cell counts for each group and expressed relative to the each sample treated with PBS, Ad-luc, or Ad-mda7 alone (set to 100%). Tumor but not normal cells treated with Ad-mda7/sulindac were significantly inhibited compared with PBS and Ad-luc treatments (P=0.001). The inhibitory effects mediated by sulindac were dose dependent. Bars, SE. C. Analysis of apoptotic cells by FACS. Tumor cells (A549 and H1299) and normal (CCD-16) cells were treated with PBS, Ad-luc, or Ad-mda7 in the presence of various doses of sulindac. 72 h after treatment, cells were stained with propidium iodide, and subjected to FACS analysis. The percentages of apoptotic cells were determined by quantifying cells in the sub-G₁ phase. The mean values of duplicate samples are shown; similar results were observed in at least two independent experiments. Bars, SE. D. Subcutaneous H1299 tumor-bearing nude mice were divided into groups (n=8/group). Animals treated with Ad-mda7/sulindac demonstrated significant tumor growth inhibition compared with animals treated with PBS, sulindac, Ad-mda7, or Ad-luc/sulindac. Ad-mda7 (3×10⁹vp) was administered by intratumoral injection thrice a week and sulindac (40 mg/kg) by i.p. injection daily. Tumor volumes given represent the mean for each group per time point. Bars, SE.

FIG. 50. Adenoviral transduction of five ovarian cancer cell lines (MDAH2774, OVCAR 420, DOV 13, HEY, and SKOV3-ip) were determined by infecting the cells with Ad-GFP.

FIG. 51. Inhibition of cell proliferation in ovarian cancer cell lines MDAH 2774 and OVCA 420 following infection with Ad-mda-7.

FIG. 52. Flow cytometric analysis demonstrates a marked increase in the percentage of the G₂/M population in two of five ovarian cancer cell lines that showed significant growth suppression, MDAH 2774 and OVCA 420.

FIG. 53. Cell survival in MDA-MB-486 breast cancer cells.

FIG. 54. Effect of Ad-mda7 administration before treatment with radiation in A549 tumor growth (A) and mice survival (B). A549 cells (5×. 10⁶) were grown as xenograft tumors in nude mice. Tumor-bearing mice were treated with radiation (5 Gy), Ad-mda7 (3×10¹⁰ vp in three fractions) or a combination of the two, then tumor volumes were measured as described in Example 27. Animals were sacrificed when tumors reached 15 mm in diameter or ulcerated. Data are presented as the means±SE (A).

FIG. 55. Effect of various regimens of combination therapies in A549 tumor growth. Tumor-bearing mice were treated as follows: control, Ad-mda7 (day 1) plus irradiation (day 6), Ad-mda7 (day 5) plus irradiation (day 6) or irradiation (day 6) plus Ad-mda 7 (day 7).

FIG. 56. Immunohistochemical analysis of TUNEL. Apoptosis in the tumor were detected after treatment (day 8) by TUNEL staining, and apoptotic cells were counted under a light microscope (×400 magnification), and the apoptosis index was calculated as a percentage of at least 1000 cancer cells.

FIG. 57. The protein expression of VEGF, bFGF and IL-8 were analyzed by immunohistochemistry for positive staining. Subcutaneous tumors were harvested on day 14. Positive staining cells were counted under a light microscope (×400 magnification), and the positive percent was calculated as a percentage of at least 1000 cancer cells (A, B, C).

FIG. 58. Micro vessel density was determined by counting CD31 positive vascular structures.

FIG. 59. Clonogenic survival of HUVECS. After growth factor starvation for 12 hours, HUVECS were exposed to MDA7 protein (10 ng/ml)(A), angiostatin (100 ng/ml; B), or endostatin (100 ng/ml; C) for 12 hours. Then cells were irradiated (0-6 Gy), harvested and plated in regular medium. Colonies were stained 14 days later and the surviving fraction was determined. Data are shown as the mean±SE of three separate experiments.

FIG. 60. Clonogenic survival of A549 cells (A) and CCD16 cells (B). Cells were serum starved for 12 hours and treated with conditioned medium containing mda7 protein (10 ng/ml). Twelve hours later, cells were irradiated, harvested and plated in regular medium. After incubation for 14 days, colonies were counted and survival.

FIG. 61. Targeting plasmid constructs, including full length, cytoplasm, nucleus, and endoplasmic reticulum (ER).

FIG. 62. ER targeting of MDA-7 blocks colony formation.

FIG. 63. ER-targeted MDA-7 is pro-apoptotic.

FIG. 64. Growth inhibition caused by Ad-mda7 in ovarian cancer cell lines.

FIG. 65. Cell cycle analysis of Ad-mda7 treated ovarian cancer cells. A: MDAH 2774; B: OVCA 420.

FIG. 66. Induction of apoptosis by Ad-mda7 in ovarian cancer cells.

FIG. 67. MDA-7/IL-24 inhibits tumor cell migration. Lung tumor cells (A549 and Hi299) were treated with Ad-luc or Ad-mda7. Cells were harvested 6 h after transfection and seeded into the upper chamber of a Transwell unit. A: After 48 h, the membrane was fixed and stained with crystal violet, and the number of cells that had migrated to the lower side of the well was counted under bright field microscopy (upper panel; ×200 magnification). Cells treated with Ad-mda7 were significantly (P=0.002) less able to migrate than cells treated with PBS or Ad-luc (lower panel). B: Analysis of cell viability 24 h and 48 h after treatment showed no significant inhibition of tumor cell proliferation. Bars denote standard error.

FIG. 68. MDA-7/IL-24 inhibits tumor cell invasion. Lung tumor cells (H1299 and A549) were treated with PBS, Ad-luc (2500 vp/cell), or Ad-mda7 (2500 vp/cell) or treated with 10 μM LY 294002 or 1 μg/ml MMP-II inhibitor. After 6 h, cells were harvested, counted, and added into the upper wells of Matrigel-coated wells. Cells were allowed to invade by incubation at 37° C. After 48 h, cells were fixed and stained with crystal violet. Cells that migrated to the lower side of the well were observed and counted under a light microscope at ×200 magnification. The number of invading cells per treatment was counted in a blind fashion and recorded as the average of three separate experiments. Cells treated with Ad-mda7 showed less invasion (P=0.001) than cells treated with PBS or Ad-luc. Inhibitory effect mediated by MDA-7 was similar to the inhibitory effect observed with LY 294002 and MMP-II inhibitor. Bars denote standard error.

FIG. 69. MDA-7/IL-24 inhibits lung metastases. A549 lung tumor cells were treated with PBS, Ad-luc, and Ad-mda7 ex vivo. After 6 h, cells were harvested, washed, resuspended in PBS, and injected into female nude mice via the tail vein. There were five animals in each group. Three weeks after the injection of the tumor cells, the animals were euthanized by CO₂ inhalation, and the lung tumor nodules were counted under a dissecting microscope. Significantly (P=0.01) fewer lung tumors were observed in animals injected with tumor cells treated with Ad-mda7 than in animals injected with tumor cells treated with PBS or Ad-luc. Results are the mean of two separate experiments. Bars denote standard error.

FIG. 70. MDA-7/IL-24 inhibits lung metastases. Mice bearing experimental A549 lung metastasis were either untreated (control), or treated with DOTAP:Chol.CAT, or DOTAP:Chol-mda7 complex. Animals were treated daily for six-doses via tail vein injection. Three weeks after the last treatment animals were euthanized and the number of lung tumors counted. Animals treated with DOTAP:Chol-mda7 complex demonstrated significant inhibition of lung metastasis compared to animals that were untreated or treated with DOTAP:Chol-CAT complex (P=0.001). Bars denote standard error

FIG. 71A-C. DOTAP:Chol-mda-7 complex suppresses growth of subcutaneous tumors. Subcutaneous tumor-bearing (A549 or UV223m) nude mice and C3H mice were divided into groups and treated daily for a total of six doses (50 μg/dose), as follows: no treatment, PBS, DOTAP:Chol-LacZ complex or DOTAP:Chol-CAT complex, and DOTAP:Chol-mda-7 complex. A, A549. B, UV2237m. Each time point represents the mean tumor volume for each group. Bars represent standard errors. C. Subcutaneous tumors were harvested 48 hours after treatment and analyzed for MDA-7 protein expression. In tumors treated with the DOTAP:Chol-mda-7 complex, 18% of A549 tumor cells and 13% of UV2237m tumor cells produced the MDA-7 protein, while control tumors produced no MDA-7 protein.

FIG. 72. MDA-7 induces apoptotic cell death following treatment with the DOTAP:Chol-mda-7 complex. Subcutaneous tumors (A549, and UV2237m) from animals receiving no treatment, PBS, DOTAP:Chol-LacZ or DOTAP:Chol-CAT complex, or DOTAP:Chol-mda-7 complex were harvested and analyzed for apoptotic cell death by TUNEL staining. The percentages-of cells undergoing apoptotic cell death (13% for A549 and 9% for UV2237m) in tumors treated with DOTAP:Chol-mda-7 complex were significantly higher (P=0.001) than in the other treatment groups. Bars denote standard deviation.

FIG. 73. DOTAP:Chol-mda-7 complex inhibits tumor vascularization. Subcutaneous tumors (A549, and UV2237m) that were either untreated or treated with PBS, DOTAP:Chol-LacZ or DOTAP:Chol-CAT complex, or DOTAP:Chol-mda-7 complex were stained for CD31 and subjected to semi-quantitative analysis. CD31-positive endothelial staining was significantly lower (P=0.01) in DOTAP:Chol-mda7-treated tumors than in the tumors of other treatment groups. Bars denote standard deviation.

FIG. 74. DOTAP:Chol-mda-7 complex inhibits experimental lung metastases. Lung tumor (A549, UV2237m)-bearing nu/nu or C3H mice were treated daily for a total of six doses (50 μg/dose) with PBS, DOTAP:Chol-CAT complex or DOTAP:Chol-mda-7 complex. Metastatic tumor growth was significantly inhibited (P=<0.05) in both nude mice and C3H mice that were treated with DOTAP:Chol-mda-7 complex compared with that in the two control groups. Bars denote standard deviation.

### DESCRIPTION OF ILLUSTRATIVE EMBODIMENTS

A. MDA-7

The compositions and methods of the present invention employ MDA-7 polypeptides and nucleic acids encoding such polypeptides. MDA-7 is another putative tumor suppressor that has been shown to suppress the growth of cancer cells that are p53-wild-type, p53-null and p53-mutant. Also, the observed upregulation of the apoptosis-related B gene in p53 null cells indicates that MDA-7 is capable of using p53-independent mechanisms to induce the destruction of cancer cells. Applicants' observed that adenoviral-mediated overexpression of MDA-7 led to the rapid induction and activation of double stranded RNA-activated serine threonine kinase (PKR) with subsequent phosphorylation of eIF-2α, other PKR target substrates and apoptosis induction. Specific inhibition of PKR by 2-aminopurine (2-AP) in lung cancer cells abrogates Ad-mda7 induced PKR activation, PKR substrate target phosphorylation and apoptosis induction. As evidenced by PKR null fibroblasts, Ad-mda7 apoptosis is dependent on a functional PKR pathway. These characteristics indicate that MDA-7 has broad therapeutic, prognostic and diagnostic potential as an inducer of PKR and, consequently, an enhancer of an induced immune response.

PKR exerts antiviral and anticellular functions, and is involved in regulating a number of physiologic processes that include cell growth and differentiation (U.S. Pat. No. 6,326,466; Feng et al., 1992; Petryshyn et al., 1988; Petryshyn et al., 1984; Judware et al., 1991), tumor suppression (Koromilas et al., 1992; Meurs et al., 1993), and modulation of signal transduction pathways (Kumar et al., 1994).

Upregulation of PKR leads to the induction of apoptosis in various cancer cell lines. Furthermore, in myelodysplasias, critical tumorigenic deletions of the IRF-1 gene on chromosome 5q (Beretta et al., 1996) appear associated with decreased PKR levels and immunohistochemical analyses of lung and colorectal cancers demonstrate an association with PKR expression and prolonged survival (Haines et al., 1992). PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phophorylate various substrate targets, which are important in growth control and apoptosis induction (Saelens et al., 2001; Sudhakar et al., 2000). Stimulation of the immune system has been linked to apoptosis (Albert et al., 1998; Chen et al., 2001; Saif-Muthama et al., 2000; Restifo, 2001). Further, artifical induction of apoptosis has been demonstrated to enhance the immunogenicity of a vaccine due to the stimulatory effect of dendritic cells that became activated by transfection of the apoptotic cells (Sasaki et al., 2001; Chattergoon et al., 2000).

Mda-7 mRNA has been identified in human PBMC (Ekmekcioglu et al., 2001), and no cytokine function of human MDA-7 protein was reported. MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine¹⁷. The rat MDA-7 homolog C49a (Mob-5) is 78% homologous to the mda-7 gene and has been linked to wound healing (Soo et al. 1999; Zhang et al., 2000). Mob-5 was also shown to be a secreted protein and a putative cell surface receptor was identified on ras transformed cells (Zhang et al., 2000). Therefore, homologues of the mda-7 gene and the secreted MDA-7 protein are expressed and secreted in various species. However, no data has emerged to show MDA-7 has cytokine activity. Such activity has ramifications for the treatment of a wide variety of diseases and infections by enhancing immunogenicity of an antigen.

The mda-7 cDNA (SEQ ID NO:1) encodes a novel, evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. The protein sequence shows no significant homology to known proteins with the exception of a 42 amino acid stretch that is 54% identical to interleukin 10 (IL-10). Structural analysis has determined that MDA-7 (IL-BKW or IL-20) displays the structural characteristics of the cytokine family (WO 98/28425, incorporated herein by reference). The structural characteristics and limited identity across a small stretch of amino acids implies an extracellular function for MDA-7. The expression of MDA-7 is inversely correlated with melanoma progression as demonstrated by increased mRNA levels in normal melanocytes as compared to primary and metastatic melanomas as well as decreased MDA-7 expression in early vertical growth phase melanoma cells selected for enhanced tumor formation in nude mice. Additional information and data, regarding MDA-7 can be found in patent application Ser. Nos. 09/615,154, 10/017,472, 60/404,932, 60/370,335, 60/361,755 and the U.S. non-provisional patent application entitled “Methods for Enhancing Immune Induction Involving MDA-7” filed on Mar. 3, 2003 in the name of Sunil Chada, Abujiang Pataer, Abner Mhashilkar, Rajagopal Ramesh, Jack Roth, and Steve Swisher, all of which applications are herein incorporated by reference in their entireties.

Additional studies have shown that elevated expression of MDA-7 suppressed cancer cell growth in vitro and selectively induced apoptosis in human breast cancer cells as well as inhibiting tumor growth in nude mice (Jiang et al., 1996 and Su et al., 1998). Jiang et al. (1996) report findings that mda-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2). Mda-7 overexpression in normal cells (HMECs, HBL-100, and CREF-Trans6) showed limited growth inhibition indicating that mda-7 transgene effects are not manifest in normal cells. Taken together, the data indicates that growth inhibition by elevated expression of MDA-7 is more effective in vitro in cancer cells than in normal cells.

Su et al. (1998) reported investigations into the mechanism by which MDA-7 suppressed cancer cell growth. The studies reported that ectopic expression of MDA-7 in breast cancer cell lines MCF-7 and T47D induced apoptosis as detected by cell cycle analysis and TUNEL assay without an effect on the normal HBL-100 cells. Western blot analysis of cell lysates from cells infected with adenovirus mda-7 (“Ad-mda7”) showed an upregulation of the apoptosis stimulating protein BAX. Ad-mda7 infection elevated levels of BAX protein only in MCF-7 and T47D cells and not normal HBL-100 or HMEC cells. These data lead the investigators to evaluate the effect of ex vivo Ad-mda7 transduction on xenograft tumor formation of MCF-7 tumor cells. Ex vivo transduction resulted in the inhibition of tumor formation and progression in the tumor xenograft model.

The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growning tumor with neccessary nutrients. Endostatin and angiostatin are examples of two such therapies (WO 00/05356 and WO 00/26368).

Applicants have discovered a method of inhibiting angiogenesis. This new method comprises the administration of a nucleic acid encoding human mda-7. Ad-mda7 has the ability to inhibit endothelial differentiation when added to proliferating endothelial cells in vitro. The anti-angiogenic effects of elevated mda-7 expression make this molecule an ideal gene therapy treatment for angiogenesis-related diseases, especially cancer. Administration of a nucleic acid encoding mda-7, via viral or non-viral vectors, to anti-angiogenic target cells, which can comprise endothelial cells, as well as administration to tumor cells is contemplated. This combinaiton treatment allows the clinician to not only rely on the direct transduction of a tumor cell but also on the effect of inhibiting angiogenesis. Thus, starving and attacking the tumor by using two separate modalities that may be delivered to different target cell population.

Angiogenesis-related diseases include, but are not limited to, angiogenesis-dependent cancer, including, for example, solid tumors, blood-borne tumors such as leukemias, and tumor metastases; benign tumors, for example hemangiomas acoustic neuromas, neurofibromas, trachomas, and pyogenic granulomas; rheumatoid arthritis; psoriasis; ocular angiogenic diseases, for example, diabetic retinopathy, retinopathy of prematurity macular degeneration, corneal graft rejection, neovascular glaucoma, retrolental fibroplasia. Rubeosis; Osler-Webber Syndrome; myocardial angiogenesis; plaque neovascularization; telangiectasia; hemophiliac joints: angiofibroma; and wound granulation. The endothelial cell proliferation inhibiting methods of the present invention are useful in the treatment of disease of excessive or abnormal stimulation of endothelial cells. These diseases include, but are not limited to, intestinal adhesions, atherosclerosis. scleroderma, and hypertrophic scars, i.e., keloids. They are also useful in the treatment of diseases that have angiogenesis as a pathologic consequence such as cat scratch disease (Rochele minalia quintosa) and ulcers (Helobacter pylori).

The methods of the present invention are useful for treating endothelial cell-related diseases and disorders. A particularly important endothelial cell process is angiogenesis, the formation of blood vessels, as described above. Angiogenesis-related diseases may be treated using the methods described in present invention to inhibit endothelial cell proliferation by elevated expression of MDA-7.

Though not adhering to a particular theory regarding the operability of these constructs, there is a notable amino acid homology of mda-7 to EL-10 and across species in the D-helical region, located at the C-terminus, which is implicated in receptor binding. Thus, molecules preferably containing this 30-35 amino acid region are particularly preferred.

Thus, in one embodiment of the present invention, the treatment of angiogenesis-related disease involves the administration of a therapeutic peptide or polypetpide. In another embodiment, treatment involves administration of a nucleic acid expression construct encoding mda-7 to target, comprising diseased cells or endothelial cells. It is contemplated that the target cells take up the construct, and express the therapeutic polypeptide encoded by nucleic acid, thereby inhibiting differentiation in the target cells. Cells expressing MDA-7 in turn can secrete the protein which may interact with neighboring cells not transduced or infected by an expression construct. In this way the complex interactions needed to establish new vasculature for the tumor is inhibited and treatment of the tumor accomplished.

In another embodiment of the present invention, it is contemplated that an angiogenesis-related disease may be treated with a MDA-7, or constructs expressing the same. Some of the angiogenesis-related diseases contemplated for treatment in the present invention are psoriasis, rheumatoid arthritis (RA), inflammatory bowel disease (IBD), osteoarthritis (OA) and pre-neoplastic lesions in the lung.

In yet another embodiment, the treatment of a wide variety of cancerous states is within the scope of the invention. For example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, lymphoma, brain, colon or bladder. In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment.

In certain embodiments of the present invention, the mda-7 is provided as a nucleic acid expressing the MDA-7 polypeptide. In specific embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹³, 10¹⁴, 10¹⁵ or higher pfu or viral particles. In certain embodiments, the viral vector is an adenoviral vector, a retroviral vector, a vaccinia viral vector, an adeno-associated viral vector, a polyoma viral vector, an alphaviral vector, a rhabdoviral vector, or a herpesviral vector. Most preferably, the viral vector is an adenoviral vector. In other specific embodiments, the nucleic acid is a non-viral vector.

In certain embodiments, the nucleic acid expressing the polypeptide is operably linked to a promoter. Non-limiting examples of promoters suitable for the present invention include a CMV IE, dectin-1, dectin-2, human CD11c, F4/80, SM22 or MHC class II promoter, however, any other promoter that is useful to drive expression of the mda-7 gene or the immunogene of the present invention, such as those set forth herein, is believed to be applicable to the practice of the present invention.

Preferably, the nucleic acid of the present invention is administered by injection. Other embodiments include the administering of the nucleic acid by multiple injections. In certain embodiments, the injection is performed local, regional or distal to a disease or tumor site. In some embodiments, the administering of nucleic acid is via continuous infusion, intratumoral injection, intraperitoneal, or intravenous injection. In other embodiments, the nucleic acid is administered to the tumor bed prior to or after; or both prior to and after resection of the tumor. Alternatively, the nucleic acid is administered to the patient before, during, or after chemotherapy, biotherapy, immunotherapy, surgery or radiotherapy. Preferably the patient is a human. In other embodiments the patient is a cancer patient.

B. Nucleic Acids, Vectors and Regulatory Signals

The present invention concerns polynucleotides or nucleic acid molecules relating to the mda-7 gene and its gene product MDA-7. Additionally, the present invention is directed to polynucleotides or nucleic acid molecules relating to an immunogenic molecule. These polynucleotides or nucleic acid molecules are isolatable and purifiable from mammalian cells. It is contemplated that an isolated and purified MDA-7 nucleic acid molecule, either the secreted or full-length version, that is a nucleic acid molecule related to the mda-7 gene product, may take the form of RNA or DNA. Similarly, the nucleic acid molecule related to the immunogenic molecule may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding MDA-7” refers to a nucleic acid segment that contains MDA-7 coding sequences, yet is isolated away from, or purified and free of, total genomic DNA and proteins. When the present application refers to the function or activity of a MDA-7-encoding polynucleotide or nucleic acid, it is meant that the polynucleotide encodes a molecule that has the ability to enhance an immune response. Further, a “polynucleotide encoding an immunogen” refers to a nucleic acid segment that contains an immunogenic coding sequences, yet is isolated away from, or purified and free of, total genomic DNA and proteins. When the present application refers to the function or activity of an immunogene encoding an immunogen, it is meant that the polynucleotide encodes an immunogenic molecule that has the ability to induce an immune response in the body of a human.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). There may be times when the full or partial genomic sequence is some. Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode a MDA-7 polypeptide; a human MDA-7 polypeptide is a specific embodiment. Consequently, the present invention also encompasses derivatives of MDA-7 with minimal amino acid changes, but that possess the same activity.

The term “gene” is used for simplicity to refer to a functional protein, polypeptide, or peptide-encoding nucleic acid unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding MDA-7 or another therapeutic polypeptide such as the immunogen may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO:1 (MDA-7 encoding sequence).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a MDA-7 protein, polypeptide or peptide that includes within its amino acid sequence a contiguous amino acid sequence in accordance with, or essentially as set forth in, SEQ ID NO:2, corresponding to the MDA-7 designated “human MDA-7” or “MDA-7 polypeptide.”The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about.85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 will be sequences that are “essentially as set forth in SEQ ID NO:2” provided the biological activity of the protein is maintained. In particular embodiments, the biological activity of a MDA-7 protein, polypeptide or peptide, or a biologically functional equivalent, comprises enhancing an immune response. Further, in particular embodiments, the biological activity of an immunogen, an immunogenic molecule that is a protein, polypeptide or peptide, or a biologically functional equivalent, comprises immunogenecity, which refers to the molecule's ability to induce an immune response in the body of a human. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:1. Again, DNA segments that encode proteins, polypeptide or peptides exhibiting MDA-7 activity will be most some.

In particular embodiments, the invention concerns isolated nucleic acid segments and recombinant vectors incorporating DNA sequences that encode MDA-7 polypeptides or peptides that include within its amino acid sequence a contiguous amino acid sequence in accordance with, or essentially corresponding to MDA-7 polypeptides. In other embodiments, the invention relates to an isolated nucleic acid segment and recombinant vectors incorporating DNA sequences that encode an immunogen, protein, polypeptide or peptides that include within its amino acid sequence a contiguous amino acid sequence in accordance with, or essentially corresponding to the immunogen.

Vectors of the present invention are designed, primarily, to transform cells with a therapeutic mda-7 gene under the control of regulated eukaryotic promoters (i.e., inducible, repressable, tissue specific). Also, the vectors may contain a selectable marker if, for no other reason, to facilitate their manipulation in vitro. However, selectable markers may play an important role in producing recombinant cells.

Tables 1 and 2, below, list a variety of regulatory signals for use according to the present invention.

TABLE 1
Inducible Elements

| Element | Inducer | References |
|---|---|---|
| MT II | Phorbol Ester (TPA) | Palmiter et al., 1982; Haslinger and |
| | Heavy metals | Karin, 1985; Searle et al., 1985; |
| | | Stuart et al., 1985; Imagawa et al., |
| | | 1987; Karin et al., 1987; Angel et |
| | | al., 1987b; McNeall et al., 1989 |
| MMTV (mouse | Glucocorticoids | Huang et al., 1981; Lee et al., 1981; |
| mammary tumor | | Majors and Varmus, 1983; Lee et |
| virus) | | al., 1984; Ponta et al., 1985 |
| β-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TFA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | Ela, SV40 Large T Antigen | Taylor et al., 1989; Taylor and |
| | | Kingston, 1990a, b |
| Proliferin | Phorbol Ester-TPA | Mordacq and Linzer, 1989 |
| Tumor Necrosis | PMA | Hensel et al., 1989 |
| Factor | | |
| Thyroid | Thyroid Hormone | Chatterjee et al., 1989 |
| Stimulating | | |
| Hormone α Gene | | |

TABLE 2
Other Promoter/Enhancer Elements

| Promoter/Enhancer | References |
|---|---|
| Immunoglobulin Heavy Chain | Banerji et al., 1983; Gillies et al., 1983; |
| | Grosschedl and Baltimore, 1985; Atchinson and |
| | Perry, 1986, 1987; Imler et al., 1987; Neuberger et |
| | al., 1988; Kiledjian et al., 1988; |
| Immunoglobulin Light Chain | Queen and Baltimore, 1983; Picard and Schaffner, |
| | 1985 |
| T-Cell Receptor | Luria et al., 1987, Winoto and Baltimore, 1989; |
| | Redondo et al., 1990 |
| HLA DQ α and DQ β | Sullivan and Peterlin, 1987 |
| β-Interferon | Goodbourn et al., 1986; Fujita et al., 1987; |
| | Goodbourn and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., 1989 |
| Muscle Creatine Kinase | Jaynes et al., 1988; Horlick and Benfield, 1989; |
| | Johnson et al., 1989a |
| Prealbumin (Transthyretin) | Costa et al., 1988 |
| Elastase I | Omitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987, Tronche et al., 1989, 1990 |
| α-Fetoprotein | Godbout et al., 1988; Campere and Tilghman, |
| | 1989 |
| γ-Globin | Bodine and Ley, 1987; Perez-Stable and |
| | Constantini, 1990 |
| β-Globin | Trudel and Constantini, 1987 |
| c-fos | Cohen et al., 1987 |
| c-HA-ras | Triesman, 1985; Deschamps et al., 1985 |
| Insulin | Edlund et al., 1985 |
| Neural Cell Adhesion Molecule | Hirsch et al., 1990 |
| (NCAM) | |
| a₁₋Antitrypain | Latimer et al., 1990 |
| H2B (TH2B) Histone | Hwang et al., 1990 |
| Mouse or Type I Collagen | Rippe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh |
| | and Lockett, 1985; Firak and Subramanian, 1986; |
| | Herr and Clarke, 1986; Imbra and Karin, 1986; |
| | Kadesch and Berg, 1986; Wang and Calame, |
| | 1986; Ondek et al., 1987; Kuhl et al., 1987 |
| | Schaffner et al., 1988 |
| Polyoma | Swartzendruber and Lehman, 1975; Vasseur et al., |
| | 1980; Katinka et al., 1980, 1981; Tyndell et al., |
| | 1981; Dandolo et al., 1983; Hen et al., 1986; |
| | Campbell and Villarreal, 1988 |
| Retroviruses | Kriegler and Botchan, 1983; Kriegler et al., |
| | 1984a, b; Bosze et al., 1986; Miksicek et al., 1986; |
| | Celander and Haseltine, 1987; Thiesen et al., |
| | 1988; Celander et al., 1988; Chol et al., 1996; |
| | Reisman and Rotter, 1989 |
| Papilloma Virus | Campo et al., 1983; Lusky et al., 1983; Spandidos |
| | and Wilkie, 1983; Spalholz et al., 1985; Lusky |
| | and Botchan, 1986; Cripe et al., 1987; Gloss et |
| | al., 1987; Hirochika et al., 1987, Stephens and |
| | Hentschel, 1987 |
| Hepatitis B Virus | Bulla and Siddiqui, 1988; Jameel and Siddiqui, |
| | 1986; Shaul and Ben-Levy, 1987; Spandau and |
| | Lee, 1988 |
| Human Immunodeficiency Virus | Muesing et al., 1987; Hauber and Cullan, 1988; |
| | Jakobovits et al., 1988; Feng and Holland, 1988; |
| | Takebe et al., 1988; Berkhout et al., 1989; Laspia |
| | et al., 1989; Sharp and Marciniak, 1989; Braddock |
| | et al., 1989 |
| Cytomegalovirus | Weber et al., 1984; Boshart et al., 1985; Foecking |
| | and Hofstetter, 1986 |
| Gibbon Ape Leukemia Virus | Holbrook et al., 1987; Quinn et al., 1989 |

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In some embodiments, the promoter for use in the present invention is the cytomegalovirus (CMV) promoter. This promoter is commercially available from Invitrogen in the vector pcDNAIII, which is some for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylate cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

Compositions and methods of the invention are provided for administering the compositions of the invention to a patient. 1. Viral Transformation

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

C. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Protamine

Protamine may also be used to form a complex with an expression construct. Such complexes may then be formulated with the lipid compositions described above for administration to a cell. Protamines are small highly basic nucleoproteins associated with DNA. Their use in the delivery of nucleic acids is described in U.S. Pat. No. 5,187,260, which is incorporated by reference. U.S. patent application Ser. No. 10/391,068 (filed Mar. 24, 2003), which pertains to methods and compositions for increasing transduction efficiency of a viral vector by complexing the viral vector with a protamine molecule, is specifically incorporated by reference herein.

2. Non-Viral Delivery

In addition to viral delivery of the nucleic acid encoding a MDA-7 protein, the following are additional methods of recombinant gene delivery to a given host cell and are thus considered in the present invention.

a. Lipid Mediated Transformation

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

C. Proteins, Peptides and Polypeptides

1. Biologically Functional Equivalents

The present invention is directed to methods and compositions of MDA-7 polypeptides. In certain embodiments, the MDA-polypeptides are used in the treatment of diseases associated with angiogenesis, such as cancer. In certain embodiments, the MDA-7 polypeptide is directly provided. In specific embodiments, the MDA-7 polypeptide is provided before therapy. In specific embodiments, the MDA-7 polypeptide is administered at the same time as administration of an immunogenic molecule, such as an antigen, for purposes of immune therapy. In other specific embodiments, the MDA-7 polypeptide is provided after therapy, and in some instances, after providing an immunogenic molecule for purposes of treating, diagnosing or prognosing induction of an immune response. The terms “protein” and “polypeptide” are used interchangeably herein.

Additional embodiments of the invention encompass the use of a purified protein composition comprising MDA-7 protein, truncated versions of MDA-7, and peptides derived from MDA-7 amino acid sequence administered to cells or subjects for the inhibition of angiogenesis. Truncated molecules of MDA-7 include, for example, molecules beginning approximately at MDA-7 amino acid residues 46-49 and further N-terminal truncations. Specifically contemplated are molecules start at residue 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206. In additional embodiments, residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48 are included with other contiguous residues of MDA-7, as shown in SEQ ID NO:2.

As will be understood by those of skill in the art, modification and changes may be made in the structure of a MDA-7 polypeptide or peptide, an immunogenic molecule, or an immungene product and still produce a molecule having like or otherwise desirable characteristics. For example, certain amino acids may be substituted for other amino acids in a protein structure without appreciable loss of interactive binding capacity with structures such as, for example, antigen-binding regions of antibodies or binding sites on molecules such as Tat and RNA polymerase II. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. It is thus contemplated by the inventors that various changes may be made in the sequence of HIV polypeptides or peptides (or underlying DNA) without appreciable loss of their biological utility or activity.

In terms of functional equivalents, the skilled artisan also understands it is also well understood by the skilled artisan that inherent in the definition of a biologically-functional equivalent protein or peptide, is the concept of a limit to the number of changes that may be made within a defined portion of a molecule that still result in a molecule with an acceptable level of equivalent biological activity. Biologically-functional equivalent peptides are thus defined herein as those peptides in which certain, not most or all, of the amino acids may be substituted. In particular, where small peptides are concerned, less amino acids may be changed. Of course, a plurality of distinct proteins/peptides with different substitutions may easily be made and used in accordance with the invention.

It is also well understood that where certain residues are shown to be particularly important to the biological or structural properties of a protein or peptide, e.g., residues in the active site of an enzyme, or in the RNA polymerase II binding region, such residues may not generally be exchanged. This is the case in the present invention, where residues shown to be necessary for inducing an immune response should not generally be changed, which is contemplated for both the MDA-7 polypeptide and the immunogene product.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred,some, those which are within ±1 are particularly preferred,some, and those within ±0.5 are even more particularly preferred.some.

It is also understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred,some, those which are within ±1 are particularly preferred,some, and those within ±0.5 are even more particularly preferred.some.

While discussion has focused on functionally equivalent polypeptides arising from amino acid changes, it will be appreciated that these changes may be effected by alteration of the encoding DNA, taking into consideration also that the genetic code is degenerate and that two or more codons may encode the same amino acid. A table of amino acids and their codons is presented belowhereinabove for use in such embodiments, as well as for other uses, such as in the design of probes and primers and the like.

2. Synthetic Peptides

The compositions of the invention may include a peptide modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

The present invention describes MDA-7 peptides for use in various embodiments of the present invention. For example, specific peptides are assayed for their abilities to elicit an anti-angiogenic response. In specific embodiments that the peptides are relatively small in size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

The compositions of the invention may include a peptide modified to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides which include all L-amino acids, all D-amino acids, or a mixture thereof. The use of D-amino acids may confer additional resistance to proteases naturally found within the human body and are less immunogenic and can therefore be expected to have longer biological half lives.

3. In vitro Protein Production

In addition to the purification methods provided in the examples, general procedures for in vitro protein production are discussed. Following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshney, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogene product, and more specifically, an protein having immunogenic activity. Other examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

4. ER-Targeting Sequences

The polypeptides of the present invention include one or more endoplasmic reticulum targeting sequences. The final location of a protein within a cell depends upon targeting sequences encoded within the sequence of a protein. In the simplest case, the lack of a signal directs proteins to the default pathway which is the cytoplasm. Proteins destined to be retained in the ER must have certain signal peptides to retain the protein in the ER. The polypeptides of the present invention may or may not include additional amino acid residues at the N-terminal or C-terminal.

The ER is a network of membrane-enclosed tubules and sacs (cistemae) that extends from the nuclear membrane throughout the cytoplasm. The secretory pathway of proteins is as follows: rough ER→Golgi→secretory vesicles→cell exterior.

For proteins to be secreted, the protein must generally travel from the ER to the Golgi. However, there are certain proteins that must be maintained within the ER, such as BiP, signal peptidase, protein disulfide isomerase. Specific localization signals target proteins to the ER.

Certain proteins are retained in the ER lumen as a result of the presence of the ER targeting sequence Lys-Asp-Glu-Leu (KDEL, in the single-letter code) at their carboxy terminus. If this sequence is not part of the protein, the protein is instead transported to the Golgi and secreted from the cell. The presence of the KDEL sequence or the KKXX sequence at the carboxy terminus (KKXX sequences) results in retention of proteins in the ER. The presence of these sequences results in binding of the protein to specific recycling receptors in the membranes of these compartments and are then selectively transported back to the ER.

Protein export from the ER occurs not only by bulk flow, but by a regulated pathway that specifically recognizes targeting signals that mediate selective transport of proteins to the Golgi apparatus. The presence of a 16- to 30-residue ER signal sequence directs the ribosome to the ER membrane and initiates transport of the protein across the ER membrane.

ER signal sequences are usually located at the N-terminus of the protein. These targeting sequences frequently contains one or more positively charged amino acids followed by a continuous stretch of 6-12 hydrophobic residues. Signal sequences are usually cleaved from the protein while it is still growing on the ribosome. The specific deletion of several of the hydrophobic amino acids from a signal sequence or a mutation of one of them to a charged amino acid results in failure of the protein to cross the ER membrane into the lumen. The addition of random N-terminal amino acid sequences will cause a cytosolic protein to be translocated to the ER lumen, indicating that the hydrophobic residues form a binding site that is critical for ER targeting.

The endoplasmic reticulum targeting sequence may include any number of amino acid residues, as long as these amino acid residues target the destination of the polypeptide to the endoplasmic reticulum. The polypeptides of the present invention may include a single ER targeting sequence, or more than one ER targeting sequence. Additional information pertaining to ER targeting signals can be found in Invitrogen Catalog Nos. V890-20, V891-20, V892-20, and V893-20, “pShooter Vector Manual I (pEF/myc vectors),” on the internet at invitrogen.com/content/sfs/manuals/pshooter_pef_man.pdf, which is hereby incorporated by reference in its entirety. Reviews of signal sequence recognition and protein targeting to the ER can also be found in Walter and Johnson, 1994; Koch et al., 2003; and Kabat et al., 1987, which are also specifically incorporated by reference herein.

5. Antibodies

Another embodiment of the present invention are antibodies, in some cases, a human monoclonal antibody immunoreactive with the polypeptide sequence of MDA-7 (SEQ ID NO:1). It is understood that antibodies can be used for inhibiting or modulating MDA-7. In addition, the antibody may be useful in passive immunotherapy for cancer. All such uses of the said antibody and any antigens or epitopic sequences so discovered fall within the scope of the present invention. The discussion below applies to the use of antibodies against MDA-7 in methods of the invention.

a. MDA-7 Antigenic Sequences

As another way of effecting modulation of MDA-7 in a subject, peptides corresponding to one or more antigenic determinants of the MDA-7polypeptides of the present invention also can be prepared so that an immune response against MDA-7is raised. Thus, it is contemplated that vaccination with a MDA-7peptide or polypeptide may generate an autoimmune response in an immunized animal such that autoantibodies that specifically recognize the animal's endogenous MDA-7protein. This vaccination technology is shown in U.S. Pat. Nos. 6,027,727; 5,785,970, and 5,609,870, which are hereby incorporated by reference.

Such peptides should generally be at least five or six amino acid residues in length and will preferably be about 10, 11, 12, 13, 14, 15, 16, 17, 18, 19, 20, 25 or about 30 amino acid residues in length, and may contain up to about 35-50 residues. For example, these peptides may comprise a MDA-7 amino acid sequence, such as 5, 6, 7, 8, 9, 10, 11, 12, 13, 14, 15, 16, 17, 18, 19, 20, 21, 22, 23, 24, 25, 30, 35, 40, 45, and 50 or more contiguous amino acids from SEQ ID NO:2. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

U.S. Pat. No. 4,554,101, incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the MDA-7 sequence disclosed herein in SEQ ID NO:2.

Numerous scientific publications have also been devoted to the prediction of secondary structure, and to the identification of epitopes, from analyses of amino acid sequences (Chou & Fasman, 1974a, b; 1978a, b; 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PepPlot® (Brutlag et al., 1990; Weinberger et al., 1985), and other new programs for protein tertiary structure prediction (Fetrow & Bryant, 1993). Another commercially available software program capable of carrying out such analyses is MacVector (EBI, New Haven, Conn.).

In further embodiments, major antigenic determinants of an MDA-7 polypeptide may be identified by an empirical approach in which portions of the gene encoding the MDA-7 polypeptide are expressed in a recombinant host, and the resulting proteins tested for their ability to elicit an immune response. For example, PCR™ can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR™ cloning methodology.

The use of such small peptides for antibody generation or vaccination typically requires conjugation of the peptide to an immunogenic carrier protein, such as hepatitis B surface antigen, keyhole limpet hemocyanin or bovine serum albumin, or other adjuvants discussed above (adjuvenated peptide). Alum is an adjuvant that has proven sufficiently non-toxic for use in humans. Methods for performing this conjugation are well known in the art. Other immunopotentiating compounds are also contemplated for use with the compositions of the invention such as polysaccharides, including chitosan, which is described in U.S. Pat. No. 5,980,912, hereby incorporated by reference. Multiple (more than one) MDA-7 epitopes may be crosslinked to one another (e.g., polymerized). Alternatively, a nucleic acid sequence encoding an Fortilin peptide or polypeptide may be combined with a nucleic acid sequence that heightens the immune response. Such fusion proteins may comprise part or all of a foreign (non-self) protein such as bacterial sequences, for example.

Antibody titers effective to achieve a response against endogenous MDA-7 will vary with the species of the vaccinated animal, as well as with the sequence of the administered peptide. However, effective titers may be readily determined, for example, by testing a panel of animals with varying doses of the specific antigen and measuring the induced titers of autoantibodies (or anti-self antibodies) by known techniques, such as ELISA assays, and then correlating the titers with MDA-7-related cancer characteristics, e.g., tumor growth or size.

One of ordinary skill would know various assays to determine whether an immune response against MDA-7 was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al., 1991, hereby incorporated by reference.

D. Methods of MDA-7 Purification

The present invention provides for methods of purification of MDA-7. The following methods and similar methods known to one of ordinary skill in the art can be used to practice the methods of purification of MDA-7 disclosed herein.

1. Gel Electrophoresis

Gel electrophoresis is a well-known technique that can be used in the purification procedure. Agarose, agarose-acrylamide or polyacrylamide gel electrophoresis using standard methods (Sambrook et al., 2001) can be utilized in the purification process.

2. Chromatographic Techniques

Alternatively, chromatographic techniques may be employed to effect isolation and purification of MDA-7. There are many kinds of chromatography which may be used in the present invention: adsorption, affinity, partition, ion-exchange and molecular sieve, and many specialized techniques for using them including column, paper, thin-layer and gas chromatography (Freifelder, 1982).

3. Immunological Reagents

Certain aspects of the claimed invention involve use of immunological reagents. In certain embodiments of the claimed invention, immunological reagents are used in the purification of preparations of MDA-7. Antibodies, which are discussed herein, are contemplated for use with the present invention.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen—either a peptide, portion of a polypeptide, or an entire polypeptide, such as MDA-7, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

4. Immunodetection Methods

In still further embodiments, the present invention concerns immunodetection methods for binding, purifying, removing, quantifying and/or otherwise generally detecting biological components such as MDA-7 expressed message(s), protein(s), polypeptide(s) or peptide(s). Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference. These techniques are well-known to those of skill in the art.

E. Pharmaceutical Formulations and Delivery

In certain embodiments of the present invention, methods involving delivery of an expression construct encoding a MDA-7 protein are contemplated. In some embodiments, the method is directed to delivery of an expression construct encoding an immunogen. Alternatively, the expression construct comprises sequence encoding both the MDA-7 polypeptide and the immunogen. Examples of diseases and conditions involving an immune response include diseases that are prevented or treated with a vaccine. Including lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, breast cancer, bladder cancer and any other diseases or condition related to an immune response that may be treated by administering a MDA-7 polyprotein to enhance an induced immune response.

An “effective amount” of the pharmaceutical composition, generally, is defined as that amount sufficient to detectably and repeatedly to achieve the stated desired result, for example, to ameliorate, reduce, minimize or limit the extent of the disease or its symptoms. More rigorous definitions may apply, including elimination, eradication or cure of disease.

1. Administration

In certain specific embodiments, it is desired to kill cells, inhibit cell growth, inhibit metastasis, decrease tumor or tissue size and otherwise reverse or reduce the malignant phenotype of tumor cells, induce an immune response, or inhibit angiogenesis using the methods and compositions of the present invention. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation.

Direct injection, intratumoral injection, or injection into the tumor vasculature is specifically contemplated for discrete, solid, accessible tumors or other accessible target areas. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as single dose comprise about 0.1 to about 0.5 ml volumes. The viral particles may advantageously be contacted by administering multiple injections to the tumor or targeted site, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising MDA-7 or an MDA-7-encoding construct together with or in the absence of an immunogenic molecule. The perfusion may be continued post-resection, for example, by leaving a catheter implanted at the site of the surgery. Periodic post-surgical treatment also is envisioned.

Continuous perfusion of an expression construct or a viral construct also is contemplated. The amount of construct or peptide delivered in continuous perfusion can be determined by the amount of uptake that is desirable.

Continuous administration also may be applied where appropriate, for example, where a tumor or other undesired affected area is excised and the tumor bed or targeted site is treated to eliminate residual, microscopic disease. Delivery via syringe or catherization is some. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, immune condition, target site, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor or affected area being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor or targeted site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher.

Protein may be administered to a patient in doses of or of at least 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml.

2. Injectable Compositions and Formulations

In some embodiments, the method for the delivery of an immunogenic molecule, an expression construct encoding a MDA-7 protein, MDA-7 protein, and/or an immunogen is via systemic administration. However, the pharmaceutical compositions disclosed herein may alternatively be administered parenterally, subcutaneously, directly, intratracheally, intravenously, intradermally, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

c. Adjuvants

As is also well known in the art, the immunuogenicity of an immunogenic molecule, immunogen or peptide composition can be enhanced by the use of non-specific stimulators of the immune response, known as adjuvants. Suitable adjuvants include all acceptable immunostimulatory compounds, such as cytokines, toxins, or synthetic compositions. In the present invention, the administering of an effective amount of a MDA-7 polypeptide enhances an immune response, thereby functioning as an adjuvant. Further, in other embodiments, a molecule that increases expression of PKR is considered to enhance an immune response and can be an acceptable immunostimulatory compound in the present invention.

However, other adjuvants may be used in addition to MDA-7 and they include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion. MHC antigens may even be used.

Exemplary, adjuvants include complete Freund's adjuvant (a non-specific stimulator of the immune response containing killed Mycobacterium tuberculosis), incomplete Freund's adjuvants and aluminum hydroxide adjuvant.

It is contemplated that in addition to MDA-7, other compounds with adjuvant activity may be included in certain aspects of the invention. Adjuvants, functions and mechanisms of delivery are well known in the art. Non-limiting examples of other adjuvants include Adjumer™ (i.e., PCPP salt; polyphosphazene); Adju-Phos (i.e., Aluminum phosphate gel); Algal Glucan (i.e., b-glucan; glucan); Algammulin (i.e., Gamma inulin/alum composite adjuvant); Alhydrogel (i.e., Aluminum hydroxide gel; alum); Antigen Formulation (i.e., SPT, AF); Avridine® (i.e., N,N-dioctadecyl-N′,N′-bis(2-hydroxyethyl)propanediamine; CP20,961); BAY R1005 (i.e., N-(2-Deoxy-2-L-leucylamino-b-D-glucopyranosyl)-N-octadecyldodecanoylamidehydroacetate); Calcitriol (i.e., 1a, 25-dihydroxyvitamin D3; 1,25-di(OH)2D3; 1,25-DHCC; 1a, 25-dihydroxycholecalciferol); Calcium Phosphate Gel (i.e., Calcium phosphate); Cholera holotoxin (CT) and Cholera toxin B subunit (CTB) (i.e., CT; CTB subunit; CTB); Cholera toxin A1-subunit-ProteinA D-fragment fusion protein (i.e., CTA1-DD gene fusion protein); CRL1005 (i.e., Block Copolymer P1205); Cytokine-containing Liposomes (i.e., Cytokine-containing Dehydration Rehydration Vesicles.); DDA (i.e., Dimethyldioctadecylammonium bromide; dimethyldistearylammonium bromide (CAS Registry Number 3700-67-2)); DHEA (i.e., Dehydroepiandrosterone; androstenolone; prasterone); DMPC (i.e., Dimyristoyl phosphatidylcholine; 1,2-dimyristoyl-sn-3-phosphatidyl choline; (CAS Registry Number 18194-24-6)); DMPG (i.e., Dimyristoyl phosphatidylglycerol; sn-3-phosphatidyl glycerol-1,2-dimyristoyl, sodium salt (CAS Registry Number 67232-80-8)); DOC/Alum Complex (i.e., Deoxycholic Acid Sodium Salt; DOC/Al(OH)3/mineral carrier complex); Freund's Complete Adjuvant (i.e., CIA; FCA); Freund's Incomplete Adjuvant (i.e., IFA;FIA); Gamma Inulin; Gerbu Adjuvant; GM-CSF (i.e., Granulocyte-macrophage colony stimulating factor; Sargramostim (yeast-derivedrh-GM-CSF)); GMDP (i.e., N-acetylglucosaminyl-(β1-4)-N-acetylmuramyl-L-alanyl-D-isoglutamine (CAS Registry Number 70280-03-4)); Imiquimod (i.e., 1-(2-methypropyl)-IH-imidazo[4,5-c]quinolin-4-amine; R-837; S26308); ImmTher™ (i.e., N-acetylglucosaminyl-N-acetyhnuramyl-L-Ala-D-isoGlu-L-Ala-glycerol dipalmitate; DTP-GDP); Immunoliposomes Containing Antibodies to Costimulatory Molecules (i.e., lmmunoliposomes prepared from Dehydration-Rehydration Vesicles (DRVs)); Interferon-g (i.e., Actimmune® (rhIFN-gamma, Genentech, Inc.); immune interferon; IFN-g; gamma-interferon); Interleukin-1β (i.e., IL-10; IL-1; human Interleukin 1β mature polypeptide 117-259); Interleukin-2 (i.e., IL-2; T-cell growth factor; aldesleukin (des-alanyl-1, serine-125 human interleukin 2); Proleukin®; Teceleukin®); Interleukin-7 (i.e., IL-7); Interleukin-12 (i.e., IL-12; natural killer cell stimulatory factor (NKSF); cytotoxic lymphocyte maturation factor (CLMF)); ISCOM(s)™ (i.e., Immune stimulating complexes); Iscoprep 7.0.3.^{™;} Liposomes (i.e., Liposomes (L) containing protein or Th-cell and/or B-cell peptides, or microbes with or without co-entrapped interieukin-2, BisHOP or DOTMA; A, [L (Antigen)]); Loxoribine (i.e., 7-allyl-8-oxoguanosine); LT-OA or LT Oral Adjuvant (i.e., E. coli labile enterotoxin protoxin); MF59; MONTANIDE ISA 51 (i.e., Purified IFA; Incomplete Freund's adjuvant.); MONTANIDE ISA 720 (i.e., metabolizable oil adjuvant); MPL™ (i.e., 3-Q-desacyl-4′-monophosphoryl lipid A; 3D-MLA); MTP-PE (i.e., N-acetyl-L-alanyl-D-isoglutaminyl-L-alanine-2-(1,2-dipalmitoyl-sn-glycero-3-(hydroxy-phosphoryloxy)) ethylamide, mono sodium salt); MTP-PE Liposomes (i.e., MTP-PE Antigen presenting liposomes); Murametide (i.e., Nac-Mur-L-Ala-D-Gln-OCH3); Murapalmitine (i.e., Nac-Mur-L-Thr-D-isoGIn-sn-glyceroldipalmitoyl); D-Murapalmitine (i.e., Nac-Mur-D-Ala-D-isoGln-sn-glyceroldipalmitoyl); NAGO (i.e., Neuraminidase-galactose oxidase); Non-Ionic Surfactant Vesicles (i.e., NISV); Pleuran (i.e., b-glucan; glucan); PLGA, PGA, and PLA (i.e., Homo-and co-polymers of lactic and glycolic acid; Lactide/glycolide polymers; poly-lactic-co-glycolide); Pluronic L121 (i.e., Poloxamer 401); PMMA (i.e., Polymethyl methacrylate); PODDS™ (i.e., Proteinoid microspheres); Poly rA:Poly rU (i.e., Poly-adenylic acid-poly-uridylic acid complex); Polysorbate 80 (i.e., Tween 80; Sorbitan mono-9-octadecenoate poly(oxy-1,2-ethanediyl) derivatives); Protein Cochleates; QS-21 (i.e., Stimulon™ QS-21 Adjuvant); Quil-A (i.e., Quil-A saponin, Quillaja saponin); Rehydragel HPA (i.e., High Protein Adsorbency Aluminum Hydroxide Gel; alum); Rehydragel LV (i.e., low viscosity alluminum hydroxide gel; alum); S-28463 (i.e., 4-Amino-otec,-dimethyl-2-ethoxymethyl-1H-imidazo[4,5-c]quinoline-1-ethanol); SAF-1 (i.e., SAF-m; Syntex Adjuvant Formulation); Sclavo peptide (i.e., I L-1b 163-171 peptide); Sendai Proteoliposomes, Sendai-containing Lipid Matrices (i.e., Sendai glycoprotein-containing vesicles; fusogenic proteoliposomes; FPLs); Span 85 (i.e., Arlacel 85, sorbitan trioleate); Specol; Squalane (i. e., Spinacane; Robane® ;2,6,10,15,19,23 -hexamethyltetracosane); Squalene (Spinacene; Supraene; 2,6,10,15,19,23-hexamethyl-2,6,10,14,18,22 tetracosahexaene); Stearyl Tyrosine (i.e., Octadecyl tyrosine hydrochloride); Theramide™ (i.e., N-acetylglucosaminyl-N-acetylinuramyl-L-Ala-D-isoGlu-L-Ala-dipalmitoxy propylamide (DTP-DPP)); Threonyl-MDP (i.e., Termurtide™ ; [thrl]-MDP; N-acetyl muramyl-L-threonyl-D-isoglutamine); Ty Particles (i.e., Ty-VLPs, (Virus Like Particles)); Walter Reed Liposomes (i.e., Liposomes containing lipid A adsorbed to aluminum hydroxide, [L(Lipid A+Antigen)+Alum]).

In addition to adjuvants, it may be desirable to coadminister biologic response modifiers (BRM), which have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

3. Vaccines

The present invention includes methods and compositions for preventing the development of cancer or precancer. As such, the invention contemplates vaccines for use in both active and passive immunization embodiments. Immunogenic compositions, proposed to be suitable for use as a vaccine, may be prepared most readily directly from purified MDA-7 prepared in a manner disclosed herein. Preferably the antigenic material is extensively dialyzed to remove undesired small molecular weight molecules and/or lyophilized for more ready formulation into a desired vehicle.

The preparation of vaccines that contain MDA-7 sequences as active ingredients is generally well understood in the art by analogy, as exemplified by U.S. Pat. Nos. 5,958,895, 6,004,799, and 5,620,896, all incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants that enhance the effectiveness of the vaccines.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The MDA-7 protein (or fragments thereof) or a nucleic acid encoding all or part of MDA-7 may be formulated into the vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, amonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A), or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

4. Combination Treatments

In certain embodiments, the compositions and methods of the present invention involve an MDA-7 polypeptide, or expression construct coding therefor, in combination with other agents or compositions to enhance the effect of MDA-7 or to increase any therapeutic, diagnostic, or prognostic effect for which the MDA-7 is being employed. These compositions would be provided in a combined amount effective to achieve the desired effect, for example, the killing of a cancer cell or the inhibition of angiogenesis. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In one embodiment of the present invention, it is contemplated that mda-7 gene therapy is used in conjunction with immune therapy intervention, in addition to other pro-apoptotic, anti-angiogenic, anti-cancer, or cell cycle regulating agents. Alternatively, the therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, for example gene therapy is “A” and the immunogenic molecule given as part of an immune therapy regime, such as an antigen, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic expression constructs of the present invention to a patient will follow general protocols for the administration of such compounds, taking into account the toxicity, if any, of the vector. It is expected that the treatment cycles would be repeated as necessary. It also is contemplated that various standard therapies, as well as surgical intervention, may be applied in combination with the described therapy.

In specific embodiments, it is contemplated that an antic-cancer therapy, such as chemotherapy, radiotherapy, immunotherapy or other gene therapy, is employed in combination with MDA-7 therapy, as described herein.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. No. 5,760,395 and U.S. Pat. No. 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

c. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with MDA-7. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000).

As discussed earlier, examples of immunotherapies currently under investigation or in use are immune adjuvants e.g., Mycobacterium bovis, Plasmodium falciparum, dinitrochlorobenzene and aromatic compounds (U.S. Pat. No. 5,801,005; U.S. Pat. No. 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. No. 5,830,880 and U.S. Pat. No. 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). It is contemplated that one or more anti-cancer therapies may be employed with the MDA-7 therapies described herein.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie and Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond.

d. Gene Therapy

In yet another embodiment, a combination treatment involves gene therapy in which a therapeutic polynucleotide is administered before, after, or at the same time as an MDA-7 polypeptide or nucleic acid encoding the polypeptide. Delivery of an MDA-7 polypptide or encoding nucleic acid in conjunction with a vector encoding one of the following gene products may have a combined therapeutic effect on target tissues. A variety of proteins are encompassed within the invention, some of which are described below. Table 3 lists various genes that may be targeted for gene therapy of some form in combination with the present invention.

i) Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors, like ErbB. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Ab1 and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii) Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, pl6 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP}1. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zacl, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii) Regulators of Programmed Cell Death

Apoptosis, or programmed cell death, is an essential process for normal embryonic development, maintaining homeostasis in adult tissues, and suppressing carcinogenesis (Kerr et al., 1972). The Bcl -2 family of proteins and ICE-like proteases have been demonstrated to be important regulators and effectors of apoptosis in other systems. The Bcl -2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl -2 protein now is recognized to be a member of a family of related proteins, which can be categorized as death agonists or death antagonists.

Subsequent to its discovery, it was shown that Bcl -2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs'surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

TABLE 3
Oncogenes

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-α/ |
| | Virus; ALV promoter | Squamous cell | Amphiregulin/ |
| | Insertion; amplified | Cancer; glioblastoma | Hetacellulin receptor |
| | Human tumors | | |
| ERBB-2/NEU/ | Transfected from rat | Amplified breast, | Regulated by NDF/ |
| HER-2 | Glioblastomas | Ovarian, gastric | Heregulin and EGF- |
| | | cancers | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | Human colon cancer | | Factor) receptor |
| MET | Transfection from | | Scatter factor/HGF |
| | Human osteosarcoma | | Receptor |
| RET | Translocations and point | Sporadic thyroid cancer; | Orphan receptor Tyr |
| | mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine leukemia | | Src family; T cell |
| | Virus) promoter | | signaling; interacts |
| | Insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Abl |
| CRK | CT1010 ASV | | Adapted SH2/SH3 |
| | | | interact Ab1 |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, | HaRat SV; Ki RaSV; | Point mutations in many | Signal cascade |
| N-RAS | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | Thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | Transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + | Translocation/fusion | Acute myeloid leukemia | Gene fusion of DNA- |
| ELI/MEN | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian leukosis | | RB?; regulate |
| | Virus | | apoptosis? |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| FHIT | Fragile site 3p14.2 | Lung carcinoma | Histidine triad-related |
| | | | diadenosine 5′,3′′′′- |
| | | | P¹.p⁴ tetraphosphate |
| | | | asymmetric hydrolase |
| hMLI/MutL | | HNPCC | Mismatch repair; MutL |
| | | | Homologue |
| HMSH2/MutS | | HNPCC | Mismatch repair; MutS |
| | | | Homologue |
| HPMS1 | | HNPCC | Mismatch repair; MutL |
| | | | Homologue |
| hPMS2 | | HNPCC | Mismatch repair; MutL |
| | | | Homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xerodema | Excision repair; photo- |
| | | pigmentosum; skin | product recognition; |
| | | cancer predisposition | zinc finger |

e. Immunogenic Polypeptides/Peptides and Nucleic Acids

In yet another embodiment, the immunogenic molecule is a provided as part of a therapy regime. The immunogenic molecule may be provided directly or it may be provided as an expression vector encoding the immunogenic molecule. Delivery of a vector encoding mda-7 in conjuction with a second vector encoding one of the following gene products will have a combined inducing effect on target tissues. Alternatively, a single vector encoding both genes may be used.

(i) Antigens

In certain embodiments, the present invention is directed to improving immune therapy. An immune response against a tumor antigen can also be implemented with MDA-7. Tumor antigens include PSA, CEA, MART, MAGE1, MAGE3, gp100, BAGE, GAGE, TRP-1, TRP-2, PMSA, Mycobaterium tuberculosis soluble factor (Mtb), phenol soluble modulin (PSM), CMV-G, CMV-M, EBV capsid-EB nuclear antigen (EBNA), gp120, gp41, tat, rev, gag, toxa antigen, rubella antigen, mumps antigen, alpha-fetoprotein (AFP), adenocarcinoma antigen (ART-4), CAMEL, CAP-I, CASP-8, CDC27m, CDK4/m, CEA, CT, Cyp-B, DAM, ELF2M, ETV6-AMLI, ETS G250, GnT-V, HAGE, HER2/neu, HLA-A*0201-R1701, HPV-E7, HSP 70-2M, HST-2, hTERT, ICE, KIAA 0205, LAGE, LDLR/FUT, MC1R, MUCI, MUM-1, MUM-2, MUM-3, NA88-A, NY-ESO-I, p15, Pml/RARalpha, PRAME, PSM, RAGE, RU1, RU2, SAGE, SART-1, SART-3, TEL/AML1, TPI/m, or WT1. Uses for inducing a response against tumor antigens are specifically contemplated and can be found in U.S. Pat. Nos. 5,552,293 and 6,132,980, which are specifically incorporated by reference.

3. Vaccines

The present invention includes methods and compositions for preventing the development of cancer or precancer. As such, the invention contemplates vaccines for use in both active and passive immunization embodiments. Immunogenic compositions, proposed to be suitable for use as a vaccine, may be prepared most readily directly from purified MDA-7 prepared in a manner disclosed herein. Preferably the antigenic material is extensively dialyzed to remove undesired small molecular weight molecules and/or lyophilized for more ready formulation into a desired vehicle.

The preparation of vaccines that contain MDA-7 sequences as active ingredients is generally well understood in the art by analogy, as exemplified by U.S. Pat. Nos. 5,958,895, 6,004,799, and 5,620,896, all incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants that enhance the effectiveness of the vaccines.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The MDA-7 protein (or fragments thereof) or a nucleic acid encoding all or part of MDA-7 may be formulated into the vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin, mixture with bacterial cells such as C. parvum or endotoxins or lipopolysaccharide components of Gram-negative bacteria, emulsion in physiologically acceptable oil vehicles such as mannide mono-oleate (Aracel A), or emulsion with a 20% solution of a perfluorocarbon (Fluosol-DA®) used as a block substitute may also be employed.

In many instances, it will be desirable to have multiple administrations of the vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays.

F. Identification of an Immunogenic Molecule

The present invention exploits the observation that MDA-7 up-regulates the interferon induced, ds-RNA dependent serine/threonine protein kinase (PKR). PKR appears to mediate anti-tumorigenic activity through the activation of multiple transduction pathways culminating in growth inhibition and apoptosis induction. Activation of these pathways occurs after the latent, inactive homodimeric form is induced by activating signals to undergo conformational changes leading to auto-phosphorylation and activation (Vattem et al., 2001). Once activated, PKR is able to phophorylate various substrate targets, which are important in growth control and apoptosis induction (Saelens et al., 2001; Sudharkar et al., 2000).

The activation of PKR is a critical event in Ad-mda7 apoptosis. The inhibition of PKR with the specific threonine/kinase inhibitor, 2 amino-purine (2-AP) led to almost complete reversal of Ad-mda7 apoptosis and abrogation of eIF-2α phosphorylation and protein synthesis inhibition. The inhibition of protein synthesis may be critical to the induction of apoptosis possibly because of regulation of one or more short-lived proteins involved in apoptosis inhibition. Alternatively, other pathways controlled by PKR may be important such as those involved in regulation of NF-κB, p53, MEK, IRF-1 or FADD (Jagus et al., 1999; Gil et al. 1999; Cuddihy et al., 1999; Balachandran et al., 1998).

Even though multiple pathways may be involved, PKR activation is critical for Ad-mda7 apoptosis since MEFs lacking PKR were unable to undergo apoptosis as opposed to MEFs with wild-type PKR. This inhibition of apoptosis appeared specific to mda-7 since transduction of MEFs lacking PKR with the pro-apoptotic Ad-Bak vector lead to unimpaired apoptosis. A model for these observations was synthesized in which MDA-7 and PKR are upstream of the pro-apoptotic Bak gene in the apoptosis cascade. In this model, MDA-7 induces PKR activation which leads to various cellular pathways that then induce caspase activation and apoptosis induction. Bak, being downstream of PKR, is not dependent on PKR activation to induce apoptosis. The data also indicated BID cleavage and caspase 8 activation, which is consistent with other work in the art that have demonstrated that PKR apoptosis is often mediated through activation of Fas, FADD, caspase-8 and BID (Balachandran et al., 1998).

Thus, adenoviral-mediated overexpression of MDA-7 led to the rapid induction and activation of PKR with subsequent phosphorylation of eIF-2α, other PKR target substrates and apoptosis induction. Specific inhibition of PKR by 2-AP in lung cancer cells abrogates Ad-mda7 induced PKR activation, PKR substrate target phosphorylation and apoptosis induction. As evidenced by PKR null fibroblasts, Ad-mda7 apoptosis is dependent on a functional PKR pathway. These results indicate a novel role for the multi-functional PKR gene as a critical mediator of Ad-mda7 apoptosis. Further, because PKR has been described herein as critical to MDA-7, induced apoptosis, and which has been suggested to induce an immune response, the present invention in certain embodiments contemplates inducing PKR expression to enhance an immune response, the data indicate that MDA-7 polypeptide is capable of enhancing an immune response.

In other embodiments, the methods of the present invention are directed to identifying immunogenic molecules. In particular, the present invention is useful in enhancing an immune response against a previously unidentified immunogenic molecule or a molecule possessing immunogencity at a level that is, for example, below the limit of detection of conventional immune detection methods.

The invention is further directed to methods of prognosing a candidate patient for immunotherapy. A diagnostic test according to the present invention can evaluate whether a patient is a candidate for long-term non-progression by assaying for an immune response against an immunogenic molecule, such as an antigen. Another diagnostic test encompassed by the present invention can evaluate whether a subject is a candidate for a treatment method that prevents the diseases and conditions involving an immune response.

In one embodiment, the present invention includes a diagnostic test that determines whether a subject can exhibit an immune response against an immunogenic molecule. In another embodiment, a diagnostic test is employed to determine whether a subject exhibits an increased activity of a T-cell, a NK cell, or a macrophage. In another embodiment, the diagnostic method is employed to determine whether a subject exhibits an increased cytokine concentration. In either case, if the subject does, the present invention includes eliciting an immune response using compositions described herein. In further embodiments, a subject who either exhibits or can exhibit an induced immune response is administered a treatment method to enhance the immune response.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### MDA-7 is a Novel Ligand that Regulates Angiogenesis Via the IL-22 Receptor

Materials and Methods

1. Cell Culture

The human non-small cell lung cancer (NSCLC) cell line A549 (adenocarcinoma) and human embryonic kidney cells (293) obtained from the American Type Culture Collection (ATCC; Rockville, Md.) were grown in Hams/F12 medium (A549) and Dulbecco's modified Eagle's medium (293) supplemented with 10% fetal bovine serum (GIBCO-BRL, Grand Island, N.Y.). The HUVEC and HMVEC were purchased from Clonetics (Walkerville, Md.) and were grown in endothelial cell basal medium with 5% fetal bovine serum and additional reagents supplied as a “bullet kit” by the manufacturer. Endothelial cells were used at passage 3-9.

2. Production and Purification of Secreted MDA-7 Protein

MDA-7 protein was produced by transfecting 293 cells with a eukaryotic expression vector carrying the full-length mda-7 cDNA. After transfection was completed, cells were selected in hygromycin (0.4 μg/ml) for 14 days. The stable cell line (293-mda-7) was tested for production of soluble MDA-7 (sMDA-7) protein by western blot analysis and by ELISA. An aliquot of 10⁶ cells (293-mda-7), as determined by ELISA, produced approximately 30-50 ng/ml of sMDA-7 in 24 h. To purify the sMDA-7 protein on a large scale, 293-mda-7 cells were grown to 90% confluency in 150-mm tissue culture plates. The tissue culture supernatant was collected and pooled for protein purification by affinity chromatography, as described previously (Caudel et aL., 2002). The size and purity of the sMDA-7 protein were determined by silver stain gel and by Western blot analyses.

3. Endothelial Cell Proliferation Assay

To test the effect of sMDA-7 protein on cell proliferation, endothelial cells (HUVEC, HMVEC) were serum-starved overnight. The next day, cells were seeded in 2-well chamber slides (1−10⁴/well). The cells were allowed to adhere and spread for 4-6 h, and fresh medium containing 1 ng/ml of bFGF as a proangiogenic stimulator, and various concentrations of sMDA-7/IL-24 (1, 5, 10, and 50 ng/ml) was added. Cells treated with PBS served as controls. Cells were then harvested 3 days after treatment and cell proliferation determined by trypan blue exclusion assay method as previously described (Saeki et aL, 2000). The effect of sMDA-7 on lung tumor cell (H1299, and A549) proliferation was also evaluated. The experimental conditions were the same as described above for endothelial cells except that tumor cells were not stimulated with bFGF. Tumor cells treated with Ad-mda7 (3000 vp/cell) served as positive control.

4. Endothelial Cell Differentiation Assay

Endothelial cell differentiation (tube formation) assays were done using the in vitro angiogenesis assay kit (Chemicon, Temecula, Calif.). Briefly, HUVEC and HMVEC were grown to 80% confluency, collected, resuspended in growth medium, and plated at a concentration of 2−10⁴ cells/well in a 96-well plate coated with Matrigel (Chemicon, Temecula, Calif.). Cells were treated with sMDA-7 protein (1, 5, 10, and 50 ng/ml), or a preparation immunodepleted of sMDA protein for 24 h at 37° C. Cells treated with PBS served as negative controls in these experiments. The ability of sMDA-7 to inhibit tube formation was determined and quantitated by counting the number of tubes under bright-field microscopy.

For experiments involving comparative studies, cells were treated with equimolar concentrations of sMDA-7 (5, 10, and 300 ng/ml), recombinant human endostatin (5.2, 10.4, and 315 ng/ml; Calbiochem, La Jolla, Calif.), recombinant IFN-γ (4.5, 9, and 268 ng/ml; R&D systems, Minneapolis, Minn.) or recombinant IP-10 (2.4, 4.5, and 134 ng/ml; R&D systems, Minneapolis, Minn.) and analyzed for tube formation assay as described above. All samples were tested in duplicate. Experiments were repeated at least 5-6 times.

For receptor blocking studies, HUVEC grown in six-well plates were pretreated with IL-22R1 blocking antibody (1 ng/ml and 5 ng/ml). Following overnight incubation, cells were harvested, washed, and plated in Matrigel coated 96-well plates. Fresh IL-22R1 blocking antibody and sMDA-7 was added to the wells in a 1:1 ratio (1 ng/ml of IL-22R1 antibody:1 ng/ml of sMDA-7) or 1:5 ratio (1 ng/ml of IL-22R1 antibody:5 ng/ml of sMDA-7) and incubated at 37° C. After overnight incubation, the plates were examined for tube formation. All other experimental procedures were the same as described above. For experiments involving endostatin or IP-10, higher concentrations of these proteins were used (endostatin, 315 ng/ml; IP-10, 134 ng/ml) that demonstrated inhibitory activity in tube formation assay. The relative amount of IL-22R1 used for experiments involving endostatin was 315 ng/ml (1:1 ratio) and 134 ng/ml for experiments involving IP-10 (1:1 ratio). All other experimental procedures were the same as described previously. For blocking studies using anti-IP-10 or anti-IFN-γ neutralizing antibodies (R & D Systems), experiments were conducted as described above for receptor studies except that HUVEC were treated with the appropriate neutralizing antibody (1 μg and 5 μg/ml) prior to treatment with sMDA-7 (300 ng/ml).

5. Endothelial Cell Migration Assay

Cell migration assays were performed using HUVEC. Cells were starved overnight in basal medium containing 0.5% fetal bovine serum, collected, resuspended in the same medium, and seeded at a concentration of 10⁵ cells/well on the upper surface of a 24-well transwell insert with a pore size of 8 μm (Millipore, Cambridge, Mass.). The insert was placed in a six-well plate that contained medium plus PBS, medium plus VEGF (100 ng/ml) or VEGF plus sMDA-7 (10 or 50 ng/ml). The plates containing the transwell insert were incubated at 37° C. overnight to allow migration. The next day, the wells were disassembled, membranes were fixed in crystal violet, and the number of cells that had migrated to the lower wells was counted under high-power magnification (×40).

6. Determination of IP-10 and IFN-γ Production

Recent studies have demonstrated that treatment of PBMC with sMDA-7 results in secretion of IFN-γ (Caudell et al., 2002). Furthermore, IFN-γ is a potent inducer of IP-10 (Majumder et al., 1998). Both IFN-γ and IP-10 have been reported to possess antiangiogenic activity (Fathallah-Shaykh et al., 2000; Angiolillo et aL., 1996). Studies were conducted to determine whether the antiangiogenic activity of sMDA-7 was mediated by IFN-γ or IP-10. HUVEC were seeded in 6-well plates (1×10⁵/well) and treated with sMDA-7 (10 ng/ml). Cell culture supernatant was collected at 6 h, 24 h, and 48 h after treatment, centrifuged at 1200 rpm, and analyzed for IP-10 and IFN-γ protein production using commercially available ELISA kits. Assays were performed as recommended by the manufacturer (R&D systems, Minneapolis, Minn.). Cells treated with recombinant IFN-γ (4.5 ng/ml) served as positive control for the IP-10 assay, while cells treated with Ad-mda7 (3000 vp/cell) served as positive control for IFN-γ assay. Cells treated with PBS served as negative controls in these experiments. Samples were analyzed in quadruplicate, and data represented as the average value for each concentration of sMDA-7 tested.

7. Western Blot Analysis

Recent studies have demonstrated activation of STAT-3 expression in HACAT cells as a measure of sMDA-7 binding to its receptors (Dumoutier et al., 2001; Wang et al., 2002). Therefore, studies were conducted to determine the activation of STAT-3 expression in endothelial cells after treatment with sMDA-7. HUVEC were seeded in 6-well plates (5×10⁵ cells/well) and treated with sMDA-7 (10 ng/ml). Untreated cells served as negative controls. Cells were harvested at 4 h and 24 h after treatment and analyzed for STAT-3 expression by western blot analysis as previously described (Mhashilkar et al., 2001; Pataer et al., 2002). Phosphorylated STAT-2 (pSTAT-3) protein was detected using rabbit anti-human pSTAT-3 antibody (1:1000, Cell Signaling Technology, Beverly, Mass.) and horseradish peroxidase-labeled secondary antibody (Amersham Biosciences, Piscataway, N.J.). Finally, the proteins were visualized on enhanced chemiluminescence film (Hyperfilm, Amersham Biosciences, Piscataway, N.J.) by application of Amersham's enhanced chemiluminescence western blotting detection system. STAT-3 protein expression level was quantitated after normalization with total STAT-3 protein expression using Image Quant software (Molecular Dynamics, Amersham Pharmacia Biotech, Piscataway, N.J.).

8. Immunofluorescence Assay

Activation of STAT-3 was also determined by immunofluorescence assay. HUVEC seeded in two-well chamber slides (1×104 cells/well) was treated with PBS (control) or with sMDA-7 (10 ng/ml) for 4 h, washed in PBS, fixed in cold acetic acid, and stained for pSTAT-3 (pSTAT-3) using rabbit anti-human pSTAT-3 antibody (1:1000, Cell Signaling Technology, Beverly, Mass.) and rhodamine-labeled anti-rabbit secondary antibody (1:5000; Molecular Probes, Eugene, Oreg.). Slides were mounted using anti-fade mounting reagent (Vector Laboratories, Burlingame, Calif.). Pictures were taken through a fluorescence microscope 1-2 h after staining.

9. In Vivo Assessment of Antiangiogenic Activity Using the Matrigel Plug Assay

To determine the antiangiogenic activity of sMDA-7, an in vivo angiogenesis assay was performed. Briefly, sMDA-7 (12.5 ng) and bFGF (60 ng) was mixed with 500 μl of Matrigel (Beckton Dickinson, Bedford, Mass.) on ice and injected subcutaneously into athymic nude mice. Animals receiving Matrigel containing only bFGF (60 ng) served as positive controls and animals receiving Matrigel containing no growth factor served as negative controls. Each group comprised of five animals and the experiments were performed twice. Animals were sacrificed 10 days after injection. The Matrigel plugs were recovered, photographed, and subjected to hemoglobin analysis as previously described (Pessaniti et al., 1992).

10. Effects on Xenograft Tumors in Nude Mice

Parenteral 293 cells and 293-mda-7 cells were first tested for their ability to form tumors. Aliquots of 10⁶ cells were injected subcutanously into the lower right flank of athymic BALB/c female nude mice and the implantation site monitored for 1 month. No tumors formed at this cell concentration, so subsequent experiments were performed using this cell number. For in vivo mixing experiments, human lung tumor cells (A549) grown to 90% confluency were trypsinized, washed, and resuspended in sterile phosphate-buffered saline at a concentration of 5×10⁶/ml. The tumor-cell suspension was mixed with an equal number (5×10⁶/ml) of parental 293 cells or with 293-mda-7 cells, gently vortexed, and injected subcutaneously in nude mice (10⁶ cells/animal) as described above. Each group comprised of eight animals, and the experiments were done twice. Tumor growth was monitored and measured as described previously (Saeki et al., 2002). At the end of the experiment, animals were euthanized by CO₂ inhalation, and tumors were harvested for histopathological analysis, western blot analysis, and for CD31 and TUNEL staining.

To evaluate the systemic effect of sMDA-7 on tumor growth, subcutaneous tumors were established by injecting A549 tumor cells (5×10⁶ cells) into the lower right flank of nude mice. When the tumors were 50-60 mm³ in size, the animals were assigned to one of two groups of 10 mice each. One group of animals was injected with Matrigel containing parental 293 cells (1×10⁶), and the other group injected with Matrigel containing 293-mda-7 cells (1×10⁶). The Matrigel containing the cells was injected subcutaneously into the upper right flank of the tumor bearing mice. The effect of sMDA-7 on tumor growth was monitored as described above. At the end of the experiment, animals were euthanized, and tumors were harvested for further analyses as described above. All the animal experiments described were performed at least 2 times, and the differences in the tumor growth were tested for statistical significance.

11. Immunohistochemical Analysis

Tumor tissues were stained for CD31 and TUNEL as previously described (Saeki et al., 2002). Tissue sections stained without primary antibody or stained with an isotypic antibody served as negative controls. Tissue sections were analyzed and quantitated, and the results interpreted in a blind fashion.

12. Statistical Analysis

Student's t test was used to calculate the statistical significance of the experimental results. A P value less than 0.05 was considered to be statistically significant.

### Example 2

### sMDA-7 Inhibits Endothelial Cell Differentiation but Not Cell Proliferation

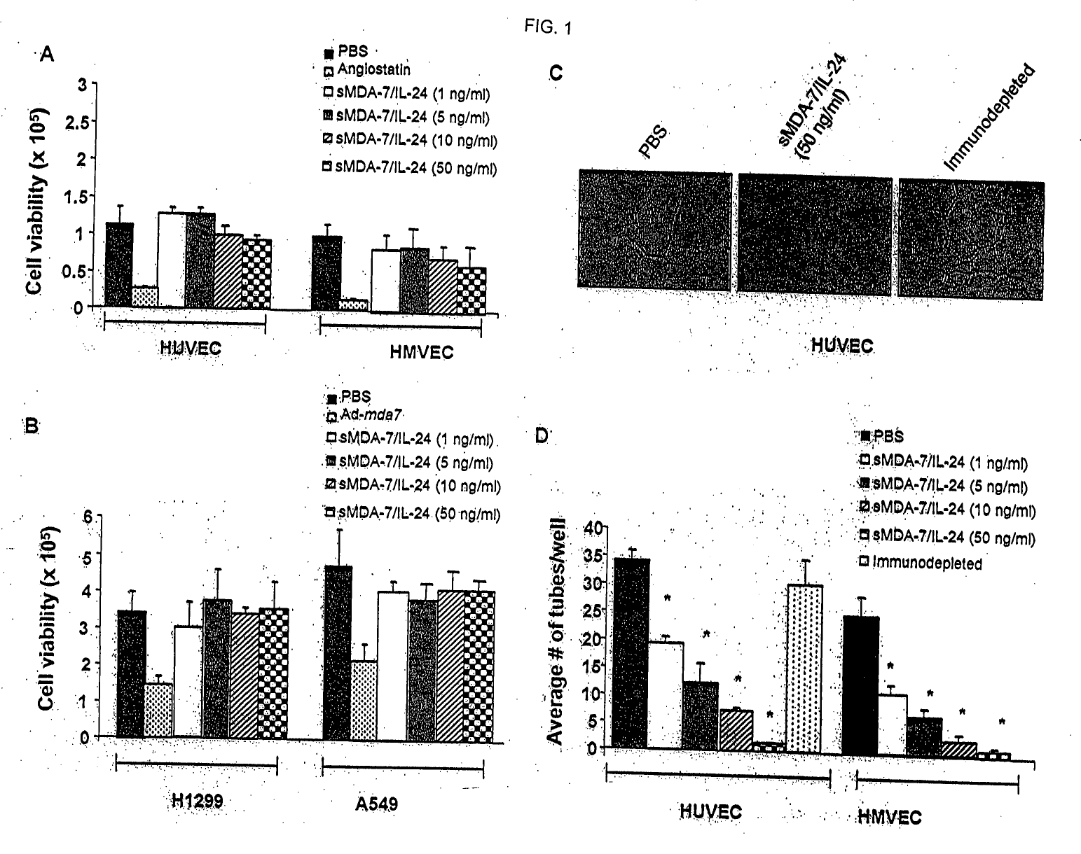

In preliminary studies, the inhibitory effect of sMDA-7 on endothelial cell proliferation using HUVEC and HMVEC was tested. Treatment of cells with various concentrations (1, 5, 10, and 50 ng/ml) of sMDA-7 resulted in no significant antiproliferative activity compared to PBS treated control cells (FIG. 1A, B). However, treatment of HUVEC and HMVEC with the above mentioned concentrations of sMDA-7 significantly inhibited (P=0.001) the formation of capillary tube-like structures by both types of endothelial cells (FIG. 1C, FIG. 1D). The inhibitory effect was observed at all concentrations and was dose-dependent, with a virtually complete abrogation of tube formation occurring at concentrations above 10 ng/ml (FIG. 1D).

To rule out the unlikely possibility that the inhibition of endothelial cell tube formation by sMDA-7 protein was due to unrelated proteins in the preparation, depletion experiments were performed. Immunodepletion of sMDA-7 protein from the test preparation prior to its addition to HUVEC resulted in complete restoration of endothelial cell tube formation (FIG. 1C, FIG. 1D). These data show that the observed inhibitory activity in the endothelial cell assays was due to sMDA-7 and suggest that sMDA-7 possesses potent antiangiogenic activity.

### Example 3

### sMDA-7 is More Potent than Endostatin in Inhibiting Endothelial Cell Differentiation

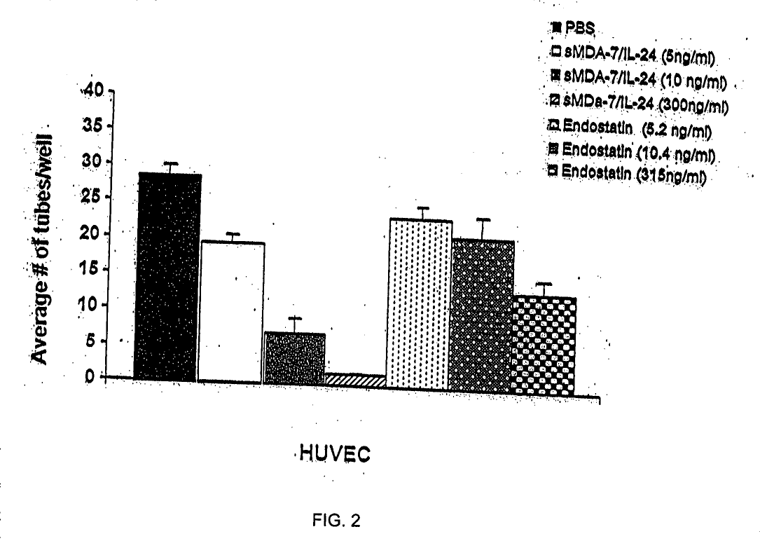

The inhibitory activity demonstrated by sMDA-7 was compared with endostatin in tube-formation assays. HUVEC were treated with equimolar concentrations of sMDA-7 or endostatin. sMDA-7 but not endostatin significantly (P=0.001) inhibited tube formation at low concentrations compared to control cells (FIG. 2). However, endostatin significantly inhibited tube formation (40-50% over control; P=0.001) compared to control cells at high concentrations (315 ng/ml), demonstrating that the endostatin protein used was functional (FIG. 2). These results indicate that sMDA-7 is a much more potent antiangiogenic agent than endostatin.

### Example 4

### sMDA-7 Inhibits Endothelial Cell Migration

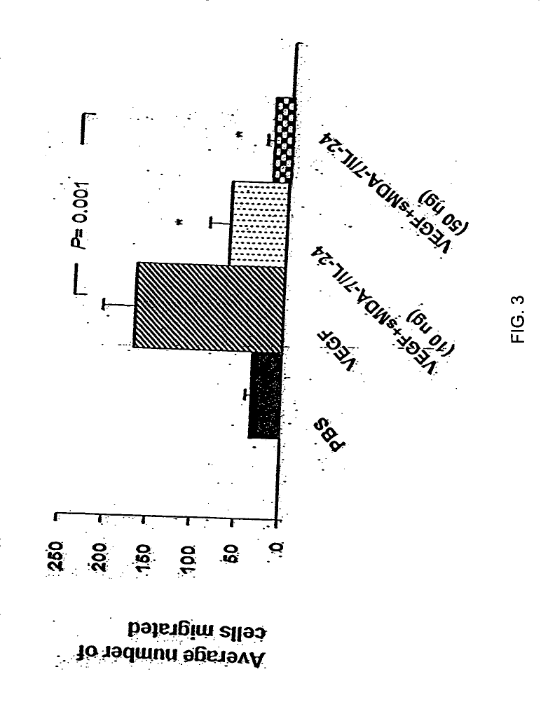

To determine if sMDA-7 inhibited endothelial cell migration, studies were conducted to examine the effect of VEGF on cell migration. sMDA-7 inhibited endothelial cell migration significantly (P=0.001) in response to VEGF (FIG. 3). No inhibitory effect on control cell migration was observed that did not contain sMDA-7/IL-24. Inhibition occurred in a dose-dependent manner, with complete inhibition occurring at 50 ng/ml (FIG. 3). sMDA-7/EL-24 demonstrated a similar inhibitory activity when bFGF was used as an inducer.

### Example 5

### Inhibition of Endothelial Cell Differentiation by sMDA-7 is Not Medicated by IFN-γ or IP-10

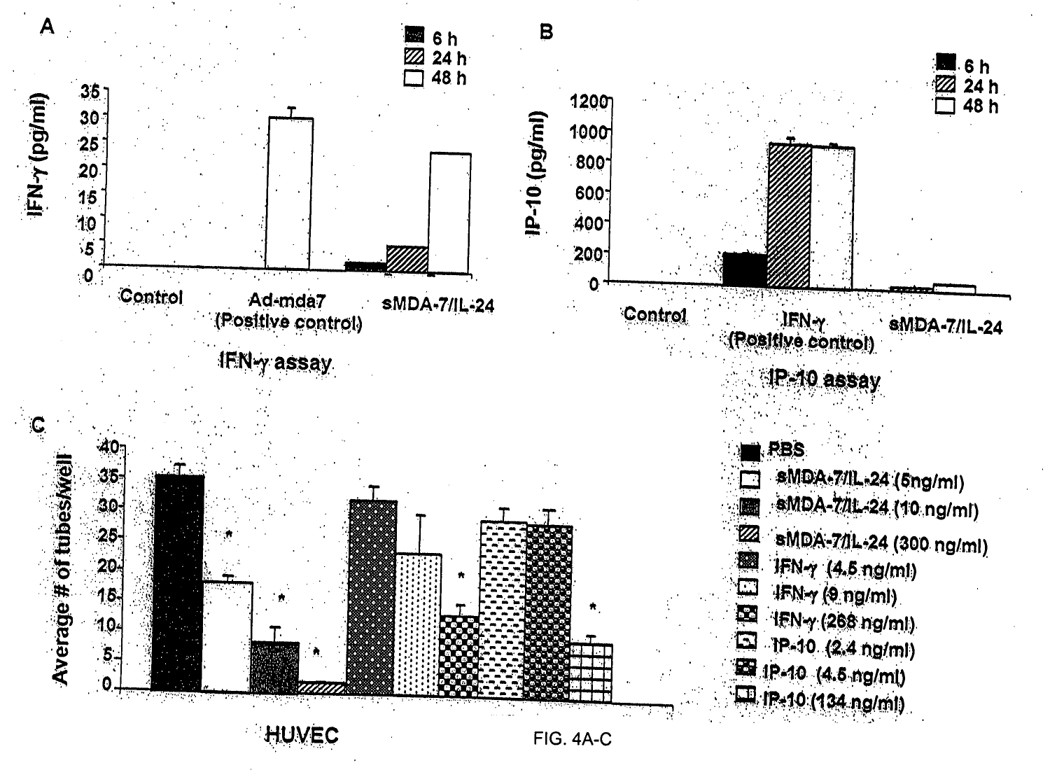

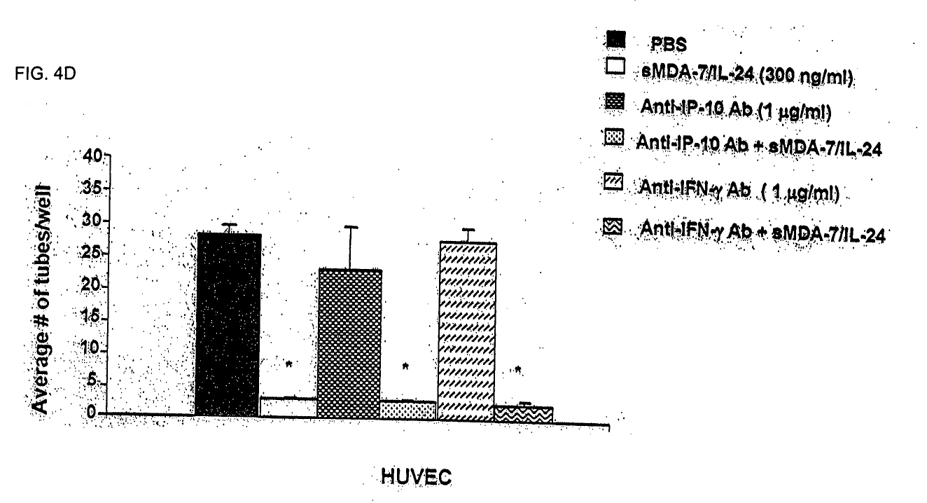

Production of IFN-γ by human PBMC upon treatment with sMDA-7 has recently been reported (Caudell et al., 2002). Based on this report, studies were conducted to evaluate whether inhibition of tube formation by sMDA-7 was mediated via WFN-γ, or IP-10 production. Tissue culture supernatants from PBS treated and sMDA-7 treated HUVEC cells were collected at various times and analysed for IFN-γ and IP-10 by ELISA. sMDA-7 induced secretion of IFN-γ (<30 pg/ml) and IP-10 (<32 pg/ml) in a 48 h period compared to control cells (FIG. 4A, FIG. 4B). To further test whether the low amounts of IFN-γ or IP-10 induced by sMDA-7 was responsible for the observed inhibitory effects on HWEC tube formation comparative studies were performed. A direct comparison of the inhibitory activity of sMDA-7 with IFN-γ or IP-10 at equimolar concentrations showed that higher concentration of IFN-γ. (268 ng/ml) or IP-10 (134 ng/ml) was required compared to sMDA-7 (10 ng/ml) to significantly inhibit HUVEC tube formation (P=0.01; FIG. 4C). Additionally, the inhibitory activity of sMDA-7 on HUVEC tube formation was not lost in the presence of anti-IP-10 or anti-IFN-γ neutralizing antibodies (P=0.001; FIG. 4D). These results indicate that sMDA-7 is more potent than IFN-γ and IP-10 in vitro and that sMDA-7 mediated inhibitory activity on HUVEC tube formation is not due to IFN-γ or IP-10.

### Example 6

### sMDA-7 Activates STAT-3 Expression and Mediates Its Inhibitory Activity Via Its Receptor

1. sMDA-7/IL-24 Activates STAT-3 Expression

Recent studies have demonstrated the activation of STAT-3 in HACAT cells and in PBMCs upon receptor engagement by sMDA-7 (Dumoutier et al., 2001; Wang et al., 2002). Based on these reports, it is hypothesized that activity of sMDA-7 on endothelial cells was receptor-mediated and upon receptor binding would activate STAT-3. Western blot analysis and immunofluorescence assay showed that the addition of sMDA-7 to HUVEC increased the expression levels of the phosphorylated form of STAT-3 (pSTAT-3) protein in as little as 4 h, and it persisted even at 24 h after treatment. The increase in pSTAT-3 expression was 2-3 times higher than in PBS treated control cells. There was also increased nuclear localization of pSTAT-3 protein in HUVEC after treatment with sMDA-7/IL-24. In contrast no changes in STAT-3 expression were observed in untreated control cells. Furthermore, STAT-3 activation was inhibited in the presence of anti-MDA-7 antibody indicating receptor-mediated activation.

2. sMDA-7 Mediates Its Inhibitory Activity Via Its Receptor

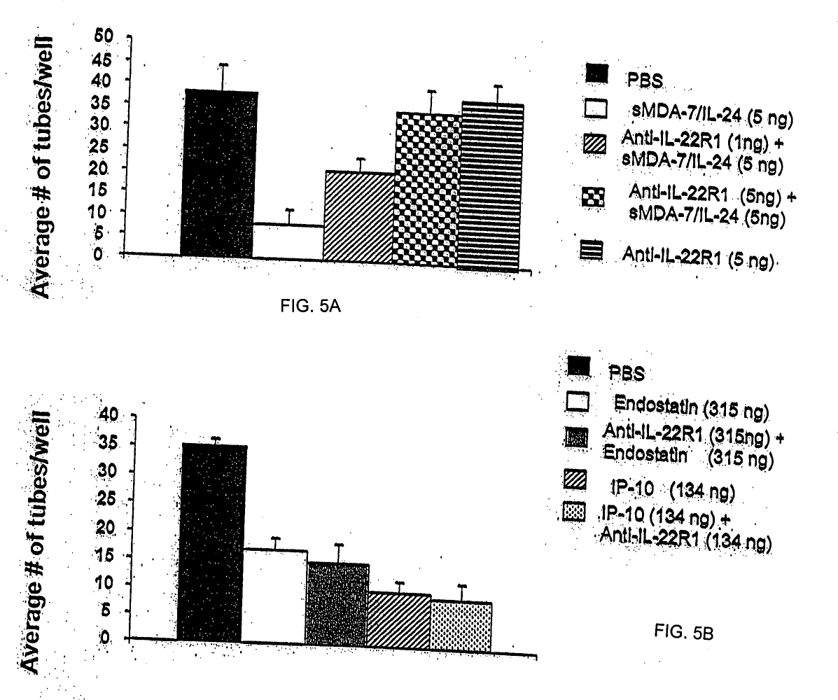

Two related receptors for sMDA-7 have recently been identified (Dumoutier et al., 2001; Wang et al., 2002). sMDA-7 can bind to either of the two receptor complexes, IL-20R1/IL-20R2 (IL-20 receptor) and IL-22R1 and IL-20R2 (IL-22 receptor). Based on these reports, studies were performed to determine whether the sMDA-7 mediated inhibitory effects on endothelial cells was receptor-mediated. Endothelial differentiation using a blocking antibody against IL-22R1 in the presence or absence of sMDA-7 was evaluated (FIG. 5A, FIG. 5B). sMDA-7 (5 ng/ml) alone completely inhibited tube formation in HUVEC, whereas no inhibition was observed in untreated control cells (FIG. 5A). However, pretreatment of HUVEC with IL-22R1 blocking antibody significantly (P=0.001) abrogated the inhibitory effects of sMDA-7 on tube formation, and in a dose-dependent manner (FIG. 5A). The addition of 1 ng/ml of blocking antibody to HUVEC (1:5 ratio) only partially restored tube formation (<60%), whereas the addition of 5 ng/ml of the blocking antibody (1 :1 ratio) completely restored it (>90%). Blocking antibody alone did not significantly affect the ability of HUVEC to form tubes. Furthermore, pSTAT-3 protein expression increased significantly after sMDA-7 protein was added to HUVEC, whereas sMDA-7-mediated pSTAT-3 expression did not increase in the presence of IL-22R1 antibody. These results indicate that sMDA-7 mediated its inhibitory effect on endothelial cell tube formation occur via IL-22R1.

To test the specificity of this inhibition, HUVEC were treated with high concentrations of IP-10 or endostatin in the presence of IL-22R1 antibody. Treatment with IP-10 or endostatin significantly inhibited HVEC tube formation even in the presence of IL-22R1 antibody (P=0.001; FIG. 5B) compared to PBS treated control cells. These results demonstrate that IL-22R1 antibody specifically inhibited sMDA-7/IL-24-mediated activity but not that of endostatin, IFN-γ or IP-10.

### Example 7

### In Vivo Models to Study Angiogenesis and Tumor Growth

1. sMDA-7 Inhibits Angiogenesis in the Matrigel Plug Model

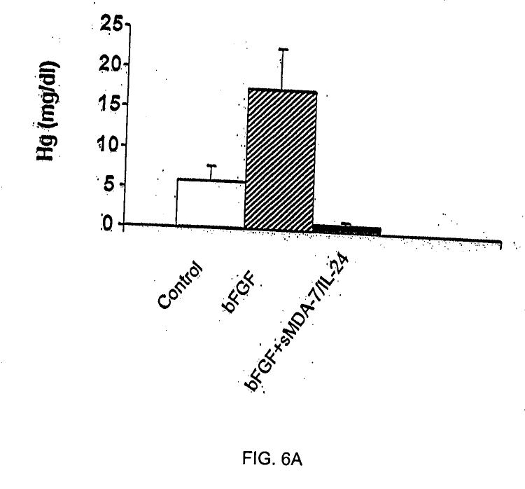

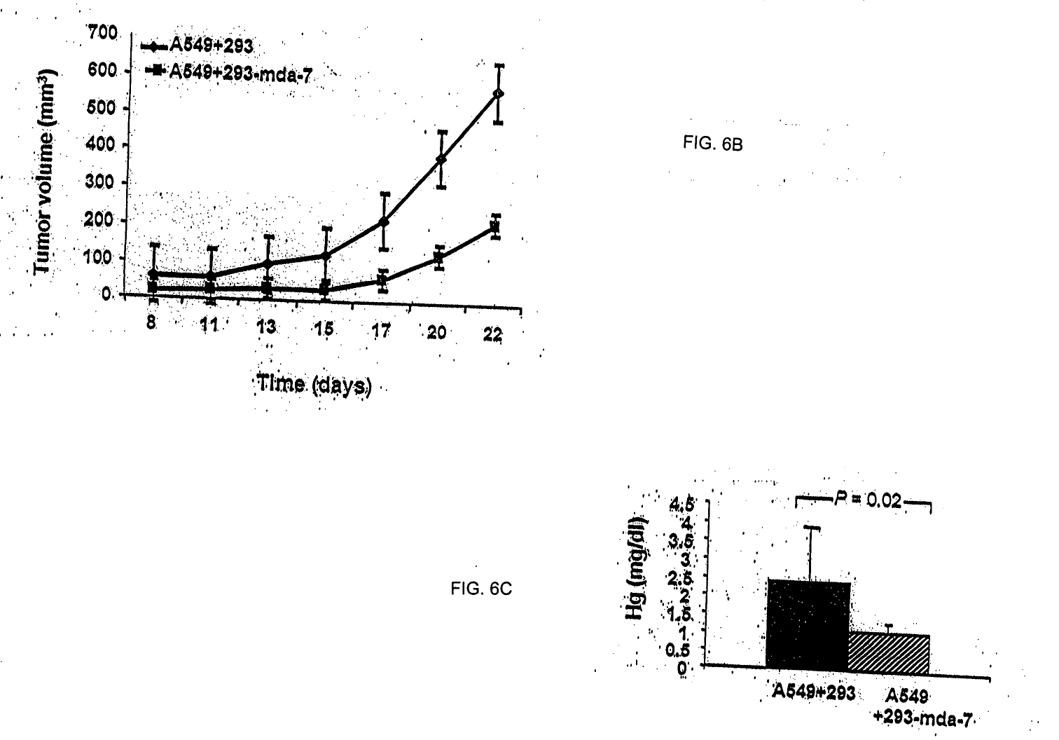

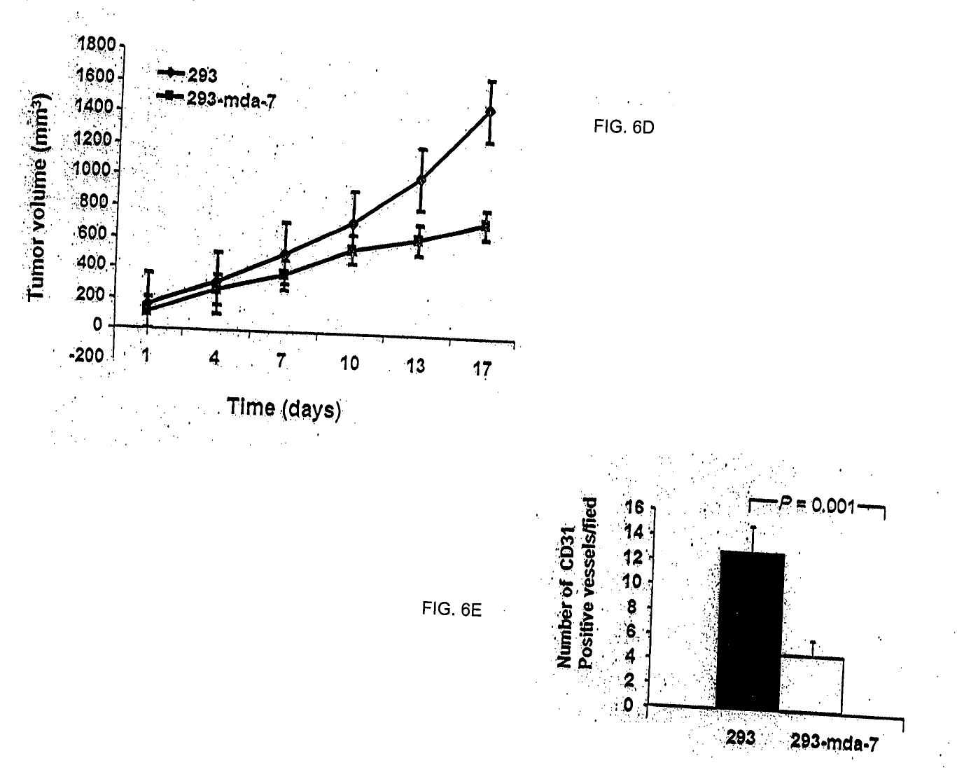

sMDA-7 encapsulated into Matrigel containing bFGF was implanted subcutaneously into nude mice. Matrigel containing bFGF alone and Matrigel containing PBS served as positive and negative controls respectively. bFGF-induced angiogenesis was significantly inhibited in the presence of sMDA-7/IL-24 (P=0.0001; FIG. 6A) when compared to Matrigel that contained only bFGF and Matrigel that contained PBS.

2. sMDA-7 Inhibits Subcutaneous Xenograft Tumor Growth In Vivo

Human lung tumor (A549) cells were mixed (1:1 ratio) with either parental 293 cells (control animals) or 293 cells producing sMDA-7 protein (293-mda-7) and injected subcutaneously into the lower right flanks of mice. Tumor growth was significantly less in the animals that received a mixture of A549 and 293-mda-7 cells (FIG. 6B) than in the animals that received a mixture of A549 and parental 293 cells (P=0.001). Injection of 293 or 293-mda-7 cells alone did not form tumors in nude mice. The animals were euthanized on day 22 postimplantation, and the tumors were harvested and further evaluated. Western blot analysis demonstrated that MDA-7 protein was expressed in tumors that contained 293-mda-7 cells. No MDA-7 protein expression was detected in the control tumors that contained parental 293 cells. Histopathologic examination of the tumor tissues did not reveal any significant differences in the tumor cell proliferative index or tumor cell infiltration between the control and experimental animals. However, tumors that contained 293-mda-7 cells demonstrated less vascularization by CD31 staining than did control tumors that contained parental 293 cells. TUNEL staining of tumor tissues from experimental animals demonstrated endothelial cells and tumor cells undergoing apoptotic cell death. In contrast, no TUNEL-positive staining was observed in control tumor tissues. Additionally, hemoglobin level was significantly (P=0.02) lower in tumors that contained 293-mda-7 cells than in the tumors that contained parental 293 cells (FIG. 6C). Reduction in CD31 staining and decreased hemoglobin levels indicated sMDA-7 inhibited angiogenesis.

3. sMDA-7 Systemically Inhibits Subcutaneous Xenograft Tumor Growth In Vivo

Studies were conducted to determine whether sMDA-7 produced by 293-mda-7 cells can systemically inhibit tumor growth. Mice were inoculated subcutaneously with A549 tumor cells in the lower right flank. When the tumors reached 50-100 mm³, 293 cells producing sMDA-7 protein (293-mda-7 cells) or parental 293 cells (control) were encapsulated in Matrigel and implanted subcutaneously in the upper right flank. Tumor measurement was initiated after implantation of 293 cells. The growth of A549 lung tumor xenografts was significantly less (P=0.001) in the mice treated with 293-mda-7 cells than in the control group (FIG. 6D). Compared with tumor growth in the control mice, the growth of the tumors in mice implanted with the encapsulated 293-mda-7 cells was suppressed by 40-50%. To confirm that the inhibitory effect was due to sMDA-7, serum samples from animals were tested for MDA-7 protein by western blot analysis and ELISA. Intense banding of sMDA-7 at the expected 40-kDa size was observed in the serum of animals implanted with 293-mda-7 cells by western blot analysis. However, faint bands were also observed in the serum of control animals, indicating some cross-reactivity with mouse serum proteins. The serum levels of circulating sMDA-7 detected by ELISA 3 days postimplantation was approximately 50 ng/ml.

At the end of the experiment, tumors and injected Matrigel containing 293-mda-7 cells were harvested and evaluated. Gross examination of the tumors indicated that the tumor growth in animals that received 293-mda-7 cells was inhibited. Histopathologic analysis of the tumor tissues demonstrated no differences between the specimens from animals receiving 293 cells and those from animals receiving 293-mda-7 cells. Additionally, the tumors from mice treated with 293-mda-7 were significantly (P=0.001) less vascular than were the tumors from mice treated with parental 293 cells, as evidenced by CD31-positive staining (FIG. 6E). Immunohistochemical analysis of the Matrigel from animals receiving 293-mda-7 cells demonstrated MDA-7 protein expression. In contrast, MDA-7 was not detected in the Matrigel recovered from animals receiving parental 293 cells. These results demonstrate that sMDA-7 systemically inhibited tumor growth by inhibiting angiogenesis.

### Example 8

### Ad-MDA-7 Induces Apoptosis and Activates the Immune System in Patients with Advanced Cancer

Study Design and Patient Criteria

In an ongoing Phase I dose-escalating clinical trial, mda-7 was administered via intratumoral injection to patients with advanced carcinoma using a non-replicating adenoviral construct (Ad-mda7). Patients had histologically confirmed carcinoma with at least one lesion that was accessible for needle injection that was surgically resectable, a Kamofsky performance status of ≧70%, and acceptable hemotologic, renal and hepatic function. Patients with active CNS metastases, chronic immunosuppressive use, or prior participation in a therapy requiring the administration of adenovirus were excluded from participation.

Patients with surgically resectable advanced cancers received single intratumoral injections of 2×10¹⁰ to 2×10¹² viral particles (vp) (FIG. 7). To date, eight cohorts (18 patients) have completed enrollment. To characterize the effects of intratumoral mda-7 treatment, injected lesions were surgically excised at 24-96 h post-injection, serially sectioned, and analyzed for vector DNA and RNA distribution, morphology, MDA-7 protein expression, apoptotic activity, microvessel density, number of Ki-67 positive cells, as well as iNOS and β-catenin expression.

### Example 9

### Effects of Intratumoral Ad-MDA7

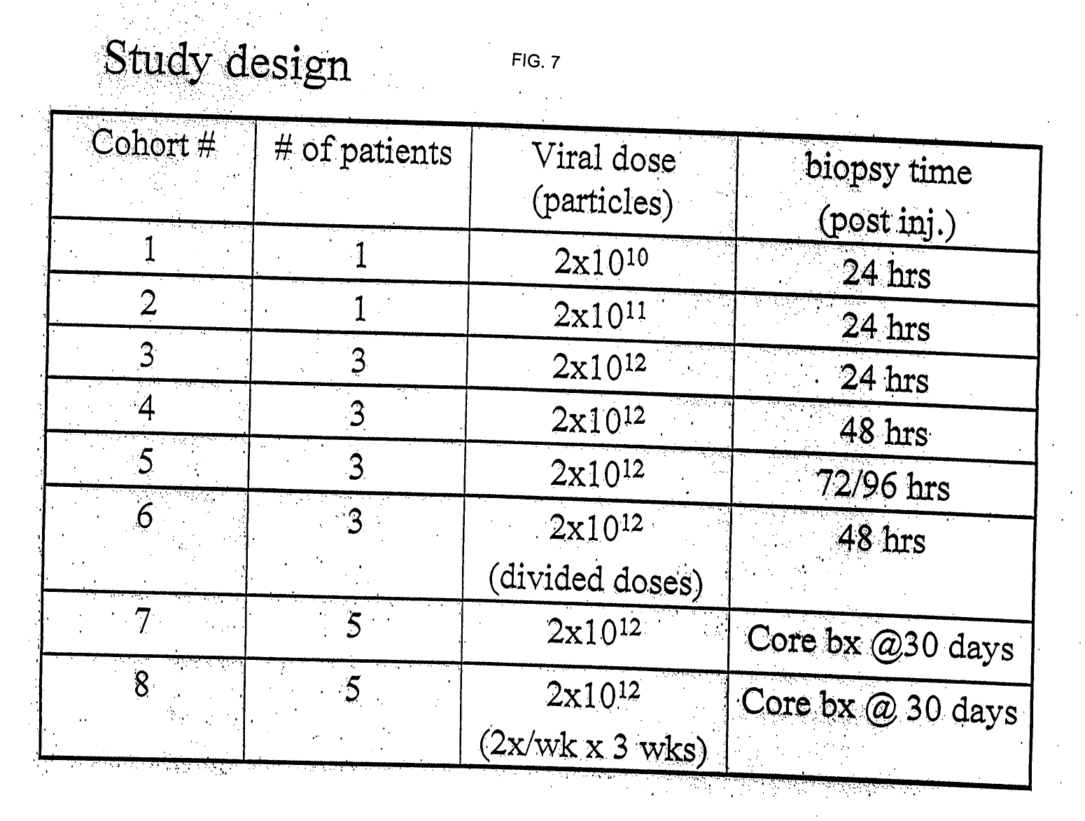

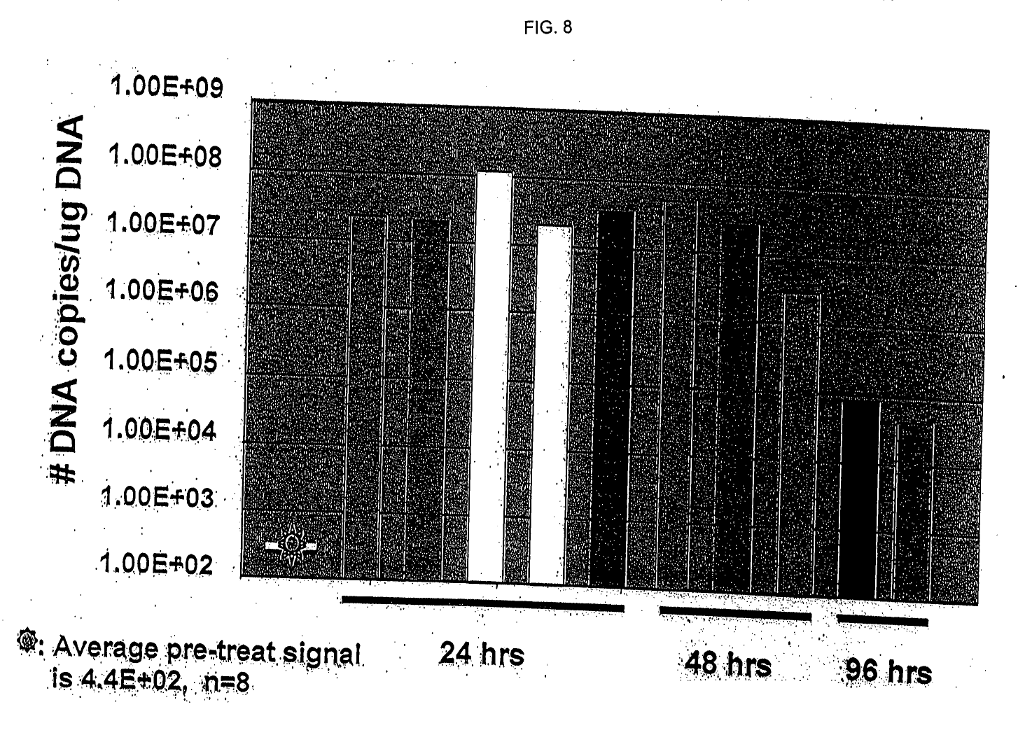

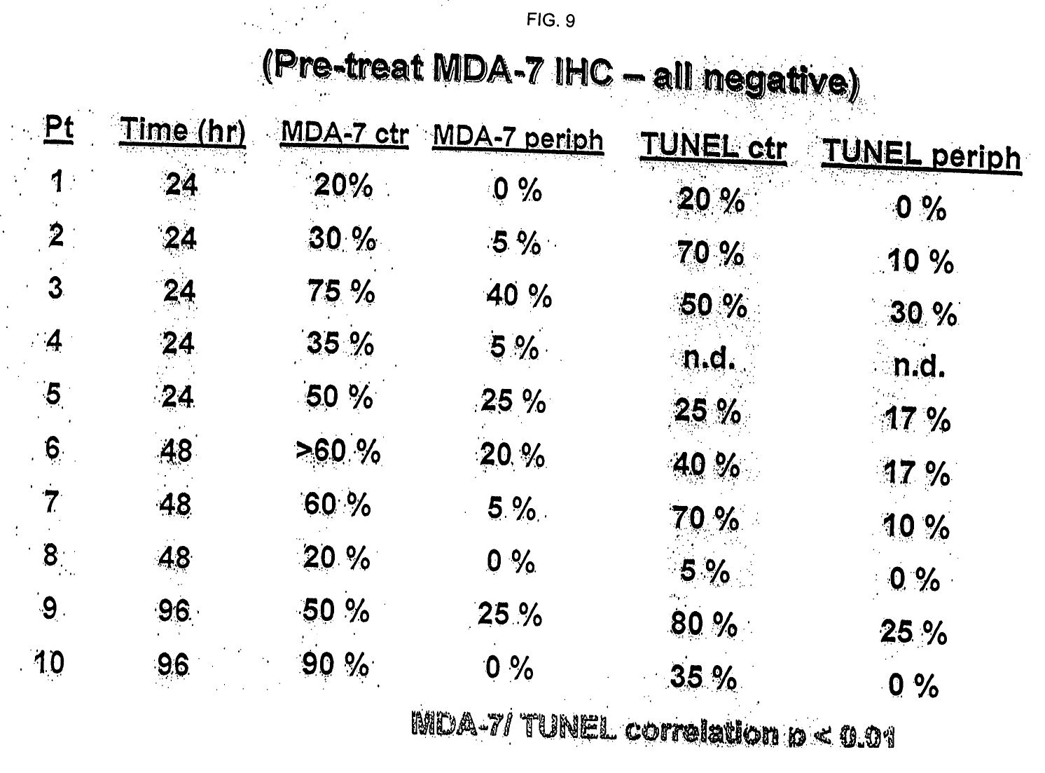

Ad-mda7 appears to be safe and well tolerated with pain at the injection site, transient low grade fever and mild flu-like symptoms being the primary toxicities. These effects were seen more consistently with higher doses of Ad-mda7. All effects resolved by 48 hours post injection. By DNA PCR analysis, Ad-mda7 copy number ranged from 7×10⁶/μg DNA in low dose treated patients to up to 4×10⁸/μg DNA in patients who received high dose (FIG. 8). The highest vector copy number was located at the center of the injected lesion, although vector DNA could routinely be detected in sections up to 1 cm from the injection point. mRNA distribution mirrored DNA distribution. By IHC analysis, strong MDA-7 protein expression was found in all injected lesions. Up to 80% of MDA-7 positive cells were found at the center of the high dose-injected tumor, as compared with up to 20% positive staining cells following low dose injection. Non-injected controls were uniformly negative. Further, areas of MDA-7 expression exhibited increased apoptotic activity as defined by TUNEL staining. Apoptosis was most intense in the center of the lesions, with up to 70% of cells being positive; while sections in the periphery also showed a heightened TUNEL reaction compared with uninjected lesions (FIG. 9). A marked reduction and/or redistribution of β-catenin expression from the nucleus to the plasma membrane was seen in 8 of 8 Ad-mda7-treated tumor lesions tested and was consistent with preclinical findings. Markedly reduced iNOS expression was also observed in the limited number of melanoma cases entered into the trial. Microvessel density decreased near the injection site but was difficult to quantify. Thus, Ad-md17 intratumoral injections are well telerated. Within 24 hours of injection there is a dose-dependent increase in MDA-7 protein expression and a marked increase in apoptotic cells, which correlates with distance from the injection site. By 72 to 96 hours, MDA-7 expression and apoptosis are decreasing (FIG. 8). By 30 days post injection, MDA-7 expression and apoptotic activity have ceased.

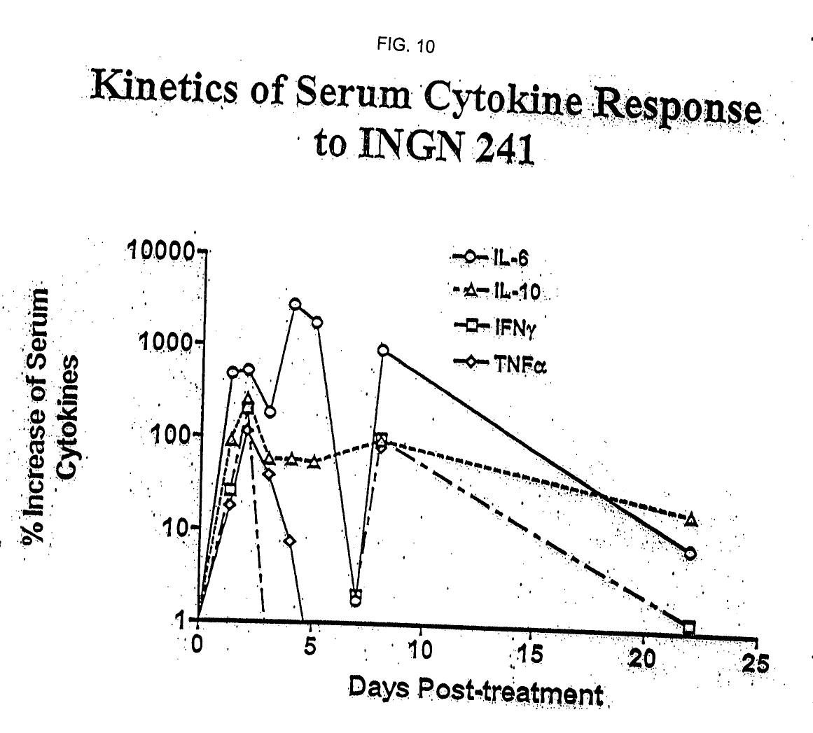

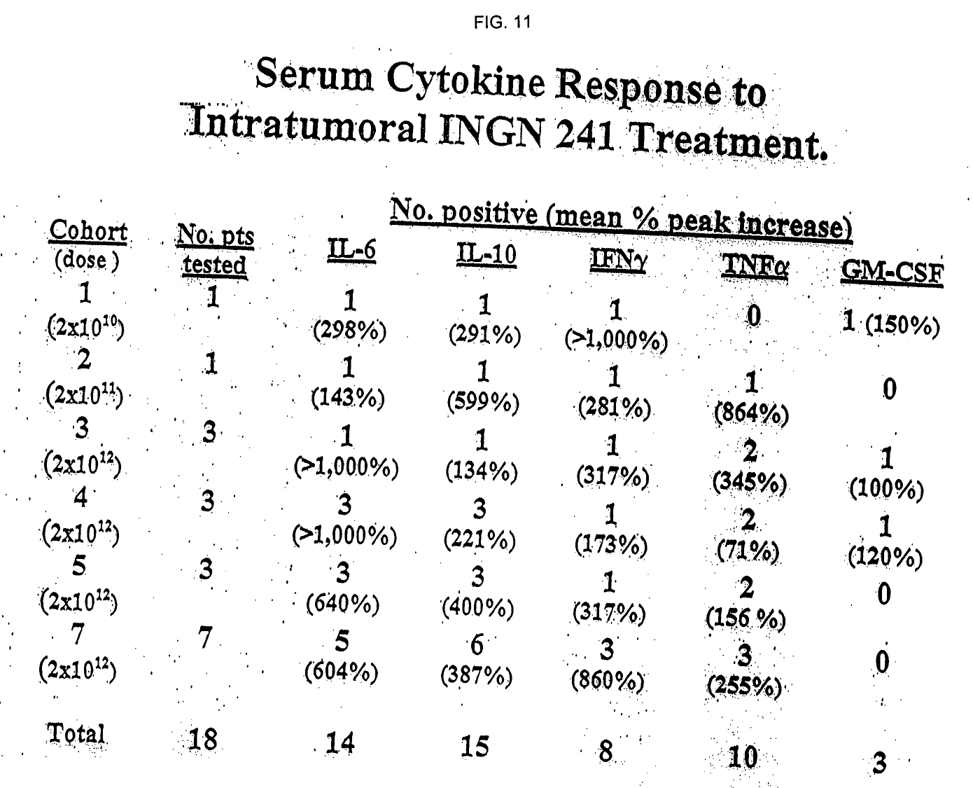

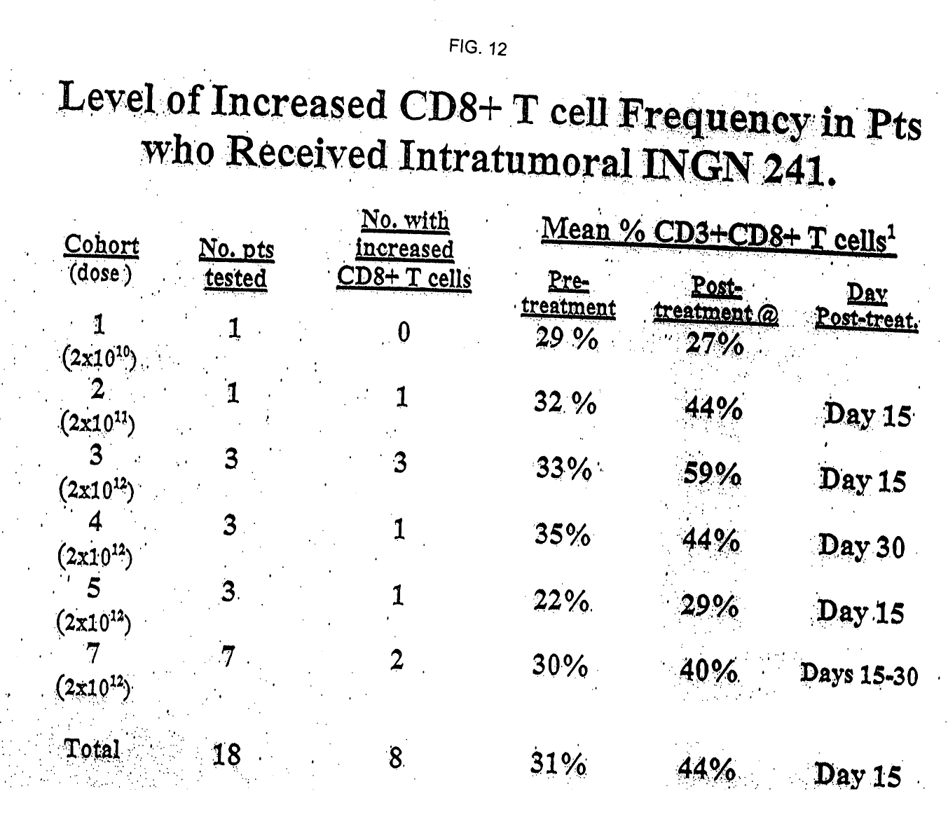

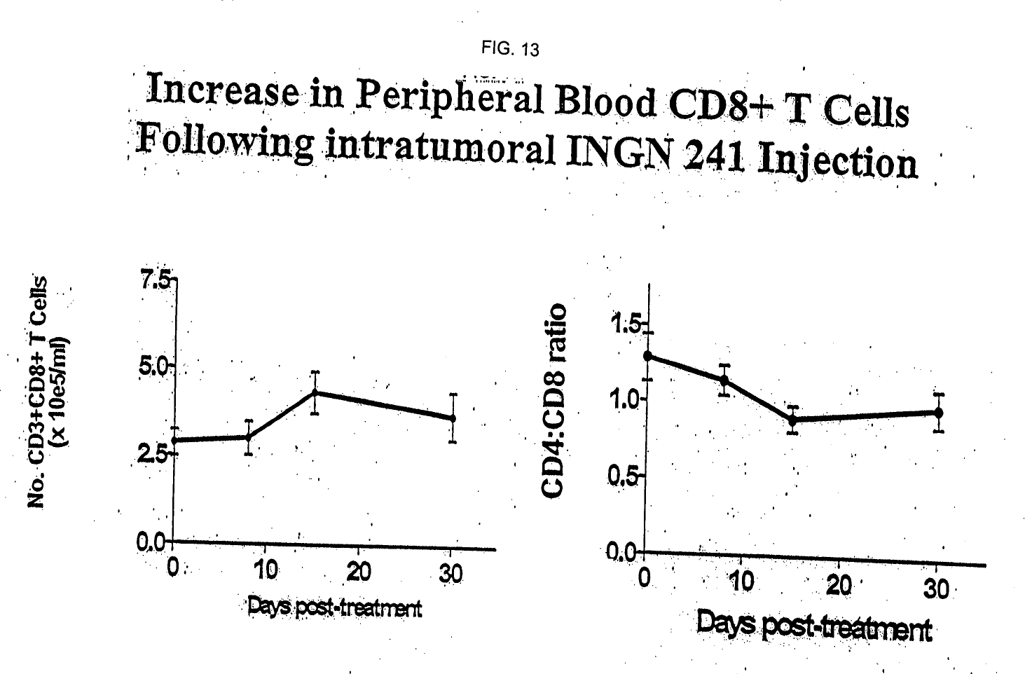

Systemic immune responses to Ad-mda7 were analyzed via serum cytokines and lymphocyte subsets. A majority of patients exhibited transient increases in systemic cytokines (IL-6, 14/18 patients tested; IL-10, 15/18; γIFN, 8/18; TNFα, 10/18) (FIG. 10, FIG. 11). Some high dose patients also exhibited increased intratumoral expression of IL-6, γIFN and IL-10 cytokine mRNAs. Further, CD3+ CD8+ T cells were increased by 30±13% at day 15 following mda-7 treatment (FIG. 12, FIG. 13). These findings suggest that MDA-7 increases systemic T_{H}1 cytokine production and mobilizes CD8+ T cells. After Ad-mda7 injection, circulating IL-6, IFN-gamma, IL-10 and TNF-alpha substantially increased and then fell to baseline levels by day 30. Cytokine increases correlate with increases in CD8+ cells and inversion of CD4/CD8 ratios. Thus, the results suggest immune activation by Ad-mda7 and is consistent with the pro-TH1 activity of rhMDA-7 in culture.

### Example 10

### Antibody Production

Recombinant his-tagged MDA-7 protein was produced in E. coli and was purified on a nickel NTA agarose column. The material was bound to the nickel resin in a batch mode for 45 minutes and then poured into a column and the eluate was run through the column bed. The material was washed with 10 mM Tris pH 8.0 containing 0.5% chaps and finally eluted off of the column with 10 mM Tris pH 8.0 plus 400 mM imidazole. The eluted MDA-7 was dialyzed against 10 mM Tris pH 8.0. The final product was shown to be a single band with a molecular weight of approx. 23 kDa. The amino terminal protein sequence was shown to be correct and purity was estimated to be greater than 90%.

This material was injected into rabbits using the following protocol: 400 mg MDA-7 protein with IFA and 100 mg of MDP was injected subcutaneously, 3 weeks later 200 ug MDA-7 protein with IFA was injected and 3 weeks after that another 100 mg of MDA-7 protein was injected intravenously. The titer of antiserum was shown to be greater than 1/100,000 based on an ELISA assay. Animals were boosted as needed.

The MDA-7 protein was coupled via sulfhydryl linkage to a solid support resin. The resin and bound protein was thoroughly washed. This washed material was used to make an MDA-7 column for antibody purification. The rabbit polyclonal sera was diluted 1:1 with 20 mM Tris buffer pH 8.0 and filtered through a 0.2-micron filter before being pumped onto the MDA-7 column. The column was then washed with the same 20 mM Tris buffer pH 8.0 until the absorbance returned to baseline. The antibody was eluted off the column with 0.1 M acetic acid. The eluent containing the antibody was immediately adjusted back to pH 8.0. This affinity-purified antibody was then dialyzed against 10 mM Tris pH 8.0 and concentrated.

### Example 11

### Purification and Characterization of Secreted MDA-7 Using Polyclonal Antibodies

1. Affinity Column Production

Different polyclonal antibodies against human MDA-7 from rabbit serum were first purified. Frozen rabbit serum samples were thawed and diluted 1:1 with sterile 1× PBS buffer. The diluted samples were individually exposed in bath method at 4° C. overnight with gentle rocking to 2 mls Protein A-Sepharose (SIGMA). Four different columns were generated. The resin was washed with 10 column volumes of 20 mM sodium phosphate dibasic (61 mls) to make a pH of 7.0 The column was eluted with 3 column volumes of 0.15 M NaCl (pH 3.0) in three aliquots and neutralized with 0.5M HEPES. A Bradford Protein Assay (BioRad) was used to quantify the eluted antibody. The antibody was then exchanged into 0.1 M NaHCO₃ (pH 8.3) containing 0.5 M NaCl, by dialyzing overnight in a 10,000 MWCO dialysis cassette.

To activate the dried CNBr-Sepharose, 1 gram was washed with 10-15 column volumes with 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. In each case, approximately 80-90 milligrams of antibody was recovered after purification and buffer exchange. Then 5 mls of swollen activated CNBr-Sepharose was incubated with 80-90 milligrams of purified antibody in 0.1 M NaHCO₃, pH 8.3, for 4 hours at room temperature with gentle rotation.

Antibody binding efficiency was determined by Bradford Protein assay, and in each case was greater than 95% of the antibody bound to the activated CNBr-Sepharose. After coupling, non-reacted groups were blocked by washing 25-30 column volumes in 0.1 M Tris, pH 8.0. The column was then washed with serial washes of 0.1 M Tris, pH 8.0, 0.5 M NaCl, 5× column volumes 5 times, alternating with 0.1 M acetate buffer, pH 4.0, 0.5 M NaCl. Protein estimation was performed on the washes and no protein was detected.

2. Affinity Chromatography Purification

Stably transfected 293 T cells that secrete soluble, glycosylated MDA-7 were obtained and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycine, (20 mg/ml stock). Then 400 mls of supernatant was harvested every 2-3 days and concentrated with an AMICON stirred cell over a 10,000 molecular weight cutoff membrane. 50 mls of concentrated supernatant was exposed in batch method to 5 mls bed volume of antibody-CNBr-sepharose, (affinity resin) for 2 days at 4° C. with gentle rocking. The affinity resin was then placed in a Pharmacia XK 26 column and the supernatant passed through three times to ensure maximum binding of antigen to antibody. The affinity resin was washed with 5×20 mls 0.1 M Tris pH 8.0 by gravity flow. MDA-7 was eluted with 3×5 mls 1 M NaCl, 0.1 M Glycine, pH 3.0 and immediately neutralized with 0.5 mls HEPES buffer. Immediately after elution and neutralization, 2 mgs of human albumin was added to protect against protein loss. The eluted protein was then concentrated over 10,000 molecular weight cutoff spin columns (AMICON), and exchanged into sterile 1× PBS. Then 1-1.5 mls of 1× PBS exchanged affinity purified protein was exposed to 200 microliters 3× washed Protein-A Sepharose (SIGMA) for 2 hours at room temperature with rotation, or over night at 4° C. with rotation. Protein A exposure absorbs antibody that leaches into the elution fraction.

Four different polyclonal antibodies, whose production is described herein, were tested in affinity purification. Size resolution purification (see Size Exclusion) was employed to removed significant contaminating protein from the supernatant prior to affinity purification, the most abundant of which was bovine serum albumin (BSA). However, exposure of MDA-7 isolated in this fashion failed to permit the antibody on the column to retain MDA-7. This was probably due to BSA blocking non-specific binding sites that could retain MDA-7 in the absence of BSA. MDA-7 is a highly glycosylated protein it is considered very capable of sticking to plastic and other surfaces.

Removal of BSA from MDA-7 containing supernatant inhibits purification of MDA-7 by affinity chromatography. Most protein was present in the flow through. No MDA-7 protein is retained on the affinity column until elution. Affinity purifications that contained significant amounts of BSA, (2-3 mgs/ml by silver stain) retained biological function for longer than the purifications wherein the BSA contamination was significantly less. Affinity purification in the presence of BSA permits the retention of MDA-7 on the affinity column until elution with high molar NaCl and low pH. Affinity purification by polyclonal affinity resin resulted in multiple lots with relatively similar amounts of MDA-7. Coomassie analysis indicated relatively low quantities of contaminating protein. Purification of MDA-7 of greater than about 20% homogeneity was observed.

Affinity purification was repeatable and enriched the MDA-7 to relative purity by coomassie stain analysis of 12% polyacrylamide gels. By intensity of bands detected on the Western blot, more MDA-7 was retained with longer exposure of the antigen to the affinity resin. There was little difference between the method of exchange into 1× PBS, when comparing the dialysis cassette and the spin columns.

3. Anion Exchange Purification

Two to three lots of affinity purified MDA-7 were pooled and exchanged into 50 mM MES, pH 5.0 in a 10,000 MWCO dialysis cassette from 2-12 hrs at room temperature. Protein was then loaded onto a 5 ml bed volume anion exchange column at a flow rate of 1 ml/minute. 10 mls of flow through were taken and the bound protein was eluted with a step gradient of 1 M NaCl in 50 mM MES, pH 5.0. The elution program began with a 10 ml wash of 50 mM MES, pH 5.0 at flow rate of 2 mls/min. The first step elution was from 0 M to 0.25 M NaCl in 5 minutes with a 5 minute wash at 50 mM MES, 0.25 M NaCl, pH 5.0. The second gradient step was from 0.25 M NaCl to 0.5 M NaCl in 5 minutes followed by a 5 minute wash. The final elution was from 0.5 M NaCl to 1 M NaCl. MDA-7 was retained on to column until elution with 0.9-1.0 M NaCl; MDA-7 was purified to about 90%-95% homogeneity.

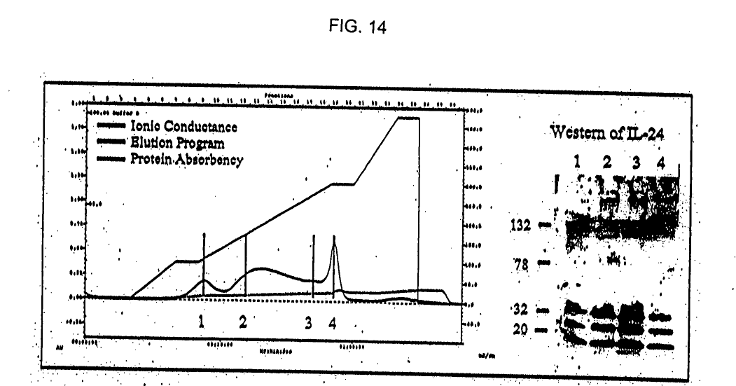

The unglycosylated protein of 18 KDa did not bind to the anion exchange column at pH 5.0. Silver stain analysis of fractions from post-affinity anion exchange of MDA-7 revealed that the unglycosylated form of MDA-7 is not associated with the co-purifying glycosylated proteins. The native MDA-7 complex appears to contain at least three proteins of molecular weight 31, 28 and 27/26. Previously, an attempt was made to purify MDA-7 utilizing a one step anion exchange purification, wherein the supernatant containing MDA-7 was exchanged into 50 mM MES, pH 6.0. One step anion exchange purification demonstrated that each peak from the anion exchange column contains MDA-7 detected by polyclonal anti-MDA-7 on western blot (FIG. 14). Purification by this method failed to significantly enrich for MDA-7 at any range of ionic strength, as MDA-7 leached from the column at all molarities of NaCl employed.

4. Size Exclusion Chromatography

A 200 ml bed volume size exclusion chromatography column was generated utilizing S200 Sephadex (Pharmacia) poured into an XK 26 1 meter column (Pharmacia). The column was allowed to gravity settle, and was then packed at 3.5 mls/min with a BioRad BioLogic Workstation.

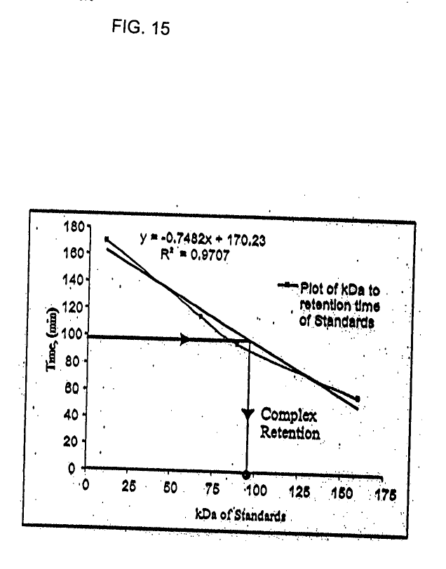

To determine the apparent molecular weight of MDA-7 secreted by the 293 t cells, protein molecule weight standards, (mouse IgG 5 mgs, Alkaline Phosphatase 3 mgs, BSA 10 mgs, and human beta2microglobulin 3 mgs) were combined to determine the relative retention times. Elution times of the purified proteins relative to molecular weights were plotted and an R² value of 0.97 derived. 200 mls of 293 t supernatant containing MDA-7 was concentrated over a 10,000 MWCO filter in an AMICON stirred cell down to 10 mls and loaded at 2 mls/min in 1× PBS on the size resolution column. Fractions were taken every 5 mls. Relative retention times was determined by Western blot analysis of sequential samples and compared to the line derived from the known standards. An apparent molecular weight of 80-100 kDa was assigned to the associated MDA-7. Less than 0.1% of the total MDA-7 present was found to be in monomeric 31 kDa form. FIG. 15 shows a comparison of retention time to molecular weight. MDA-7 complex was eluted at between a molecular weight of about 85-95 kDa.

6. Size, Anion, and Lectin Purification

Lectin purification over a ConcanavalinA-Sepharose column was employed in an attempt to purify MDA-7. However, no net increase in relative purity was achieved. Combinatorial purifications, wherein size exclusion, anion, and lectin purification methods, were utilized in all combinations to enrich for MDA-7. However, no combination of these methods provided for greater purification of MDA-7 than affinity chromatography followed by anion chromatography. These results demonstrate that MDA-7 can be purified to at least 90-95% homogeneity by affinity and anion exchange chromatography.

### Example 12

### Purification and Characterization of Secreted MDA-7 Using Monoclonal Antibodies

1. Antibody Production

The hybridoma clone, designated 7G11F.2 (monoclonal antibody), was determined to produce antibody that was the most effective at detecting IL-24/mda-7 positive cells by intracellular FACS analysis of stably transfected 293 t cells that had been treated with Brefeldin A. Based upon these preliminary data, this clone was utilized to produce 5 liters of supernatant. Briefly cells, (7G 11F.2) were seeded at 1×10⁶ cells/ml in 50mls of DMEM supplemented with containing 10% Fetal Calf Serum with 1:100 L-glutamine, 1:100 pen/strep and 1:100 HEPES. Cells were seeded and permitted to grow for 10 days, then the supernatant was harvested.

2. Antibody Purification

Supernatant was clarified of cells by centrifugation at 2000 rpm for 10 minutes and decanted. The clarified supernatant was then sterile filtered over a 0.22 micro cellulose acetate filter and concentrated with an Amicon Stirred Cell under nitrogen over a YMCO 30 kDa membrane to 50 mls. The concentrated supernatant was exposed to rProtein G cross-linked to sepharose, (Sigma) o/n at 4° C. The antibody was eluted with 1 M NaCl pH 3.0, 3 column volumes in three aliquots and neutralize with 0.5 M HEPES. To remove contaminating bovine IgG, the resulting eluate was exchanged into 1× PBS containing 0.4 M NaCl (total), via dialysis cassette (Pierce/Endogen, YMCO 30 kDa). The protein was exposed to rProtein A crosslinked to sepharose, (Sigma) o/n 4° C. The flow through from the column was taken, as the protein A binds the bovine IgG with higher affinity than the mouse IgG1a. Relative purity was determined by analysis on SDS PAGE and taken to be 90% pure, (7G11F.2) with the contaminating protein wholy comprised of bovine IgG. Bradford Protein Assay, (BioRad), was used to quantify eluted antibody. The antibody was then exchanged into 0.1 M NaHCO₃, pH 8.3 containing 0.5 M NaCl, by dialyzing overnight in a 10,000 MWCO dialysis cassette.

3. Affinity Column Production

To activate dried CNBr-Sepharose, 1 gram was washed with 10-15 column volumes of 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. 25 mgs of antibody, (7G11F.2) was recovered after purification and buffer exchange. 2 mls of swollen, activated CNBr-Sepharose was incubated with the purified antibody in 0.1 M NaHCO3, pH 8.3 for 4 hours at room temperature with gentle rotation.

Antibody binding efficiency was determined by Bradford Protein Assay; greater than 95% of the antibody bound to the activated CNBr-Sepharose.

After coupling, non-reacted groups were blocked by washing 25-30 column volumes in 0.1 M Tris pH 8.0. Finally the column was washed with serial washes of 0.1 M Tris pH 8.0, 0.5 M NaCl, 5× column volume 5 times alternating with 0.1 M acetate buffer, pH 4.0, 0.5 M NaCl. Protein estimation was performed on the washes and no protein was detected.

4. Affinity Purification

Stably transfected 293 t cells that secrete soluble, glycosylated IL-24 were obtained from Introgen, Inc. and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycine, (20 mg/ml stock). 400 mls of supernatant is harvested every 2-3 days and concentrated with an Amicon stirred cell over a 10,000 molecular weight cutoff membrane. 50 mls of concentrated supernatant is exposed in batch method to 5 mls bed volume of antibody-CNBr-sepharose, (affinity resin) for 2 days at 4° C. with gentle rocking. The affinity resin was placed in a Pharmacia XK 26 column and the supernatant passed through three times to ensure maximum binding of antigen to antibody. The affinity resin was washed with 5×20 mls 0.1 M Tris pH 8.0 by gravity flow. IL-24 was eluted with 3×5 mls 1 M NaCl, 0.1 M Glycine, pH 3.0 and immediately neutralized with 0.5 mls HEPES buffer. Immediately after elution and neutralization, 2 mgs of Human Albumin was added to protect against protein loss. The eluted protein was then concentrated over 10,000 molecular weight cutoff spin columns, (Amicon) and exchanged into sterile 1× PBS. 1-1.5 mls of 1× PBS exchanged affinity purified protein was exposed to 200 microliters 3× washed rProtein-A Sepharose, (Sigma) for 2 hours at room temperature with rotation, or overnight at 4° C. with rotation. Protein A exposure absorbed antibodies that leached into the elution and its removal is crucial for maintaining IL-24 function.

The 7G11F.2 monoclonal antibody column retained similar amounts of IL-24/mda-7 as the polyclonal columns in Example 11.

### Example 13

### MDA-7 and Pancreatic Cancer Cells

1. Ad-mda7 Directly Kills and Radiosensitizes Pancreatic Cancer Cells

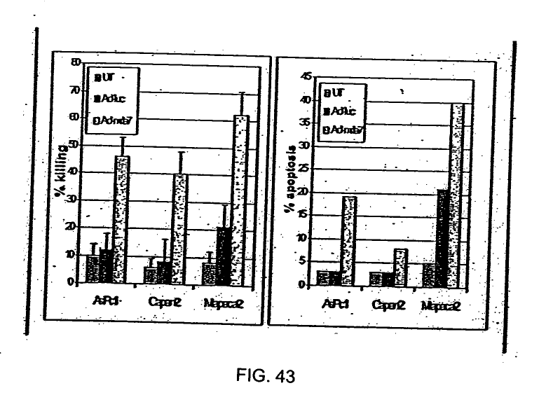

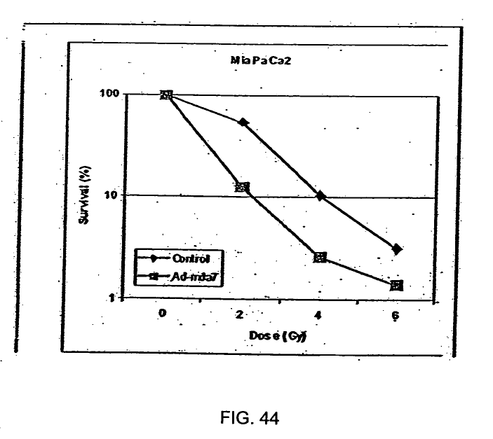

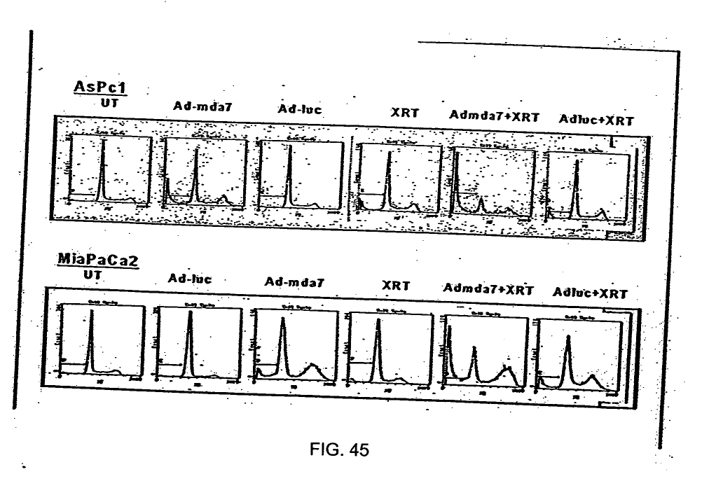

An assessment of 4 different pancreatic cancer cell lines indicated that most of these lines are highly infectable with Ad-mda7. In addition, Ad-mda7 was shown to directly kill and induce apoptosis in three of these four cell lines (FIG. 43). Two lines with the greatest response, MiaPaCa2 and AsPc1, were chosen for further analysis and different experiments were conducted to assess whether Ad-mda7 radiosensitized these cells. The MiaPaCa2 cells were pre-treated with Ad-mda7 and then irradiated them 48 hrs later. A dramatic radiosensitization was observed when clonogenic survival was the endpoint (FIG. 44). Finally, apoptosis was assessed on the basis of sub G1/G0 DNA content by FACS analysis (FIG. 45). This experiment showed a greater than additive induction of apoptosis when Ad-mda7 and radiation were combined.

2. MDA-7 Protein Activates STAT3 and Directly Kills Pancreatic Cancer Cells

MiaPaCa2 pancreatic cancer cells were treated with purified recombinant human MDA-7 protein, as described in Example 11. By immunofluorescence, substantial activation (phosphorylation) of STAT3 and concomitant movement of p-STAT3 to the nucleus were observed. The STAT3 activation was blocked in the presence of anti-MDA-7 antibodies. STAT3 activation was evident within 30 minutes of MDA-7 treatment, suggesting that MiaPaCa2 cells possess the receptor(s) for MDA-7, and ligand-receptor engagement occurred.

In another study, MDA-7 protein treatment induced dose-dependent killing of MiaPaCa2 cells. Pancreatic tumor cells possess the receptors for MDA-7, and upon MDA-7 binding, STAT3 signaling is induced which results in death of the tumor cell.

### Example 14

### Selective Induction of Cell Cycle Arrest and Apoptosis in Prostate Cancer Cells

Materials and Methods

1. Cell Lines and Cell Culture

The human prostate cancer cell lines DU 145, LNCaP, and PC-3 were obtained from American Type Culture Collection (ATCC; Rockville, Md., U.S.A.) and grown in RPMI-1640 medium with 10% fetal bovine serum, antibiotics and L-glutamine (GIBCO-BRL; Grand Island, N.Y., U.S.A.). The normal prostate epithelial cell line (PrEC) was obtained from Clonetics (San Diego, Calif., U.S.A.) and grown in PrEBM medium with supplements according to supplier's instructions.

2. Virus Construction and Transduction Efficiency

Construction and production of the replication-deficient adenoviral (Ad5) vector carrying the mda-7 or luciferase (luc) gene has been previously described (Saeki et al., 2000; Mhashilkar et al., 2001). Preliminary experiments using an adenoviral vector encoding green fluorescent protein (Ad-GFP) showed that an adenovirus dose delivered at a multiplicity of infection (MOI) of 3000 can infect more than 93.4% of DU 145 and PC-3 cells, 76.2% of LNCaP cells, and 82% of PrEC cells. Therefore, the inventors used Ad-mda7 or Ad-luc at an MOI of 3000 in all subsequent studies.

3. Cell Proliferation Assay

All of the cell lines were plated in six-well tissue culture plates at a density of 1 . 105 cells/well. Tumor cells were then treated with Ad-mda7 or Ad-luc or treated with 0.1 M phosphate-buffered saline (PBS) as a mock control. Cells in each treatment group were plated in triplicate and cultured for 4 days. Then, at designated time points, cells were harvested via trypsinization and stained with 0.4% trypan blue (GIBCO-BRL; Grand Island, N.Y., U.S.A.) to reveal dead cells. Viable cells were then counted using a hemocytometer. For apoptotic staining, cells were stained with Hoechst 33258 at 72 h after infection and analyzed as previously described(Saeki et al., 2000, 2002).

4. Cell-Cycle Analysis

To determine the effect of Ad-mda7 on cell cycle, cells were seeded in 10-cm culture dishes (5-10×10⁵ cells/dish) and treated with Ad-mda7, Ad-luc or treated with 0.1M PBS. At specific times after treatment, cells were harvested via trypsinization, washed once with ice-cold 0.1 M PBS, fixed with 70% ethanol and stored at −20° C. Cells were then washed twice with ice-cold 0.1 M PBS and treated with RNase (30 min at 37° C., 500 units/ml; Sigma Chemicals; St. Louis, Mo., U.S.A.), and DNA was stained with propidium iodide (PI) (50 μg/ml; Boehringer Mannheim; Indianapolis, Ind., U.S.A.). The cell-cycle phase and apoptotic rate (cells at sub-G0/G1 phase) were analyzed using a fluorescent activated cell sorter (EPICS XL-MCL, Beckman Coulter, Inc., Fullerton, Calif., U.S.A.).

5. Mitotic Index

For mitotic index determination, cells were harvested at 72 h after treatment with Ad-mda7. Cells were fixed and stained with PI as described for cell cycle analysis analyzed by fluorescence microscopy. For each sample, at least 500 cells were randomly counted at high magnification (×40) by fluorescence microscopy, and mitotic cells were visually identified by their lack of a nuclear membrane and by evidence of chromosome condensation.

6. Immunoblot Analysis

Tumor cells (DU 145 and LNCaP) treated with Ad-mda7, Ad-luc or PBS were harvested at 72 h after treatment and cell extracts prepared for western blot analysis as previously described (Saeki et al., 2000). The following antibodies were used as primary antibodies: Anti-MDA-7 antibody (Introgen Therapeutics, Inc., Houston, Tex., U.S.A.) caspase-3; PARP; and cyclin E (Pharmingen; San Diego, Calif., U.S.A.); cyclin A, β-actin (Sigma Chemicals; St. Louis, Mo., U.S.A.); NFkB, Chk1, Cdc2, phosphospecific-Jak1 p27Kip1, phosphospecific-JNK, phosphospecific-STAT3 (Santa Cruz Biotechnology; Santa Cruz, Calif., U.S.A.); phosphospecific-Tyk2, phosphospecific-STAT1, and Cdc25C (Cell Signaling Technology, Inc, Boston, Mass., U.S.A.); cyclin B1 (Lab Vision Corp., Fremont, Calif., U.S.A.); Chk2 (Novus Biologicals, Littleton, Colo., U.S.A.); p21WAF1 (Oncogene Research Products, Boston, Mass., U.S.A.).

7. Statistical Analysis

Student's t-test was used to calculate the statistical significance of the experimental results. The significance level was set at P<0.05.

Results

1. MDA-7 Expression Following Ad-mda7 Treatment in Human Prostate Cancer and Epithelial Cells

To detect exogenous MDA-7 expression in cells, DU 145, LNCaP, PC-3, and PrEC cells were grown in six-well tissue culture plates (1×10⁵ cells/well) and treated with Ad-mda7 and Ad-luc. Cells treated with PBS served as negative controls. At 24 h, 48 h and 72 h after infection, cell lysates were prepared and evaluated for protein expression by western blot analysis. MDA-7 expression was detected in all cell lines treated with Ad-mda7 compared to cells that were treated with PBS and with Ad-luc. MDA-7 protein expression was observed to be time dependent with maximum expression observed between 48 h to 72 h. No endogenous MDA-7 expression was detected in the cell lines tested.

2. Inhibition of Cell Proliferation in Prostate Cancer Cells Due to Overexpression of MDA-7

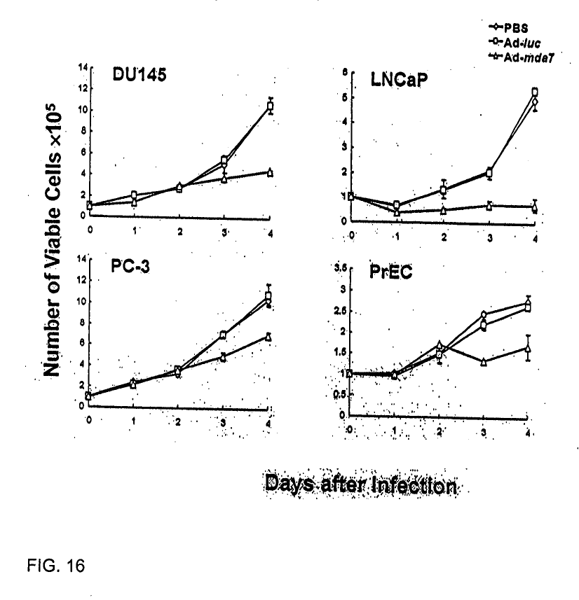

To determine the effect of Ad-mda7 treatment on cell proliferation, tumor cells (DU 145, LNCaP, and PC-3) and normal cells (PrEC) were treated with PBS, with Ad-luc, or with Ad-mda7. Cells were harvested at various time points after treatment and analyzed for cell viability. A significant inhibition of cell proliferation (P≦0.01) was observed starting from day 3 in all the cell lines treated with Ad-mda7 when compared with that in control cells treated with Ad-luc or treated with PBS. In PC-3 and PrEC cells, the suppression of cell proliferation was observed to less than that seen in DU 145 and LNCaP cells suggesting that these cells may be less sensitive to Ad-mda-7 (FIG. 16). However, analysis of cell proliferation at later time points (days 5 and 6) demonstrated PC-3 cells to be more sensitive to Ad-mda7 than PrEC cells.

3. Induction of Apoptosis in Prostate Cancer Cells due to Overexpression of MDA-7

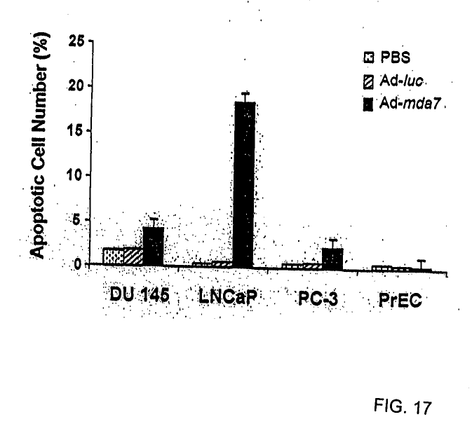

To determine whether treatment with Ad-mda7 induced apoptosis, cells treated with Ad-mda7, with Ad-luc, or with PBS were subjected to flow cytometric analysis. Tumor cells (DU 145, LNCaP, PC-3) treated with Ad-mda7 demonstrated an increase in the number of cells in sub-G0/G1 phase, an indicator of apoptosis when compared to cells that were treated with Ad-luc or with PBS (FIG. 17). However, the number of apoptotic cells (5-18%) varied between the tumor cell lines. In contrast, normal cells (PrEC) treated with either Ad-mda7 or Ad-luc did not demonstrate a significant change in the number of cells in sub-G0/G1 phase when compared to PBS treated cells. To further confirm these results, cells were stained with Hoechst 33258 at 72 h after infection. Tumor cells, not normal cells treated with Ad-mda7, demonstrated condensed and fragmented nuclei, an indicator of apoptosis. No changes were observed in any of the control cells treated with Ad-luc. These results indicate that MDA-7 may selectively inhibit tumor cells but not normal cells by inducing apoptosis.

4. Induction of G2 Cell Cycle Arrest due to Overexpression of MDA-7

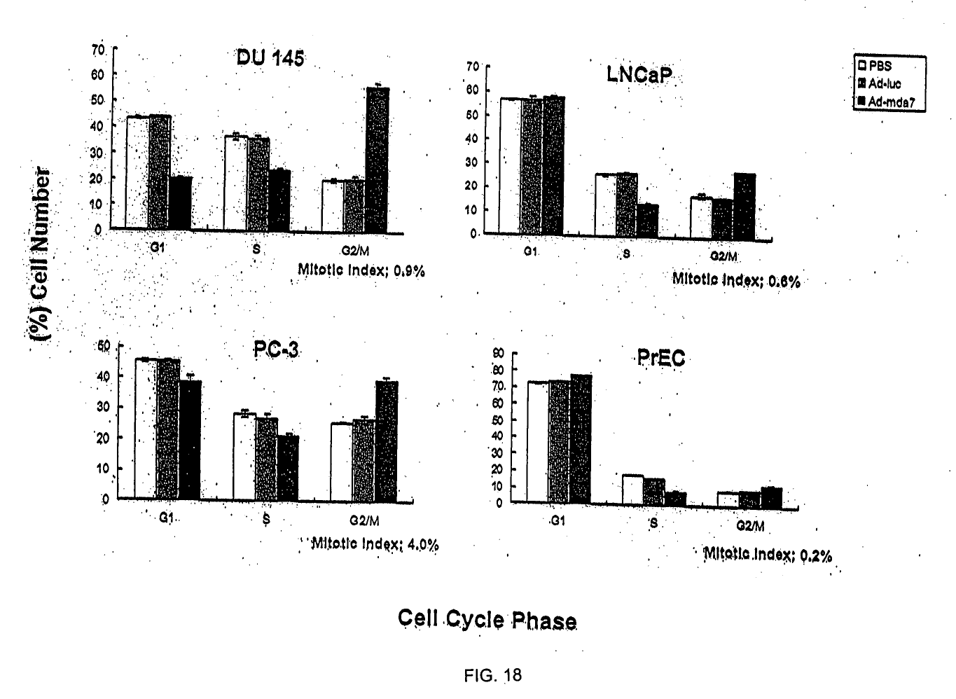

To determine whether MDA-7 is capable of inducing G2/M cell-cycle arrest in prostate cancer cells, as reported in previous studies of human lung, breast and melanoma cancer cell lines, (Saeki et aL., 2000; Mhashilkar et al., 2001; Lebedeva et al., 2002) cell-cycle phases were analyzed by flow cytometry. Cell-cycle analysis indicated an increase in the number of tumor cells in the G2/M population at 72 h after treatment with Ad-mda7 as compared with tumor cells treated with Ad-luc or with PBS (FIG. 18). Unlike tumor cells, normal cells treated with Ad-mda7 demonstrated no significant increase in the number of cells in G2/M-phase when compared to control cells. These results suggest that MDA-7 may have selectively affected tumor cells. Furthermore, analysis for mitotic index demonstrated that MDA-7 induces G2—but not M-phase arrest in tumor cells (FIG. 18).

5. MDA-7 Modulates Intracellular Signaling Pathways in Prostate Cancer Cells Resulting in Apoptotic Cell Death

The intracellular signaling mechanism that may participate in the MDA-7 induced apoptosis in prostate tumor cells (DU 145 and LNCaP) was next evaluated. An increase in the phosphorylated form of Stat1 (pSTAT-1) and JNK (PJNK) was observed in both DU 145 and LNCaP cells treated with Ad-mda7 compared to cells that were treated with PBS and Ad-luc. In contrast, a decrease in the phosphorylated form of STAT-3 (pSTAT-3) and NFkB was observed in both tumor cell lines treated with Ad-mda7. The only difference observed between the two tumors cell lines was in the expression of JAK1 and Tyk2. In DU 145 cells, Ad-mda7 treatment resulted in decreased pJAK1 expression and increased pTyk2 expression when compared to control cells. In contrast, Ad-mda7 treatment resulted in increased pJAK1 expression and decreased pTyk2 expression in LNCaP cells indicating that the initiation of signaling may differ between the two cell lines.

Further analysis of the downstream targets, namely caspases, revealed activation of caspase-9, caspase-3, and PARP at 72 h after Ad-mda7 treatment in both DU 145 and LNCaP cells. These results demonstrate that Ad-mda7 can modulate intracellular signaling pathways in prostate cancer cells leading to the induction of apoptosis via the caspase cascade.

6. G2 Cell Cycle Arrest by MDA-7 is Associated with Downregulation of Cdc25C

To investigate the mechanism by which MDA-7 significantly induces G2 arrest in prostate cancer cells, proteins related to the G1/S and G2/M cell cycle checkpoints were evaluated by Western blot analysis. Ad-mda7 treated DU 145 and LNCaP cells demonstrated reduced expression of both phosphorylated and nonphosphorylated Cdc25C and decreased expression of Chk1 and Chk2, and cyclin B1 proteins that are associated with G2/M phase. No significant changes in these proteins were observed in cells that were treated with PBS or treated with Ad-luc. Examination of Cdc2 demonstrated a slight decrease in Cdc2 expression in LNCaP cells but not in DU 145 cells treated with Ad-mda7. Furthermore, cyclin A but not cyclin E was observed to be reduced in both the cell lines treated with Ad-mda7. Examination of additional proteins related to G1/S and/or G2/M cell-cycle checkpoint that are modulated by MDA-7 demonstrated increased expression of p27 and p21 in LNCaP cells but not in DU 145 cells. This increase in p27 and p21 expression in LNCaP cells that are wild-type for p53 gene is probably due to enhanced p53 expression since no change in the expression of these proteins was observed in p⁵³ mutant DU 145 cells. These results indicate that MDA-7 induces G2 cell-cycle arreast by down regulating G2/M related proteins and are consistent with the cell cycle analysis described above.

### Example 15

### Ad-mda7 Radiosensitizes Cancer Cells

Materials and Methods

1. Cell Culture, Vectors, and Chemicals

The human NSCLC cell lines, A549 (wt-p53/wt-Rb) and H1299 (del-p53/wt-Rb), and normal human lung fibroblast lines (NHLF), CCD-16 and MRC-9, were obtained from the American Type Culture Collection (ATCC). All cell lines were maintained as specified by ATCC.

The recombinant adenoviral vector (Ad-mda7) contains the CMV promoter, wild-type mda-7 cDNA, and an SV40 polyadenylation signal in a minigene cassette inserted into the E1-deleted region of modified Ad5. Adenovirus-mediated luciferase (Ad-Luc) was used as a control vector. These vectors have been described previously (Mhashilkar et al., 2001). The preparations were tested for and determined to be free of replication-competent adenovirus and mycoplasma.

Curcumin and Nocodazole were purchased from Sigma-Aldrich (Poole, UK). Stock solutions of curcumin (10 mM) were prepared freshly on the day of the experiment by dissolving the compound in ethanol. Mock-treated cells received the same concentration of ethanol. It was then diluted into medium at a concentration of 10 μM. Stock solutions of Nocodazole (5 mg/ml) were prepared by dissolving the compound in DMSO. It was then was diluted into medium (200 ng/ml).

2. Gene Delivery

In vitro transfection studies for all cell lines were performed by plating 2×10⁵ cells in T25 flasks. Forty-eight hours after plating, cells were incubated for 1 hour with purified vector in 1 ml of medium without serum. After 1 hour, fresh medium supplemented with 10% FBS was added to the flask. Serum-free medium was used for mock transfection. Cells were further incubated for 48 hours before survival curves were generated.

3. Radiation and Clonogenic Assay

Cells were irradiated with a high-dose rate ¹³⁷Cs unit (3.7 Gy/minute) at room temperature in T25 flasks. Irradiation was performed 48 hours after vector treatment. The effectiveness of treatments was assessed by clonogenic assays. Briefly, monolayers of A549, H1299, CCD-16, and MRC-9 cells were treated in T25 flasks as described above, and after various doses of irradiation the cells were trypsinized and counted. Known numbers of cells were then replated in 100 mm culture dishes and returned to the incubator to allow macroscopic colony development. Colonies were counted after 10-14 days, and the percent plating efficiency and surviving fractions following given treatments were calculated based on the survival of nonirradiated cells treated with either mock infection, Ad-Luc or Ad-mda7. The vector treatments used were adjusted for each line to yield identical reductions in platting efficiency with Ad-mda7, i.e. 80%. The vector concentrations used, therefore, were 1000 vp/cell for A549, 250 vp/cell for H1299, and 1500 vp/cell for CCD-16 and MRC-9 cells. These treatments produced nearly 100% transfection efficiency. Some of the experiments on A549 cells used a different lot of Ad-mda7 vector which required 2000 vp/cell to achieve the same transfection efficiency.

4. Apoptotic Index and Cell Cycle Analysis

Apoptosis was quantified by flow cytometry using the APO-BRDU™ Kit (Pharmingen, San Diego, Calif.). Briefly, 2×10⁶ cells were fixed with 1% paraformaldehyde in PBS for 15 minutes at room temperature, washed twice with PBS, and stored in 70% ethanol at −20° C. For analysis, cells were incubated in the DNA labeling solution overnight at room temperature. Fluorescent labeled anti-BrdU antibody solution was added, and the cells incubated in the dark for 30 minutes at room temperature. The stained cells were analyzed by flow cytometry with the use of an EPICS flow cytometer (Coulter Corp., Hialeah, Fla.). All steps were performed according to the manufacturer's recommendations. An analysis region was set based on the negative control, and the percentage of labeled cells was calculated from this region.

Apoptotic indices were analyzed 2 days after irradiation with 5 Gy or 4 days after infection. This time course was based on preliminary indications of the time for maximum apoptotic response. As before, infections with either Ad-mda7 or Ad-Luc were performed 48 hours before irradiation.

5. Western Analysis

Briefly, cells were scraped from the plates, washed with PBS, and lysed in cell lysis buffer. Thirty micrograms of protein were electrophoretically separated on either 8% (for pRb), 12% (for Cyclin B1, p-c-Jun, Fas, Bax and p53), or 15% (for MDA-7) SDS-polyacrylamide gels and transferred to polyvinylide difluoride membrane (Millipore, Bedford, Mass.). Mouse monoclonal antibodies for pRb, Cyclin B1 (Pharmingen, San Diego, Calif.), p53 (DAKO, Carpinteria, Calif.), Fas (Santa Cruz Biotechnology, Santa Cruz, Calif.) and rabbit polyclonal antibody for Bax (Santa Cruz Biotechnology, Santa Cruz, Calif.), MDA-7 (Introgen Therapeutics Inc., Houston, Tex.), JNK-1 (Promega, Madison, Wis.) were used as primary antibodies. Primary antibody for p-c-Jun, specific for c-Jun p39 phosphorylated on serine-63, was obtained from Santa Cruz Biotechnology. Primary antibody for JNK-1 detects the phosphorylated, active form of stress-activated protein kinase (SAPK), also known as c-Jun N terminal kinase, JNK. The membranes were enhanced by chemiluminescence using ECL™ western blot detection reagents (Amersham Corp, Arlington Heights, Ill.) according to the manufacturer's instructions. Total cellular proteins applied to each lane were adjusted to equal concentration with BCA protein assay reagent (Bio-Rad Laboratories, Richmond, Calif.), and were confirmed with coomassie brilliant blue staining method.

Results

1. Ad-mda7 Enhances Radiosensitivity of NSCLC Cells, but Not NHLF Lines

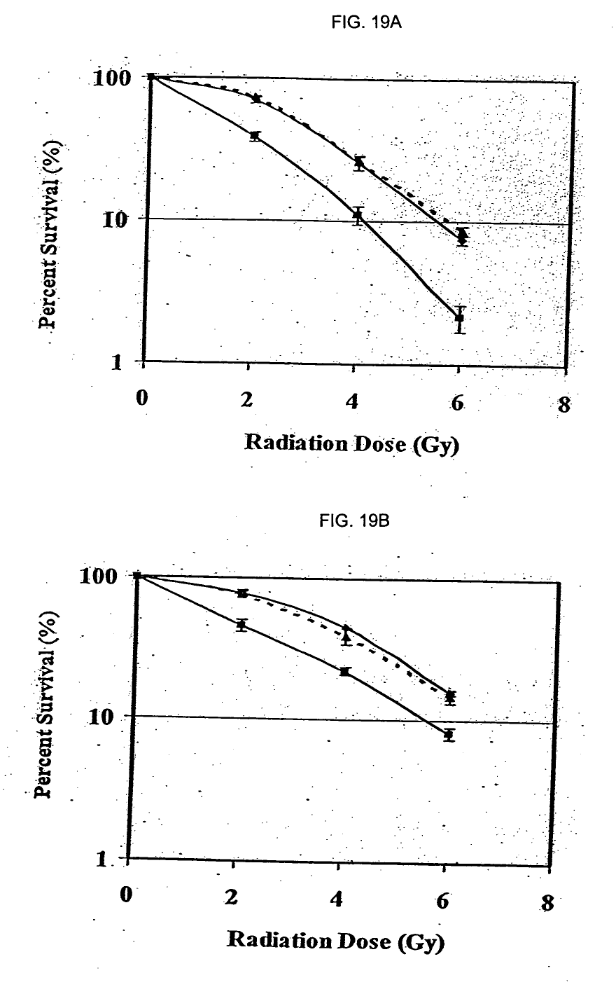

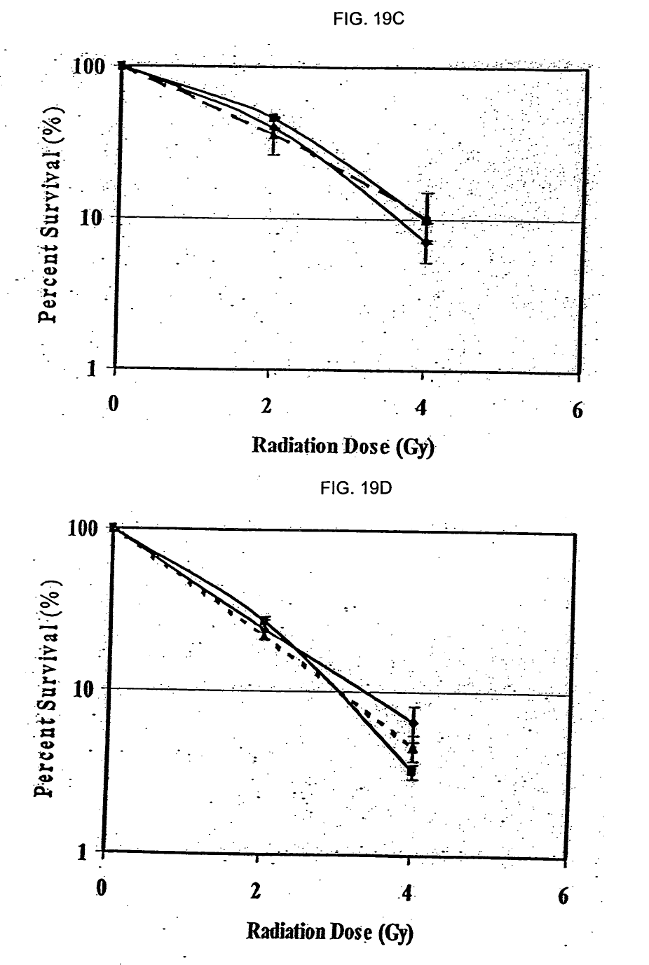

Whether Ad-mda7 infection sensitizes NSCLC cells to irradiation in vitro was tested. Clonogenic assays were performed on two NSCLC lines, A549 and H1299 and two normal human lung fibroblast (NHLF) cell lines, CCD-16 and MRC-9. These lines were infected with either Ad-mda7 or Ad-Luc (control vector) and irradiated 48 hours later. The 48-hour time course was based on cell cycle analysis that demonstrated maximum G2 arrest in this time frame (see below). As shown in FIG. 19, Ad-mda7 radiosensitized both NSCLC cell lines even at the clinically relevant dose of 2 Gy. For example, the percent survival for A549 cells at 2 Gy was reduced from 69.8%±3.1 to 38.5%±3.2, (FIG. 19A) and a dose reduction factor (DRF) calculated at the 50% survival level for Ad-mda7 plus radiation in A549 cells was 1.93. The percent survival for H1299 cells at 2 Gy was reduced from 78.2%±3.7 to 45.7%±4.5 (FIG. 19B) and the DRF for H1299 cells was 2.06. The control vector, Ad-Luc, had no sensitizing effect for either A549 or H1299 cells when used at identical vector concentrations. On the other hand, Ad-mda7 did not radiosensitize the NHLF lines at the clinically relevant dose of 2 Gy. The percent survival for CCD-16 cells treated with radiation alone and radiation plus Ad-mda7 at 2 Gy was 43.6%±7.0 and 45.4%±3.4, respectively (FIG. 19C) and for MRC-9 cells was 24.2%±3.4 and 27.2%±1.6, respectively. (FIG. 19D).

2. Ad-mda7 Induces Apoptosis in NSCLC Cells but Not Normal Cells

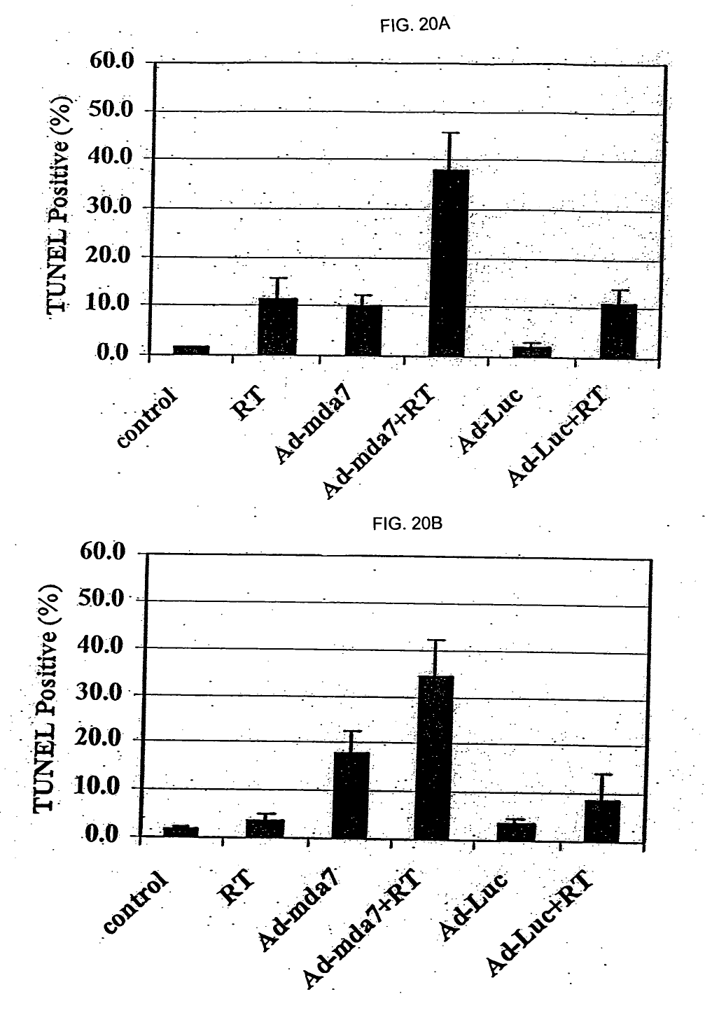

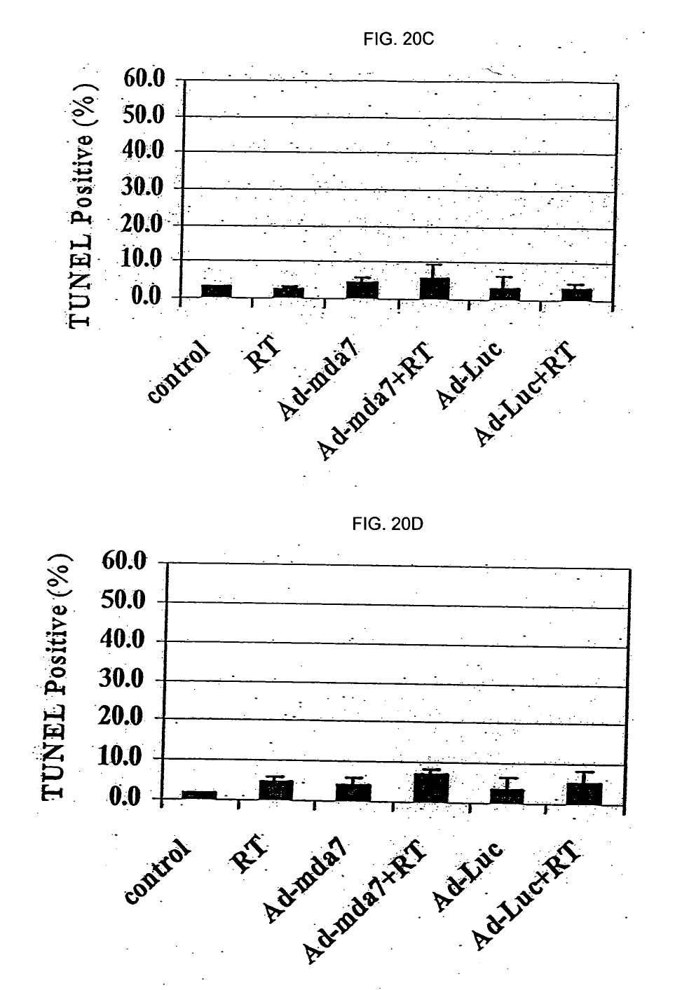

TUNEL assay was used to measure the level of apoptosis (FIG. 20). The percent TUNEL-positive cells in A549 cells (FIG. 20A), H1299 cells (FIG. 20B), CCD-16 cells (FIG. 20C), and MRC-9 (FIG. 20D) treated with either mock infection, 5 Gy alone, Ad-Luc alone, Ad-Luc plus 5 Gy, Ad-mda7 alone, or Ad-mda7 plus 5 Gy are shown in FIG. 20. Radiation alone resulted in an increase to 11% in the proportion of TUNEL-positive cells compared to control in the A549 cells. This effect was less apparent in the H1299 cells. As expected, Ad-mda7 infection alone modestly increased the proportion of TUNEL-labeled cells to 10% in A549 cells and 18% in the H1299 cells. However, the combination of Ad-mda7 and radiation produced a greater-than-additive increase in TUNEL-positive cells in both NSCLC lines achieving levels of 38% and 35% in A549 and H1299 cells respectively. This enhancement of radiation-induced apoptosis was not evident when Ad-mda7 was replaced with Ad-Luc. On the other hand, TUNEL-positive cells for CCD-16 (FIG. 20C) and MRC-9 (FIG. 20D) treated with Ad-mda7 alone were not substantially increased compared with controls and the combination treatment, Ad-mda7 plus 5 Gy, only slightly increased the proportion of TUNEL-positive cells in the NHLF lines.

3. Ad-mda7 Arrests Cells in the G2/M Phase of the Cell Cycle

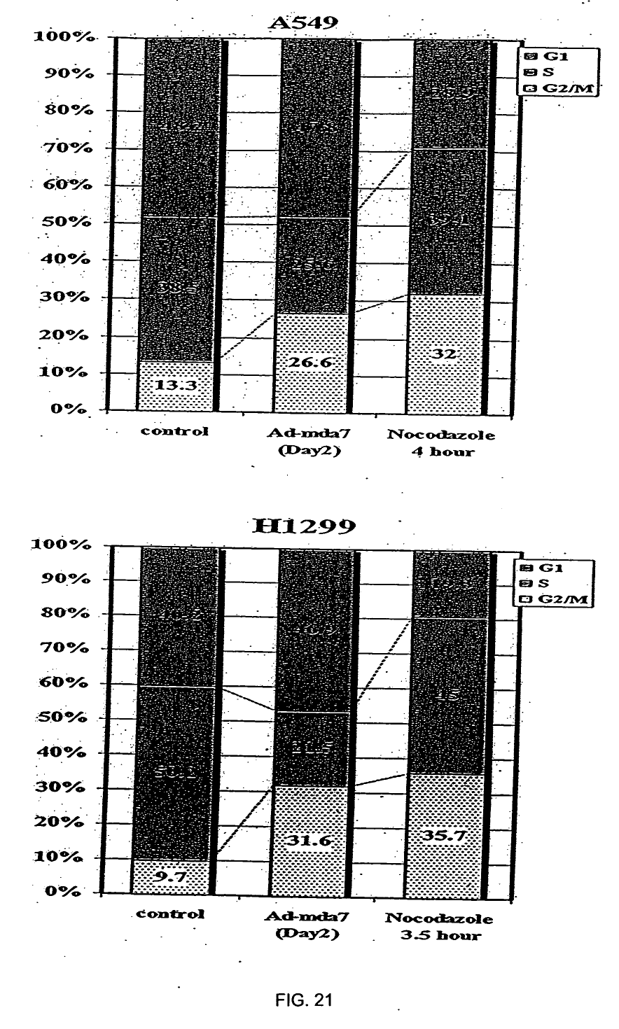

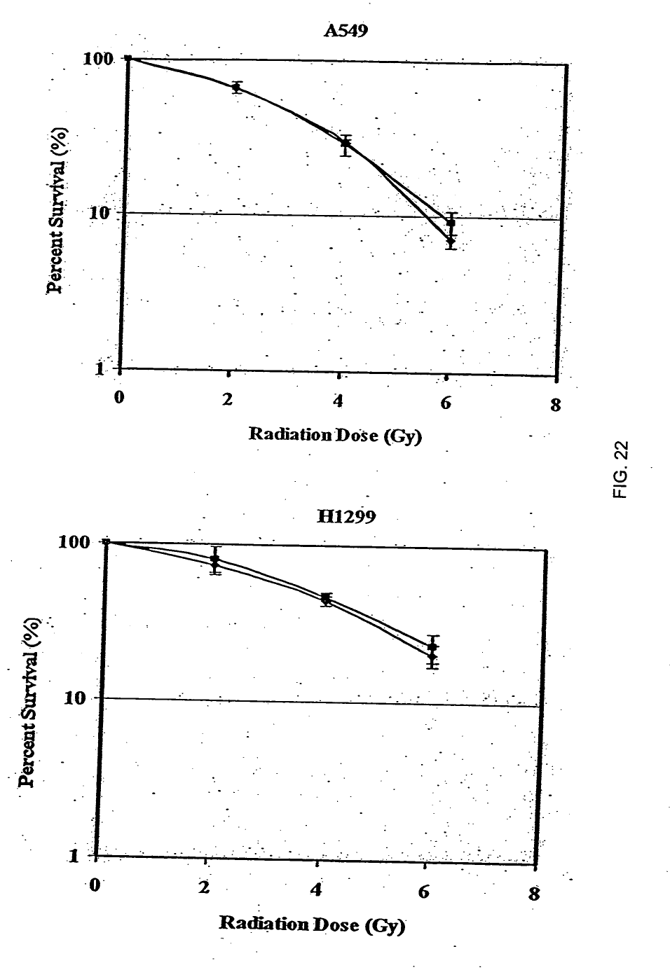

A previous report indicated that Ad-mda7 suppresses proliferation and induces a G2/M cell cycle arrest in NSCLC cell lines (Saeki et al., 2000). These effects were validated and the expression of two proteins known to be involved in cell cycle regulation, pRb and cyclin B 1, was examined. Western blot analysis demonstrated that MDA-7 protein began to be expressed in both A549 and H1299 cell lines by 24 hours after infection. Multiple bands were evident due to the detection of glycosylated forms of the protein (Wang et al., 2002; Lebedeva et al., 2002). As MDA-7 protein began to be expressed following Ad-mda7 administration, expression of pRb declined within 2-4 days in A549 and H1299 cells. In contrast, cyclin B 1 expression was slightly upregulated by day 2, but these levels declined to levels below control by day 3 in A549 or day 4 in H1299 cells. These results suggested that the cells may accumulate in G2/M phase approximately 2 days following Ad-mda7 transfection. This was confirmed by flow cytometry analysis of A549 and H1299 cells at 2 days following Ad-mda7 treatment (FIG. 21). Whether G2/M arrest by itself plays a role in enhancing the radiosensitivity of these cells was investigated. Nocodazole, a drug that reversibly blocks microtubule polymerization, was used to accumulate A549 and H1299 cells in G2/M. The treatment schedule for Nocodazole (200 ng/ml) to induce the same degree of G2/M arrest compared to Ad-mda7 was 4 hours for A549 cells and 3.5 hours for H1299 cells. The radiosensitivity of A549 and H1299 cells treated with Nocodazole compared to controls using clonogenic assays was then determined. The results shown in FIG. 22 indicate that G2/M arrest by itself, at least to the degree mediated by Ad-mda7, does not enhance the radiosensitivity of NSCLC cells.

4. Ad-mda7 Enhances Radiosensitivity Independent of p53, Bax and Fas

The expression of p53, Bax, and Fas protein in A549 and H1299 was analyzed following treatment with either radiation alone, Ad-mda7 alone, Ad-mda7 plus radiation, Ad-Luc, or Ad-Luc plus radiation. The p53 protein levels dramatically increased in A549 cells treated with either radiation or Ad-mda7 but not with Ad-Luc. Fas protein expression in A549 cells treated with either radiation or Ad-mda7 was also increased and this effect was consistent with a dependence on wild-type p53 since Fas protein expression in H1299 cells (which do not express p53) was not enhanced by these treatments. On the other hand, Bax protein expression was not significantly changed in either cell line by these treatments. Thus, since A549 and H1299 cells are equally radiosensitized by Ad-mda7, neither p53, Fas, or Bax correlated with radiosensitization by Ad-mda7.

5. Ad-mda7 Enhances the Expression of p-c-Jun Protein

It has been reported that radiation-induced apoptosis requires the activation of c-Jun N terminal Kinase (JNK) (Chen et al., 1996a; Chen et al., 1996b). The questions of whether Ad-mda7 was able to activate JNK and whether this correlated with radiosensitization were addressed. Rb, p-c-Jun and JNK-1 protein levels were determined in A549, H1299, and CCD-16 cell lines treated with either radiation alone, Ad-mda7 alone, Ad-mda7 plus radiation, Ad-Luc, or Ad-Luc plus radiation. Rb protein expression was dramatically reduced in A549 and H1299 cells treated with Ad-mda7, but not changed with control vector in these cell lines. The expression of both p-c-Jun and JNK-1 was enhanced in A549 and H1299 cells treated with Ad-mda7. In CCD-16 cells, however, Rb protein expression was slightly reduced, but expression of p-c-Jun and JNK-1 was not enhanced by Ad-mda7 treatment. These results are consistent with the possibility that Ad-mda7 mediates radiosensitivity and enhances apoptosis through the activation of JNK-1 and the subsequent activation of p-c-Jun.

6. Curcumin Abrogates Ad-mda7 Mediated Radiosensitization

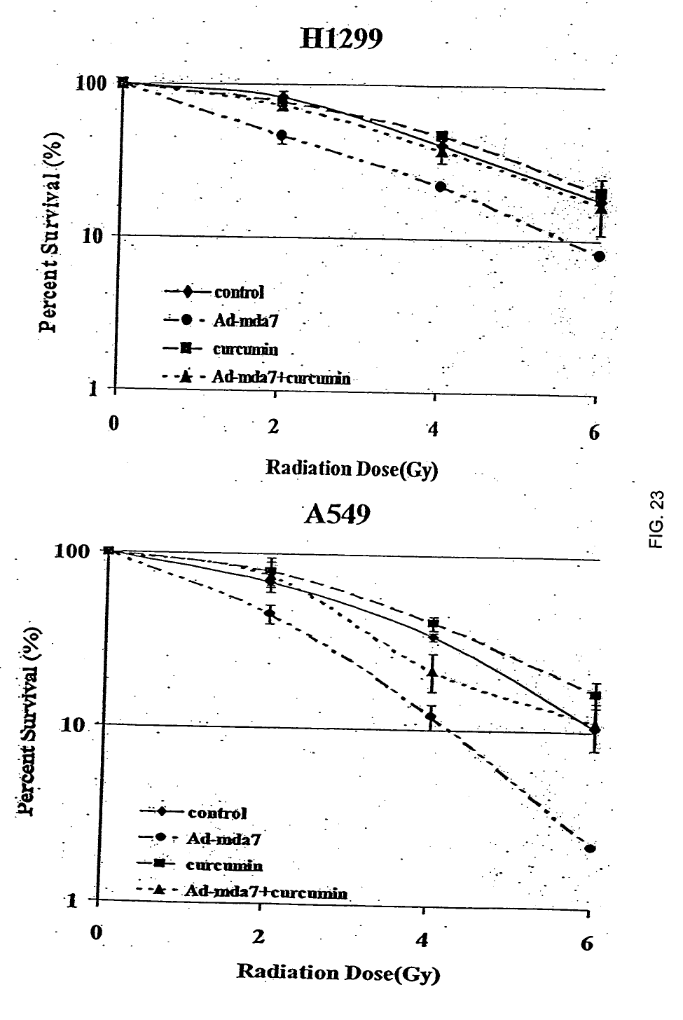

Curcumin, a dietary pigment responsible for the yellow color of curry, has been reported to inhibit JNK activation (Chen and Tan, 1998). Therefore, the expression of p-c-Jun protein was determined in A549 and H1299 cells treated with either radiation alone, curcumin alone, Ad-mda7 alone, radiation plus curcumin, radiation plus Ad-mda7, or radiation plus curcumin plus Ad-mda7. Curcumin when used alone enhanced p-c-Jun expression as did Ad-mda7 used alone. However, curcumin reduced Ad-mda7 mediated activation of p-c-Jun in irradiated and unirradiated cells. To examine whether curcumin inhibits Ad-mda7 mediated radiosensitivity, the inventors performed clonogenic assays using A549 and H1299 lines. Cells were infected with Ad-mda7 and irradiated 48 hours later. As shown in FIG. 23, curcumin abrogated Ad-mda7 radiosensitization in both cell lines.

### Example 16

### Bystander Effect of MDA-7 Protein Against Melanoma Cells

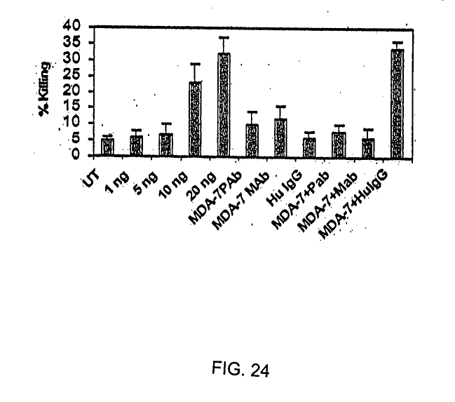

rhMDA-7 (IL-24) protein was purified from 293-mda7 cells using affinity chromatography. Various lots of protein ranged from 30% ->80% purity based upon silver stain. The rhMDA-7 protein was applied to melanoma cell lines, and cells assessed for viability using the Trypan blue assay. As shown in FIG. 24, rhMDA-7 protein caused dose-dependent death in melanoma cells. Treatment of melanoma cells with rhMDA-7 results in rapid activation (via phosphorylation) of STAT3. The cytotoxicity observed in melanoma cells is inhibited by anti-MDA-7 antibodies. FIG. 24 shows that both polyclonal rabbit anti-MDA-7 and monoclonal anti-MDA-7 antibodies inhibit rhMDA-7 mediated killing, whereas control human IgG has no effect. In parallel studies, the anti-MDA-7 antibodies also inhibited MDA-7-mediated STAT3 activation.

The mechanism of the anti-tumor activity of rhMDA-7 was also evaluated. Melanoma cells were treated with rhMDA-7 protein and assessed for apoptosis using the TUNEL assay. As shown in Table 4, 5 of 6 melanoma lines treated with 40 ng/ml rhMDA-7 for 3 days demonstrated cytotoxicity after rhMDA-7 treatment. These lines also showed elevated apoptosis induction. These novel data demonstrate that melanoma cells are susceptible to direct cell killing by MDA-7 protein. Thus, it is anticipated that Ad-mda7 transduction of tumor cells will cause active secretion of MDA-7 protein which can then kill neighboring cells. These studies were performed with purified rhMDA-7 protein. The melanoma cells have also been treated with supernatant from 293-mda7 cells or control 293 cells. Only the 293-mda7 supernatant causes cell killing.

TABLE 4

| | | | % TUNEL |
|---|---|---|---|
| | | % Dead | Positive |
| A375 | +media | 0.0 | 0.9 |
| | +rhMDA-7* | 26.6 | 18.78 |
| A375.S2 | +media | 0.0 | 5.0 |
| | +rhMDA-7 | 22.2 | 12.3 |
| MeWo | +media | 3.0 | 2.22 |
| | +rhMDA-7 | 20 | 12.11 |
| SK-Mel-1 | +media | 0.0 | 0.85 |
| | +rhMDA-7 | 5.2 | 5.0 |
| WM35 | +media | 4.7 | 2.43 |
| | +rhMDA-7 | 17.6 | 17.88 |
| WM793 | +media | 4.7 | 3.44 |
| | +rhMDA-7 | 5.2 | 9.88 |

### Example 17

### Negative Association of Melanoma Differentiation-Associated Gene (mda-7) and Inducible Nitric Oxide Synthase (iNOS) in Human Melanoma: MDA-7 Regulates iNOS Expression in Melanoma Cells

Materials and Methods

The protein expression of mda-7 decreases to nearly undetectable levels in metastatic melanoma. In contrast, expression of inducible nitric oxide synthase (iNOS) is increased in advanced stages of melanoma, and iNOS expression has been proposed as a potential prognostic marker in this disease. Thus, expression of these molecules in the same tumor appears to exhibit reciprocal characteristics. It is hypothesized that the relative ratios of these melanoma progression molecules may define either tumor progression or tumor suppression in human melanoma. The first goal of this study was to determine whether MDA -7 expression in melanoma negatively correlates with iNOS expression. The second goal was to determine whether iNOS expression could be regulated by MDA-7 expression in melanoma cells.

1. Patient Samples

The tumor samples used in this study consisted of primary cutaneous melanomas and melanoma metastases from various sites. Formalin-fixed, paraffin-embedded sections of melanoma tumors were obtained from the Melanoma and Skin Cancer Core Laboratory of the M. D. Anderson Cancer Center.

2. Cell Culture

Metastatic melanoma cell lines, A375 and A375.S2, were obtained from American Type Culture Collection (Rockville, Md.). Radial growth phase and vertical growth phase melanoma cell lines, WM35, WM793, and their more invasive subclones were provided by Dr. Robert Kerbel (Sunnybrook Health Science Center, Toronto, Ontario, Canada). The highly metastatic melanoma cell line MeWo was provided by Dr. David Menter (M. D. Anderson Cancer Center). Melanoma cell lines used in this study were maintained in RPMI 1640 (Life Technologies, Inc., Grand Island, N.Y.) supplemented with 10% fetal bovine serum (Life Technologies, Inc.), 100 units/ml penicillin, 100 μg/ml streptomycin, 2 mM L-glutamine, and HEPES buffer (Life Technologies, Inc.). Cells were either treated with purified MDA-7 at 1-20 ng/ml, or infected with Ad-mda7 or control Ad-luc for in vitro studies.

3. Purification of Human MDA-7

The full-length cDNA of MDA-7 was cloned into the pCEP4 FLAG vector (Invitrogen, San Diego, Calif.) containing the CMV promoter. The plasmid was transfected into HEK 293 cells, and stable subclones were isolated using hygromycin (0.4 μg/ml). Supemnatant containing the secreted MDA-7 was supplemented with protease inhibitors (1 μ/ml leupeptin, 1 μg/ml pepstatin, and 0.5 mM phenylmethylsulfonyl fluoride) and 0.05% sodium azide, and was concentrated 10-fold with an Amicon stirred cell (Amicon, Beverly, Mass.) on an YM 10 membrane. Ten-ml aliquots of concentrated supernatant were separated over an S200 Superdex prep grade column (Amersham Pharmacta, Piscataway, N.J.) in 1× PBS (pH 7.4), and fractions identified to contain MDA-7 by Western blot and ELISA were pooled. After buffer exchange on an Amicon stirred cell to 50 mM 4-morpholinepropanesulfonic acid (pH 6), a second purification step was performed using a Bio-Rad S column. Column conditions consisted of a 0-90-mM NaCl gradient, a 5-min hold at 90 mM NaCl, a 30-min 90-250-mM gradient at 1 ml/min, and a 5-min hold at 250 mM NaCl. The entire purification was conducted at 4° C., and MDA-7 was identified using ELISA and Westem blotting procedures. The final samples contained at least 300 ng/ml MDA-7 as determined by ELISA. Individual lots of partially purified MDA-7 were tested for endotoxin using the QCL 1000 quantitative chromogenic LAL kit (BioWhittaker, Walkersville, Md.).

4. Gene Transfer

Replication-deficient human type 5 adenovirus (Ad5) carrying the mda-7 gene was obtained from Introgen Therapeutics (Houston, Tex.). The mda-7 gene was linked to an internal CMV-EE promoter and followed by SV40 polyadenylation. Ad-Luc and Ad-CMV polyadenylation (luciferase and empty vector, respectively), were used as control viruses. Cells were plated 1 day before infection. Melanoma cells were infected with adenoviral vectors (Ad-mda7 or Ad-luc) using 1000-5000 viral particles per cell. Experimental conditions were optimized to achieve MDA-7 protein expression by >70% of cells, based on results of immunohistochemical staining.

5. Reagents

Anti-iNOS mouse monoclonal antibody Transduction Laboratories, Lexington, Ky.) was used for iNOS immunohistochemistry and confirmed as being cross-reactive between species. Affinity-purified polyclonal rabbit antibodies to MDA-7 were provided by Introgen Therapeutics. IRF-1 and IRF-2 polyclonal antibodies were purchased from Santa Cruz Biotechnology Inc. (Santa Cruz, Calif.). Phospho-Statl (Tyr701) and Phospho-Stat3 (Tyr705) antibodies were obtained from Cell Signaling Tech. (Beverly, Mass.). Pre-immune normal mouse IgG (Vector Laboratories, Burlingame, Calif.) was used as a negative control. Antivimentin antibody (BioGenex Laboratories, San Ramon, Calif.) was used as a positive control for all of the melanoma staining.

6. lmmunohistochemistry

Immunohistochemical labeling was performed on 10% formalin-fixed, paraffin-embedded melanoma tissue, cut 4-6 μm thick. Sections were placedon silanized slides (Histology Control Systems, Glen Head, N.Y.), deparaffinized in xylene, and rehydrated in descending grades (from 100 to 85%) of ethanol. To enhance the immunostaining and restore the maximal antigenicity of cytokines, sections then were placed in antigen unmasking solution Vector Laboratories) and microwaved intermittently for up to 10 min to maintain a boiling temperature. After the slides were cooled at room temperature for 30 min, they were washed in distilled water and PBS. After this initial preparation, the slides were removed from PBS and covered with 3% H₂0₂ (Sigma Chemical Co., St. Louis, Mo.) in methanol to block endogenous peroxidase activity. All of the incubations were carried out at room temperature in a humidified covered slide chamber. The slides were washed in PBS before incubation in PBS containing 0.05% Triton X-100 (Sigma Chemical Co.) for 15 min to permeabilize the cells. An avidin-biotin-peroxidase complex kit (Vectastain; Vector Laboratories) was then used to detect staining. After the slides were incubated for 30 min with the blocking serum, the primary antibodies at various dilutions (1:100 to 1:200) were added, and the slides were incubated for 60 min at room temperature. The slides were then washed, incubated for 30 min with secondary biotinylated antibody, washed again, and then incubated for 30 min with the avidin-biotin-peroxidase complex reagent. After the slides were washed in PBS, the immunostaining was developed with the use of 3-amino-9-ethylcarbazole as a chrornagen for 15 min. Slides were counterstained with hematoxylin (Vector Laboratories) and mounted with Aqua-Mount (Lemer Laboratories, Pittsburgh, Pa.). For each sample, vimentin and isotype-matched control IgG served as positive and negative primary antibody controls, respectively. The specificity and sensitivity of these antibodies have been published previously (Ekmekcioglu et al., 2000; Lebedeva et al., 2002). All of the tissue samples from a given patient were immunolabeled in the same experiment.

7. Immunohistochemistry Scoring

Immunolabeling was scored separately for two variables: first for number of positive cells, second for the overall intensity of immunoreactivity of the positive cells. Scoring for number of positive cells was defined as follows: (0) is for <5% positive cells; (1) is for 5-50% of positive cells; (2) is for 50-90% of positive cells; and finally, (3) is for >90% of positive cells. Intensity scoring was defined as follows: (0) is for no staining; (1) is for light staining; (2) is for moderate staining; and (3) is for intense staining. The slides were interpreted by two independent readers.

8. Immunoblotting Assays

Two×10⁶ cultured melanoma cell lines were rinsed twice in ice-cold PBS and lysed in 60 μl of lysis buffer [25 mM Tris, 140 mM NaCl, and 1% NP40 (pH 7.5)] containing 5 mM EDTA, 0.2 mM orthovanadate, 10 mM NaF, leupeptin, aprotinin, and phenylmethylsulfonyl fluoride for 10 min on ice. Equal amounts of total protein (measured with DC Protein Assay Reagent; Bio-Rad Labs, Hercules, Calif.) were loaded on a standard 10% SDS polyacrylamide gel, and fractionated proteins were electroblotted onto a nitrocellulose membrane. Nitrocellulose membranes were blocked for 1 h at room temperature using 5% dry milk in 1× PBS and washed three times for 5 min each in PBS containing 0.05% Tween 20 at room temperature. The membranes were incubated overnight at 4° C. in a sealed bag with a 1:2000 dilution of IRF-1 and IRF-2 polyclonal antibodies in 10 ml of 5% dry milk/0.1% Tween 20 in 1× PBS. The membranes were washed three times for 5 min each in PBS containing 0.05% Tween 20, and then incubated with peroxidase-conjugated antirabbit IgG secondary antibody (Transduction Laboratories) at 1:2000 dilution in PBS with 5% dry milk and 0.1 % Tween 20 for 45 min at room temperature. The blots were visualized using an enhanced chemiluminescence detection kit (Amersham, Arlington Heights, Ill.).

9. Statistical Analysis

Means and SDs for the iNOS and MDA-7 variables were computed. To investigate the association between iNOS and MDA-7 count and intensity measurements, the test for absence of correlation was performed using the Kendall τ-b test (Woolson, 1987).

Results

1. Melanoma Tumor MDA-7 Expression Negatively Correlates with Tumor iNOS Expression

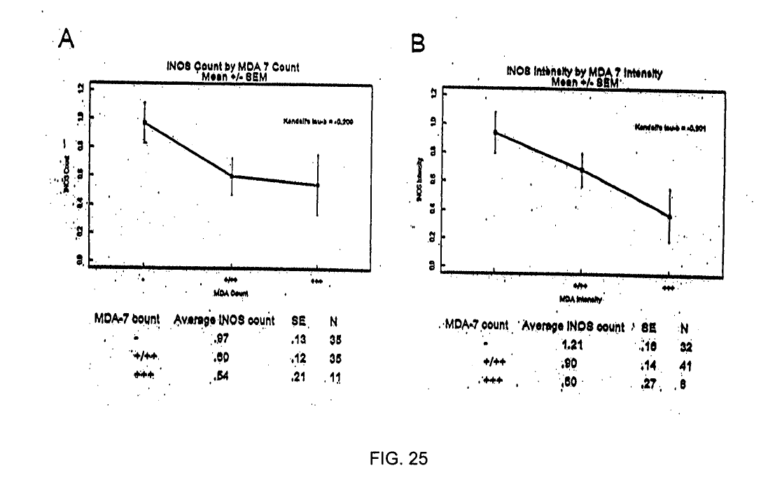

To determine whether MDA-7 expression correlated inversely with iNOS expression in the same tumor, the inventors performed immunohistochemical analyses on sequential paraffin-embedded malignant melanoma tumor sections. Thirty-eight primary melanomas and 43 metastases (total of 81 tumor samples) were analyzed in these experiments. After immunostaining with anti-MDA-7 polyclonal antibody and anti-iNOS monoclonal antibody, sample immunoreactivity based on numbers of positive cells and staining intensity was analyzed. A direct comparison between the numbers of MDA-7-staining cells and iNOS-staining cells revealed a negative association. FIG. 25A demonstrates a significant inverse correlation between numbers of cells staining positively for iNOS and for MDA-7 (correlation coefficient=−0.209, P<0.05, Kendall τ-b test). Similarly, the data were analyzed for an association between iNOS and MDA-7 by comparing the intensity of staining. FIG. 25B shows an inverse correlation between iNOS intensity and MDA-7 intensity (correlation coefficient=-0.201, P<0.05, Kendall τ-b test), reflecting a significant decrease in the average iNOS intensity as MDA-7 intensity increases. Evaulation of tumor MDA-7 and iNOS expression showed a negative association between iNOS and MDA-7 expression in sequential sections of a primary melanoma and sequential sections of a metastasis derived from it. Primary melanoma tumor MDA-7 immunoreactivity in a paired sample displays intense cytoplasmic immunolabeling, whereas both the lymph node metastasis and brain metastasis are negative. In contrast, primary melanoma tumor iNOS is absent, whereas the lymph node metastasis and brain metastasis are strongly immunolabeled.

2. Ad-mda7 and rhMDA-7 Down-Regulate INOS Expression in Human Melanoma Cell Lines

The inverse expression of MDA-7 and iNOS demonstrated by immunohistochemistry suggested a potential cause/effect relationship. Hence, the inventors performed a series of in vitro experiments to examine the possible modulation of iNOS expression by MDA-7. First, the inventors infected melanoma cell lines, A375, MeWo, WM35, and WM793 with Ad-mda-7 (500, 1000, and 2000 viral particles per cell) or with Ad-luc (1000 viral particles per cell). At baseline, these melanoma cell lines express high levels of iNOS and are negative for MDA-7. Forty-eight h after vector treatment, the cells were collected, and cytospins were prepared to analyze iNOS expression. Ad-mda7 at 1000 and 2000 viral particles per cell completely down-regulated expression of iNOS by 48 h, whereas Ad-luc infection had no effect. The dose of Ad-mda7 vector that inhibited iNOS expression did not appear to result in significant cell death during this short incubation. These experimental results indicated that gene transfer expression specifically blocked iNOS expression in melanoma cells. It has been shown previously that MDA-7 is secreted by Ad-mda7-infected melanoma cells (Lebedova et al., 2002; Mhashilkar et al., 2001). To address whether secreted MDA-7 might also contribute to iNOS regulation, the inventors incubated the melanoma cell lines with 0, 5, or 20 ng/ml of rhMDA-7 and stained for iNOS expression. rhMDA-7 at a concentration of 20 mg/ml resulted in clear down-regulation of iNOS expression by 48 h in A375 melanoma cells.

3. MDA-7 Modulates IRF-1 and IRF-2 Expression in Melanoma Cells

rh-MDA-7 protein treatment of melanoma cells resulted in potent down-regulation of iNOS expression, suggesting that MDA-7 may be functioning via a receptor-mediated pathway. It has been shown recently that MDA-7 can bind and signal through the IL-20 and receptors. Thus, the inventors predicted that the EL-20 and/or IL-22 receptor signal transduction pathways, both of which are class II cytokine receptors that involve STAT activation, would be active in melanoma cells exposed to MDA-7. Both STAT1 and STAT3 phosphorylation were initially studied by immunohistochemical labeling in melanoma samples incubated with rhMDA-7. Although no alteration in STAT1 phosphorylation was observed, there was consistent detection of up-regulation of STAT3 in MDA-7 treated MeWo cells compared with untreated cells. Similar findings were observed in the melanoma cell lines A375 and WM35, and A375.S2, and in peripheral blood mononuclear cells from a healthy donor. Increased STAT3 expression was first observed in the cytoplasm of MeWo cells treated with 20 ng/ml rhMDA-7. Moreover, in immunohistochemical labeling studies, staining for phospho-STAT3 was observed in the nuclei of these rhMDA7-treated melanoma cells.

4. MDA-7 Modulates IRF-1 and IRF-2 Expression in Melanoma Cells

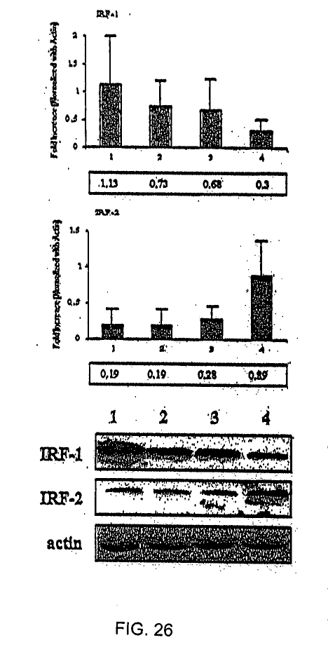

On the basis of the finding of induction of STAT3 phosphorylation after exposure to MDA-7, the downstream targets of STAT proteins were next evaluated. Two of these targets, IRF-1 and IRF-2, oppose each other in their activities in tumor cells. Of note, IRF-1 induces iNOS gene expression (Saura et al., 1999; Dell'Albani et al., 2001). To investigate the potential molecularpathways linking MDA-7 signal transduction to iNOS expression, IRF1 and IRF2 expression in melanoma cells after treatment with rhMDA-7 was evaluated. Immunoblotting for IRF-1 and IRF-2 molecules in rhMDA-7-treated cell lysates demonstrated an up-regulation of IRF-2 expression within 4 h. On the other hand, IRF-1 expression was dramatically decreased by rhMDA-7 treatment of MeWo cells within 4 h (FIG. 26). Although differences did not reach significance because of the small sample size, IRF-1 expression fell by almost 4-fold, whereas IRF-2 expression increased by 4.7-fold.

### Example 18

### Ad-MDA7 Augments Anti-Tumor Efficacy of Tamoxifen

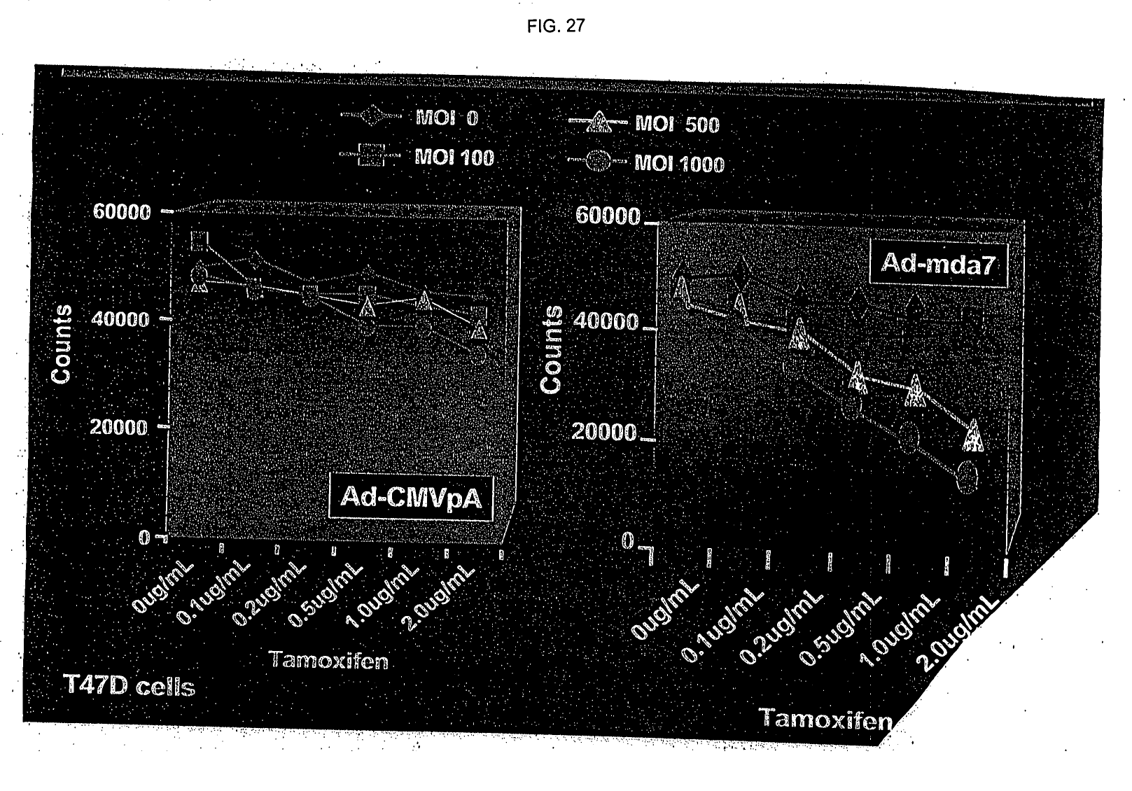

T47D cells were treated simultaneously with increasing MOIs (0-1000 vp/cell) of Ad-vectors and increasing concentration of tamoxifen (0-2 μg/ml). Four days post-treatment, the cells were analyzed for proliferation using the tritiated-thymidine incorporation assay. FIG. 27 shows that Ad-mda7 augments the anti-tumor efficacy of tamoxifen.

### Example 19

### MDA-7 Activates STAT3 in Endothelial Cells

HUVEC cells were treated with 10-20ng of purified MDA-7 protein in chamber slides (1000 cells/chamber). The MDA-7 was affinity-purified MDA-7. After 4 h the cells were washed and incubated with rabbit anti-p-Stat3 antibody (Cell Signaling, 1:1000 dilution) for 1-2 hours at 4° C. The cells were then washed 3× with PBS and treated with secondary, Texas-red conjugated anti-Rabbit-IgG (1:1000 dilution). The cells were washed and then analyzed for pStat3 nuclear staining by fluorescence microscopy. Results indicate that MDA-7 activates Stat3 in endothelial cells. Similar results were obtained using crude 293-MDA-7. Cells were simultaneously stained with Hoescht dye to visualize nuclei.

### Example 20

### Ad-MDA7 and MDA-7 Protein Rregulate Cyokine Secretion from Melanoma Cells

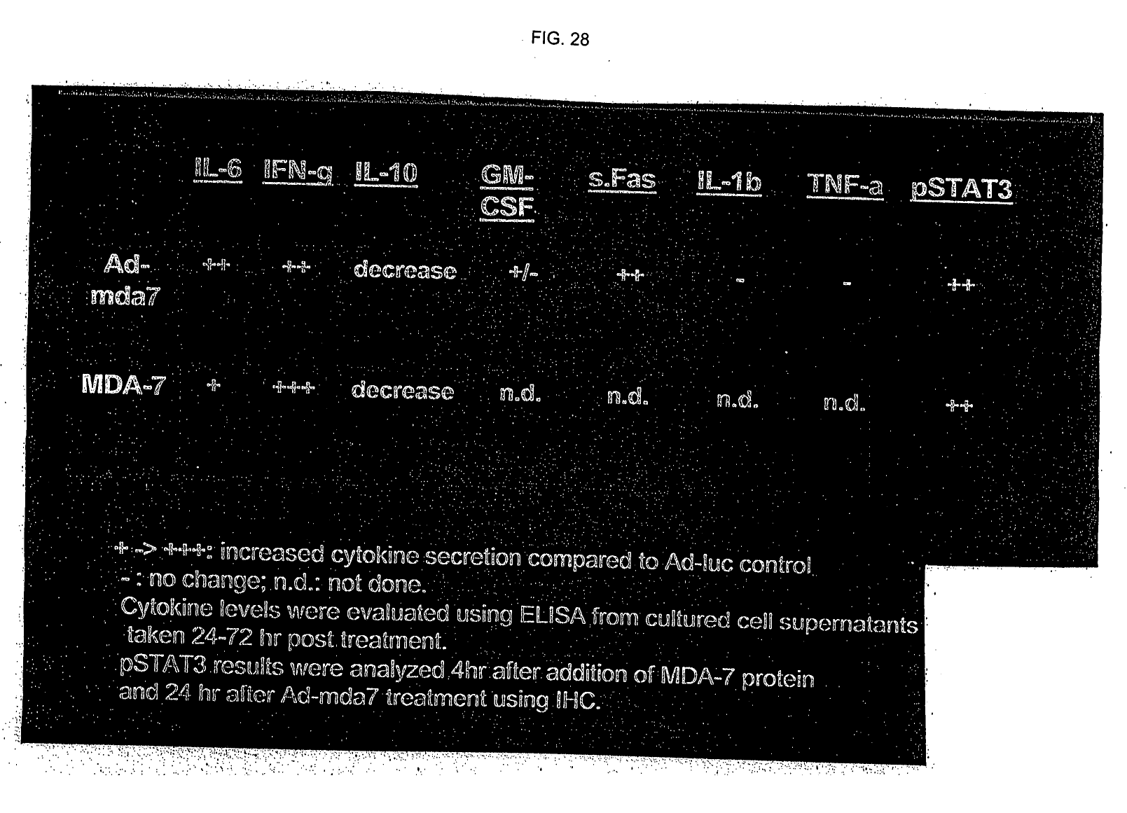

Results of a comparison of cytokine induction by Ad-mda7 or MDA-7 protein treatment of melanoma cells are shown in FIG. 28.

### Example 21

### Effect of Ad-MDA7 on A549 Lung Metastases

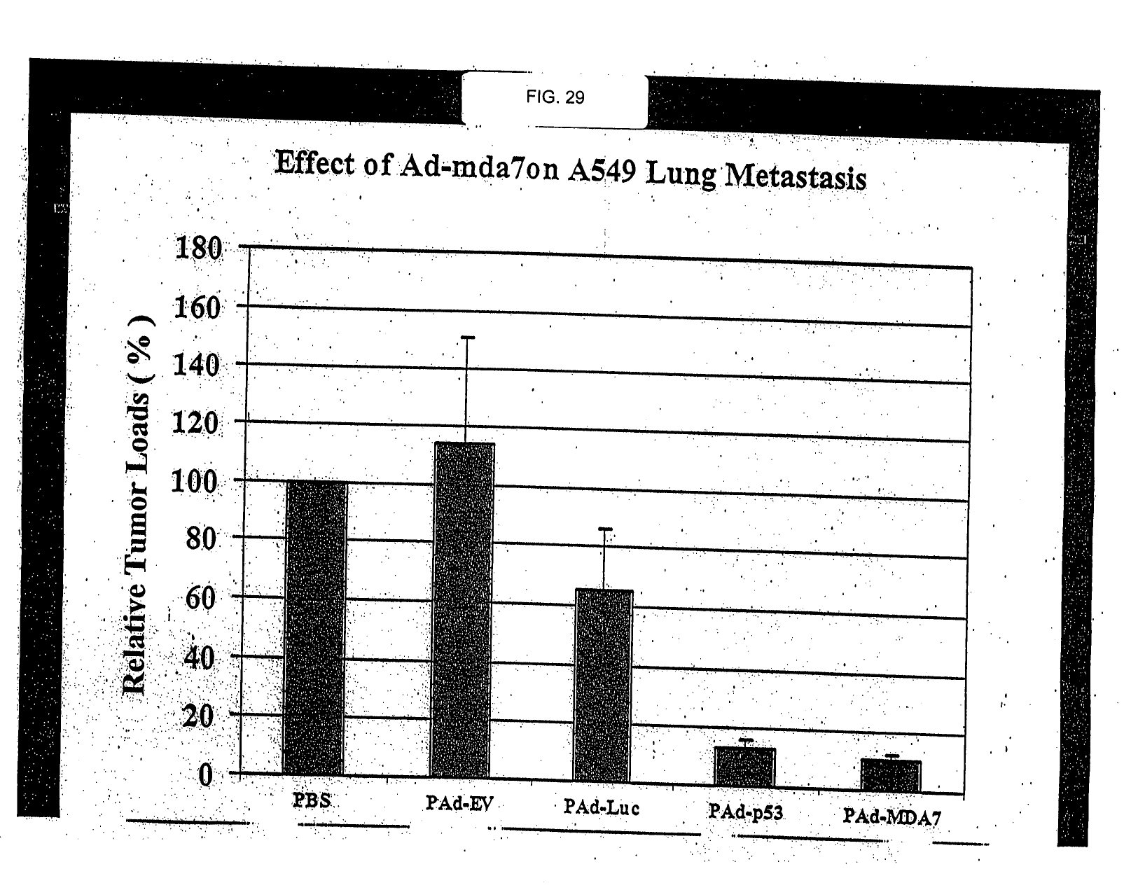

A549 lung cancer cells were injected intravenously into nude mice to establish lung metastases. Ad vectors (Ad-empty (Ad-EV); Ad-luc, Ad-p53, and Ad-mda7) were complexed with protamine and injected intravenously into nude mice and tumor burden in the lungs was measured. Results are shown in FIG. 29.

### Example 22

### MDA-7 Selectively Inhibits Vascular Smooth Muscle Cell Growth and Migration

Material and Methods

1. Cell Culture and Cell Counts

PAC-1 SMC were maintained at 37° C., 5% CO₂ in DMEM (GIBCO/BRL, Life Technologies) supplemented with 10% FBS. PAC1 cells were used at passage level 70-85. PAC1 cells were growth-arrested by 0.1% FBS for at least 24 hours. Normal rat aortic smooth muscle cells (RASMC) were used at passage 10-20. Primary human coronary artery SMC (HCASMC) were obtained from a commercial vendor and grown in SmGM-2 medium (Clonetics, San Diego, Calif.) and used at passage 5-10. Cell viability after virus-transduction was measured by manually counting live cells (in triplicate) with a hemocytometer (Fisher) at the indicated time points.

2. Trypan Blue Exclusion

Cell viability was determined using the Trypan-blue exclusion assay. Briefly, total cells (suspension+trypsinized) were mixed 1:1 with Trypan blue solution (Gibco-BRL) and then observed under a hemocytometer via light microscopy. The percent of blue cells (dead cells) were counted (an average count of 3-5 fields were made).

3. Adenovirus Transduction

Recombinant, replication-defective adenovirus directing the expression of either human mda-7 (Ad-mda7) or luciferase (Ad-Luc) (Mhashilkar et al., 2001), Ad-RSV-beta-ga1 and Ad-SM22-β-ga1 have been described (Kim et al., 1997). PAC-1 SMC were grown in 6-well plates or 100 mm dishes in complete medium. When the cells were 50%-90% confluent, the medium was changed to DMEM containing 2% FBS, and stock virus preparations diluted in the above medium if necessary and inoculated onto the cell monolayers at the indicated multiplicity of infection (MOI; pfu/cell). After 1 hour virus adsorption at 37° C. with rotation by hands every 10 minutes, complete medium was added to the transduced cultures and cells were incubated at 37° C. for the indicated times. As a control in some experiments, an identical group of cells was left untransduced but incubated in DMEM containing 2% FBS for 1 hour with rotation every 10 minutes. X-ga1 histochemistry was performed as described (Kim et al., 1997).

4. Northern Blotting

Total RNA was isolated from the PAC1 cells transduced with virus using acid phenol extraction method (Chomczynski and Sacchi, 1987). 10 μg of total RNA was electrophoresed in 1.2% agarose gel containing formaldehyde, transferred to a nylon membrane (Zeta Probe, BioRad Laboratory), and hybridized with ³²P-labeled human mda-7 cDNA fragment. After hybridization, the nylon membrane was washed and exposed for autoradiography. GAPDH probe was also used to hybridize with the stripped membrane to detect the GAPDH mRNA as a control for equal loading.

5. Western Blotting

Following experimental treatment, PAC1 cells were harvested in lysis buffer. Cell lysates were then sonicated, and centrifuged at 14,000×g at 4° C. for 15 minutes. Supernatants were transferred and protein concentrations of samples were assessed using Bio-Rad protein assay kit. In some experiments, conditioned medium were also collected for analysis of protein secretion before the cells were lysed. 30-50 μg of cellular protein or 10-20 μl of conditioned medium were fractionated on 10-12% SDS-PAGE and transferred to immobilon-P membranes (Millipore). Membranes were subsequently probed with protein-specific antisera MDA-7 antibody (1:1000, Introgen Therapeutics, Houston, Tex.) and other apoptotic antibodies (BAK, BAX, BCL-2, BCL-xL, 1:1000, Santa Cruz, Calif.). pSTAT-3 protein was detected using rabbit anti-human pSTAT-3 antibody (1:1000, Cell Signalling Technology, Beverly, Mass.). Equivalence of protein loading was assessed using anti-β-tubulin antibody. Immunologically identified proteins were recognized using alkaline phosphatase-conjugated, species-specific IgG and Enhanced Chemiluminescence (PIERCE).

6. Analysis of Apoptosis

PAC1 cells were analyzed for apoptosis, using the ApoAlert Annexin V-FITC kit (CLONTECH). Briefly, virus-transduced cells were harvested by trypsinization and washed extensively with PBS and binding buffer. The cells were then incubated with Annexin V-FITC reagent diluted in binding buffer for 30 minutes at room temperature in dark with flicking every 10 minutes. Cells were washed twice with PBS and processed for FACS analysis. PAC1 cells were also analyzed for apoptosis, using DAPI staining assay. Briefly, virus-transduced cells at each time point washed with PBS, fixed in 4% paraformaldehyde and incubated with 300 nM DAPI diluted in PBS at room temperature for 1-4 minutes. The cells were then rinsed in PBS and visualized by fluorescence microscopy and the apoptotic cells were determined by analysis of nuclei and expressed as the number of apoptotic nuclei in a total cell number of 400 nuclei ×100%, as described previously (Dimmeler et al., 1997). PAC1 cells were analyzed for caspase-3 activity using Apo-ONETM Homogeneous Caspase-3/7 Assay kit (Promega, Madison, Wis.) according to the manufacturer's protocol. Briefly, the virus-transduced cells were lysed in hypotonic buffer (25 mM HEPES, PH 7.5, 5 mM MgCl, 5 mM EDTA, 5 mM DTT, 2 mM PMSF, 10 μg/mL Pepstatin, 10 μg/mL Leupeptin) by freeze-thaw twice followed by a centrifugation at 13,500 rpm for 15 minutes at 4° C. Supernatants were transferred to new tubes for activity assay. Another set of experimental cells was harvested in NHE buffer for quantification of total protein. For each reaction, 25 μL of the cell lysate was used to incubate with 25 μL of Homogeneous Caspase 3/7 Reagent (substrate diluted in buffer) at room temperature for 4 hours with continuous shaking in the presence or absence of 150 nM caspase-3 inhibitor (Ac-DEVD-CHO). The fluorescence was measured in a fluorescence plate reader at an excitation wavelength of 485 nm and an emission wavelength of 535 nm. The analysis of caspase 3 activity was performed by normalization of the measured fluorescence with total protein and expressed as Units/10 μg total protein.

7. Flow Cytometry

Apoptosis of cells after viral transduction and integrin expression on the cell surface was assessed by flow cytometry. PAC1 cells transduced with viruses were harvested by trypsinization, washed with PBS and fixed with cold 70% ethanol for 12 hours. The cells were centrifuged, washed with PBS twice and resuspended in PBS followed by incubation with propidium iodide (PI) at final concentration of 50 μg/mL and RNAse at 20 μg/mL at room temperature. An identical group of cells as a control was left untransduced but followed by the same treatment as described above. Treated cells prepared as a single suspension of 105 cells/mL of PBS were then evaluated by FACS analysis using a FACSCalibur flow cytometer (Becton Dickinson, San Jose, Calif.). The percentage of cell apoptosis was determined using the program of Modfit apoptosis analysis (Becton Dickinson, San Jose, Calif.). Three separate experiments were performed with three different populations of cells. Flow cytometric analysis was also performed to evaluate the integrins protein expression on the cell surface, as described (Li et al., 2001).

8. Cell Migration Analysis

The rate of migration of PAC1 cells was determined by using a scratch wound assay as described (Huang and Kontos, 2002). Briefly, PAC1 cells were grown in 60-mm plates until 90% confluent then either left untransduced or transduced with viruses for 24 hours. The cell monolayer was disrupted with a sterile pipette tip to create a cell-free zone after starvation for 24 hours. The cells were then treated with or without 10% FBS. 24 hours after treatment, cells were visualized under an Olympus IX-70 microscope connected to a camera. PAC1 cell migration was quantified by measuring the width of the cell-free zone (distance between the edges of the injured monolayer) at 3 distinct positions with a manually set ruler.

9. STAT-3 Activation

Cells are plated in chamber slides and treated with MDA-7 protein for 60 minutes and then washed thoroughly with PBS (3×). The cells are then fixed with methanol:acetic acid (95:5 vol:vol) and stained with anti-pStat3 monoclonal antibody (1:1000 dilution; Cell Signalling) for 1 h at 4° C. The cells are then washed 3× with PBS and treated with secondary antibody (1:1000 dilution; Texas Red-conjugated-Rabbit anti mouse IgG; Sigma) for 1 h at 4° C. The cells are then washed and examined under fluorescence microscopy.

10. Statistical Analysis

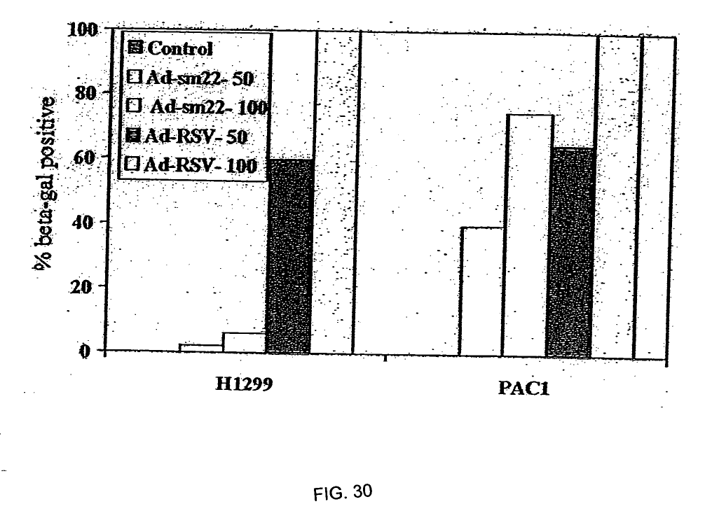

All values are expressed as mean±SEM. All graphical data are representative of at least 3 independent experiments, except FIG. 30, which had 2 replicates. Tests for significance of differences were made by ANOVA or Student's t test as appropriate. Significance was determined as p<0.05

Results

1. Characterization of PAC1 SMC

PAC 1 cells were isolated from rat pulmonary arterial smooth muscle cells (SMC) and were derived based upon their stable maintenance of differentiated properties through multiple subcultures (Rothman et al., 1986; Firulli et al., 1998). The cells used in this study have been multiply passaged from a clonal isolate (and were used at passage 70-85). Previous studies have demonstrated that PAC1 serve as a good model for SMC differentiation as they express a wide variety of SMC-specific markers and exhibit functional responses to various physiologic stimuli (see Table 5) (Firulli et al., 1998; Rothman et al., 1994). PAC1 cells express a similar complement of SMC markers to normal rat aortic smooth muscle cells (RASMC), whereas these markers are not generally expressed in L6 skeletal myoblasts or normal human umbilical vascular endothelium (HUVEC) (see Table 5).

The transduction efficiency of adenovirus in PAC1 SMC was first evaluated. As shown in FIG. 30, PAC1 SMC transduced with Ad-RSV-β-ga1 at an MOI of 50 pfu/ cell resulted in approximately 60% beta-ga1 positive cells (FIG. 30). To further confirm the SMC phenotype of PAC1 cells, they were transduced with Ad-SM22-β-gal, a vector containing a smooth muscle-specific promoter (SM22α) driving expression of beta-galactosidase (Kim et al., 1997). The PAC1 cells were compared to a highly transducible lung cancer cell line. Using Ad-RSV-beta-ga1 as a control vector, H1299 NSCLC cells were transduced with similar efficiency to PAC1 cells. However, PAC1 cells were transduced 10-fold more efficiently than H1299 cells after treatment with Ad-SM22-beta-ga1 (FIG. 30). Comparably low β-ga1 expression was also seen with other tumor cells, demonstrating the SMC lineage of PAC1 cells. Transduction with Ad-mda7 also resulted in high level transgenic protein expression. The efficient transduction of PAC1 cells was further evaluated by analysis of cell surface molecules implicated in adenoviral transduction. PAC1 cells expressed high levels of CAR and alpha v integrin on the cell surface by FACS.

TABLE 5

| SMC MARKER | PAC1 | RASMC | L6 MYOBLASTS | HUVEC |
|---|---|---|---|---|
| SM Calponin | + | + | − | − |
| SM Myosin | +/− | +/− | − | − |
| SM22α | + | + | − | − |
| SM α-actin | + | + | +/− | − |
| Tropoelastin | + | + | − | − |

2. Human MDA-7 Expression in PAC1 SMC Transduced with Ad-mda7

No endogenous mda-7 mRNA was detected in PAC 1 growing in complete medium. However, both mda-7 mRNA and MDA-7 protein were found in PAC1 transduced with Ad-mda7 with no detection of mda-7 mRNA or protein in control virus-treated cells. MDA-7 protein was detected in the conditioned medium suggesting that Ad-mda7 transduced PAC1 secrete soluble MDA-7 protein. This finding is consistent with previous studies in human tumor cell lines transduced with Ad-mda7 (Mhashilkar et aL, 2001). Consistent with previous studies using human tumor cells (Mhashilkar et al., 2001), MDA-7 was secreted from PAC1 cells as a larger protein than the intracellular form, which is indicative of some post-translational modification (e.g., glycosylation). Analysis of secreted MDA-7 protein levels indicates a temporal increase in protein secretion from PAC1 cells after Ad-mda7 transduction at 100 pfu/cell (MOI) over 3 days. A dose-dependent increase in MDA-7 protein was also observed in both cell lysate and conditioned medium from PAC1 cells transduced with increasing MOI of Ad-mda7. These studies establish the feasibility in effecting very large increases in MDA-7 expression in PAC1 SMC.

3. Ectopic MDA-7 Expression Inhibits PAC1 SMC Growth

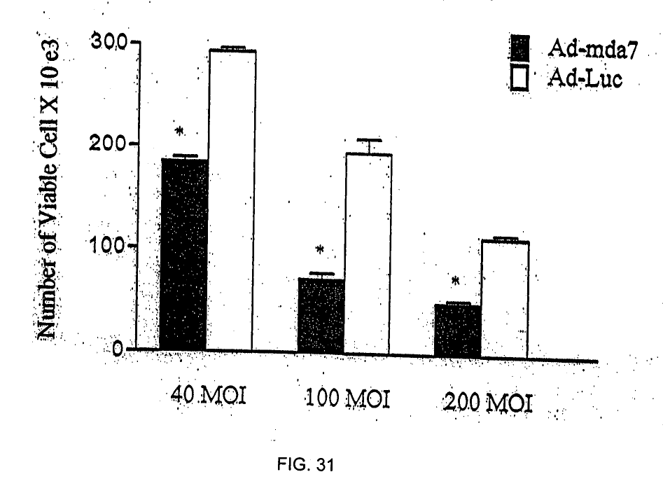

Whether forced expression of mda-7 had inhibitory effects on the growth of PAC1 cells was investigated next. PAC 1 SMC were transduced with Ad-mda7 or Ad-Luc at 0, 40, 100, and 200 MOI and viable cells were counted 3 days following transduction. One representative study is shown in FIG. 31. PAC 1 SMC transduced with Ad-mda7 exhibited significant decreases in the number of viable cells as compared to Ad-Luc transduced cells. A maximal inhibitory effect of mda-7 expression on PAC1 cell growth was observed at 100 MOI (p=0.02 compared to Ad-Luc); at higher MOIs, Ad-Luc showed toxicity. Thus, all subsequent studies used Ad-mda7 at 100 MOI.

4. MDA-7 Expression Enhances PAC1 SMC Apoptosis

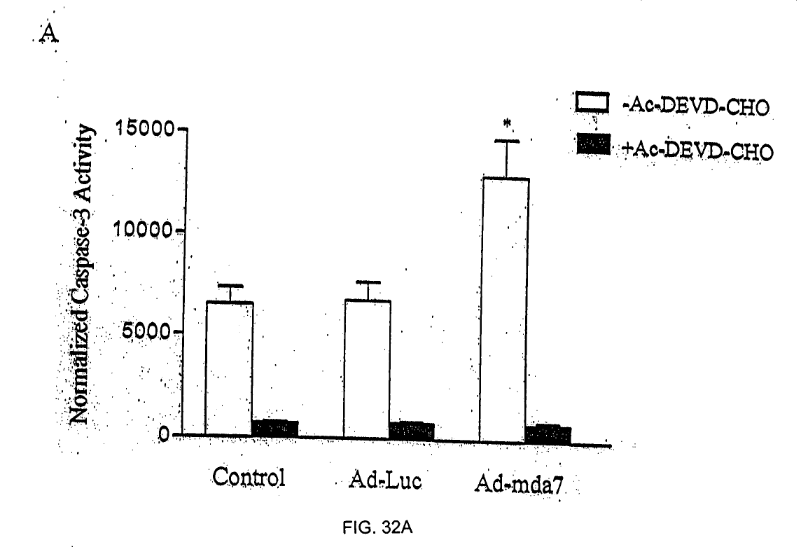

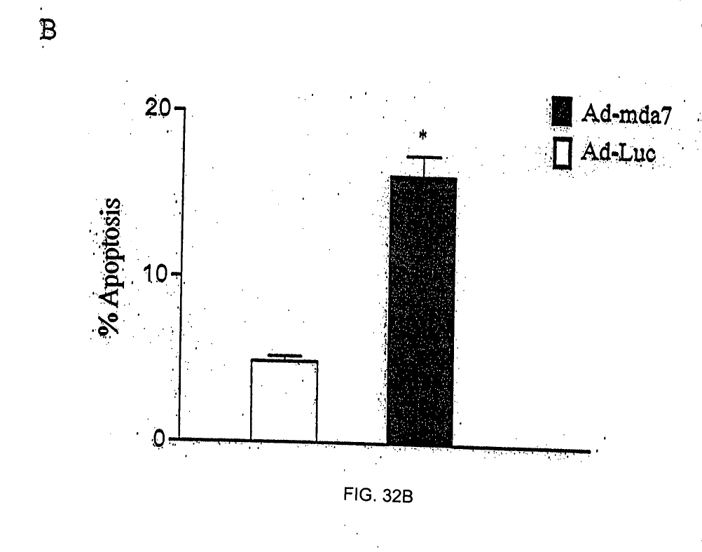

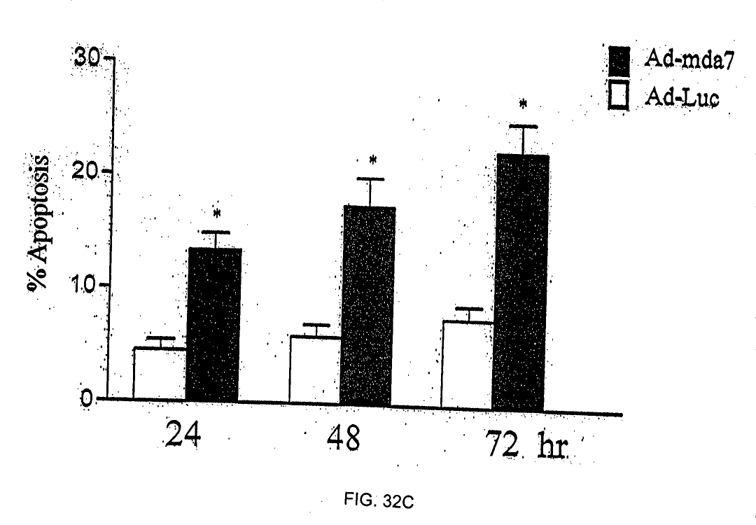

Previous studies demonstrated that Ad-mda7 induces apoptosis in a variety of human tumor cell lines derived from breast, colon, and lung (Mhashilkar et al., 2001; Saeki et al., 2002). It is likely that inhibition of PAC1 cell growth by MDA-7 was mediated in part by an increase in apoptosis. Initial studies were performed to determine caspase-3 activity, a member of the cysteine aspartic acid-specific protease (caspase) family, which plays key effector roles in apoptosis in mammalian cells (Nicholson et al., 1995). A significant increase in caspase 3 activity was observed in Ad-mda7 treated PAC1 cells compared to untreated or Ad-Luc treated cells (p<0.05 compared to Ad-Luc) (FIG. 32A). The caspase-3 activity was specifically inhibited by the caspase-3 inhibitor (Ac-DEVD-CHO). These findings suggest that overexpression of mda-7 activates caspase 3 and promotes apoptosis in PAC 1 cells.

To quantify the apoptotic effect of MDA-7 expression in PAC1 SMC, the inventors next examined the phospholipid, phosphatidylserine (PS), present on the cell surface using Annexin V staining in conjunction with FACS analysis as a measure of early apoptosis. Compared to control Ad-Luc transduced cells, Ad-mda7 treatment resulted in significant increase (p<0.05) in the number of apoptotic PAC1 SMC as early as 24 hours after transduction (FIG. 32B). DAPI staining assay was performed to quantify the effect of Ad-mda7 on late apoptosis by analyzing nuclear condensation and disruption. Ad-mda7 transduction resulted in significant increases (p<0.05) in the number of apoptotic cells at each time point compared with control virus (FIG. 32C). In another independent measure of apoptosis, the inventors noted Ad-mda7 treated cells exhibited a profound increase in the number of cells in the sub G0/G1 phase of the cell cycle. Together these findings suggest that enhanced apoptosis may contribute to the inhibition of PAC 1 SMC growth by Ad-mda7.

Previous studies showed that changes in the levels of BAX, BAK and the ratio of BAX to BCL-2 proteins may be important mediators in the induction of apoptosis in cancer cells by mda-7 (Lebedeva et al., 2002; Su et al., 1998; Madireddi et al., 2000). To determine if these apoptosis-associated molecules contribute to programmed cell death mediated by ectopic mda-7 in PAC1 cells, the levels of the various proteins were determined by Western blotting 6-72 hours post-transduction with Ad-mda7. Upregulation of proapoptotic (BAK, BAX) proteins occurred in PAC1 SMC 72 hour after transduction with 100 MOI Ad-mda7, although BAK protein appears to increase as early as 24 hr. Ad-Luc treatment for 72 hours showed no change in protein levels. In contrast, anti-apoptotic (BCL-2) protein expression decreased in PAC1 cells 72 hours after transduction with 100 MOI Ad-mda7 with only minor change in BCL-xL protein. These results suggest that increases in the levels of pro-apoptotic (BAK, BAX) versus anti-apoptotic (BCL-2, BCL-xL) proteins resulting from transduction with Ad-mda7 may trigger late apoptosis in PAC1 cells. Note that apoptosis is detectable within 24 hours of Ad-mda7 treatment (FIG. 32B, and 32C), consistent with the increase in BAK levels, but not BAX. Thus, BAK pro-apoptotic protein may be the initiator of apoptosis in PAC1 cells.

5. MDA-7 Inhibits PAC1 SMC Migration

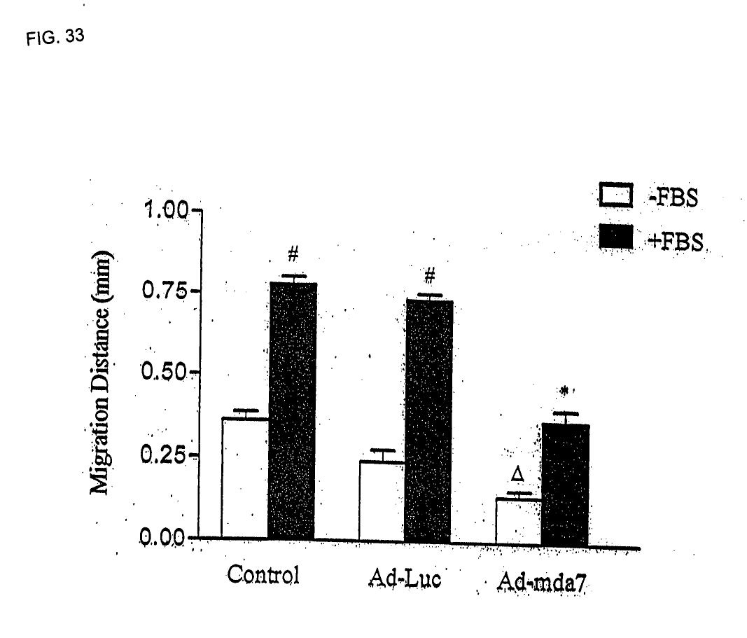

Cellular migration is another key process in the evolution of a neointima in vascular pathologies. To investigate whether mda-7 expression alters PAC1 SMC migration, the migration of adenovirus-transduced or untransduced PAC1 SMC was measured after scrape wounding the monolayer. Stimulation with serum significantly increased the migration of PAC1 into the wound. In contrast, mda-7 over expression significantly inhibited both basal (p<0.05) and FBS-stimulated (p<0.01) PAC1 cell migration at 100 MOI (FIG. 33). Thus mda-7 can block migration even in the absence of serum-stimulation.

6. Ad-mda7 Does Not Inhibit Growth of Normal SMC Cells

Primary human coronary artery SMC (HCASMC; passage 5-10) and normal rat aortic SMC (RASMC; passage 10-20) were transduced with Ad-mda7 or Ad-luc at various MOIs. Western blot analysis demonstrated that MDA-7 protein was produced intracellularly and also secreted from both normal types of SMC, at levels comparable to that seen with PAC1 SMC. No MDA-7 was endogenously expressed in the normal SMC or after Ad-Luc treatment. Similar levels of MDA-7 protein were expressed in HCASMC and RASMC as PAC1 cells after Ad-mda7 transduction with 100 MOI. However, cell viability studies showed no loss of cell viability after Ad-mda7 treatment of HCASMC or RASMC. Similar results were observed using cell cycle analysis as an independent assay for apoptosis induction (see Table 6). Only PAC1 SMC showed an increase in sub G0/G1 cells after Ad-mda7 treatment at 100 MOI for 24 hours. Thus the inhibition of cell growth and induction of apoptosis observed in rat PAC1 SMC is not observed in normal rat SMC or primary human SMC.

To try and understand the differential activity of mda-7 over-expression in PAC1 cells versus normal rat and human SMC, chromosome spreads of the PAC1 cells used in this study were evaluated. Karyotyping of passage 70-85 PAC1 cells revealed fundamental differences in chromosomal banding compared to earlier reports of PAC 1 or RASMC karyotypes (Firulli et al., 1998). Specifically, the PAC1 cells showed trisomy 20, a translocation at chromosome 11 resulting in a larger p arm, and an additional marker chromosome of unknown origin. Thus, there are substantial chromosomal abnormalities in the strain of PAC 1 cells susceptible to MDA-7-mediated cell death.

TABLE 6

| Cell Type | Ad-Luciferase | Ad-MDA7 |
|---|---|---|
| PAC1 | 8.16% | 24.07% |
| HCASMC | 18.1% | 18.49% |
| RASMC | 0.98% | 0.87% |

7. MDA-7 Effects Cell Death through an Intracellular Pathway

To determine whether the selective death induced with Ad-mda7 in PAC1 SMC was due to a surface receptor-mediated process or through some intracellular pathway, cell death and STAT-3 activation (a measure of MDA-7/IL-24 receptor activation) was measured following stimulation of PAC1 SMC and RASMC with recombinant MDA-7 (rMDA-7). No statistically significant increase in cell death was observed in PAC1 SMC stimulated with rMDA-7. Consistent with this finding was our inability to demonstrate any evidence for STAT-3 activation in these cells. These results suggest that Ad-mda7 mediates selective cell death in PAC1 SMC through an intracellular mechanism.

### Example 23

### Clinical Trial Results and Information Regarding Ad-MDA7 Administration and Expression

Material and Methods

1. Patient Criteria

Histologically confirmed carcinoma with at least one lesion that was accessible for needle injection and then surgically resectable (phase 1 patients). Karnofsky performance status of greater than 70%. Acceptable hematologic, renal and hepatic function. No patients with active CNS metastases, chronic immunosuppressive use, or prior participation in a therapy requiring the administration of adenovirus were allowed.

2. Quantitative PCR, RT-PCR

Samples were tested using a TaqMan™ based assay. The assay detects a 109 nt amplicon located between the 3′ region of the CMV promoter and the 5′ region of the mda-7 gene. The assay is specific for detecting INGN 241 (Ad-MDA-7 as described in U.S. applications Ser. Nos. 09/615,154, 10/017,472, and 10/378,590, which are all incorporated by reference) in both DNA and RNA.

3. Immunohistochemistry

Serial slices of INGN 241 injected tumors were analyzed for apoptotic activity as defined by the TUNEL reaction (Deadend Colorimetric Apoptosis Detection System, Promega).

4. MDA-7 Protein

Serial slices of INGN 241 injected tumors were analyzed by automated IHC using mda-7 reactive rabbit antibodies (Ab 506-71) supplied by Introgen Therapeutics.

Results

An open-label, Phase I, single dose, dose-escalation study of INGN 241, administered via intratumoral injection to patients with advanced carcinoma was conducted. An evaluation was done to focus on radius of diffusion of the INGN 241 vector, and its resultant protein and biological effects within the tumor mass.

A reference point of injection was marked by including a dye in the administered product.

Also conducted was a phase 2 pilot at the optimal dose to determine both local tumor regression and possible distant effects.

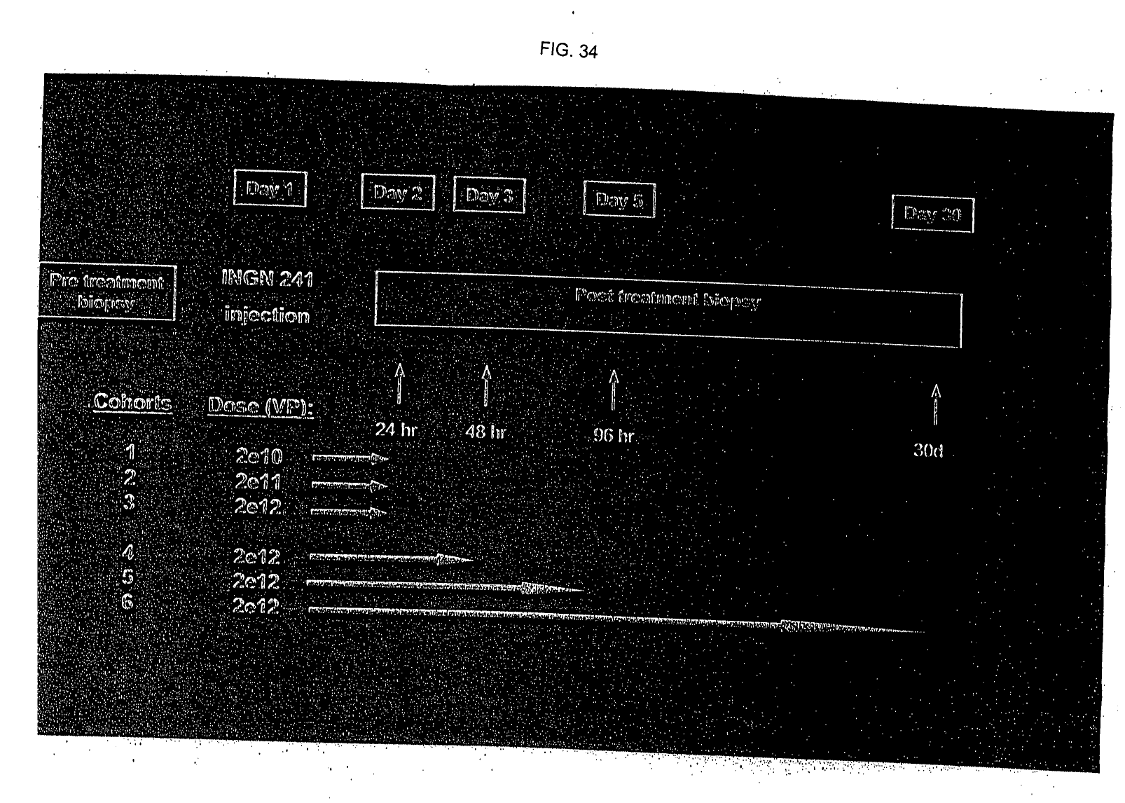

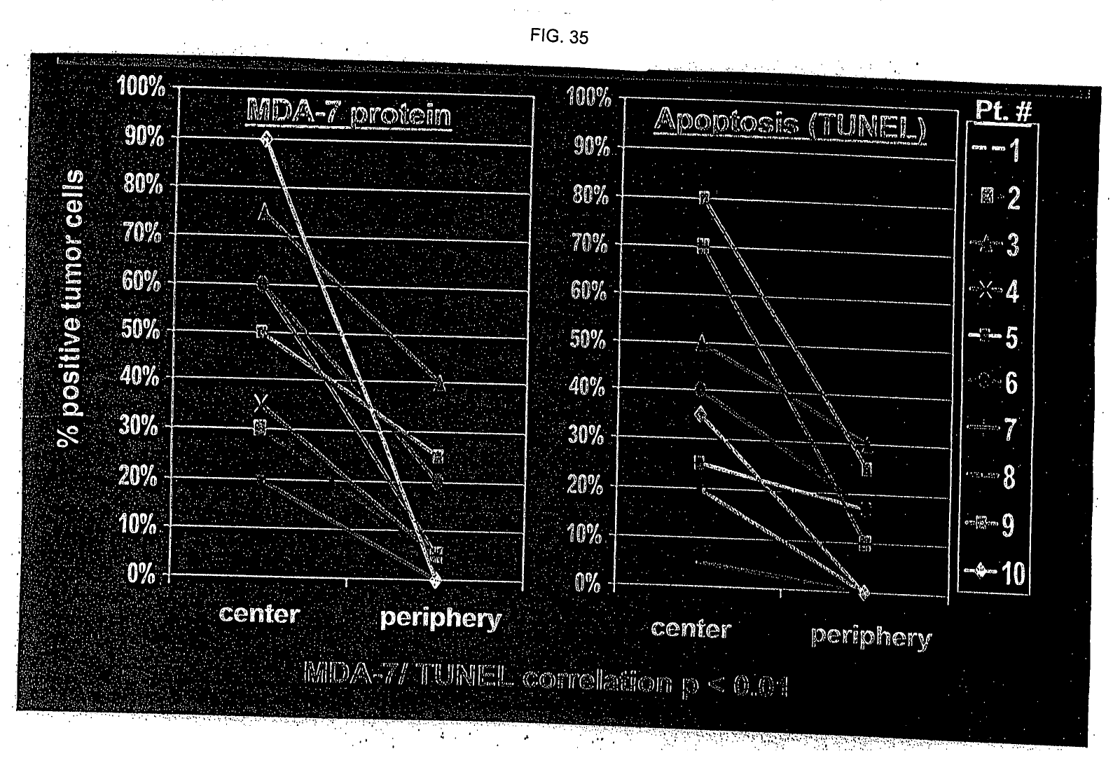

As shown in FIG. 34, cohorts were given different concentrations and time courses of INGN 241. Concentrations of virus were 2×10¹⁰, 2×10¹¹, or 2×10¹² virus particles (vp) per administration. After 24 hours of the last injections, tumors were harvested and sectioned. One side was subject to immunohistochemistry while the other side was subject to RT-PCR to evaluate RNA and DNA concentrations. In one patient given 2×10¹² vp in a 5 cm×5 cm lesion, the center section (within approximately 6 mm from injection site) contained 4.7×10⁸ copies of MDA-7 DNA/μg and 5.6×10⁷ copies of MDA-7 RNA/μg were detected. The section approximately 6 mm from the injection site contained 1.2×10⁶ copies of MDA-7 DNA/μg and 4.6×10⁷ copies of MDA-7 RNA/μg. The section approximately 12 mm from the injection site contained approximately 1.1×10⁶ copies of MDA-7 DNA/μg and 5.0×10³ copies of MDA-7 RNA/μg, while the section 18 mm from the injection site had about 1.9×10⁵ copies of MDA-7 DNA/μg and 9.8×10³ copies of MDA-7 RNA/μg. Immunostaining and TUNEL analysis of a section appproximately 12 mm from the injection site showed that MDA-7 expression co-localized with areas of apoptosis. See also FIG. 35.

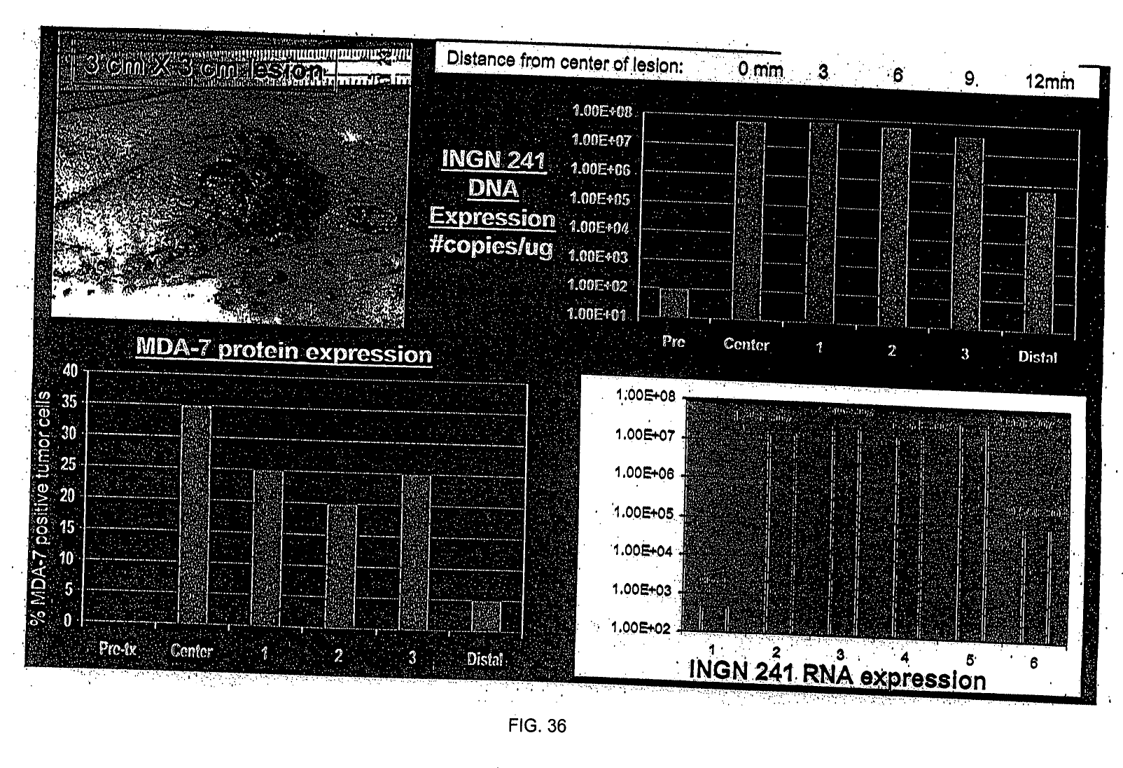

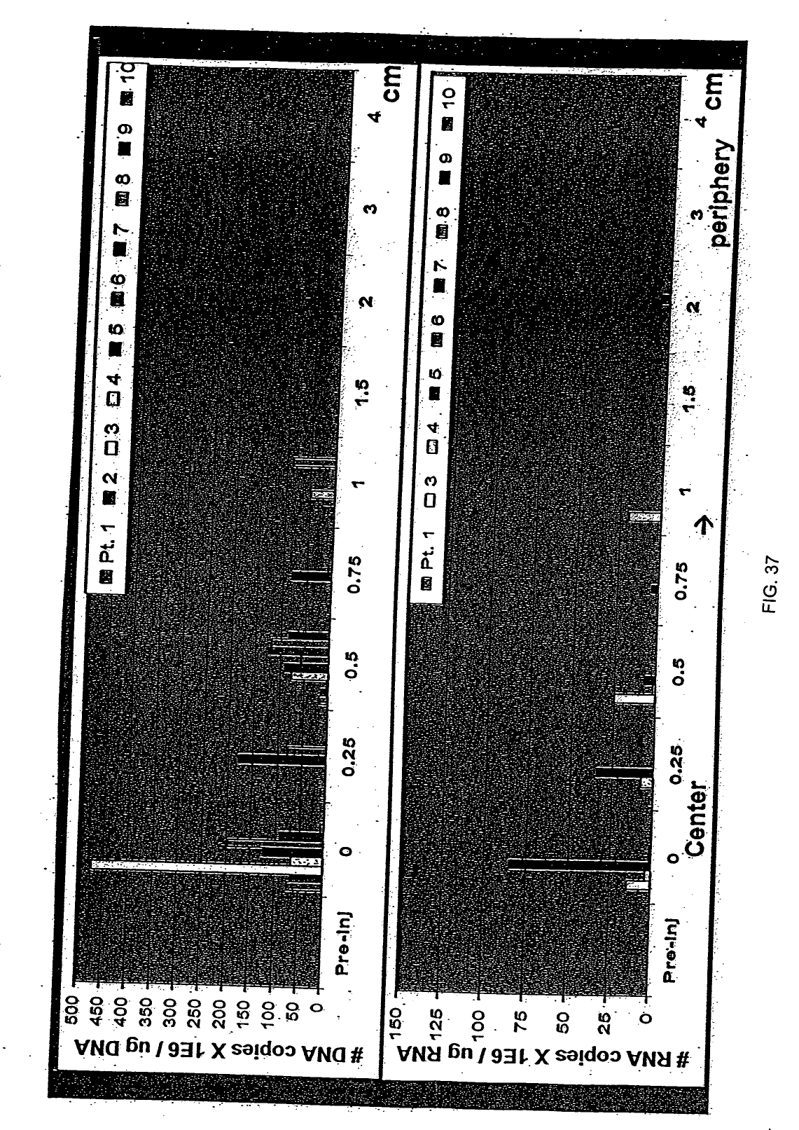

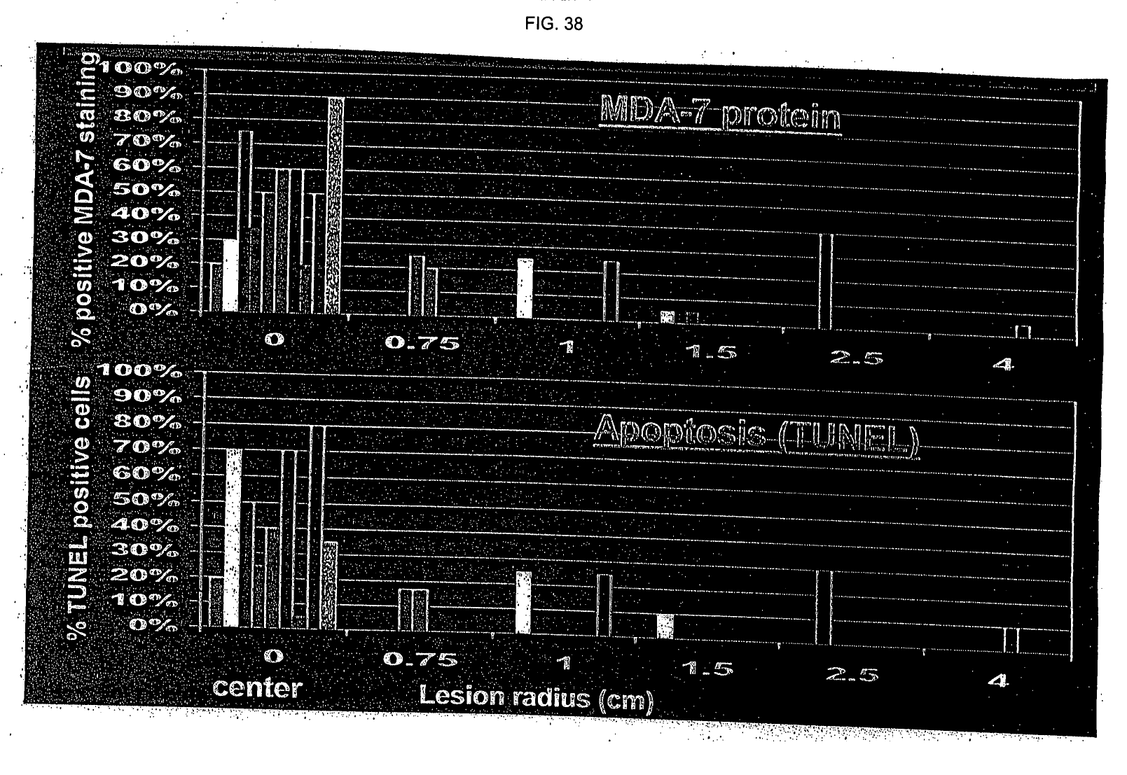

A different patient with a melanoma was evaluated for expression levels of MDA-7 DNA, RNA, and protein with respect to distance from injection site. See FIG. 36. Multiple patients were analyzed for expression levels (FIG. 37) and the correlation between expression levels and apoptosis (FIG. 38).

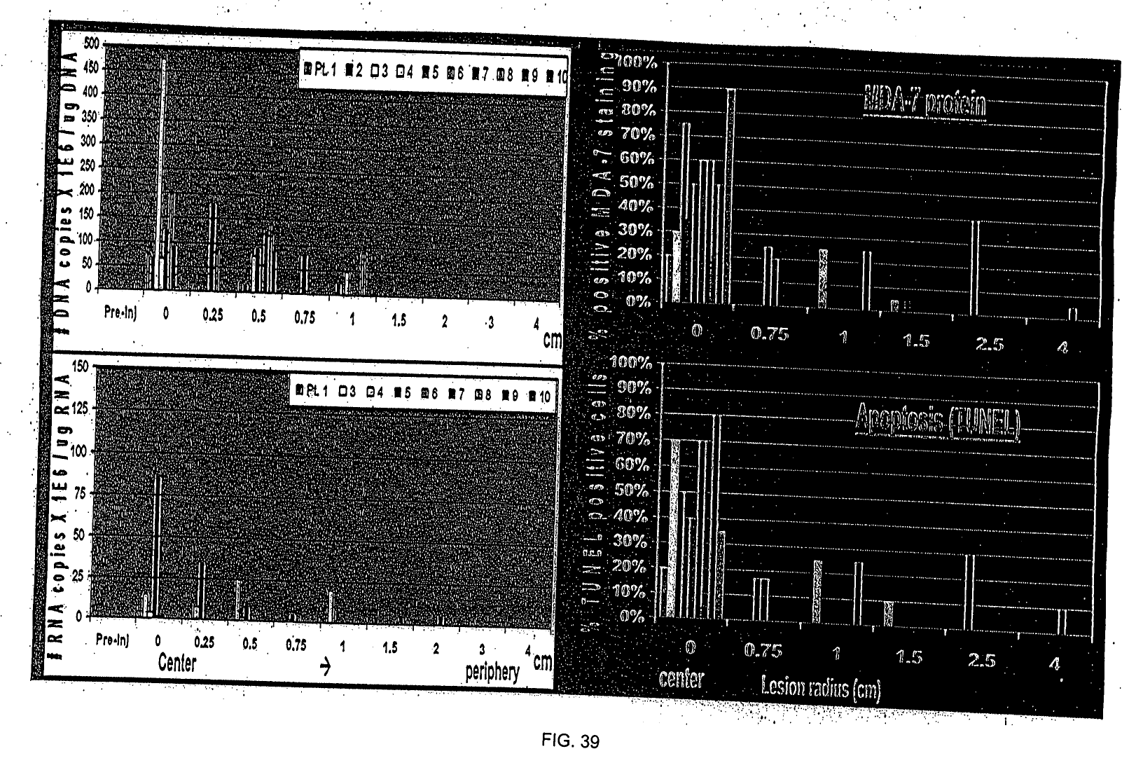

The spread of MDA-7 and its effect on apoptosis was also determined and evaluated (FIG. 39).

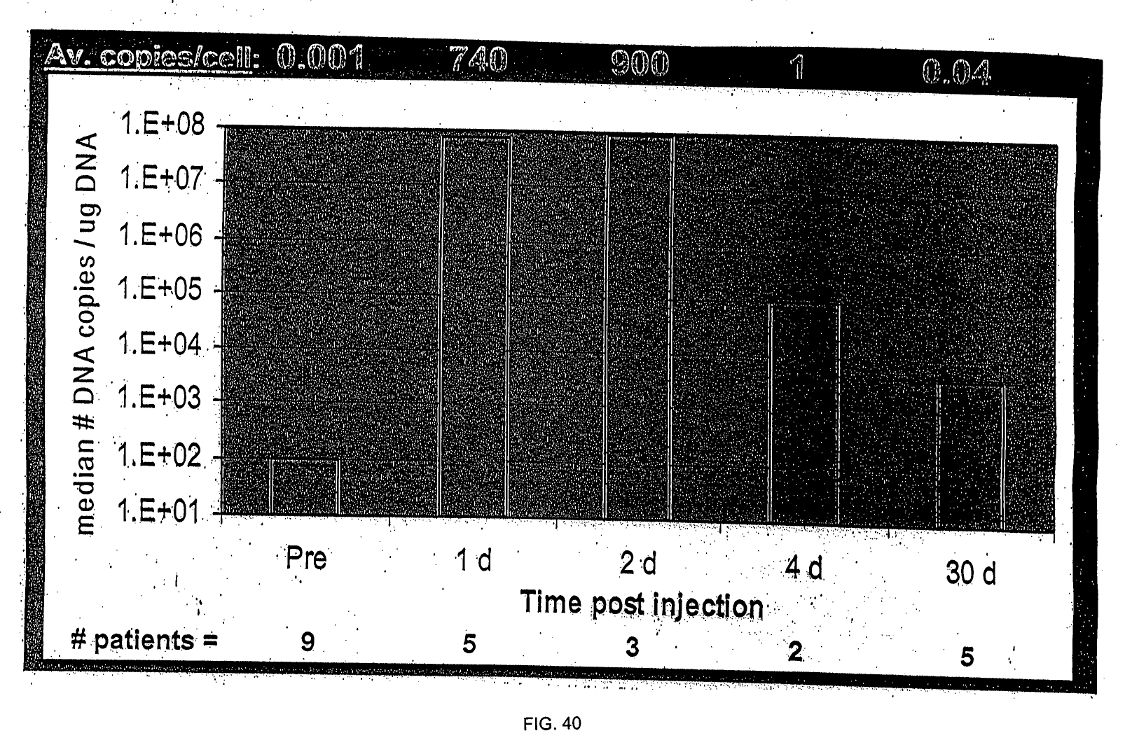

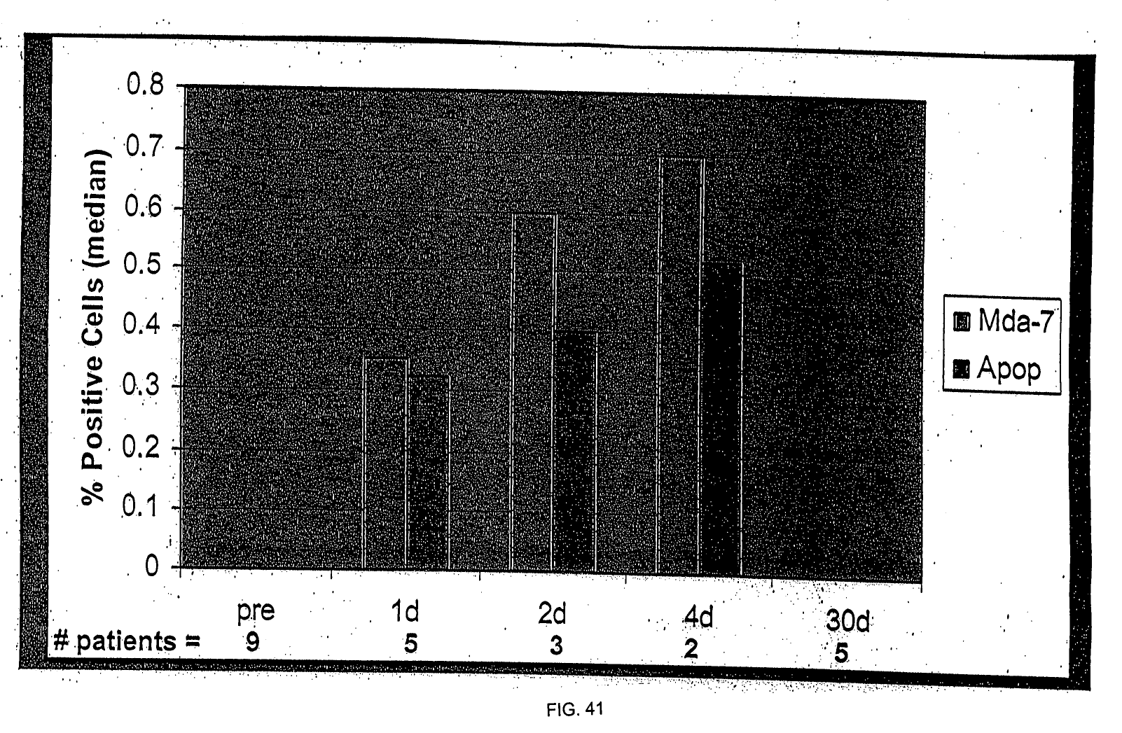

A time course of DNA concentration was conducted at 1 day, 2 days, 4 days, and 30 days post injection (FIG. 40). Similarly, a time course of protein expression and its correlation with apoptosis was done (FIG. 41). A time-dependent increase in MDA-7 protein expression and a marked increase in apopotic cells, which are significantly correlated with distance from the point of injection, was observed. MDA-7 protein and apoptosis were measurable beyond the point of detection of vector DNA, suggesting diffusible, active product. By 96 hrs, intratumoral INGN 241 DNA levels are decreasing; a 4 log decrease (median) at day 30 was observed. By 30 days post injection, MDA-7 protein expression and apototic activity could not be detected.

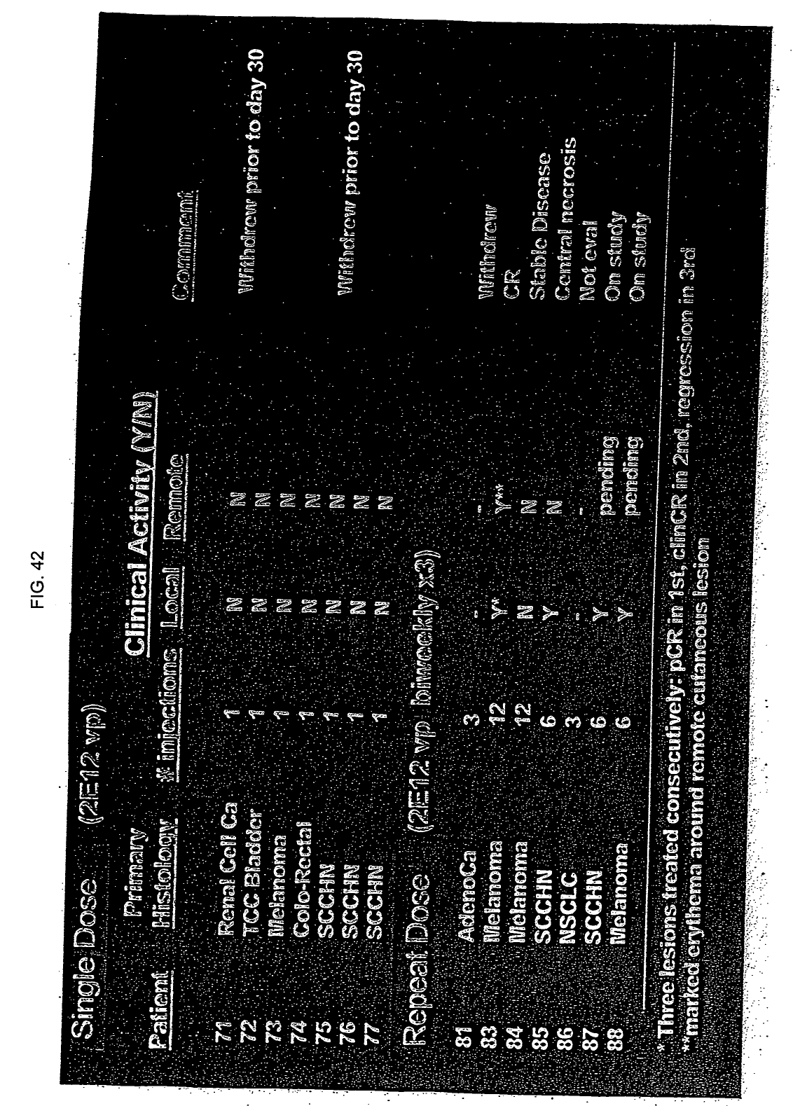

In a phase 2 clinical study, single administration of INGN 241 was compared with respect to multiple administrations (2×10¹² vp every two weeks, three times) in a variety of tumor types (FIG. 42).

Repeat intratumoral doses of INGN 241 produced objective tumor regression in a patient with melanoma (2 cm×2 cm supraclavicular mass) given six 2×10¹² VP of INGN 241 injections intratumorally.

Adverse events are shown in FIG. 41. Overall, study showed INGN 241 intratumoral injections are well tolerated.

### Example 24

### MDA-7 Induces NF-κB; Sulindac Enhances AD-MDA7-Mediated Apoptosis in Human Lung Cancer

Materials and Methods

1. Cell Lines and Cell Culture

Human NSCLC cell lines A549 (adenocarcinoma, wildtype for p53) and H1299 (large cell carcinoma, nulltype for p53) were used in some of these experiments. The normal lung fibroblast cell line CCD-16 was obtained from American Type Culture Collection (ATCC; Rockville, Md.). A549 and H1299 cells were maintained in appropriate medium as previously described (5). CCD-16 cells were cultured in alpha media supplemented with 10% fetal bovine serum (Gibco-BRL, Grand Island, N.Y.) and maintained at 37° C. in a humidified 5% CO₂ plus 95% air atmosphere.

2. Agents

Sulindac, sulindac sulfone, MG132 (a proteosome inhibitor), and cycloheximide (a protein synthesis inhibitor) were obtained from Sigma Chemical Co. (St. Louis, Mo.). Sulindac was dissolved in 1 M Tris-HCl, pH 8.0, to make 100 mM stock solution. MG132 was dissolved in DMSO to make 10 mM stock solution. These stock solution were stored frozen at −20° C.

3. Recombinant Adenoviral Vector

Ad-mda7 and Ad-luc vectors were constructed and purified as have been previously reported (Saeki et al., 2000; Mhashilkar et al., 2001). The transduction efficiencies for the cell lines were determined with an adenoviral vector carrying GFP (Ad-GFP). Transduction efficiency was greater than 80% when infected with 3000 vp/cell. On the basis of these results, cells were treated with 3000 vp/cell in all subsequent experiments.

To determine the effect of sulindac on adenovirus transduction, tumor and normal cells were infected with Ad-GFP at 100 vp/cell and analyzed for GFP expression at 24 h by FACS.

4. Cell Proliferation Assay

All three cell lines (A549, H1299, and CCD-16) were seeded in 60-mm-diameter tissue culture dishes at a density of 1×10⁵ cells/dish in triplicate. The next day, cells were treated with PBS (control), Ad-luc (3000 vp/cell; control), Ad-mda7 (3000 vp/cell; control), sulindac, or a combination of PBS plus sulindac, Ad-luc plus Sulindac, or Ad-mda7 plus Sulindac. The concentrations of sulindac tested were 0.125, 0.25, and 0.5 mM. At 72 h after the start of treatment, the cells were harvested by trypsinization, washed, and subjected to typan-blue exclusion assay as previously described (Saeki et al., 2000). Cell growth was determined by calculating the mean of the cell counts for each treatment group and expressed as a percentage of the total number of cells treated with PBS, Ad-luc, or Ad-mda7 treatment alone (set to 100%).

5. Cell Cycle Distribution and Apoptosis

Cells (5×10⁵) were seeded in a 10-cm-diameter tissue culture dish and treated with PBS, Ad-luc (3000 vp/cell), Ad-mda7 (3000 vp/cell), sulindac, or a combination of PBS plus sulindac, Ad-luc plus sulindac, or Ad-mda7 plus Sulindac. Each treatment group was tested in triplicate. The concentrations of sulindac used were the same as those for the cell-proliferation assay. At 72 h after the start of the treatment, cells were harvested, washed, and analyzed for cell cycle phases and apoptotic fraction as previously described (Saeki et al., 2000). The cell cycle phases and DNA contents were analyzed using FACScan (EPICS XL-MCL; Beckman Coulter, Fullerton, Calif.).

6. Immunofluorescence Assay Cells (1×10⁴) were seeded in 2-well chamber slides (Fisher Scientific) and treated with PBS, Ad-mda7 (3000 vp/cell), PBS plus sulindac (0.5 mM), or Ad-mda7 plus sulindac (0.5 mM). At 48 h after the start of treatment, the cells were washed with PBS and fixed in PBS buffered 4% paraformaldehyde for 30 min at room temperature. The cells were then permeabilized with 0.1% Triton X-100 and 0.1% sodium citrate for 10 min at room temperature, followed by incubation with normal goat serum. At 30 min after the start of the incubation, cells were washed with PBS and incubated with rabbit polyclonal anti-human MDA7 antibody (Introgen Therapeutics Inc., Houston, Tex.) for 1 h at 37° C. The cells were then washed three times with PBS and incubated for 1 h with goat anti-rabbit FITC-tagged secondary antibody (Vector Laboratories, Burlingame, Calif.), washed thee times in PBS, mounted with a cover-slip and observed for MDA-7 protein expression using a Nikon fluorescence microscope (Melville, N.Y.). Photomicrographs were obtained at high-power magnification.

7. Proteasome Activity Assay

Proteasome activity assays were performed as previously described (Choi et al., 2003). Briefly, H1299 cells were seeded in 6-well plates (2×10⁵ cells/well) and treated with Ad-mda7, Ad-mda7 plus sulindac, or Ad-mda7 plus MG132 (5 μM). The sulindac concentrations tested were the same as those in the other assays. At 24 h after the start of treatment, the cells were lysed in proteasome buffer (10 mM Tris-HCl, pH 7.5, 1 mM EDTA, 20% glycerol, 5 mM ATP, and 4 mM DTT), sonicated, and then centrifuged at 1300×g at 4° C. for 10 min. The upper supernatant phase was collected, and the protein concentration of cell lysates was determined as previously described (Saeki et al., 2000).

To assay the chymotrypsin-like activity of the proteasome, the fluorogenic substrate Suc-LLVY-AMC (Chemicon International, Inc., Temecula, Calif.) was used. Twenty micrograms of total protein from each treatment group described above was diluted to 100 μl in reaction buffer (25 mM HEPES, pH 7.5, 0.5 mM EDTA, 0.05% NP-40, and 0.001% SDS). Fluorogenic substrate was added to each sample and incubated at 37° C. for 1 h. The intensity of fluorescence in each sample solution was measured using a fluorescence plate reader (Dynatech Laboratories, Chantily, Va.) at 360-nm excitatory and 460-nm emission wavelengths. All readings were standardized using the fluorescence intensity of an equal volume of free 7-Amino-4-methylcoumarin (AMC) solution (50 μM). The values were expressed as percentages for control greater than the internal positive control percentages provided by the supplier.

8. Real-Time Quantitive RT-PCR

H1299 cells seeded in 6-well plates (5×10⁵/well) were treated with Ad-mda7 (3000 vp/cell) or Ad-mda7 plus Sulindac (0.125, 0.25, or 0.5 mM). Untreated cells served as controls in these experiments. At 36 h after the start of treatment, the cells were washed in PBS, trypsinzed, and resuspended in 1.0 ml of PBS. The cell suspension was transferred into 1.5 ml Eppendorf tubes and centrifuged for 5 min at 10,000 rpm at 4° C. The supernatant was discarded, and total RNA from the cell-pellet was extracted using an RNA isolation kit as described by the manufacturer (Ambion Corp., Austin, Tex.). The isolated RNA was then treated with DNase I to remove residual DNA and subsequently quantitated using a spectrophotometer at 260-nm and 280-nm wavelengths. Total RNA (0.1 μg) from each sample was reverse transcribed using a SuperScript RT kit (Invitrogen, Carlsbad, Calif.). Quantification of mda-7 mRNA was performed using real time quantitative RT-PCR. Briefly, quantitative PCR was performed in 20-μl volumes consisting of 1 μl of total RNA, 10 μl of PCR Supermix (PE Applied BioSystems, Foster City, Calif.), 0.2 μM mda-7-specific primers, and 0.1 μM of fluorescent probe. The resulting relative increase in reporter and quencher fluorescent dye emission was monitored in real time during PCR amplification using a 7700 sequence detector (PE Applied BioSystems). The two-step PCR cycling was carried out as follows: 2 min at 50° C., 10 min at 95° C., 40 cycles of 15 at 95° C., and 1 min at 60 ° C. The human GAPDH housekeeping gene was used as internal control in the amplification reactions and the primers provided by the supplier (PE Applied Biosystems).

The oligonucleotide sequences used in the assays described above are as follows:

| | |
|---|---|
| MDA-7 5′-primer, | CCCGTAATAAGCTTGGTACCG; |
| and | |
| MDA-7 3′-primer, | TAAATTGGCGAAAGCAGCTC; |
| probe, | FAM-TGGAATTCGGCTTACAAGACATGACTGTG- |
| | TAMRA. |

All reactions were performed in triplicate. After the cycling reaction was complete, a standard curve, the threshold cycling (Ct) value of each sample and its corresponding starting quantity based on the standard curve were determined using the 7700 sequence detector system software (PE Applied Biosystems). The differences in mda-7 mRNA expression among various treatment groups were expressed as the change in values over GAPDH.

9. Half-Life Analysis

H1299 cells were seeded at a density of 2×10⁵ cells in a 60-mm-diameter tissue culture dish. The next day, the cells were infected with Ad-mda7 (3000 vp/cell). At 48 h after infection, sulindac (1 mM) was either added or not added and the incubation continued. Two hours later, the protein synthesis inhibitor cycloheximide (10 μg/ml) was added to the cells and the incubation continued. Cells were harvested at 0, 3, 6, 9, 11, and 13 h after cycloheximide treatment; cell lysates were then prepared and analyzed for MDA-7 protein expression by western blot analysis as previously described (Saeki et al., 2000; Mhashilkar et al., 2001).

10. Western Blot Analysis

Cells treated with PBS, Ad-mda7, Ad-luc, sulindac, sulindac sulfone, or a combination of Ad-luc or Ad-mda7 with sulindac or sulindac sulfone were subjected to western blot analyses as previously described (Saeki et al., 2000; Mhashilkar et al., 2001). The following primary antibodies were used for detection: caspase-3 and PARP (BD Pharmingen, San Diego, Calif.); caspase-9, pJNK, and pp38 MAPK (Cell Signaling Technology Inc., Beverly, Calif.), PKR, BAX, BAK, BCL-2, BCL-XL, COX-2, and Ub (Santa Cruz Biotechnology, Santa Cruz, Calif.); β-actin (Sigma); and MDA-7 (Introgen Therapeutics). The proteins were detected using appropriate horseradish peroxidase-conjugated secondary antibodies, and visualized on enhanced chemiluminescence film (Hyperfilm; Amersham) by application of Amersham's enhanced chemiluminescence western blotting detection system.

11. In Vivo Analysis

To determine whether sulindac enhances Ad-mda7-mediated tumor growth inhibition of xenograft tumors in vivo, H1299 lung tumor cells (5×10⁶) were injected subcutaneously into the lower right flank of athymic BALB/c female nude mice (n=40). When the tumor reached 50-100 mm3 the animals were divided into groups and treated: PBS (n=8), sulindac (n=8), Ad-mda7 (n=8), Ad-luc plus sulindac (n=8), or Ad-mda7 plus sulindac (n=8). The mice were treated with Ad-luc or Ad-mda7 intratumorally (3×109 vp/dose) thrice a week. In mice receiving sulindac, 40 mg/kg was administered i.p. every day. Animals were weighed once a week to determine the body weight. Tumor growth was monitored and measured three times a week as described previously (Saeki et al., 2002; Ramesh et al., 2003). At 22-25 days after initiation of the treatment all animals were killed via CO₂ inhalation, and the tumors were removed for histopathologic examination and western blot analysis. Experiments were performed two separate times for reproducibility and statistical significance.

12. Statistical Analysis

Student's t test and ANOVA were used to calculate the statistical significance of the experimental results. A value of P<0.05 was considered statistically significant.

Results

1. MDA-7 Induces NF-κB

In the present study, it was demonstrated that adenovirus-mediated mda-7 (Ad-mda7) gene transfer in two NSCLC cells lines (H1299 and A549) resulted in NF-κB activation, as demonstrated by electromobility shift assay (EMSA). Marked activation of NF-κB was observed between 20-48 hours in cells treated with Ad-mda7 but not in control cells treated with PBS, or cells treated with Ad-luciferase. Furthermore, activation of NF-κB occurred in a dose-dependent manner, with increasing concentrations of Ad-mda7 resulting in increased NF-κB activation.

Coinciding with NF-κB activation was the degradation of an inhibitor of NF-κB, I-κBα Ad-mda7-induced NF-κB was found to be composed of p50 and p65 subunits. Ad-mda7 also was found to induce NF-κB (p65) nuclear translocation and to increase the DNA binding activity of NF-κB in a dose-dependent manner, according to EMSA done 36 and 48 hours after infection of A549 cells and 42 and 48 hours after infection of H1299 cells.

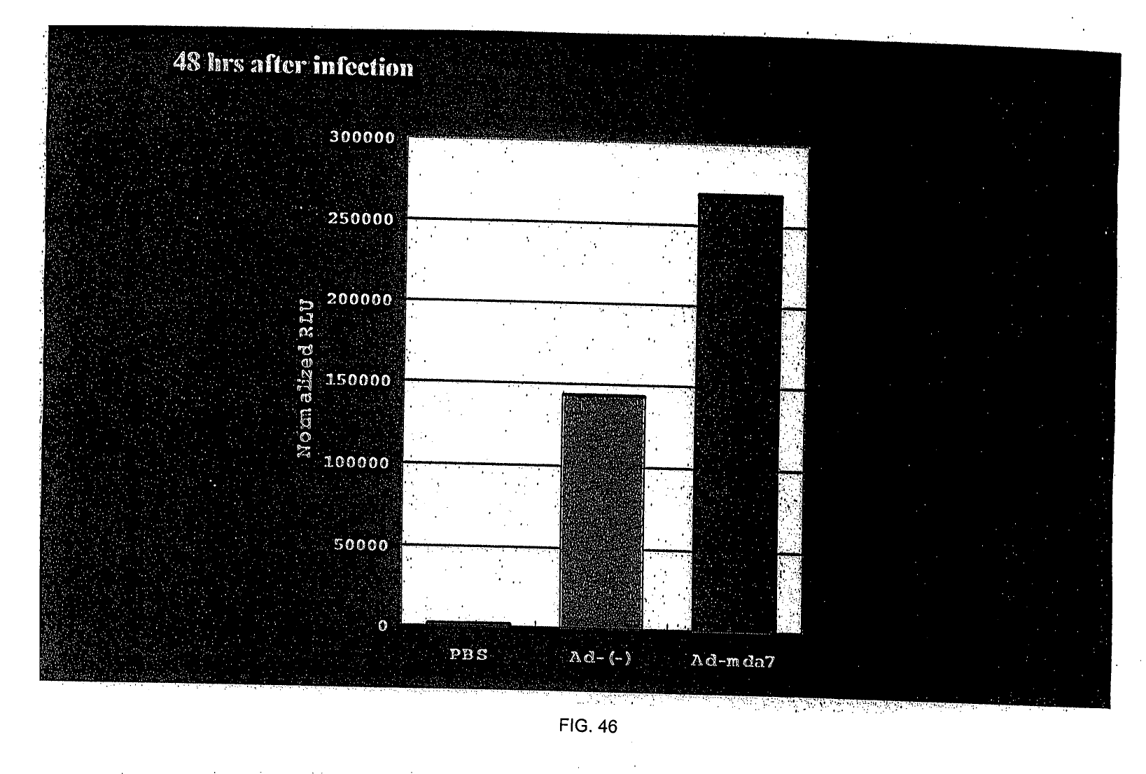

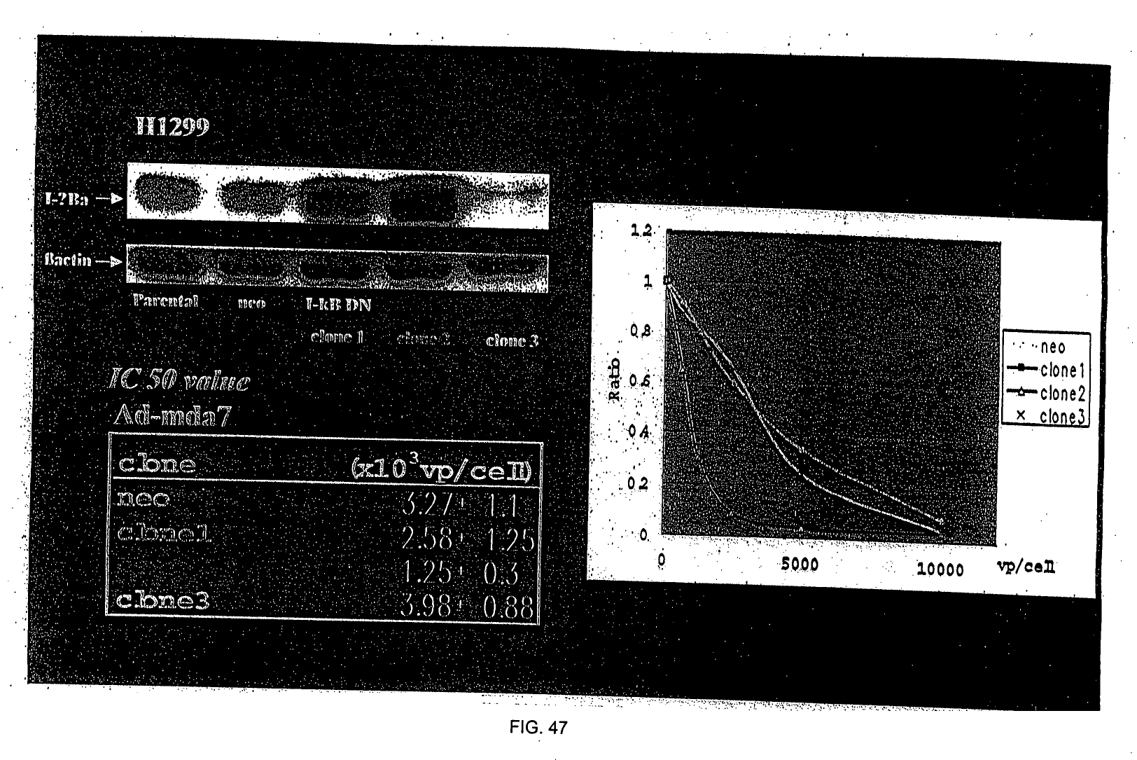

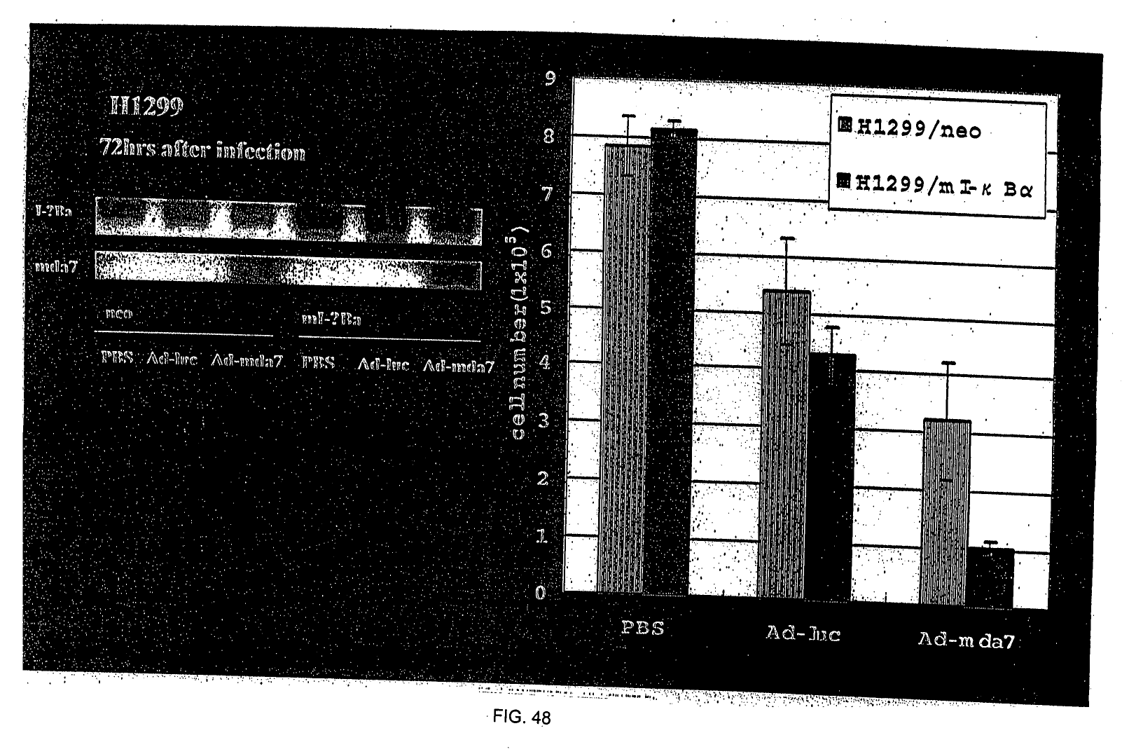

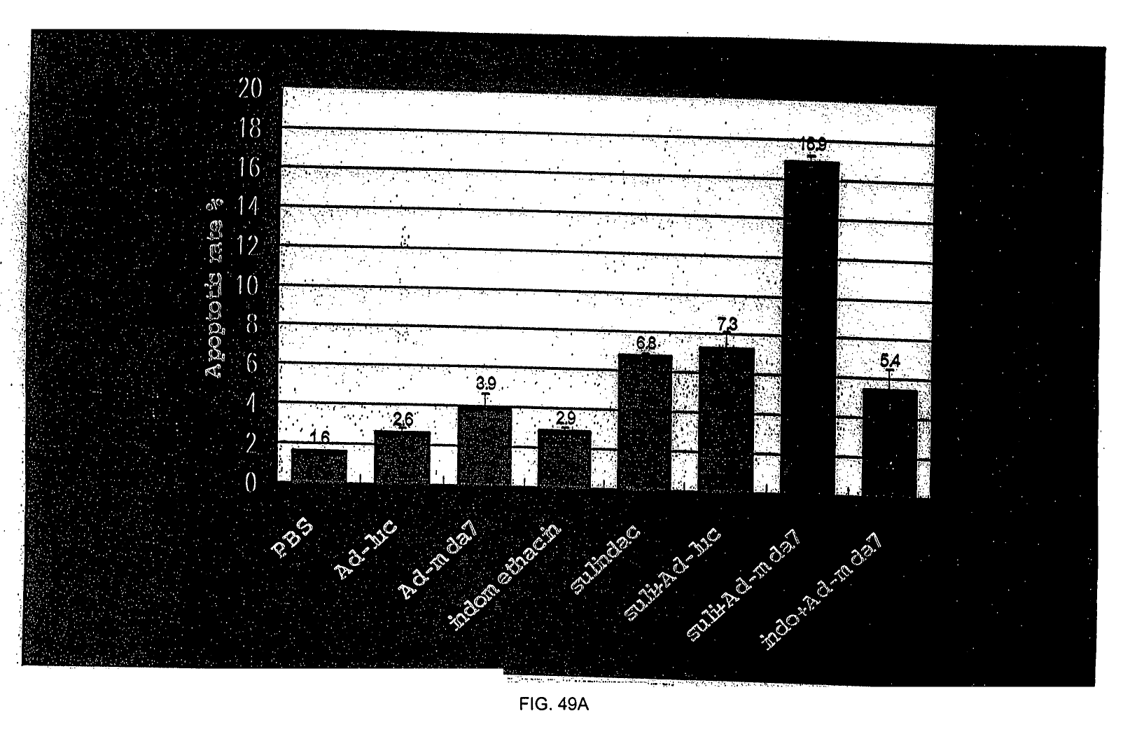

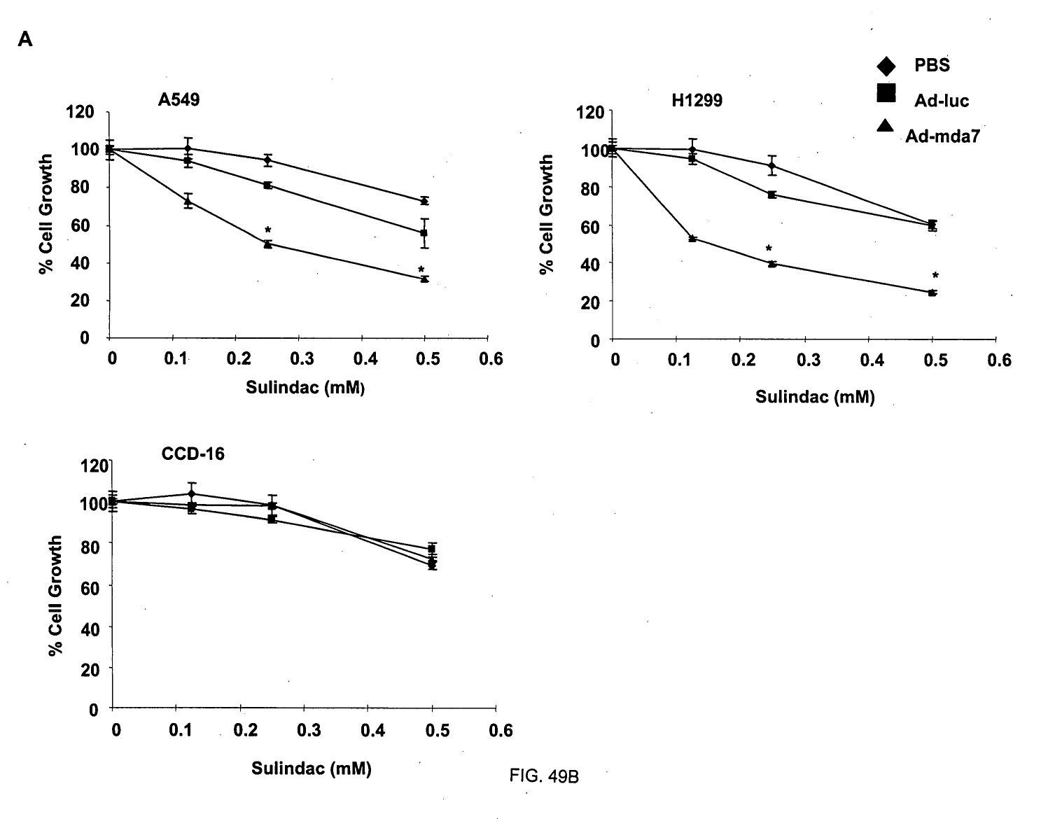

Ad-mda7 was also found to activate NF-κB-dependent reporter gene expression (FIG. 46). It was also found to have a cytotoxic effect in dominant negative I-κBα cells (FIG. 47). Ad-mda7 significantly suppressed the cell growth in dominant negative I-κBα cells (FIG. 48). Furthermore, transfection of H1299 cells with an adenoviral vector overexpressing dominant negative mutant I-κB (Ad-mIκB) significantly inhibited Ad-mda7 induced transcriptional and DNA binding activity of NF-κB, resulting in increased tumor cell apoptosis, when compared to control cells that were treated with Ad-luc.

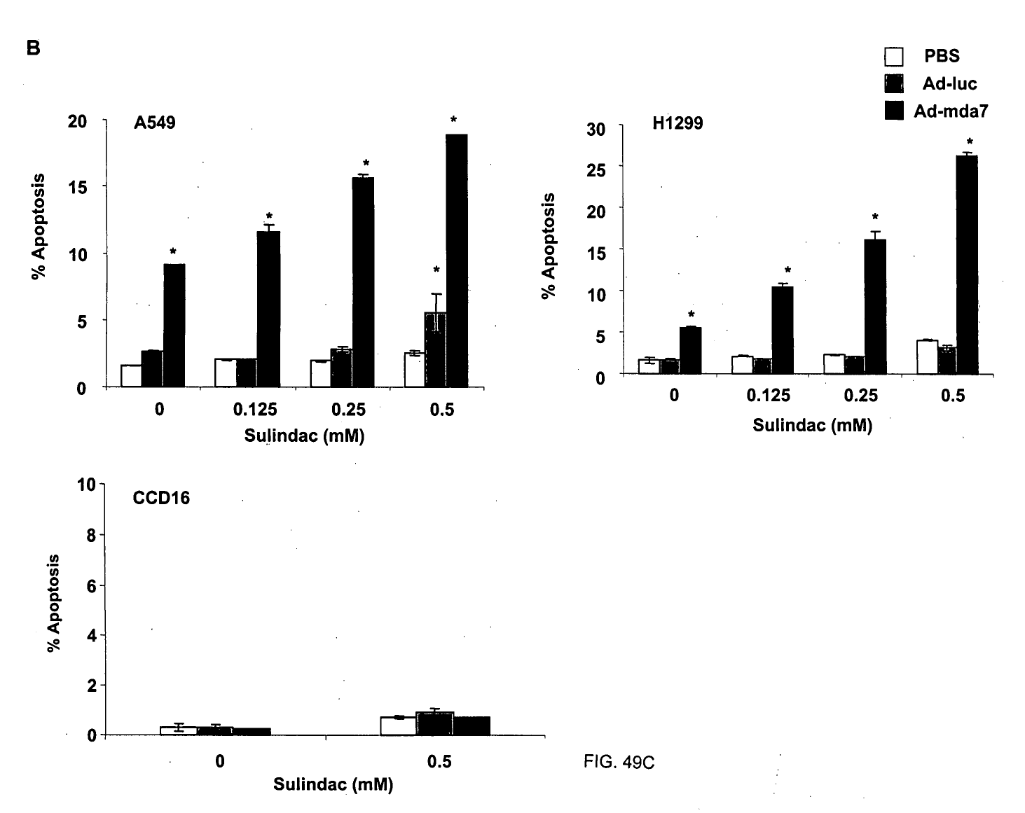

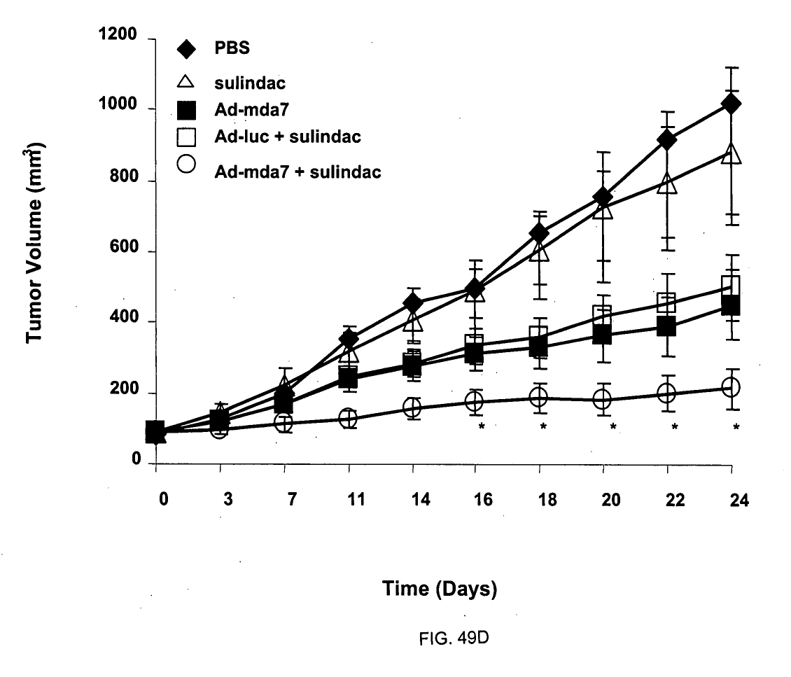

Sulindac was found to inhibit NF-κB activation, as determined by EMSA, in a dose-dependent manner. Additionally, inhibition of MDA-7 mediated NF-κB activation by Sulindac, a non-steroidal anti-inflammatory drug, resulted in a synergistic therapeutic effect (FIG. 49A). These results indicate that MDA-7 expression in lung cancer cells induces NF-κB, and its inhibition using Ad-mIκB or Sulindac enhances the therapeutic effect.

2. Sulindac Enhances Ad-mda7 Mediated Growth Inhibition in Lung Cancer Cells

Because previous studies have shown that sulindac exerts cytotoxic effects on cancer cells (Sanchez-Alcazar et al., 2003), preliminary experiments were conducted to determine the minimum cytotoxic dose of sulindac against NSCLC (A549 and H1299) cells and normal (CCD-16) cells. Treatment of these cells with sulindac at various concentrations (0.062, 0.12, 0.25, 0.5, 1, and 2 mM) demonstrated growth inhibition, with an IC₅₀ of 0.58, 0.61, and 0.94 mM in A549, H1299, and CCD-16 cells, respectively. At higher doses (1 and 2 mM), cell proliferation was inhibited in both tumor and normal cells resulting in apoptosis (data not shown). However, the inhibitory effect on tumor cell proliferation was higher (>90%) than in normal cells (60%). On the basis of these results, sulindac was tested at concentrations of less than 0.5 mM in subsequent experiments.

To investigate whether sulindac in combination with Ad-mda7 can inhibit cell proliferation and induce apoptosis, A549, H1299, and CCD-16 were treated with PBS, Ad-luc, and Ad-mda7, alone and in combination with sulindac (0.125, 0.25, or 0.5 mM). Analysis of cells 72 h after treatment demonstrated that the combination of sulindac and Ad-mda7 significantly inhibited tumor cell proliferation compared with cells that treated with Ad-mda7 or sulindac alone (P=0.001; FIG. 49B). The growth inhibitory effects produced by this combination therapy were also significant compared with the other treatment groups and were sulindac dose-dependent. In contrast, no significant growth inhibitory effects were observed in normal fibroblast cells treated with Ad-mda7 plus sulindac at any concentration tested compared with the other treatment groups. These results indicate that sulindac selectively enhances Ad-mda7-mediated inhibitory activity in tumor but not normal cells.

To further evaluate whether treatment with Ad-mda7 plus sulindac induces apoptosis, tumor and normal cells were analyzed 72 h after treatment for apoptotic changes by FACS analysis. The number of cells in sub-G₀/G₁ phase, an indicator of apoptotic changes, was significantly higher in tumor (H1299 and A549) cells treated with Ad-mda7 alone or in combination with sulindac than in normal cells subjected to these treatments (P=0.001). (FIG. 49C). However, the number of apoptotic cells among the tumor cells treated with Ad-mda7 plus sulindac was significantly greater than that among tumor cells treated with Ad-mda7 alone (P<0.01) and was sulindac dose-dependent. The numbers of apoptotic cells among cells treated with Ad-luc alone or in combination with sulindac were not significantly higher than the number of apoptotic cells among PBS-treated cells. However, among A549 tumor cells, the number of apoptotic cells was significantly increased (P=0.01) at the highest concentration of sulindac (0.5 mM) when combined with Ad-luc treatment compared with PBS-treated cells. Treatment of CCD-16 cells with Ad-mda7 or Ad-mda7 and sulindac, even at the highest sulindac concentration (0.5 mM), yielded no significant difference in the number of apoptotic cells compared with control cells (FIG. 49C). Similar enhanced growth inhibitory effects were also observed when lung cancer cells were treated with Ad-mda7 in combination with sulindac sulfone. These results demonstrate that lung tumor but not normal cells selectively undergo apoptosis when treated with Ad-mda7 and sulindac or sulindac sulfone. Furthermore, the growth inhibitory effects mediated by Ad-mda7 and sulindac occur independent of the p53 status, given that they occurred in p53-null and p53 wild-type tumor cell lines.

3. Sulindac does not Increase Ad-mda7 Transduction

The ability of therapeutic agents to enhance adenovirus transduction efficiency has previously been reported (Lin et al., 2003). Based on this report whether sulindac enhances adenoviral transduction was determined. For this purpose, tumor and normal cells were infected with Ad-GFP at 100 vp/cell and treated with sulindac at various concentrations (Table 7 below). Cells were transduced with Ad-GFP at low particle numbers since more than 80% of cells are transduced at a higher vp, making it difficult to determine the effects of sulindac on transduction. At 24 h after treatment, the cells were analyzed by flow cytometry. No significant difference in the transduction efficiency was observed between cells treated with Ad-GFP plus sulindac and cells treated with Ad-GFP alone (Table 7 below). However, transduction was increased in A549 cells that had been treated with 0.5 mM sulindac compared with the other groups treated with sulindac at the lower concentrations (P.=0.001).

TABLE 7
Transduction efficiency in lung cancer (A549 and H1299) and normal
(CCD-16) cells treated with Ad-GFP and sulindac.
Cells were treated with Ad-GFP (100 vp/cell) for 3 h, followed by
sulindac at the indicated concentrations for 24 h. Percentages of
transduction efficiency were determined by flow cytometry.

| | Sulindac (mM) | | | |
|---|---|---|---|---|
| Cell line | 0 | 0.125 | 0.25 | 0.5 |
| A459 | 48.8 ± 6.5 | 52.4 ± 0.6 | 51.5 ± 1.3 | 65.0 ± 1.5 + a |
| H1299 | 79.5 ± 2.2 | 80.0 ± 0.5 | 78.6 ± 1.2 | 82.7 ± 2.8 |
| CCD16 | 15.7 ± 0.2 | 12.1 ± 0.4 | 11.1 ± 1.0 | 13.7 ± 0.4 |

^{a}P < 0.05 compared with Ad-GFP treatment alone. No other differences between the groups shown was significant.

4. Sulindac Increases Exogenous MDA-7 Expression

To identify the mechanism by which sulindac enhances Ad-mda7-mediated growth inhibition and apoptosis in lung cancer cells, transgenic MDA-7 protein expression was examined by Western blotting. All three cell lines (H1299, A549, and CCD-16) were treated with Ad-mda7/sulindac for 36 h and analyzed for MDA-7 expression. In Ad-mda7-treated A549 and H1299 cells, sulindac markedly increased the steady-state levels of transgenic MDA-7 in a dose-dependent manner; endogenous MDA-7 expression was not detected in cells treated with either PBS or sulindac alone. Furthermore, the ability of sulindac to increase transgenic protein expression was not limited to MDA-7: sulindac increased the steady-state levels of transgenic GFP and p53 protein in tumor cells treated with Ad-GFP and Ad-p53, respectively. In contrast, in normal CCD-16 cells treated with Ad-mda7, sulindac only slightly increased exogenous MDA-7 protein expression. The effect of sulindac on exogenous GFP or p53 protein was not tested in normal cells.

To evaluate subcellular localization of MDA-7 protein, immunofluorescence studies were performed. Consistent with the Western blot data, MDA-7 expression was significantly elevated in cells treated with Ad-mda7/sulindac compared with cells treated with Ad-mda7 alone. Furthermore, the subcellular localization of MDA-7 was not altered by sulindac treatment. MDA-7 expression was not detectable in cells treated with PBS or sulindac alone. These results demonstrate that sulindac increases transgenic MDA-7 expression in a dose-dependent manner and suggest that this increase contributes to increased apoptotic activity.

To test whether the ability to increase exogenous protein expression was unique to sulindac, experiments were also carried out using sulindac sulfone. Treatment of H1299 cells with Ad-mda7 and sulindac sulfone showed increased exogenous MDA-7 expression compared to cells treated with Ad-mda7 alone. MDA-7 expression was not detected in cells that were treated with PBS or sulindac sulfone. These results demonstrate the ability of sulindac and its metabolites to increase exogenous protein expression.

5. Sulindac Enhances Ad-mda7-Mediated Apoptotic Signaling

It was previously reported that induction of Ad-mda7-mediated apoptosis in lung cancer cells was associated with activation of the caspase cascade, including cleavage of caspase-9, caspase-3, and PARP (Saeki et al., 2000; Mhashilkar et al., 2001). To determine whether treatment with Ad-mda7 and sulindac affects the caspase cascade, tumor and normal cells were analyzed for these molecular markers. Tumor (A549 and H1299) cells treated with Ad-mda7 alone or in combination with sulindac demonstrated cleavage of capase-9, caspase-3, and PARP, which are indicators of activation of the caspase cascade. The expression of cleaved caspase-9, caspase-3, and PARP corresponded to both the concentration of sulindac and the level of MDA-7 expression. Activation of caspase-9, caspase-3, and PARP was also observed in A549 (but not H1299) cells that had been treated with Ad-luc plus the highest concentration of sulindac (0.5 mM) and was consistent with the increased apoptotic fraction revealed in these cells by FACS analysis (FIG. 49C). However, the level of activation was significantly lower than that in A549 cells treated with Ad-mda7 or Ad-mda7/sulindac. The caspase cascade was not activated in either A549 or H1299 cells that were untreated or treated with sulindac alone. In CCD-16 cells, the caspase cascade was not activated in cells that were treated with Ad-mda7 alone or in combination with sulindac compared with cells that were untreated or treated with sulindac alone, Ad-luc alone, or a combination of Ad-luc plus sulindac. These results demonstrate that sulindac selectively enhances caspase cascade activation in tumor but not normal cells.

Expression of additional effector molecules upstream of the caspase cascade that are modulated by Ad-mda7. and sulindac treatments was investigated next. Previous studies have demonstrated PKR, p38MAPK, and pJNK to be important in Ad-mda7-induced apoptosis in lung cancer cells (Pataer et al., 2002; Kawabe et al., 2002; Sarkar et al., 2002). Similarly, regulation of the Bcl-2 family (Bax, Bak, Bcl-2, and Bcl-X_{L}) proteins have been shown to be critical for sulindac-induced apoptosis and independent of p53 status (Yang et al., 2003; McEntee et al., 1999). On the basis of these reports, the expression of PKR, pJNK, pp38MAPK, and several Bcl-2 family members was evaluated in H1299 cells after treatment with Ad-mda7 and sulindac. Expression of PKR, pJNK, and pp38MAPK was increased in cells treated with Ad-mda7 alone or in combination with sulindac compared with untreated, sulindac-treated, and Ad-luc-treated cells. PKR was also slightly increased in cells treated with Ad-luc plus sulindac compared with untreated, Ad-luc treated, and sulindac-treated cells. However, the PKR levels in the cells treated with Ad-luc plus sulindac were lower than those observed in cells treated with Ad-mda7 plus sulindac. The increase in PKR, pJNK, and pp38MAPK was associated with the expression levels of MDA-7 induced by sulindac. No change in the expression levels of Bax or Bak, two inducers of apoptosis, or Bcl-X_{L}, an inhibitor of apoptosis, was detected in any of the treatment groups. The expression level of Bcl-2 was slightly decreased only in cells that had been treated with Ad-mda7 and 0.5 mM sulindac. These results support the idea that the induction of apoptosis by Ad-mda7 plus sulindac depends primarily on the ability of sulindac to enhance ectopic MDA-7 expression.

The possibility that enhanced tumor cell killing with Ad-mda7 plus sulindac treatment was due to COX-2 inhibition was next investigated. Increased COX-2 expression was observed in cells treated with Ad-mda7 and Ad-mda7 plus sulindac. However, there was no significant difference in COX-2 expression levels between the two treatment groups. COX-2 expression was not observed in cells that were treated with PBS, sulindac Ad-luc, and Ad-luc plus sulindac.

6. Effects of Sulindac and Ad-mda7 Treatment on Cell Cycle

Previous studies have demonstrated that sulindac induces cell cycle arrest at G1 (Piazza et al., 1997), and that Ad-mda7 induces cell-cycle arrest at G₂/M (Saeki et al., 2000; Mhashilkar et al., 2001; Ekmekcioglu et al., 2001). On the basis of these reports the combined effects of sulindac/Ad-mda7 treatment on cell cycle regulation was investigated by FACS analysis. Tumor cells either were untreated or were treated with sulindac, Ad-luc, Ad-mda7, or Ad-mda7 plus sulindac for 72 h. As previously reported, Ad-mda7 but not Ad-luc treatment increased the number among the G₂/M phase of cell cycle in both A549 (27.2%) and H1299 (42.5%) cells (Table 8 below). Sulindac treatment alone increased the number of cells in the GI phase. In both tumor cell lines, the number of GI-phase cells was markedly increased at 0.5 mM compared with 0.125 mM sulindac (75.6% versus 64.8% in A549 and 74.6% versus 66.4% in H1299 cells, respectively). Treatment with sulindac and Ad-mda7 abrogated Ad-mda7-induced G₂/M arrest. The effect was more pronounced among cells treated with 0.5 mM sulindac in combination of Ad-mda7, resulting in a decrease in the number of G₂/M-phase cells, from 27.2% to 12.3% in A549 and from 42.5% to 32.4% in H1299 cells, respectively. Abrogation of Ad-mda7-induced G₂/M-phase arrest by sulindac was also observed at 48 h after treatment. These results demonstrate that sulindac and Ad-mda7 affect different phases of the cell cycle and that sulindac-enhanced Ad-mda-7 tumor cell killing does not occur via increased G₂/M arrest.

TABLE 8
Cell cycle distribution in lung cancer cells treated with sulindac, Ad-
mda7 or both.
Cells were treated with PBS, Ad-luc, or Ad-mda7 alone, or
combination with 0.5 mM sulindac for 72 h, followed by FACS
analysis. The percentage of cells in each cell cycle phase was
determined by analysis of the DNA content histogram. Values
are the means of duplicate samples. Similar results were
observed in at least two independent experiments.

| | A549, % | | | H1299, % | | |
|---|---|---|---|---|---|---|
| Treatment | G1 | S | G2/M | G1 | S | G2/M |
| Untreated control medium | 68.6 | 26.4 | 6.9 | 58.8 | 28.5 | 12.8 |
| Control + Sulindac 0.125 mM | 64.8 | 28.9 | 6.4 | 66.4 | 22.3 | 11.4 |
| Control + Sulindac 0.5 mM | 75.6 | 18.5 | 6.0 | 74.6 | 13.0 | 12.7 |
| Ad-mda7 | 41.3 | 31.6 | 27.2 | 44.2 | 13.5 | 42.5 |
| Ad-mda7 + Sulindac 0.125 | 35.6 | 31.8 | 32.7 | 46.9 | 14.0 | 39.2 |
| mM | | | | | | |
| Ad-mda7 + Sulindac 0.5 mM | 43.4 | 42.6 | 12.3 | 49.2 | 18.4 | 32.4 |
| Ad-luc | 73.8 | 17.1 | 7.1 | 78.3 | 9.8 | 11.9 |
| Ad-mda7 + Sulindac 0.5 mM | 70.0 | 18.5 | 11.5 | 70.4 | 9.7 | 20 |

7. Sulindac Delays Exogenous MDA-7 Protein Degradation

To assess the mechanism by which sulindac increases exogenous MDA-7 protein, the effect of sulindac on transcriptional activity and MDA-7 protein degradation was examined in H1299 cells. To determine the effect of sulindac on the transcriptional activity of Ad-mda7, quantitative real-time PCR analysis was performed using RNA samples extracted from cells that were untreated or that were treated with Ad-mda7 alone or with sulindac at various concentrations. No significant difference in mRNA levels was observed in the cells treated with Ad-mda7/sulindac compared with untreated, and Ad-mda7-treated cells.

To evaluate whether sulindac treatment regulates MDA-7 protein degradation, H1299 cells were treated with Ad-mda7 alone or in combination with sulindac for various durations, and the half-life of MDA-7 determined. The MDA-7 protein levels in the Ad-mda7-control cells decreased over time; protein degradation was complete at 11 h. In contrast, the degradation of MDA-7 protein in the cells treated with Ad-mda7 plus sulindac was delayed, as demonstrated by substantial levels of detectable protein at 13 h. Semi-quantitative analysis of the protein levels indicated that at 0-13 h, the MDA-7 protein level was 8-15 times higher in the cells treated with Ad-mda7 plus sulindac than in the Ad-mda7-treated cells. These results demonstrate that the increase in MDA-7 protein expression in cells treated with Ad-mda7/sulindac is a result of a sulindac-mediated delay of MDA-7 protein degradation.

8. Sulindac-Enhanced MDA-7 Expression is not Due to Inhibition of Proteasome Activity

Given that recent studies have demonstrated that some NSAIDs inhibit proteasome activity (Choi et al., 2003; Huang et al., 2002), whether the enhanced MDA-7 protein expression mediated by sulindac is due to its ability to inhibit proteasome activity was investigated. For this purpose, the effects of sulindac were compared to those of MG132, a known proteasome inhibitor (He et al., 2003) by Western blotting, Ub degradation assay, and proteasome enzymatic activity assay. Western blotting demonstrated that sulindac or MG132 treatment for 12 h in combination with Ad-mda7 enhanced MDA-7 protein expression compared with cells treated with Ad-mda7 alone. However, sulindac enhanced total MDA-7 protein levels, which included both nascent unglycosylated protein and MDA-7 proteins that were glycosylated at different levels, as indicated by multiple bands. In contrast, MG132 enhanced the level of nascent MDA-7 protein, albeit less strongly than did sulindac, and one glycosylated form of MDA-7 protein. Similar results were obtained at 6 and 24 h after treatment. Thus, the mechanisms by which sulindac and MG1 32 enhance MDA-7 protein appear to differ.

The ability of sulindac to inhibit proteasome activity was examined next. Western blot analysis for total ubiquitinated proteins, an indicator of inhibition of the proteasome pathway, demonstrated ubiquitinated proteins in MG132-treated but not sulindac treated cells. These results show that sulindac, unlike MG132, does not inhibit proteasome activity or the proteasome pathway. Consistent with these findings are the results of the proteasome activity assay, in which treatment with Ad-mda7 alone or with sulindac did not inhibit the proteasome activity compared with untreated control cells. In contrast, treatment with Ad-mda7 plus MG132 resulted in significant inhibition of proteasome activity (P=0.01). These results suggest that sulindac-enhanced MDA-7 protein expression is not due to inhibition of proteasome activity.

9. Sulindac Enhances Ad-mda7-Mediated Lung Tumor Growth Suppression

To determine whether Ad-mda7 plus sulindac treatment enhances tumor growth suppression, pilot in vivo experiments were conducted using a lung tumor xenograft model. Compared with mice treated with PBS, sulindac, Ad-mda7, or Ad-luc/sulindac, mice treated with Ad-mda7/sulindac showed a significant growth suppression (P=<0.001) (FIG. 49D). A significant tumor inhibition was also observed in mice that were treated with Ad-mda7 alone or Ad-luc plus sulindac compared with PBS-treated mice (P=0.03). No significant growth inhibition was observed in sulindac treated mice compared with PBS-treated mice. Furthermore, treatment related toxicity as evidenced by morbidity, loss of body-weight, and death was not observed in mice treated with Ad-mda7 plus sulindac, suggesting that the treatments were well tolerated.

Analysis of subcutaneous tumors 24 h after the last treatment with sulindac revealed that MDA-7 protein levels were 3-12 times higher in the tumors from mice treated with Ad-mda7 plus sulindac than in the tumors from the mice treated with Ad-mda7. These results demonstrate that treatment of lung tumors with Ad-mda7 plus sulindac enhances growth suppression in parallel to enhanced MDA-7 protein expression, a finding consistent with the in vitro results.

### Example 25

### Adenovirus-Mediated mda-7 Gene Transfer Induces Cell Cycle Arrest and Apoptosis in Human Ovarian Cancer Cells

Materials and Methods

1. Cell Lines and Reagents

Ovarian cancer cells OVCA 420 and MDAH 2774 were from Dr. J. K. Wolf, MD Anderson Cancer Center, Houston, Tex. Ovarian cancer cells SKOV3-ip, HEY and DOV 13 were also used in these experiments. SKOV-3 ip were grown in DMEM high glucose medium with 10% FBS. DOV 13 and HEY were maintained in RPMI 1640 with 10% FBS. MDAH 2774 and OVCA 420 were grown in minimum nonessential amino acid medium with 10% FBS.

2. Determination of Transduction Efficiency

Transduction efficiency of the adenovirus was investigated for each ovarian cancer cell line by infecting cells with adenovirus expressing the GFP gene (Ad-GFP). Ovarian cancer cells (SKOV3-ip, Hey, DOV 13, MDAH 2774 and OVCA 420) were seeded at 5×10⁵ cells per well in six-well tissue culture dishes. The following day, cells were either uninfected (mock); infected with Ad-GFP or Ad-luc. Twenty-four hours after infection, cells were washed in PBS, pelleted by centrifugation. The cells were then resuspended in PBS and vortexed before flow cytometry analysis.

3. Construction of Recombinant Adenoviral Vector

The construction and purification of mda-7 expressing replication-defective Ad-mda7 have been described (Saeki et al., 2000). Brifely, replication-deficient human type 5 adenoviral (Ad5) vectors carrying either the mda-7 gene, or a luciferase gene linked to an internal CMV-IE promoter and followed by SV40 polyadenylation (pA) signal were constructed. Viruses were propagated in 293 cells and purified by chromatography.

4. Determination of Cell Growth Rate

Tumor cells (SKOV3-ip, Hey, DOV 13, MDAH 2774 and OVCA 420) were seeded at 1×10⁵ cells per well in six-well tissue culture dishes. The following day, cells were either uninfected (mock); infected with Ad-mda7 or Ad-luc. Cells were harvested and counted on a hemocytometer on days 1,2,3,4 and 5 after infection.

5. Cell Cycle Analysis

MDAH 2774 and OVCA 420 cells (5×10⁵) were treated with Ad-luc or Ad-mda7 (3000 v.p./cell) in 6-well plates. Cells were harvested, pelleted by centrifugation, washed in PBS, and fixed overnight at −20° C. in 70% ethanol. The cells were resuspended in PBS containing RNase A (1 mg/ml), 50 μg/ml propidium iodide and vortexed before FACS analysis. Uninfected cells served as negative controls.

6. Apoptotic Cell Staining (Hoechst Staining)

Cells were seeded in 6 well plates at a density of 5×10⁵ cells per well and treated with Ad-mda7 or Ad-luc (3000 v.p./cell). Seventy-two hours after infection, cells were incubated with Hoechst No. 33342 (Sigma, St. Louis, Mo., USA) for 15 min, washed with phosphate-buffered saline (PBS) twice, and observed under a fluorescent microscope.

7. Western Blot Analysis

Cells were washed 1× with cold PBS and resuspended in lysis buffer (62.5 mM Tris-HCl, 2% SDS, 10% glycerol, 4M urea). Cell lysates were collected in an eppendorf tube and sonicated for 30 seconds and heated in a water bath at 95° C. for 5 mins and then centrifuged at 14,400 for 10 min at 4° C. The supernatants were mixed with 5% 2-mercaptoethanol and stored at −80° C. Protein concentrations were determined using the Bio-Rad protein assay system. Aliquots of cell extracts containing 50μg of total protein were resolved in 10% SDS-PAGE and transferred from gel to nitrocellulose membrane (Hybond-ECL; Amersham Pharmacia Biotech, UK) and then blocked for 1 h at room temperature (5% nonfat milk powder and 0.1% Tween 20 in TBS or PBS).

The membranes were then incubated with the primary antibodies, PKR (1:500), p53 (1:1000), phosphospecific p38 (1:1000), phosphospecific pJNK (1:1000), phosphospecific. p44/42 (1:1000), phosphospecific pAKT (1:1000), phosphospecific eIF2 antibodies (1:1000), caspase-3 (1:1000), PARP (1:500), caspase-9 (1:500). The p53 and PKR are from Santa Cruz Biotechnology, Santa Cruz, Calif., USA). The phosphospecific p38, phosphospecific pJNK, phosphospecific p44/42, phosphospecific pAKT, and phosphospecific eIF2 antibodies are from Cell signaling. Caspase-3, caspase-9 and PARP antibodies are from PharMingen.

The membranes were then incubated with horseradish peoxidase labeled secondary antibodies (Amersham). Finally, the proteins were visualized on enhanced chemiluminescence film (Hyperfilm; Amersham) by application of Amersham's Enhanced Chemiluminescence Western Blotting Detection System.

8. RNase Protection Assay (RPA)

Tumor cells (MDAH 2774) were plated at a density of 5×10⁵ in 6-well plates and treated with PBS, Ad-luc or Ad-mda7. Total RNAs from these cells were isolated at 24, 48 and 72h after treatment using Trizol reagent as described previously. The mRNA transcripts for the indicated apoptosis-related genes: caspase-8, Fas, FasL. FADD, FAF-1, TRAIL, TNFr, TRADD and RIP as well as the internal controls L32 and glyceraldehydes-3 3-phosphate dehydrogenase were analyzed using the hApo-3 Multi-Probe Probe template set (Pharmingen). Probe synthesis, hybridization, and RNase treatment were performed using RiboQuant Multi-Probe RNase Protection Assay System (PharMingen) as per manufacturer's guidelines. Protected transcripts were resolved by electrophoresis on denaturing polyacrylamide gels (5%) and exposed to hyperfilm overnight at −80° C.

9. Electrophoretic Mobility Shift Assay (EMSA)

MDAH 2774 (5×10⁵) were treated with Ad-luc or Ad-mda7 in 6-well plates. Cells were harvested at various time points (24, 48, and 72 h) and cytoplasmic and nuclear extracts were prepared and subjected to EMSA as described previously. Briefly, AP-1 consensus double-stranded oligonucleotides (Promega) were end-labeled with [γ-³²P]-ATP using T4 polynucleotide kinase. A typical binding reaction mixture contained the labeled oligonucleotide and 0.5 μg poly (dI-dC) and nuclear protein extracts (10 μg) were incubated at 25° C. for 30 min in 5× gel shift binding buffer [20% glycerol, 5 mM MgCl₂ 2.5mM EDTA, 2.5 mM DTT, 250mM NaCl, 50 mM Tris-HCl (pH 7.5)]. The complexes were resolved on nondenaturing 5% polyacrylamide gels in 0.5× Tris-borate EDTA buffer for 1 h 30min at 300 V. The bands were visualized by autoradiography and quantitated using the Image Quant software (Molecular Dynamics, Amersham-Pharmacia, Biotech, Piscatway, N.Y.).

10. Fas Promoter Analysis

MDAH 2774 cells (5×10⁵) plated in 6-well plates were transfected with a plasmid (FHR+) consisting of the luciferase gene under the control of the human Fas (CD95) promoter. Cells transfected with a plasmid (Δ6) that contained a mutation in the Fas promoter served as controls in these experiments. Transfections were performed using DOTAP:cholesterol (DOTAP:Chol) liposomes as described previously. Six-hours after transfection cells were treated with PBS, Ad-βgal, or Ad.mda-7. Cells were harvested at 12, 24, 48h after treatment, washed in PBS, and lysed in 200 μl of Reporter Lysis Buffer (Promega). Luciferase expression was determined as described previously and expressed as relative light units (RLU) per milligram of protein. Experiments were repeated atleast two times and results represented as the average of two experiments.

11. Experiments using Dominant Negative FADD Expression Vector

Tumor cells were plated in 2-well chamber slides or 6-well plates and transfected with a plasmid expression vector carrying the yellow fluorescent protein (YFP) and dominant negative FADD (YFP-dnFADD) or transfected with a plasmid vector carrying only the YFP. The YFP-dnFADD plasmid expresses YFP and FADD as a fusion protein enabling both visualization and function of the FADD protein. Cells were transfected with the plasmids encapsulated in DOTAP:Chol.liposomes as described above. Twenty-four after transfection, cells were treated with PBS, or treated with Ad-mda7. At 24h and 48 h after treatment cells were either observed under fluorescence microscope for transduction efficiency or cell lysates prepared and probed for FADD, caspase 9, and caspase 8 by western blot analysis.

For determining the effect of dnFADD on Ad-mda7 mediated apoptosis, cells were treated as described above and analyzed for the number of cells in the sub-G₀ phase, an indicator of apoptotic cells, by flow cytometry.

12. SiRNA Analysis

For siRNA analysis, Fas specific siRNA was synthesized and purified using the siRNA kit (Ambion, Austin Tex.). The following sequences were used to synthesize the siRNA:

a) Target (Fas) siRNA:

a) Target (Fas) siRNA:
5′-AAGTAAAGGTAGAGGGGGAGCCCTGTCTC-3′
5′-AAGCTCCCCCTCTACCTTTACCCTGTCTC-3′
b) Scrambled (control) siRNA
5′-AAAAGTTTCCGATACGCTTTACCTGTCTC-3′
5′-AATAAAGCGTATCGGAAACTTCCTGTCTC-3′

Cells seeded in 6-well plates were transfected with siRNA (Fas or control) using oligofectamine. Cells that were treated with empty oligofectamine alone served as control in these experiments. For analysis of the inhibitory effect of siRNA on Fas, cells were harvested 48 h after transfection, cell lysates prepared, and analyzed for Fas expression by western blot analysis. To determine the effect of siRNA on Ad-mda7 mediated apoptosis, cells were transfected with Fas specific or scrambled siRNA as described above. 48 h after transfection cells were treated with PBS or treated with Ad-mda7. Cells were harvested 24 h after treatment, fixed, and analyzed for apoptotic cells by flow cytometry.

Results

1. Transduction Efficiency in the Ovarian Cell Lines

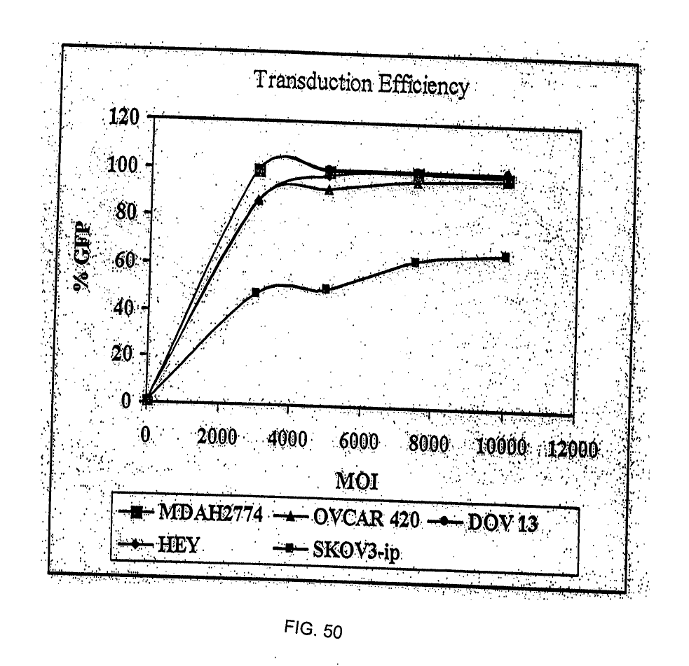

To investigate a relationship between the efficiency of the gene transfers by the virus vector, adenoviral transduction of the five ovarian cancer cell lines studied were determined by infecting the cells with Ad-GFP. The five cell lines varied in transduction efficiency, with the MDAH 2774, OVCA 420, DOV13 and Hey cell the easiest to transduce with above 90% transduction efficiency at 3000 MOI and the SKOV3-ip cells most difficult even at 10,000 MOI (FIG. 50).

2. Effect of Adenovirus-Administered mda-7 in Ovarian Tumor and Normal Fibroblast Cells

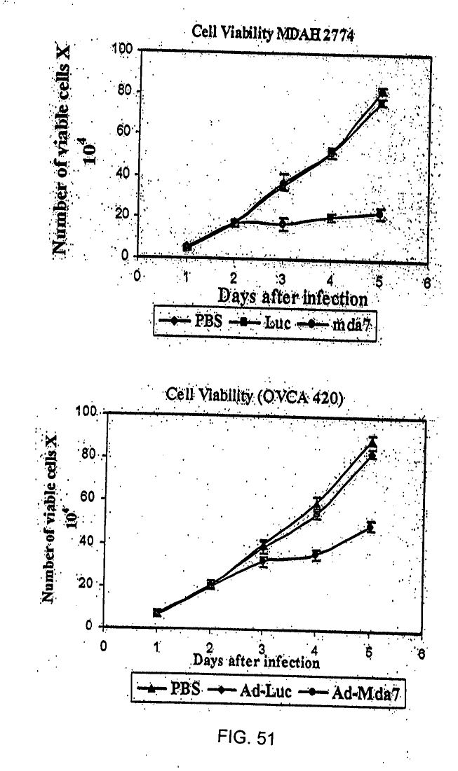

The SKOV3-ip, Hey and DOV13 tumor cell lines were not growth inhibited following mda-7 infection. In contrast, inhibition of cell proliferation was observed in MDAH 2774 and OVCA 420 infected with Admda-7, as compared with control cells infected with Ad-luc or treated with PBS. No significant growth inhibition was observed in normal human fibroblast cells following infection with Ad-Luc and Ad-mda 7 (FIG. 51).

3. MDA-7 Induces G2/M Cell Cycle Arrest and Apoptosis Selectively in Ovarian Cells

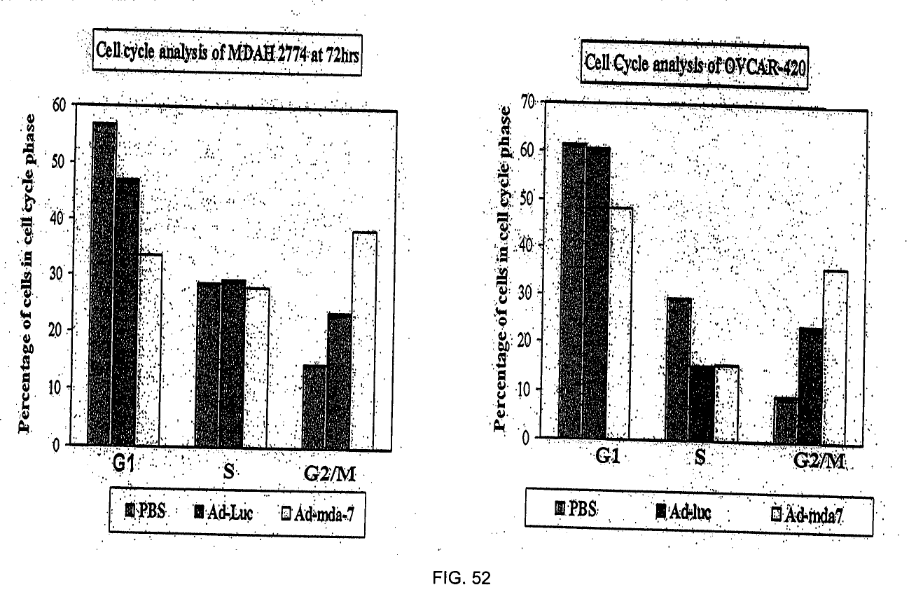

To further examine the molecular mechanisms of growth suppression, flow cytometric analysis was performed. There was found to be a marked increase in the percentage of the G₂/M population in two of the five cell lines that showed significant growth suppression, MDAH 2774 and OVCA 420 (FIG. 52). Infection with control Ad-luc did not change the percentage of cells in the G₂/M phase of the cell cycle. Following infection of Ad-mda7, MDAH 2774 and OVCA 420 tumor cells underwent apoptotic cell death. However, no changes were observed in cells infected with Ad-Luc or mock.

4. Infection of Ovarian Cells with Ad-mda7 Induces Expression of Intracellular and Secreted Protein

Ovarian cancer cells were infected with Ad-mda7 and Ad-luc. Cells were harvested at 24, 48 and 72 h after infection and extracts were prepared for western blot analysis. Extracts from mock-infected cells were used as an additional control. MDA-7 protein expression was detected in all of the Ad-mda7 infected cell lines but not in any mock-infected controls or Ad-luc infected cells.

5. Expression of mda-7 Results in Up-Regulation of PKR, pPKR, peIF2, p38, and JNK

In MDAH 2774 and OVCA 420 cells, mda-7 activated PKR, pPKR, peIF₂, p38 and JNK. When cells were infected with 3000 MOI of Ad-mda7, maximal activation of PKR and its substrate was seen at 48 hrs.

6. Caspase Cascade Activation and Cleavage of PARP Following mda-7 Expression

Treatment with Ad-mda7 led to activation of caspase-9 and caspase-3 and cleavage of PARP, a substrate for the caspase.

These results, which demonstrate the selective effect of mda-7 on ovarian cancer cells, provide support for the use of Ad-mda7 for the therapy of ovarian cancer.

7. Regulation of Various Apoptosis-Related Proteins by MDA-7

Studies have previously demonstrated activation of several apoptosis related proteins (PKR, pJNK, p38 MAPK,) by MDA-7 in human lung cancer cells and melanoma. Based on these observations activation of these proteins was investigated in ovarian cancer cells at 24 and 48 h after Ad-mda7 treatment. PKR, which has previously been shown to be critical in MDA-7 mediated killing of lung cancer cell, was activated significantly only at 48 h but not at 24h. Associated with PKR was the activation of its downstream substrate peIF2. p38MAPK, and pJNK was also significantly activated only at 48 h but not at 24 h. However, MDA-7 activated pc-Jun and pATF-2 at 24 h and continued at 48 h. These results show that MDA-7 differentially activates various signaling molecules at different time points.

8. MDA-7 Activates Fas and Fas-Related Proteins in Ovarian Cancer Cells

To test whether additional signaling events or molecules are activated/triggered earlier than those previously reported results in the initiation of apoptosis cascade, ovarian cancer cells were treated with PBS, Ad-luc, or Ad-mda7 and analyzed for apoptosis related molecules by RPA. A significant increase in Fas, Fas-L, FADD, caspase-8, and FAF1 mRNA expression was observed in Ad-mda7 treated cells compared to PBS and Ad-luc treated cells. A moderate increase in FAP expression was observed. No change in TRADD, DR3, TNF, and RIP expression was observed among the various treatment groups suggesting activation of Fas- but not TNF-related family members may be an early event. Since changes in mRNA levels do not always correlate with protein expression, Western blot analysis was performed. A significant increase in Fas, Fas-L, FAF1, and FADD protein expression was observed in Ad-mda7 treated cells compared to PBS and Ad-luc treated cells. Increase in protein expression was in agreement with the mRNA results. However, FAP protein expression was decreased in Ad-mda7 treated cells compared to PBS and Ad-luc treated cells. No change in TRADD expression was observed among the various treatment groups. These results suggest that activation of Fas-FasL by MDA-7 is an early event in ovarian cancer cells.

9. MDA-7 Activates AP-1 and NFκB

AP-1 and NFκB are major transcription factors that are activated by binding of the Jun family (c-Jun, JunD, and JunB) of transcriptional proteins as homodimer or heterodimer in association with the Fos family members or with other transcription factors such as ATF2, CREB, and NFAT. Based on this information and the ability of MDA-7 to activate c-jun and ATF-2 we speculated that MDA-7 mediated signaling involves AP-1 and or NFκB. For this purpose nuclear cell lysates were prepared at 24 and 48 h after treatment with PBS, Ad-luc or Ad-mda7 and analyzed for AP-1 and NFκB activation by EMSA. Nuclear lysates from Ad-mda-7 treated cells showed higher AP-1 and NFκB binding activity than in the Ad-luc and PBS treated cells. Increased activity was observed at both 24 and 48 h with maximum activation occurring at 48h.

10. MDA-7 Increases Fas Expression at the Cell Surface

To determine whether MDA-7 increased Fas expression at the cell surface tumor cells treated with PBS, Ad-luc or Ad-mda7 were stained with a fluorescent-labeled anti-Fas antibody and observed under a fluorescence microscope. An increase in Fas expression at the cell surface was observed in Ad-mda7 treated cells compared to PBS and Ad-luc treated cells.

11. MDA-7 Activates Fas Promoter

The ability of Ad-mda7 treatment on Fas promoter activation was next investigated. Cells transfected with a plasmid carrying the luc gene under the control of wild-type Fas promoter was significantly activated following Ad-mda7 treatment compared to cells that were treated with PBS or Ad-βgal (P=0.001). A slight increase in luciferase expression was also observed in Ad-βgal treated cells compared to PBS treated cells (P=0.04). In contrast, no significant increase in luciferase expression was observed among the various treatment groups when cells were transfected with a plasmid containing mutant Fas promoter indicating that Ad-mda7 treatment results in specific activation of wild-type Fas promoter.

12. Overexpression of Dominant Negative FADD Inhibits MDA-7 Mediated Apoptosis

Since FADD is part of the death inducing signaling complex (DISC) that is formed following FAS induced signaling, the effect of overexpression of dominant negative FADD on MDA-7 mediated apoptosis was investigated. Prior to the start of the experiment, cells were transfected with EYFP or EYFP-dnFADD plasmid and determined for transduction efficiency, and dnFADD expression. Note, dnFADD that is expressed as a EYFP fusion protein is distinguished from endogenous FADD by the shift in the banding pattern. In subsequent experiments cells were either not transfected or transfected with EYFP or EYFP-dnFADD and later treated with PBS or Ad-mda7. Cells were analyzed for apoptotic cells by flow cytometry and for caspase-9, and caspase-8 by Western blot analysis. A significant number of apoptotic cells was observed in cells treated with Ad-mda7 alone (15%; P=0.001) compared to cells treated with PBS, EYFP plasmid alone, or EYFP-dnFADD plasmid alone. However, Ad-mda7 mediated apoptosis was significantly inhibited in EYFP-dnFADD transfected cells overexpressing dnFADD. In contrast Ad-mda7 treatment in EYFP transfected cells resulted in increased apoptosis. Furthermore, Ad-mda7 treatment of parental cells or cells transfected with EYFP plasmid resulted in activation of caspase-8 and -9. In contrast, MDA-7 mediated activation of caspase-8, and -9 was inhibited in cells overexpressing dnFADD. No caspase activation was observed in cells that were treated with PBS, treated with EYFP alone, and treated with EYPF-dnFADD alone.

13. Inhibition of Fas by siRNA Suppresses MDA-7 Mediated Apoptosis

To further test whether Fas plays a role in MDA-7 mediated apoptosis, siRNA experiments were conducted. Cells were initially transfected with vector alone, scrambled siRNA, or siRNA targeted to Fas and analyzed by western blot analysis. A marked inhibition of Fas protein expression was observed in cells transfected with siRNA targeted to Fas but not in cells transfected with scrambled siRNA. Inhibition of Fas was observed in cells transfected with 200 nm siRNA. Based on these results subsequent experiments were conducted using 200 nm of Fas or scrambled siRNA. Cells transfected with Fas siRNA or scrambled siRNA were treated with Ad-mda7 and analyzed for apoptotic cells. A significant reduction in the number of MDA-7 induced apoptotic cells was observed in Fas siRNA transfected cells (9%) compared to cells transfected with scrambled siRNA (19.2%). No significant increase in apopototic cells was observed in PBS treated cells. These results demonstrate that Fas plays a role in MDA-7-mediated apoptosis in ovarian cancer cells.

### Example 26

### Tumor Growth Suppressor Gene mda-7 Induces Apoptosis and Affects the Pathway of APC/Beta-Catenin in Human Breast Cancer

Materials and Methods

MDA-MD-468 breast cancer cells were studied both in vitro and in vivo in a flank xenograft model of breast cancer. A recombinant adenovirus with the mda-7 transgene under control of the CMV promoter to express mda-7 was used (Ad-mda 7). Control cells were treated with Ad-Luc or PBS. At 48 hours, cell growth was evaluated by direct cell count and apoptosis measured by cleavage of caspase-3 and PARP. In vivo, tumors were treated by intratumoral injection of Ad-mda7, Ad-Luc, or PBS when tumors reached 100 mm³ in size. At 48 hours following treatment, tumors were harvested and apoptosis evaluated by TUNEL staining, in addition to cleavage of caspase-3 and PARP. The adenomatous polyposis coli protein (APC)/β-catenin pathway was evaluated by determining the expression of APC and β-catenin in MDA-MB-468 cells in vitro and in vivo after treatment with Ad-mda 7.

Results

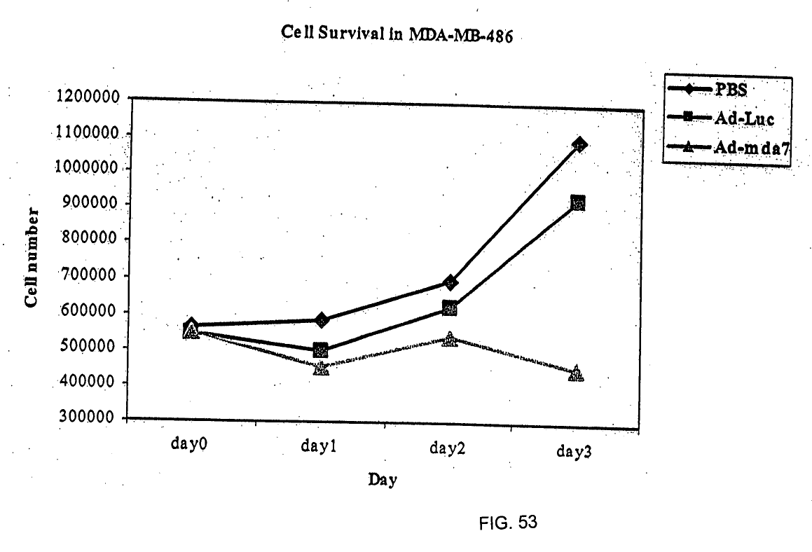

Treatment of MDA-MB-468 cells with Ad-mda7 resulted in -significant apoptosis and growth inhibition both in vitro and in vivo, when compared to control treated cells (FIG. 53; P<0.01, ANOVA). Significantly elevated levels of APC expression were observed both in vitro and in vivo after treatment with Ad-mda7. Correspondingly, the β-catenin levels in the Ad-mda7 treated cells were significantly decreased when compared with controls. These studies confirm the significant growth inhibition and apoptotic effect of Ad-mda7 on MDA-MB-468 breast cancer cells. Additionally, these data suggest that this cell death may be due to the upregulation of APC resulting in the downregulation of nuclear β-catenin following Ad-mda7 transfection. Further studies are currently underway to confirm these observations.

### Example 27

### Adenovirus Mediated MDA-7 Gene Therapy Suppresses Angiogenesis and Sensitizes Xenograft Tumors to Radiation

Materials and Methods

1. Cell Culture and Chemicals

The NSCLC cell line, A549, was obtained from the American Type Culture Collection (Rockville, Md.). A549 cells were grown in F-12 medium with 10% fetal bovine serum (HyClone, Logan, Utah) and 1% penicillin-streptomycin (Invitrogen, Grand Island, N.Y.) at 37° C. with 5% CO₂. The normal human lung fibroblast cell line, CCD16, was obtained from the American Type Culture Collection and maintained in MEM-α medium with 10% fetal bovine serum and 1% penicillin-streptomycin. Human embryonic kidney 293 cells, that were stably transfected with mda7 or control vector, were provided by Introgen Therapeutics Inc. (Houston, Tex.). 293 cells were maintained in MEM high glucose medium with 10% fetal bovine serum and 1% penicillin-streptomycin. Human umbilical vein endothelial cells (HUVECs) were obtained from Clonetics (San Diego, Calif.) and maintained in complete EGM-2 medium (Clonetics, San Diego, Calif.) per manufacture's instruction. 293 cells (1×10⁶) cultured for 24 hours yield 30 ng/ml of MDA-7 protein in their culture medium determined on the basis of ELISA assay.

Human angiostatin (kringle 1-3) and human recombinant endostatin were purchased from Calbiochem (San Diego, Calif.) and diluted into medium at a concentration of 100 ng/ml for cell treatment.

2. Animal Studies

A549 xenograft tumors were established by s.c. injection of 5×10⁶ viable cells, suspended in serum-free medium, into the hind legs of 4-5-week-old male athymic nude mice (nu/nu; Harlan). Within 10-14 days, tumors reached a size of 200 mm³ (day 0). Tumor growth delay was assessed following treatment. Tumors were measured in three orthogonal dimensions, and volume was estimated assuming an ellipsoid. Animals were sacrificed when the tumor exceeded 15 mm in a diameter or the tumor ulcerated. All animals used in these experiments were housed and maintained in the institutional facilities in accordance with regulations and standards of the US Department of Agriculture and the National Institutes of Health. The animals were used under an approved protocol reviewed by the Institutional Animal Care and Use Committee.

3. Adenovirus Production

Ad-mda7, as described in U.S. patent application Ser. No. 09/615,154, was obtained. This recombinant adenoviral vector contains the CMV promoter, wild type mda-7 cDNA, and SV40 polyadenylation signal in a minigene cassette inserted into the El-deleted region of modified Ad5. The vectors were tested for and determined to be free of replication-competent adenovirus and mycoplasma.

4. Gene Delivery

In vivo experiments were performed on s.c. xenograft tumors growing in the hind legs of nude mice. When tumors reached 200 mm³, they were treated with a total dose of 3×10⁶ vp administered in 3 equal fractions given on days 1, 3, and 5. Each injection of purified vector was diluted in a total volume of 100 μl of PBS and administrated in a single pass of a 27.5-gauge insulin needle.

5. Radiation

For in vivo treatments, animals bearing A549 xenograft tumors were irradiated while anesthetized using a ⁶⁰Co teletherapy unit. The mice were positioned in the field such that only the hind leg bearing the tumor was in the irradiation field and the rest of the body was shielded by a lead block. For in vitro treatments, cells were irradiated with a high-dose rate ¹³⁷Cs unit (3.4 Gy/min) at room temperature.

6. Immunohistochemistry

Tumors were harvested on day 8 for MDA-7 immunostaining or on day 14 for vascular endothelial growth factor (VEGF), basic fibroblast growth factor (bFGF), interleukine-8 (IL-8) or CD31 immunostaining. For VEGF, bFGF, IL-8 or MDA-7 immunostaining, 5-micron-thick sections of formalin-fixed paraffin embedded tissue were used. For CD31 immunostaining, 8-micron-thick sections were obtained from frozen sections. Sections from formalin-fixed paraffin embedded tissue were deparaffinized in xylene and rehydrated in descending grades (from 100 to 75%) of ethanol. To enhance the immunostaining, sections were then placed in antigen unmasking solution (Vector Laboratories Inc., Burlingame, Calif.) and microwaved intermittently for up to 10 min to maintain a boiling temperature. After the slides were cooled at room temperature for 30 min, they were washed in distilled water and PBS. After this initial preparation, the slides, which were obtained from either formalin-fixed paraffin embedded or frozen tissues, were covered with 3% H₂O₂ in methanol to block endogenous peroxidase activity. An avidin-biotin-peroxidase complex kit (Vector Laboratories Inc.) was then used to detect staining. After treatment with the blocking serum and endogenous avidin/biotin blocking solution (Vector Laboratories Inc.), the slides were incubated with a 1:500 dilution of rabbit polyclonal antibody to VEGF (Santa Cruz Biotech, Santa Cruz, Calif.), a 1:500 dilution of rabbit polyclonal antibody to bFGF (Sigma Chemical Co., St. Louis, Mo.), a 1:50 dilution of rabbit polyclonal antibody to IL-8 (Biosource International, Camarillo, Calif.), a 1:100 dilution of rat monoclonal antibody to mouse CD31 (PharMingen, San Diego, Calif.) or 1:250 dilution of rabbit polyclonal antibody to MDA-7 (Introgen Therapeutics, Houston, Tex.) overnight at 4° C. The slides were then washed, incubated for 30 min with secondary biotinylated antibody, washed again and then incubated for 30 min with the avidin-biotin-peroxidase complex reagent. After the slides were washed in PBS, the immunostaining was developed with the use of 3,3′-diaminobenzidine. Slides were counterstained with methyl green (Vector Laboratories, Inc.) and mounted with Permount (Fisher Scientific, Pittsburgh, Pa.).

To determine the percentage of immunostaining positive cells, at least 1000 cells/slide were counted and scored in ×400 fields (Bianco et al., 2002; Weidner et al., 1991). Microvessels were quantified using sections immunostained for CD31 according the method described by Weidner et al (1991). Microvessel density was expressed as the average of the three highest areas identified within ×400 fields.

7. TUNEL Assay

A terminal deoxynucleotidyl transferase (TdT)-mediated dUTP labeling (TUNEL) method was used for the detection of apoptotic cells. For this purpose, we used the ApopTag® Plus Peroxidase In Situ Apoptosis Detection Kit (Serological Corporation, Norcross, Ga.). The staining was performed according to the manufacturer's procedure. Sections included in the kit were stained and served as positive controls. Apoptotic cells were counted under a light microscope (×400 magnification) in randomly chosen fields, and the apoptosis index was calculated as a percentage of at least 1000 scored cells.

8. Cell Survival Analyses

Twelve hours after seeding HUVECs in 4-well plates (Nunc, Roskilde, Denmark), medium was replaced with serum/growth factor-free medium. After serum/growth factor starvation for 12 hours, cells were treated with complete medium containing either MDA-7 protein (10 ng/ml) (removed from cultures of 293 cells), angiostatin (100 ng/ml), or endostatin (100 ng/ml). Twelve hours later, cells were irradiated with ¹³⁷CS unit (3.4 Gy/min) at room temperature, trypsinized and counted. Known numbers of cells were replated in 60 mm culture dishes and incubated to allow macroscopic colony development. Colonies were counted after 14 days, and the percent plating efficiency and surviving fractions following given treatments were calculated based on the survival of non-irradiated cells.

9. Statistical Analysis

Statistical significance was determined using one way ANOVA or Student's t test, as appropriate. A difference was regarded as significant if p<0.05.

Results

1. Effect of Combination Therapy with Ad-mda7 and Radiation on A549 Xenograft Tumors

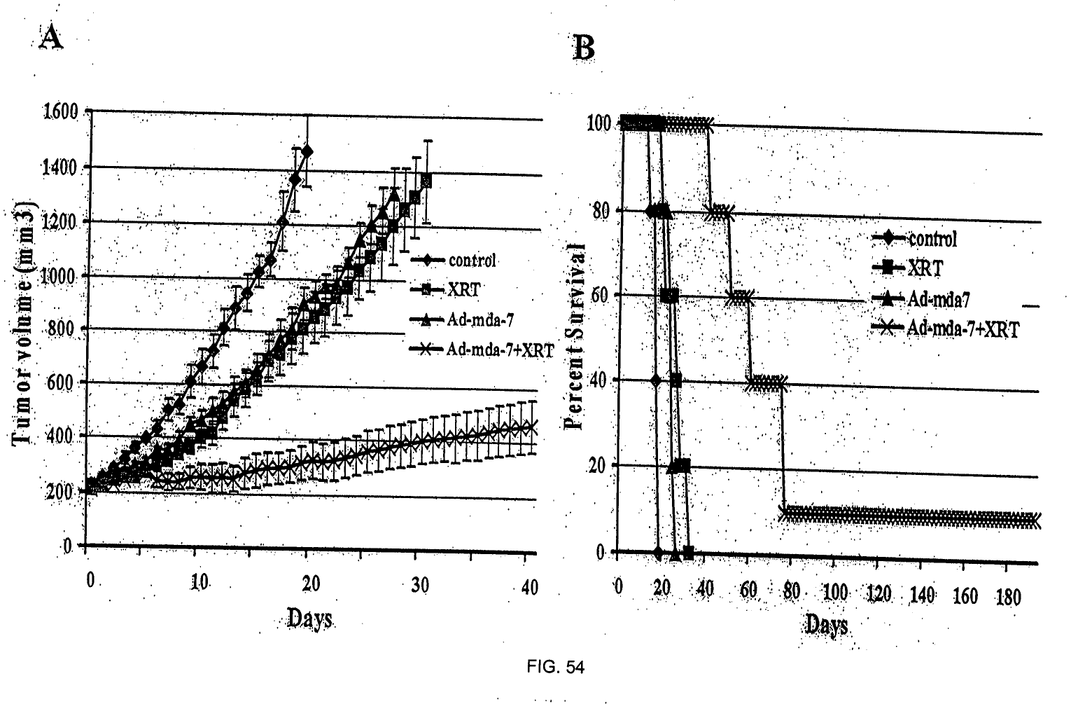

A549 cells were injected s.c. into the hind leg of athymic nude mice (n=20). When the tumors reached 200 mm3 (day 0), animals were randomized into one of four treatment groups: control (saline injection), radiation alone (5 Gy in one fraction on day 6), Ad-mda 7 alone (3×10¹⁰ vp in three fractions on days 1, 3 and 5) or combination (Ad-mda7 plus radiation). This treatment protocol was chosen based on an original study using Ad-p53 on SW620 xenografts and is identical to that used in subsequent investigations where the combination of Ad-p53 and radiation in A549 xenografts was tested (Kawabe et al., 2002; Spitz et al., 1996). This allows a comparison between Ad-p53 and Ad-mda7 for their relative abilities to radiosensitize A549 xenograft tumors. As shown in FIG. 54, treatment with either radiation alone or Ad-mda7 alone produced only a modest tumor growth delay. On the other hand, combination therapy with Ad-mda7 and radiation resulted in a substantial and long-lasting tumor growth delay (FIG. 54A). As per our animal protocol, the mice had to be killed when the tumors reached 15 mm in diameter. The time that each mouse was killed was recorded and is plotted in FIG. 54B. As can been seen, treatment with the combination greatly extended this time and one animal in this group of 5 was cured. This animal was killed after a total of 240 days. There was no evidence of tumor in this animal on necropsy.

2. Effect of Combination Therapy was Dependent on Treatment Schedule

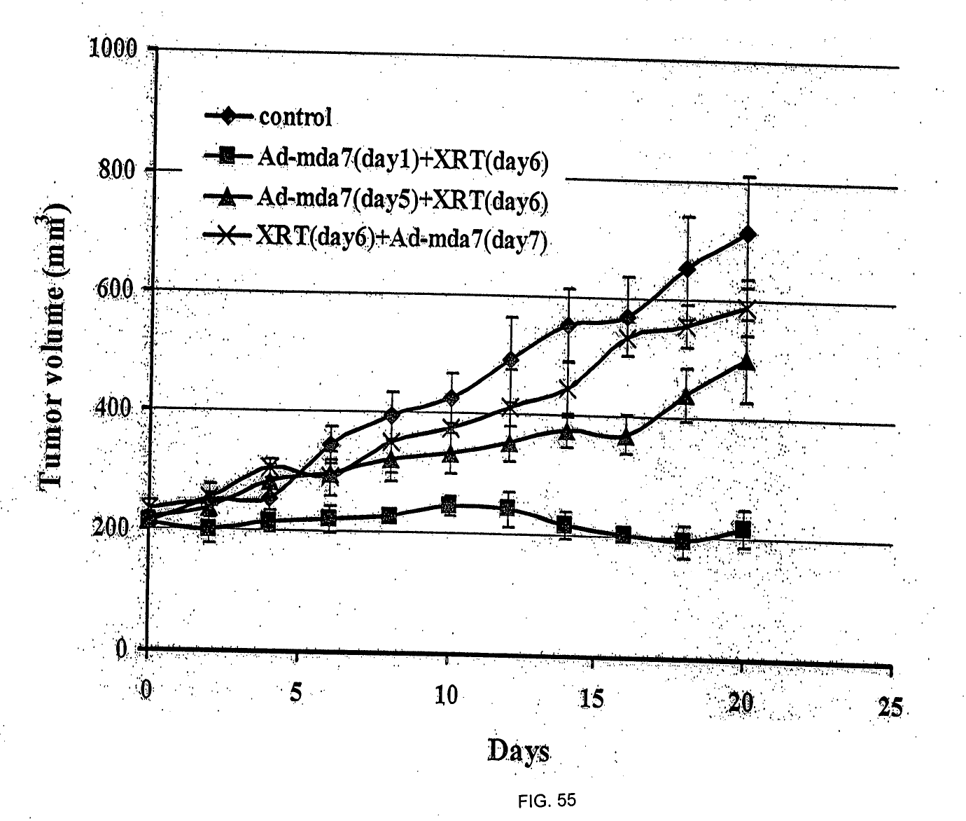

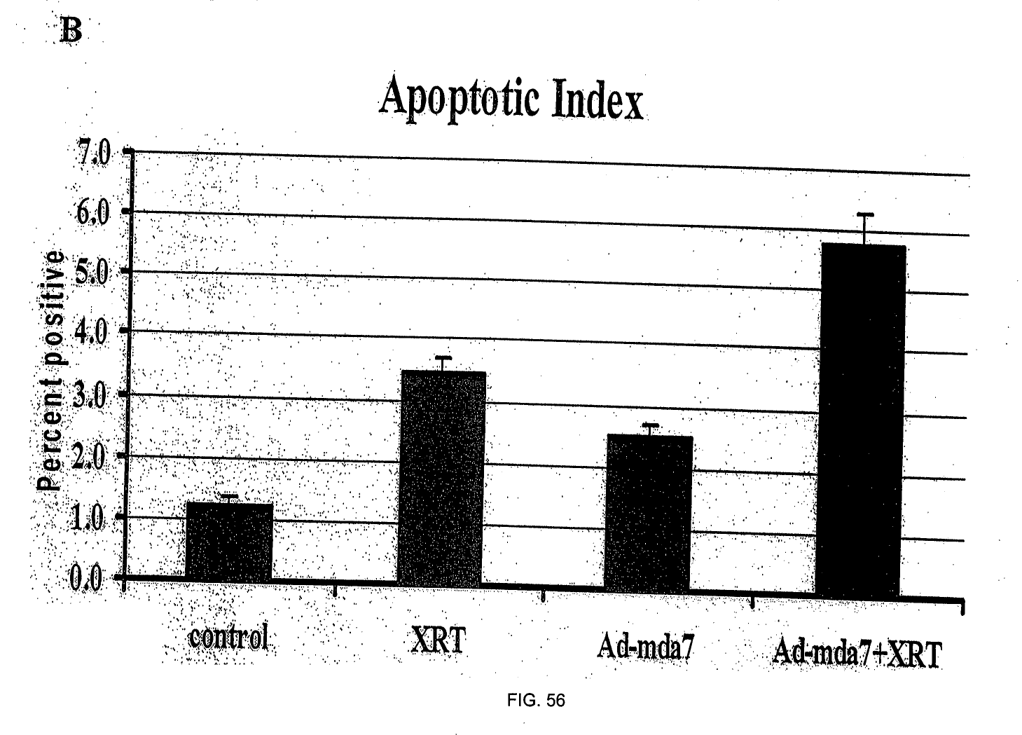

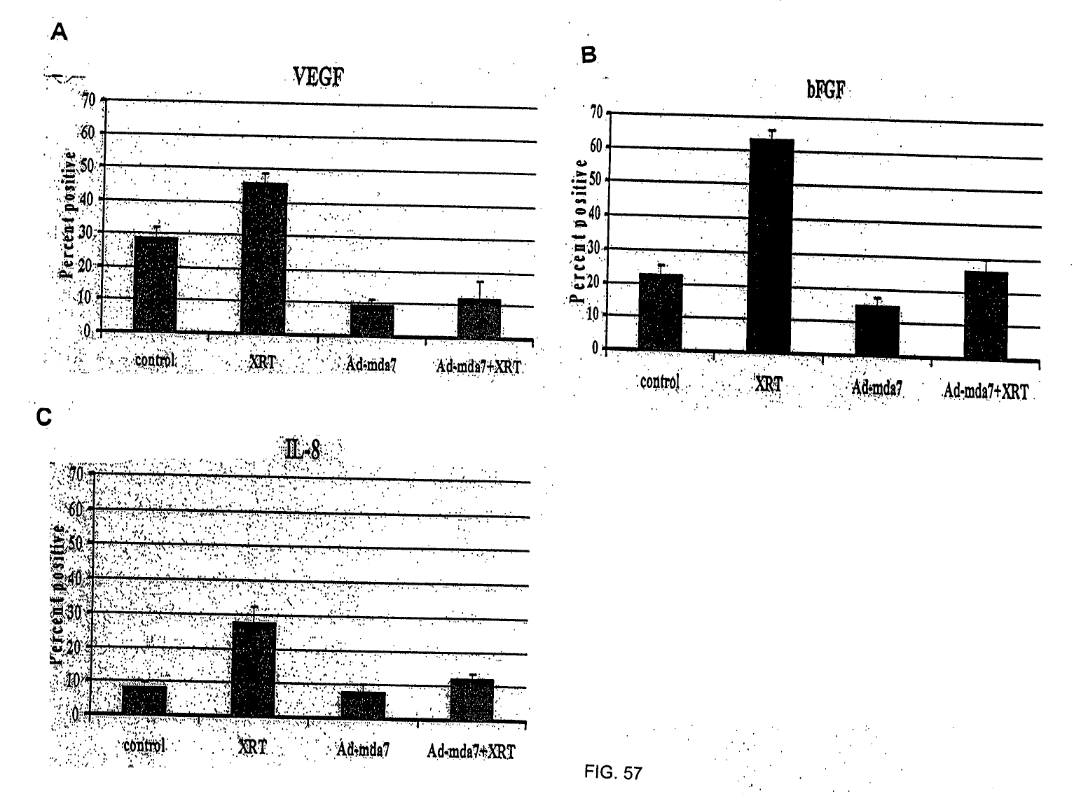

To determine an optinal regimen of combination therapy with 3×10¹⁰ Vp of Ad-mda7 in a single fraction and 5Gy radiation, the following treatments were examined: control, Ad-mda7 (day 1) plus irradiation (day 6), Ad-mda7 (day 5) plus irradiation (day 6) or irradiation (day 6) plus Ad-mda7 (day 7). The results of tumor growth delay indicated that the best timing for the combination therapy was administering Ad-mda7 followed 5 days later by 5Gy radiation (FIG. 55).

3. MDA-7 Protein Expression in Tumors after Administration of Ad-mda7

Expression of MDA-7 protein was examined by immunohistochemistry in specimens on day 8. Strong expression of MDA-7 protein in the cytoplasm was detected in the tumors after Ad-mda7 treatment whereas no specific staining was detected in the tumors that did not receive Ad-mda7. This pattern did not change in specimens from tumors that also received irradiation.

4. Combination Therapy with Ad-mda7 and Radiation Enhanced Apoptosis Induction In Vivo

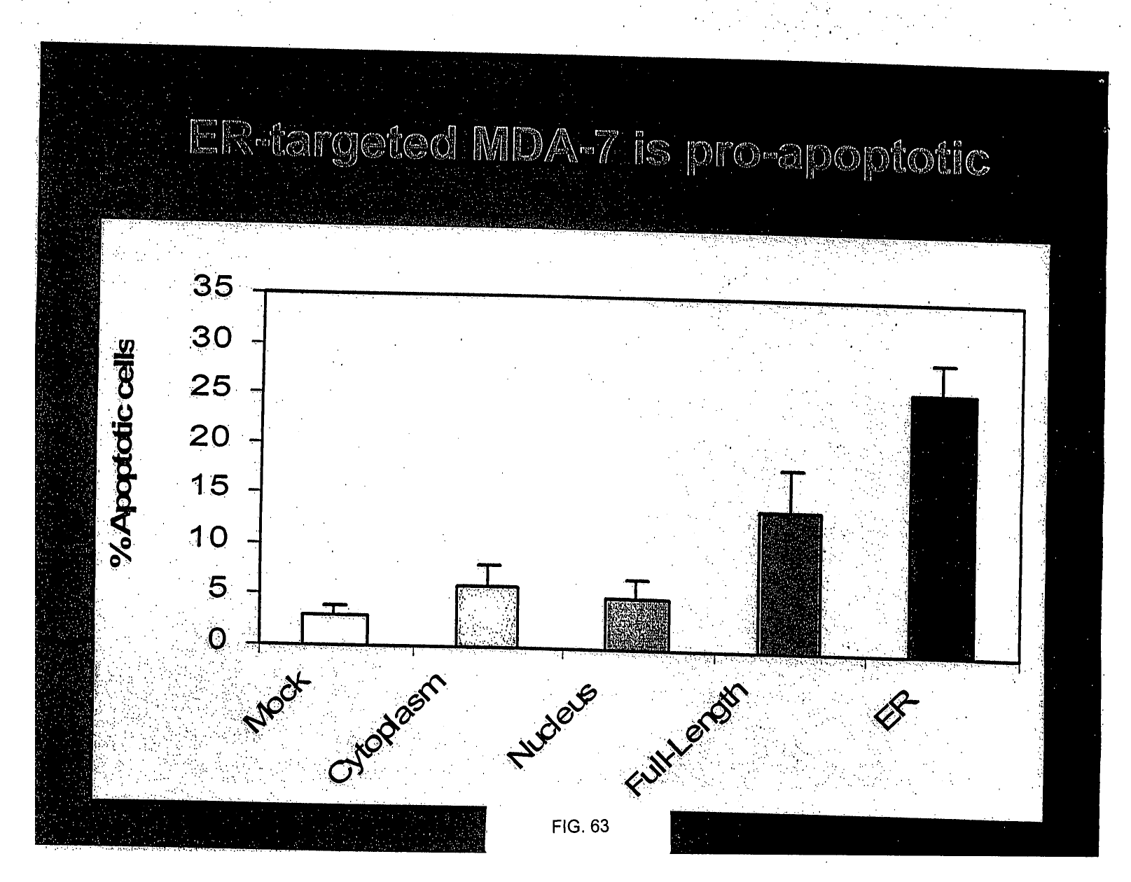

Previously, enhanced induction of apoptosis by Ad-mda7 when combined with radiation in NSCLC cells was observed when treated in vitro (Kawabe et al., 2002). To determine if a similar enhancement of apoptosis would be observed in vivo, TUNEL staining on the specimens harvested following the various treatments was performed. TUNEL-positive cells were observed scattered throughout the histological sections especially in those specimens from the treated groups. The TUNEL-positive cells were counted and the resulting values are presented in FIG. 63. Combination therapy with Ad-mda7 and radiation resulted in a slight greater-than-additive apoptotic index (4.6%) compared with radiation alone (2.2%) or Ad-mda7 alone (1.3%) after subtracting the background level of 1.2%.

5. Ad-mda7 Blocks the Radiation-Induced, Enhanced Expression of Angiogenic Factors

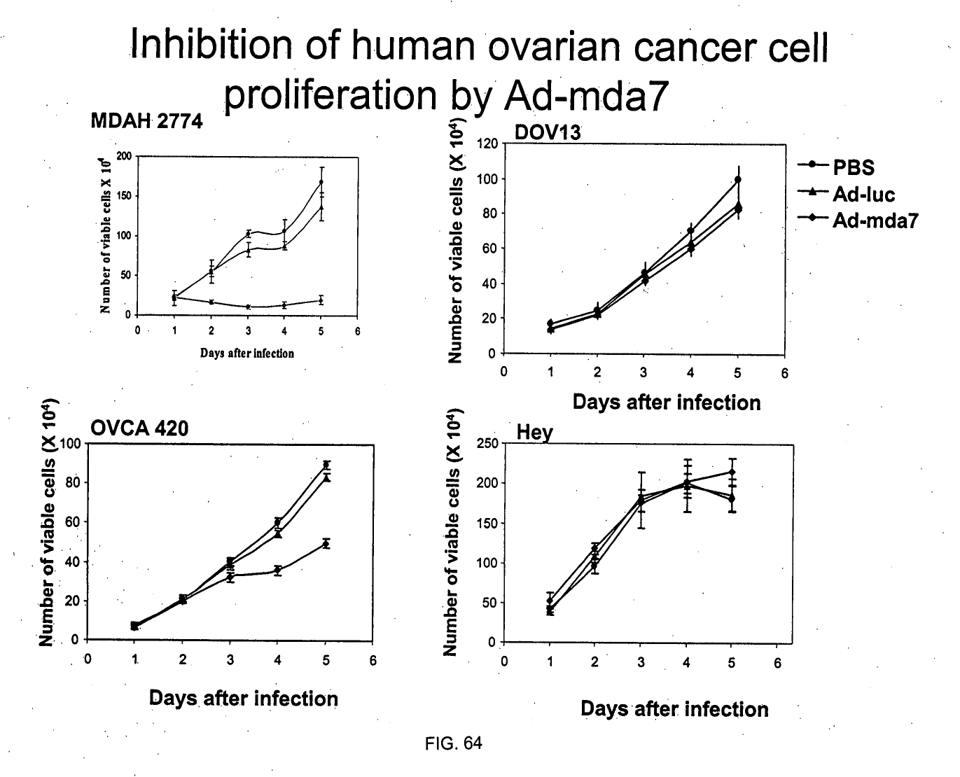

VEGF, bFGF and IL-8 protein expression were analyzed by immunohistochemistry on specimens harvested on day 14. The percentage of positive cells was scored for each specimen and the resulting values are plotted in FIGS. 64A-64C. There was some constitutive expression seen in the untreated controls for each of these angiogenesis markers. However, radiation alone substantially enhanced the expression of each of these proteins. Ad-mda7 treatment suppressed the constitutive levels of VEGF and bFGF and essentially blocked the radiation-induced, enhanced expression of all three angiogenesis markers.

6. Combined Therapy with Ad-mda7 and Radiation Suppresses Microvessel Density

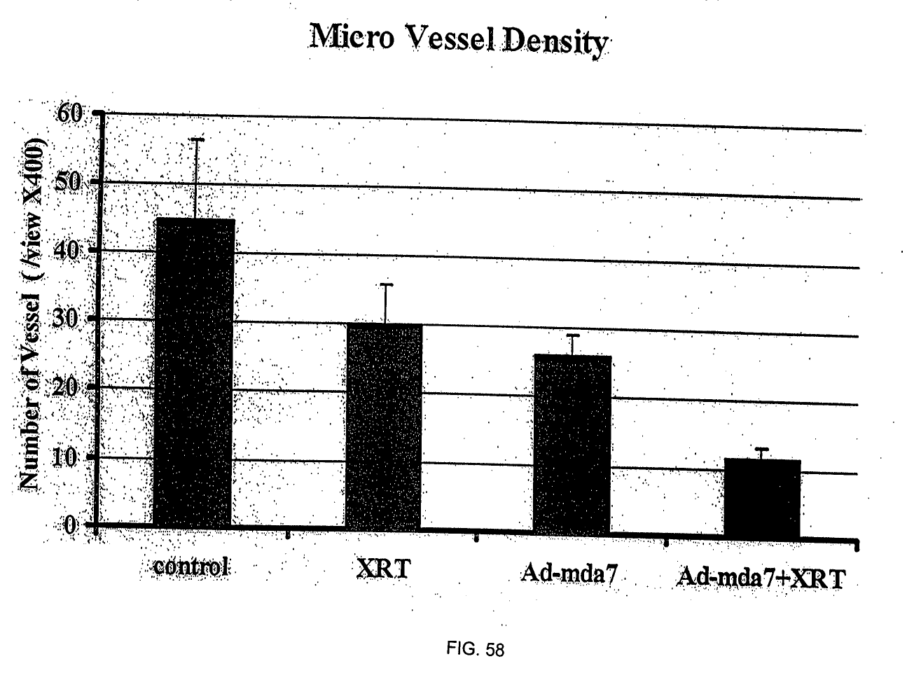

A previous report suggested that Ad-mda7 suppressed tumor growth by inhibiting angiogenesis (Saeki et al., 2002). Therefore, the antiangiogenic effects of Ad-mda7 as a single agent and in combination with radiation were evaluated. Tumors were harvested on day 14 and frozen sections were stained for CD31 (platelet endothelial cell adhesion molecule-1) to assess microvessel density. Assessment of microvessel density on CD31-stained sections showed that the average microvessel count per ×400 field in untreated, control tumors was 45±12. Both Ad-mda7 and radiation, when used as single agents, were able to partly suppress microvessel counts to 26±3 and 30±6, respectively. However, in tumors treated with the combination of Ad-mda7 and radiation, the average microvessel count was suppressed even further to 12±2 (FIG. 58). This 3.8-fold reduction of microvessel density by the combination was statistically significant compared with the other groups.

7. Recombinant, Human MDA-7 Protein Sensitizes Endothelial Cells to Radiation

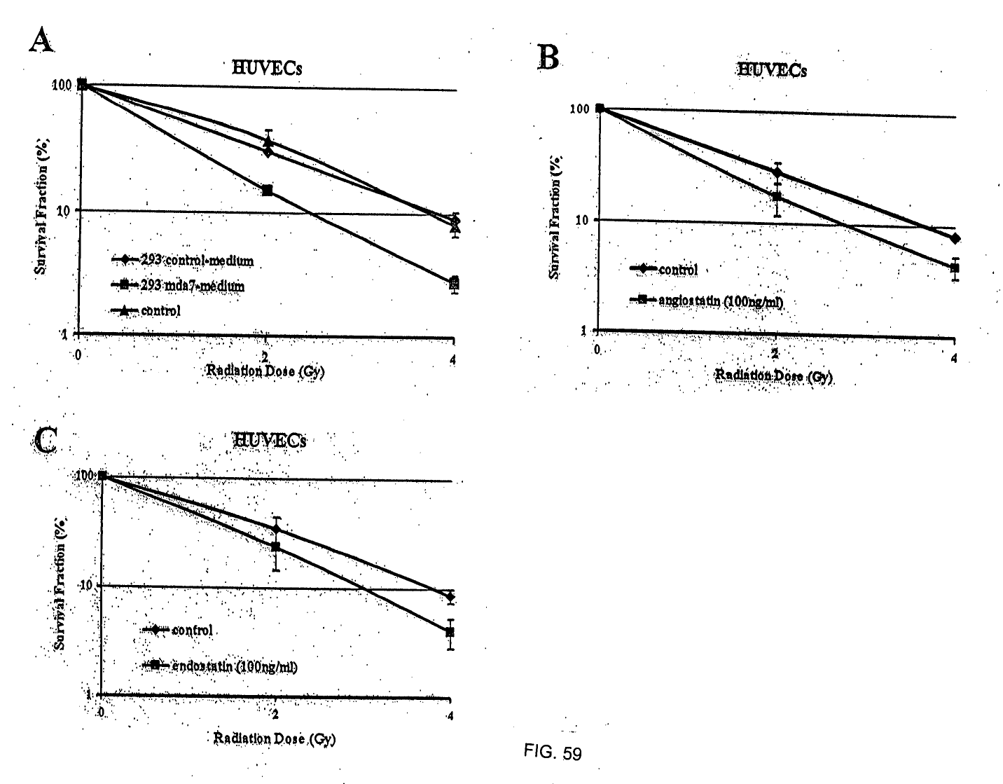

Previous studies have reported that MDA-7 protein can be secreted from Ad-mda7 infected tumor cells and functions as a cytokine, IL-24, at low concentrations (Dumoutier et al., 2001; Wang et al., 2002). Thus, it was possible that the ability of Ad-mda7 to radiosensitize the A549 xenograft tumor was due to a combination of effects that include direct radiosensitization of infected tumor cells, a suppression of angiogenesis factors, plus antiangiogenic and radiosensitizing properties of MDA-7 protein secreted by the infected tumor cells. To test the radiosensitizing effect of secreted MDA-7 protein on endothelial cells, clonogenic survival assays were performed using human umbilical vein endothelial cells (HWECs). HUVECs were pretreated with medium containing recombinant human MDA-7 protein for 12 hours prior to irradiation. This medium was derived from cultures of 293 cells that secrete MDA-7 protein. As shown in FIG. 59A, MDA-7 protein sensitized HUVECs to ionizing radiation at an estimated concentration of 10 ng/ml. As positive controls, we also pretreated HUECs with 100 ng/ml of angiostatin (FIG. 59B) or endostatin (FIG. 59C). Both angiostatin and endostatin, proteolytic fragments of plasminogen and collagen XVIII respectively, are well defined antiangiogenic agents (O'Reilly et al., 1994; O'Reilly et al., 1997). Previous reports have indicated that both angiostatin and endostatin radiosensitize endothelial cells (Mauceri et al., 1998, Hanna et al., 2000. Although both angiostatin and endostatin at 100 ng/ml produced radiosensitizing effects on HWVECs (FIG. 59B, C), MDA-7 protein appeared to be more potent in this regard.

8. Recombinant Human MDA-7 Protein does not Sensitize A549 Cells or Normal Human Fibroblast to Radiation

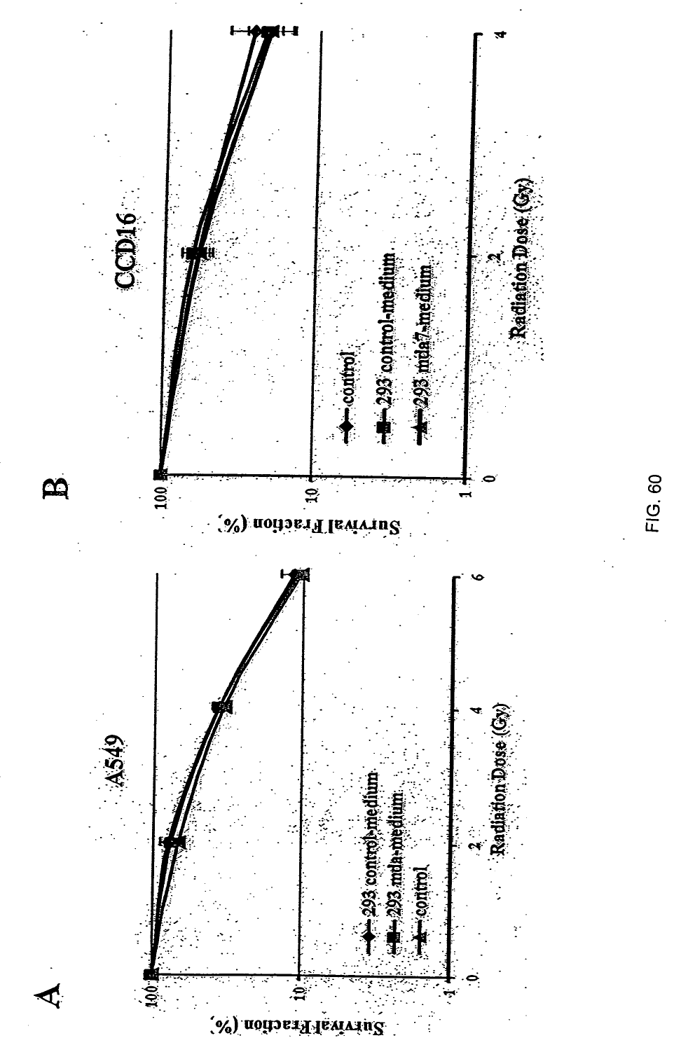

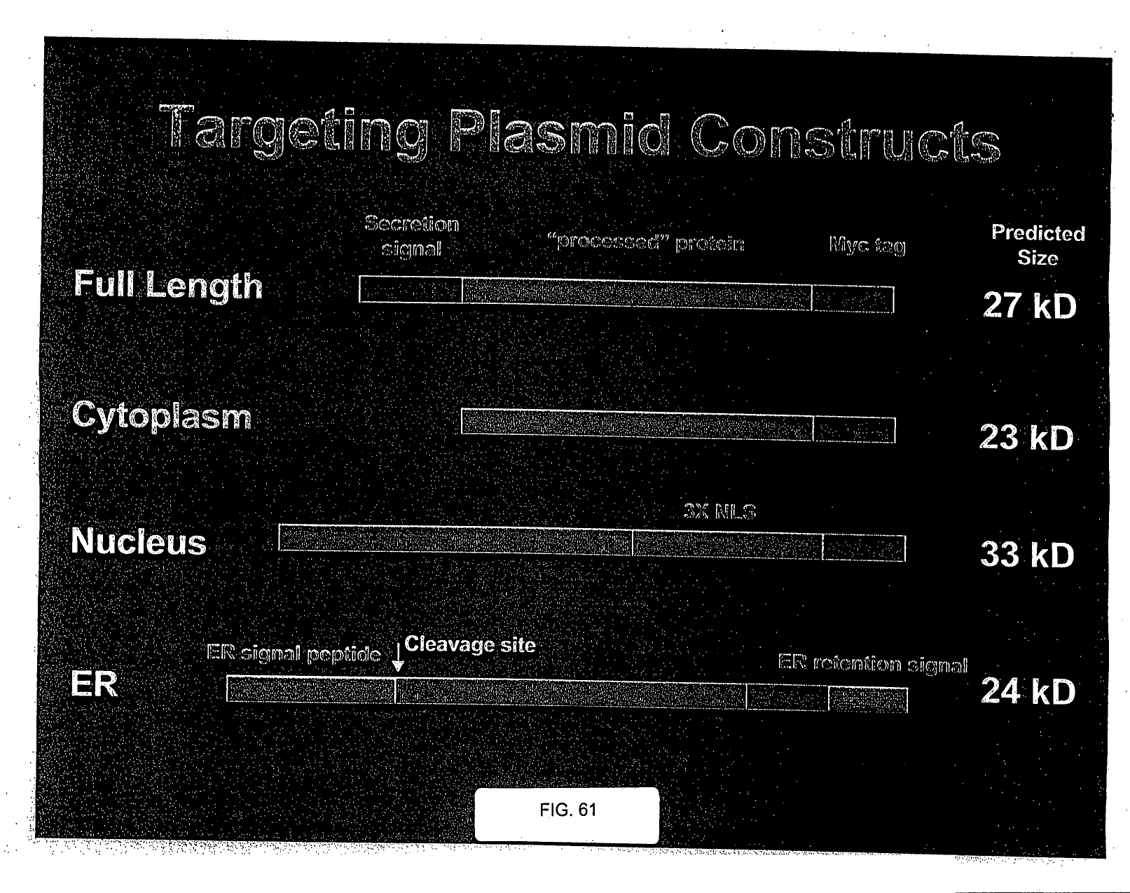

It was previously reported that Ad-mda7 radiosensitized A549 cells but not normal human lung fibroblasts cells, CCD16 cells, in vitro (Kawabe et al., 2002). To evaluate whether recombinant, human MDA-7 protein sensitizes these cells to radiation, clonogenic assays using conditioned media from stably transfected 293 cells were performed as described above. The results showed that MDA-7 protein does not produce radiosensitizing effects in either A549 cells or CCD16 cells (FIG. 60A, B).

### Example 28

### Anti-Cancer Activity of the MDA-7 Tumor Suppressor Proteins is Mediated by Signaling from the Endoplasmic Reticulum (ER)

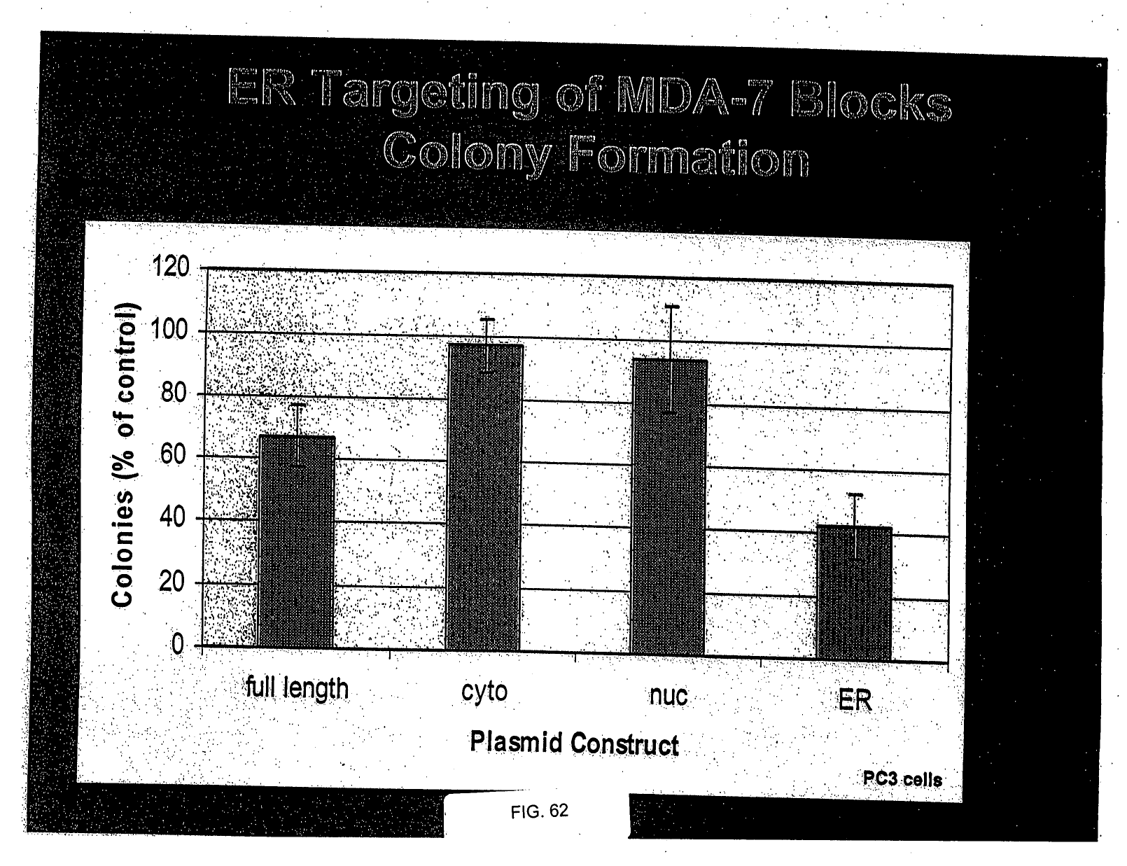

This study addresses the question of whether intracellular MDA-7 protein has enhanced killing activity if it is targeted to specific sub-cellular locations. Several plasmid constructs of mda-7 were created using vectors that target the expressed protein to various subcellular compartments, including cytoplasm, nucleus, and ER (FIG. 61). Additionally, a full-length mda-7 cDNA, including the secretion signal, was subcloned into the cytoplasmic backbone. The re-targeted vectors were evaluated for MDA-7 protein expression via transfection into lung tumor cells and all caused high levels of intracellular MDA-7 expression by western blot analysis. Subcellular re-targeting of MDA-7 protein expression was confirmed via immunohistochemistry. Using flow cytometry and colony formation assays, the ability of re-targeted MDA-7 to kill cancer cells was investigated (FIG. 62). The cytoplasmic and nuclear MDA-7 constructs did not elicit cell death, whereas full length (secreted) MDA-7 was cytotoxic. The ER-targeted mda-7 construct also elicited cell death in tumor cells (FIG. 63). Thus, it appears necessary for MDA-7 to enter the secretory pathway for it to be effective in inducing apoptosis.

### Example 29

### Cytokine Induction of MDA-7 in Human Peripheral Blood Mononuclear Cells

1. Expression of MDA-7 in Human Peripheral Blood Mononuclear Cells (PBMC)

Immunoblotting was initially used to test for presence of MDA-7/IL-24 protein after mitogenic lectin stimulation of whole PBMC populations. PBMC were isolated from the peripheral blood of normal healthy donors by centrifugation over Histopaque (Sigma, St. Louis, Mo.). Cells were cultured at a concentration of 1×10⁶ cells/ml in RPMI-1640 based media supplemented with L-glutamine, Hepes, penicillin, streptomycin, and 10% human AB serum (Pelfreez, Brown Deer, Wis.) for 72 hr in the presence of PHA-P at 5 μg/ml or LPS 10 μg/ml (both from Sigma, St. Louis, Mo.). Four hours prior to harvest Brefeldin A (BFA, Sigma-Aldrich) was added, to inhibit cytokine secretion, at a final concentration of 10 μg/ml. Cell lysates were prepared from the activated PBMC following a standard protocol. Boiled and reduced samples were separated by SDS-PAGE on a 12% gel and transferred to nitrocellulose. After blocking the membrane was incubated with the rabbit polyclonal anti-MDA-7, washed and incubated with HRP conjugated goat anti-rabbit secondary antibody. Blots were developed with ECL reagent (Amersham Pharmacia Biotech, Piscataway, N.J.). Membranes were stripped and reprobed with anti-actin antibody.

The expression of MDA-7 in 72 hour PHA and LPS stimulated PBMC was examined. The resulting activated populations were separated into CD3+, CD19+, CD56+ sub populations by magnetic cell sorting with directly conjugated antibodies and a MiniMax (Miltenyi Biotec, Sunnyvale Calif.). Expression of MDA-7 in these populations was determined by immunohistochemistry staining with the 7G9 anti MDA-7 monoclonal antibody. At this time point CD3+ cells were negative for MDA-7, while the CD19+, B cells, as well as the CD56+, NK cells, were positive for MDA-7. Given these results, experiments were set up to determine the kinetic of MDA-7 expression in activated PBMC.

2. PHA Induction of IL-24 in PBMC, Protein and mRNA

To determine the kinetics of IL-24 induction in activated cells, PBMC were stimulated with PHA and examined cells for expression of the protein at early time points. MDA-7 protein expression was measured by intracellular flow cytometry PBMC and mRNA expression by real time RT PCR.

PBMC were stimulated with 5 μg/ml PHA in media containing 10% normal human serum for the 0, 2, 6, 12, 24, and 48 hours. Cells were isolated at the designated times and protein levels measured and RNA isolated. Real time RT-PCR was performed using TaqMan OneStep procedure according to the manufacturers protocol (Applied Biosystems, Foster City Calif.). Gene specific primer/probes for MDA-7 and HPRT were purchased from Applied Biosystems. The reactions were carried out according to manufacturer's protocol using an ABI Prism 7900 HT sequence detection system and the analysis was conducted using Sequence Detection Software version 2.9. MDA-7 protein was detected by intracellular FACs analysis. Cells were treated with Brefeldin A at 10 μg/ml 4 hr prior to harvest to prohibit secretion of the cytokine. Where applicable cells were first surface stained using directly conjugated antibodies and then, to detect intracellular proteins, cells were fixed by treatment with 4% paraformaldehyde and then permeabilized with detergent, n-octyl glucopyranoside at 7 mg/ml. As the control for permeabilization, cells were also stained with a monoclonal antibody directed against the intracellular protein vimentin. Standard protocols were followed for immunofluorescence staining. Immunofluorescence was analyzed on a FACSCalibur with Cell Quest software (BD Immunosciences).

As a control for these experiments, expression of IL-2 was also analyzed. It was found that MDA-7 message follows that of IL-2, reaching peak levels at 6 to 8 hours after stimulation with a fold increase reaching 11,000 compared to unstimulated PBMCs. Protein expression, as measured by intracellular flow cytometry, follows message reaching a maximum expression at 8 hours.

3. Cytokine Induction of MDA-7

The above experiment suggests that MDA-7 mRNA peaks relatively early after PHA stimulation. To determine what cytokines may be driving this expression, PBMC were stimulated with IL-2, a major cytokine produced during PHA activation. Normal PBMC were stimulated in media containing 10% human serum. Cells were harvested at the specific times and tested for MDA-7 protein expression by intracellular FACs and MDA-7 mRNA expression by real time RT PCR.

It was found that protein expression began to increase at 4 hr. Levels of expression are lower than seen with PHA stimulation, which could reflect the strength of the mitogenic stimulation compared to the purified cytokine. It also could reflect that cells not expressing IL-2 receptors are also producing MDA-7. mRNA expression suggests biphasic kinetics with a peak at 1 hr which decreases and then increases again at 12 hr. In another experiment, neutralizing antibody to IL-2 was added to PBMC at the beginning of the PHA stimulation. Cells were harvested at 8 hr and MDA-7 protein measure by intracellular FACs and mRNA by RT PCR. MDA-7 protein and mRNA expression was blocked approximately 50% compared to the IgG control.

Other cytokines were also tested for their ability to induce MDA-7 expression in resting PBMC. PBMC were analyzed for MDA-7 expression 6 hr after stimulation with 100 U/ml of IL-2, IL-4, L-7, IL-15, or IFNγ. At this time point IL-7 and IL-15 stimulated the expression of both MDA-7 mRNA and protein.

These data suggest that IL-2, IL-7, and IL-15 are involved in the upregulation of MDA-7 expression in PBMC. These three cytokines share the common cytokine receptor gamma-chain (γc). The IL-7R is composed of a unique α-chain and γc. While the IL-2R and IL-15R are composed of three subunits: the IL-2/IL-15Rβ, the γ, and each with a unique α-chain. There is considerable evidence that cytokines that bind to receptors containing γc are involved in T cell maintenance and homeostasis. The fact that these cytokines stimulate expression of MDA-7 in PBMC suggests that MDA-7 may also be involved in T cell homeostasis.

4. Blocking of 11-24 Expression with Anti-IL2R Antibodies

To further establish whether cytokines that bind to the γc are responsible for MDA-7 expression, studies were conducted using blocking antibodies directed against the three subunits of the IL-2 receptor, IL-2Ru, IL-2/IL-15β, and γc (IL-2Rγ) (R&D Systems, Minneapolis, Minn.) to attempt to block the induction of MDA-7 expression in PHA activated PBMC. Antibodies added at the initiation of culture with PHA were used at 5 μg/ml with mouse IgG used as a control for nonspecific blocking. In three separate experiments, blocking of MDA-7 mRNA expression was observed, ranging from 0% to 24% with anti-IL2Rα, 19% to 36% with anti-IL2/IL-15Rβ and 15% to 26% with anti-IL2Rγ.

MDA-7 protein expression was blocked by the addition of anti-IL-2 monoclonal antibody at the initiation of the PHA stimulation. Thus, the expression of MDA-7 in PBMCs is induced by IL-2, IL-7, and IL-15. All-of these cytokines utilize components of the functional IL-2 receptor. These results support the concept that MDA-7 is a pro-inflammatory cytokine involved in a Th1 type immune response.

### Example 30

### MDA-7/IL-24 Mediated Killing of Human Ovarian Cancer Cells Involves the Fas/FasL Signaling Pathway

Materials and Methods

1. Cell Lines and Reagents

Human ovarian cancer cell lines OVCA 420, and MDAH 2774 were a gift from Dr. J. K. Wolf (M.D. Anderson Cancer Center, Houston, Tex.). Cell were grown in Minimum non-essential amino acid medium supplemented with 10% FBS. Human fibroblast cell line CCD-16 was purchased from ATCC (Rockville, Md.). AP-1 consensus oligonucleotides (5′-cgcttgatgagtcagccggaa-3′ (SEQ ID NO:3)) were purchased from Promega (Madison, Wis.). Adenovirus carrying the mda-7 (Ad-mda7) or luciferase (Ad-luc) gene was obtained from Introgen Therapeutics, Inc. (Houston, Tex.).

2. Determination of Transduction Efficiency

The transduction efficiency of human ovarian cancer cell lines and normal fibroblast cell line (MRC-9) were determined using an adenovirus expressing the GFP gene (Ad-GFP). Cells (MDAH 2774, OVCA 420, and MRC-9) were seeded at 5×10⁵ cells per well in six-well tissue culture dishes. The following day, cells were either uninfected (mock) or infected with Ad-GFP at 2500, 3000, 5000, 10000 viral particles per cell (vp/cell). Twenty-four hours after infection, cells were washed, resuspended in PBS and analyzed by flow cytometry analysis. At 3000 vp/cell more than 90% of the cells were transduced for all the cell lines. Hence, for all subsequent experiments described below we used a moi of 3000 vp/cell.

3. Cell Proliferation Assay

Tumor cells (MDAH 2774 and OVCA 420) and normal (MRC-9) cells were seeded at 1×10⁵ cells per well in six-well tissue culture dishes. The following day, cells were treated with PBS, Ad-luc or Ad-mda7 (3000 vp/cell). Cells were harvested on days 1, 2, 3, 4 and 5 after infection and counted by trypan-blue assay. Experiments were done atleast three separate times and the results represented as the average of the three experiments.

4. Cell Cycle Analysis

Tumor (MDAH 2774 and OVCA 420; 5×10⁵) cells were treated with PBS, Ad-luc or Ad-mda7 (3000 vp./cell) in 6-well plates and incubated at 37° C. Cells were harvested at 24, 48 and 72 h after treatment, washed in PBS, and fixed overnight at −20° C. in 70% ethanol. The cells were then resuspended in PBS containing RNase A (1 mg/ml), and 50 μg/ml propidium iodide (Sigma Chemicals, St. Louis, Mo.) and subjected to FACS analysis. Uninfected cells served as negative controls in these experiments.

5. Apoptotic Cell Staining

Cells (MDAH 2774 and OVCA 420) were seeded in 6-well plates at a density of 5×10⁵ cells per well and treated with PBS, Ad-luc or Ad-mda7 (3000 v.p./cell). Seventy-two hours after infection, cells were incubated with Hoechst No. 33342 (Sigma, St. Louis, Mo.) for 15 min, washed with PBS twice and observed under a fluorescent microscope for apoptotic cells as determined by fragmented nuclei.

6. Western Blot Analysis

Tumor cells treated with PBS, Ad-mda7 or Ad-luc were subjected to western blot analysis using techniques known to those of ordinary skill in the art. The following primary antibodies were used: PKR, phosphospecific p38, pJNK, p44/42, peIF2, caspase-9 (Cell Signaling, Boston, Mass.); caspase-3, PARP, FAF1, FADD, Fas, and FasL (PharMingen, San Diego, Calif.). The polyclonal antibodies to MDA-7 were from Introgen Therapeutics, Inc. (Houston, Tex.). The proteins were visualized on enhanced chemiluminescence film (Hyperfilm; Amersham) by application of Amersham's Enhanced Chemiluminescence Western Blotting Detection System

7. Electrophoretic Mobility Shift Assay (EMSA)

MDAH 2774 (5×105) were treated with Ad-luc or Ad-mda7 (3000 v.p./cell) in 6-well plates. Cells were harvested at various time points (24, 48, and 72h) and cytoplasmic and nuclear extracts were prepared and subjected to EMSA using techniques well-known to those of ordinary skill in the art. Briefly, double-stranded oligonucleotides consensus AP-1 (Promega) were end-labeled with [y-³²P]-ATP using T4 polynucleotide kinase. A typical binding reaction mixture contained the labeled oligonucleotide and 0.5 μg poly (dI-dC) and nuclear protein extracts (10 μg) were incubated at 25° C. for 30 min in 5× gel shift binding buffer [20% glycerol, 5mM MgCl₂ 2.5mM EDTA, 2.5 mM DTT, 250 mM NaCl, 50 mM Tris-HCl (pH 7.5)]. The complexes were resolved on nondenaturing 5% polyacrylamide gels in 0.5× Tris-borate EDTA buffer for 1 h 30 min at 300 V. The bands were visualized by autoradiography and quantitated using the Image Quant software (Molecular Dynamics, Amersham-Pharmacia, Biotech, Piscatway, N.Y.).

8. RNase Protection Assay (RPA)

Cells (MDAH 2774) were plated at a density of 5×10⁵ in 6-well plates and treated with PBS, Ad-luc or Ad-mda7. Total RNAs from these cells were isolated at 24, 48 and 72 h after treatment using Trizol reagent. The mRNA transcripts for the indicated apoptosis-related genes caspase-8, Fas, FasL. FADD, FAF-1, TRAIL, TNFr, TRADD and RIP as well as the internal controls L32 and glyceraldehyde-3-phosphate dehydrogenase were analyzed using the hApo-3 Multi-Probe Probe template set (Pharmingen). Probe synthesis, hybridization, and RNase treatment were performed using RiboQuant Multi-Probe RNase Protection Assay System (PharMingen) as per manufacturer's guidelines. Protected transcripts were resolved by electrophoresis on denaturing polyacrylamide gels (5%) and exposed to hyperfilm overnight at −80° C.

9. Fas Promoter Analysis

MDAH 2774 cells (5×10⁵) plated in 6-well plates were transfected with a plasmid (FHR+) consisting of the luciferase gene under the control of the human Fas (CD95) promoter. Cells transfected with a plasmid (Δ6) that contained a mutation in the Fas promoter served as controls in these experiments. Transfections were performed using DOTAP liposomes. Six-hours after transfection cells were treated with PBS, Ad-βgal or Ad.mda-7 (3000 vp/cell). Cells were harvested at different time points (12, 24, 48h) after treatment, washed in PBS, and lysed in 200 μl of Reporter Lysis Buffer (Promega). Luciferase expression was determined as described previously and expressed as relative light units (RLU) per milligram of protein. Experiments were repeated at least two times and results represented as the average.

10. SiRNA Analysis

SiRNA analysis was performed using methods well-known to those of ordinary skill in the art.

Results

1. Ad-mda7 Selectively Inhibits Ovarian Cancer Cell Proliferation

Ovarian cancer cells (MDAH2774, OVCA420) were infected with Ad-mda7 and Ad-luc (3000 vp/cell). Cells were harvested at various time points after infection and analyzed for MDA-7 protein expression and growth inhibitory effects. Cells that were treated with PBS served as control. Exogenous MDA-7 protein expression was observed in all the cell lines that were treated with Ad-mda7. Cells treated with PBS or Ad-luc also showed some expression. However, this is attributed to cross-reactivity of the anti-MDA7 polyclonal antibody with non-specific protein. Although MDA-7 expression was observed in all the cell lines, significant (P=0.001) growth inhibition by Ad-mda7 was observed only MDAH 2774 and OVCA 420 cells compared to PBS and Ad-luc treated cells (FIG. 64). No significant growth inhibitory effect was observed in Ad-mda7 treated Hey and DOV13 tumor cell lines.

2. MDA-7 Induces G2/M Cell Cycle Arrest and Apoptosis in Ovarian Cells

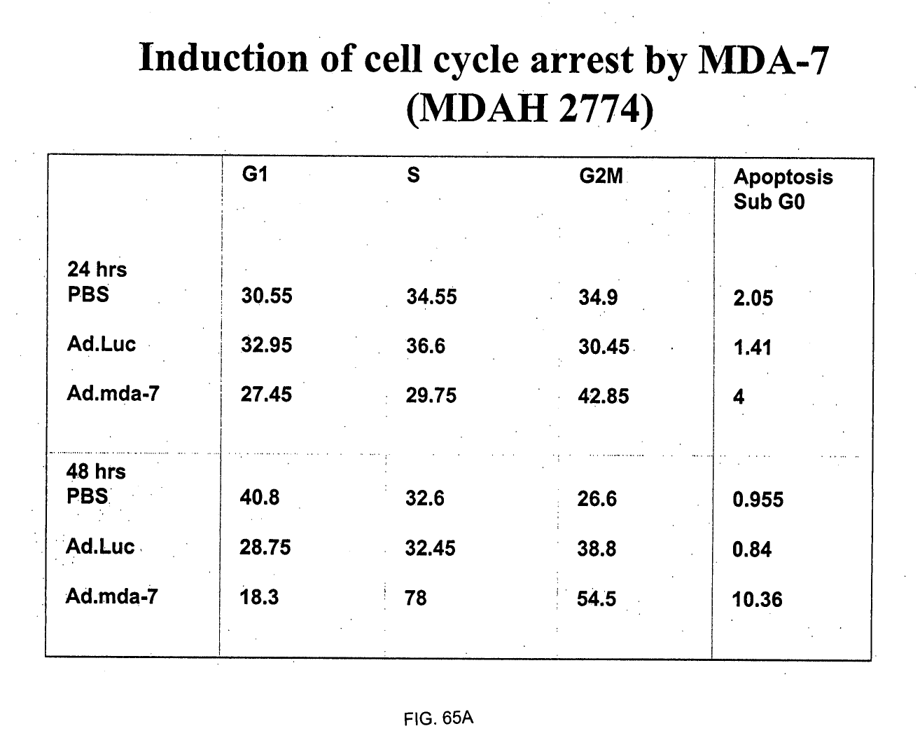

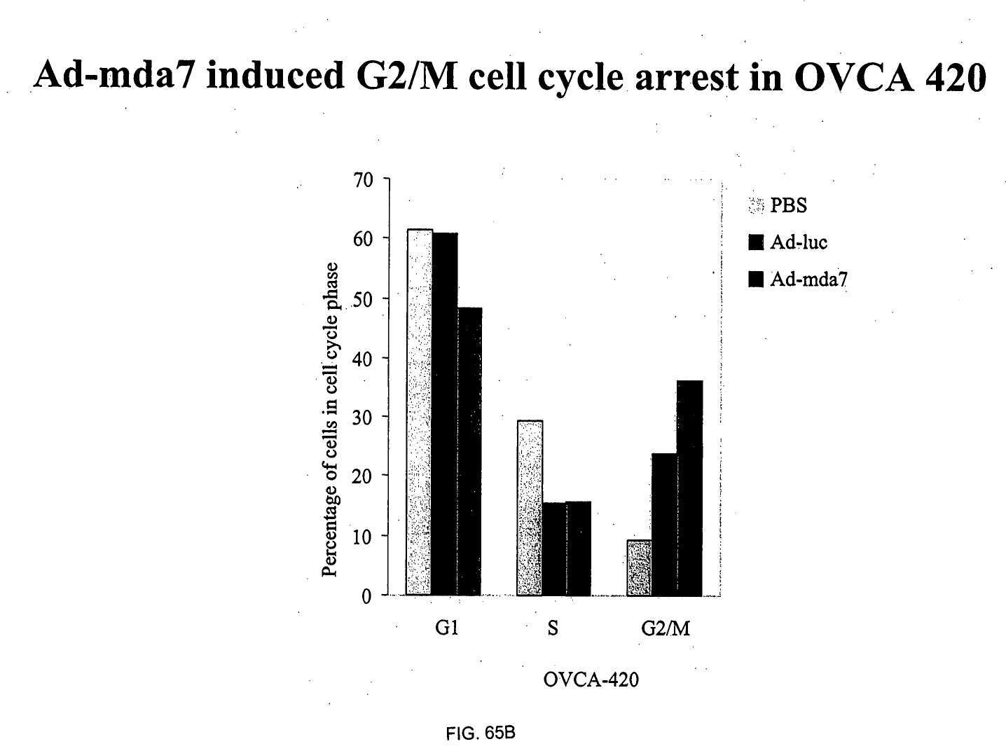

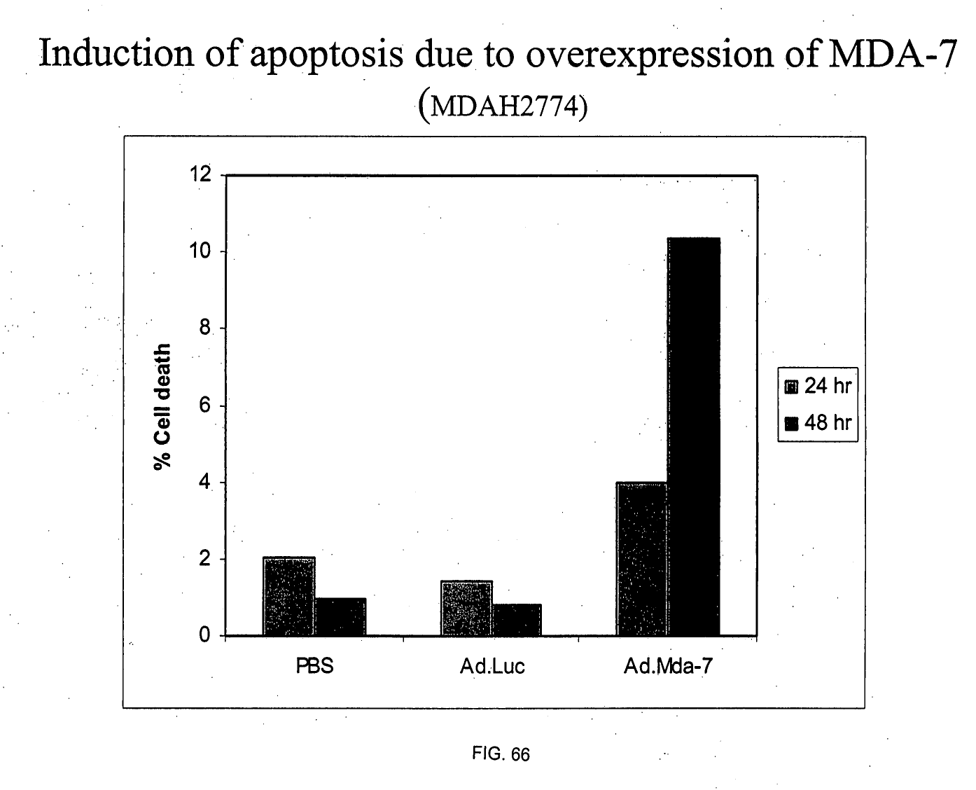

The mechanism of growth inhibition exerted by Ad-mda7 was next investigated. Ad-mda7 treated MDAH2774 (FIG. 65A) and OVCA 420 cells (FIG. 65B) demonstrated a significant increase in the number of cells in the G2/M phase compared to PBS and Ad-luc treated cells. However, no significant change in the number of cells in the G2/M phase was observed in Ad-mda7 treated Hey, DOV13 and SKOV3-ip cells. Associated with the cell-cycle arrest was the induction of apoptosis in MDAH2774 and OVCA 420 cells as evidenced by Hoechst staining (FIG. 66). No apoptotic changes were observed in cells infected with PBS or Ad-luc.

3. PKR Induces Apoptosis in Ovarian Cells

To study the molecular mechanism by which MDA-7 induced apoptosis, the expression of various signaling molecules that have previously been shown to participate in MDA-7 was analyzed. It has been suggested that PKR plays a role in dsRNA, viral and stress mediated apoptosis (Lee et al., 1994; Yeung et al., 1996; Kibler et al., 1997). It has been reported that Ad-mda7 induces apopteosis in lung cancer cell lines A549 and H1299 mediated by PKR. Therefore studies were conducted to determine whether PKR activity was increased in sensitive (MDAH 2774, OVCA 420) and resistant (Hey and DOV 13) cell lines following treatment with Ad-luc and Ad-mda-7. It was found that PKR levels dramatically increased in MDAH 2774 and OVCA 420 cells at 24-48 hrs and its substrate peIF₂ increased at 48hrs after Admda-7 infection. However, Ad.mda-7 infection of Hey and DOV13 did not induce PKR and its substrate peIF₂, which are resistant to the apoptosis-indcuing effects of Admda-7.

4. MDA-7 Treatment of MDAH 2774 Cells Induces Activation of MAPKs, JNK and p38

Experiments were carried out to determine whether MDA-7 signals to Fas via the p38 and/or the JNK pathways. The activation (phosphorylation) of JNK and/or p38 MAPK has been implicated in the pathway leading to stress-induced apoptosis in various cancer cells. The expression of phospho-JNK and phospho-38 MAPK in the MDAH 2774 and OVCA 420 was analyzed after Admda-7 treatment by western blotting. Phosphorylated JNK and p38 was significantly increased in a MDA-7 infected MDAH 2774 and OVCA 420 at 48hrs. This finding suggests the involvement of phosphorylated JNK and p38 MAPK in apoptosis. To confirm c-Jun and p-ATF₂ involvement, the level of the major AP-1 components (c-Jun and ATF-2) was determined at 24 and 48 h after infection with the Ad-mda7 and Ad-luc by Western blot analysis. Ad-mda7 significantly induced the levels of pcjun and ATF-2 at 24 and 48h. Ad-luc and Mock-infected (PBS) cells did not activate pc-Jun and pATF-2. These results suggest a potential involvement of c-Jun and pATF2 in AP-1 activation and further support the role of AP-1 activity in FasL stimulation. However, Ad-mda7 did not activate phospho-38, JNK, pc-Jun, ATF-2 and its target AP-1 in the resistant cell lines Hey and DOV 13.

5. CD95L Promoter Activity is Upregulated in MDAH 2774 Cells Upon Stimulation with MDA-7 Via Activation of an AP-1 Element

AP-1 is a major transcription factor, which consists of the Jun family (c-Jun, JunD, and JunB) or heterodimers of a Jun family member with any of the Fos family members (c-Fos, FosB, Fra-1, and Fra-2) or other transcription factors such as ATF2, CREB, and NFAT. Because all three MAPK pathways (ERK, JNK and p38) can activate AP-1, experiments were performed next to examine whether AP-1 functions as an integrating module transmitting the p38 and JNK. AP-1 binding activity to a synthesized AP-1 consensus sequence was determined by EMSA. The nuclear lysates from Ad-mda 7 infected cells have higher AP-1 binding activity at 24 and 48hrs than in the Ad-luc and PBS treated cells.

6. CD95L is Upregulated Upon Ad-mda7 Infection

These data show that CD95-CD95L interactions are important for induction of apoptosis following Ad-mda-7 infection. To gain insight into the expression status of the Fas transcripts in MDAH 2774 cells, the ribonuclease protection assay was used to measure the mRNA levels of several genes involved in sigaling cell death. It was found that Fas, FasL , FADD and caspase-8 mRNA levels increased following MDA-7 expression at 24hrs and there was no change in expression level at 33 hrs. In addition, Ad-luc and Mock treated cells showed no change in the mRNA expression levels of Fas, FasL, FADD and caspase-8. Taken together, these data support the hypothesis that MDA-7 activates FasL via a p38 and JNK dependent c-Jun-pATF2/AP-1 pathway.

7. Activation of the Caspase Cascade Following MDA-7 Expression

The downstream targets that are responsible for apoptosis were next investigated. Activation of capase 9 and caspase 3 was observed in Ad-mda7 treated MDAH 2774 and OVCA420 cells compared to cells treated with PBS or Ad-luc. Associated with the activation of the caspases was the cleavage of PARP, a substrate for the caspases.

### Example 31

### mda-7 Gene Transfer Exploits Multiple Molecular Pathways to Combat Cancer

Introduction of the melanoma differentiation-associated gene 7 (mda-7) into cells using a replication-deficient adenovirus (Ad-mda 7) results in growth inhibition and apoptosis in a broad spectrum of cancer cells, including those of breast, lung, colorectal, prostate, pancreatic, ovarian and melanoma origins. The cytotoxic activity of Ad-mda7 is tumor-selective as normal cells are resistant to MDA-7-induced death. The anti-tumor activity of Ad-mda 7 has been confirmed using multiple xenograft models in nude mice. Accumulated data indicates that MDA-7 activates genes and signaling pathways important for the onset of apoptosis (e.g., p53, BAX, TRAIL, fas, PKR, MAPK, jnk) and inhibits survival pathways (e.g., P13K).

Bioinformatics and structural analyses have now revealed that MDA-7 protein is a novel member of the interleukin-10 (IL-10) superfamily, which encompasses IL-10; -19; -20; -22 and -26. The mda-7 gene is contained with a cytokine cluster at 1q31/32. The MDA-7 protein shares the 6-helical configuration of IL-10, however, MDA-7 does not share the immunosuppressive properties of IL-10, and functions as a Th1 cytokine. MDA-7 is expressed in activated lymphocytes. MDA-7 treatment of human PBMC induces secretion of IL-6, gamma-IFN, IL-12, TNF-a and GM-CSF. Secretion of these Th1 cytokines is inhibited by IL-10. MDA-7 also binds endothelial cells and functions as a potent anti-angiogenic protein. This activity is mediated via the IL-22 receptor. Ad-mda7 or MDA-7 treatment of melanoma cells induces secretion of IL-6 and gamma IFN. Therefore, mda-7, recently classified as IL-24, is a novel IL-10 homolog with multi-modal anti-cancer properties. This unique combination of apoptosis induction, anti-angiogenesis and immune stimulation should provide a powerful approach for attacking cancer.

### Example 32

### Ectopic Production of MDA-7/IL-24 Inhibits Invasion and Migration of Human Lung Cancer Cells

Materials and Methods

1. Cell Culture

Human NSCLC cell lin A549 (adenocarcinoma) was purchased from the American Type Culture Collection (Rockville, Md.). Human large cell lung carcinoma cell line H1299 was a gift from Drs. A. Gazdar and J. D. Minna (The University of Texas Southwestern Medical Center, Dallas, Tex.). Tumor cells were maintained in RPMI-1640 medium containing 10% fetal bovine serum (FBS; GIBCO-BRL, Grand Island, N.Y.), antibiotics (GIBCO), and L-glutamine. Before the experiments were started, the absence of mycoplasma from the cells was verified. Cells were used in the log phase of growth.

2. Recombinant Adenoviral Vector

Construction of the replication-deficient human type 5 adenoviral vector carrying the MDA-7 gene (Ad-mda7) has been previously described (Saeki et al. 2000, and Mhashilkar et al., 2001). Viruses were propagated in human embryonic kidney 293 cells and purified by chromatography.

3. Cell Migration Assay

Tumor cells (Hi299 and A549) were seeded at a density of 5×10⁵ cells/well in six-well tissue culture plates. The next day, cells were infected with Ad-mda7 or Ad-luc at a multiplicity of infection of 2500 viral particles/cell. At 6 h after infection, the cells were trypsinized, washed in phosphate-buffered saline (PBS), and resuspended in serum-free RPMI-1640 medium. A cell migration assay was performed in a 24-well Transwell unit (Millipore, Cambridge, Mass.) as described previously (Ramesh et al., 2003). Briefly, polycarbonate filters with 8 μm pores were used. The lower chambers of the Transwell units were filled with serum-free medium, and the upper chambers were seeded with 1×10⁴ cells from each treatment group in triplicate wells. After 24 h and 48 h incubations, the cells that had been passed through the filter into the lower wells were counted, and the number was expressed as a percentage of the sum of the cells in the upper and lower wells. The experiments were performed four times, and the results were recorded by MDA-7 as the mean of these experiments.

In a parallel set of experiments, tumor cells subjected to various treatments as described above were subjected to cell viability assays at 24 h and 48 h, as described previously (Saeki et al., 2000; Mhashilkar et al., 2001). These experiments were performed to exclude the possibility that the inhibition of cell migration by MDA-7 was a result of cytotoxicity.

4. Cell Invasion Assay

Tumor cells (H1299 and A549) were seeded at 5×10⁵ cells/well in six-well tissue culture plates. The next day, cells were infected with Ad-mda7 or Ad-luc at an MOI of 2500 vp/cell or treated with 10 μM LY 294002 (Cell signaling, Beverly, Mass.) or 1 μg/ml MMP-II inhibitor (Santa Cruz Biotechnology, Santa Cruz, Calif.). After transfection, cultures were replenished with complete medium. At 6 h after treatment, cells were trypsinized, washed in PBS, and resuspended in serum-free RPMI-1640 medium. A cell invasion assay was performed in a 24-well Transwell unit coated with Matrigel (Becton, Dickinson and Company, Franklin Lakes, N.J.), as described previously (Stewart et al., 2002). Briefly, the lower chambers of the Matrigel-coated Transwell units were filled with serum-free medium, and the upper chambers were seeded with 1×10⁴ cells from each treatment in triplicate wells. After 24 h and 48 h incubations, the cells that had passed through the Matrigel-coated filter membrane into the lower well were counted as a measure of invasion. The invading cells were counted for each treatment and expressed as a percentage of the sum of the cells in the upper and lower wells. Experiments were performed at least three times, and the results were recorded as the mean of these experiments.

5. Gelatin Zymography Analysis

To determine the effect of Ad-mda7 treatment on MMP production, a gelatin zymography assay was performed, as described previously (Zhang et al., 2002). Briefly, tumor cells (H1299 and A549) grown in low-serum (1% FBS) medium were seeded at 5×10⁵ cells/well in six-well tissue culture plates and infected with Ad-mda7 or Ad-luc at an MOI of 2500 vp/cell. Cells treated with PBS served as a negative control in these experiments. At 6 h after infection, the culture medium was removed and replaced with fresh medium containing 1% FBS. At 24 h and 48 h after infection, cell culture supernatants were collected, clarified by centrifugation, and subjected to electrophoresis in sodium dodecyl sulfate (SDS)-polyacrylamide gels copolymerized with gelatin (Sigma Chemicals, St. Louis, Mo.). The gels were then washed and incubated with reaction buffer (50 mM Tris-HCl [pH 7.4], 0.02% NaN₃, and 10 mM CaCl₂) with constant shaking for 16 h at 37° C., stained, and destained. The protein concentration in the culture supernatant was measured to confirm that equal amounts were used for the assays. Relative activities of MMP-2 and -9 were quantified using ImageQuant software (Amersham Pharmaci Biotech, Piscataway, N.J.).

6. Immunoblotting

Immunoblotting using various antibodies was performed as described previously (Saeki et al., 2000). Briefly, cells were harvested by trypsinization and resuspended in lysis buffer (62.5 mM Tris-HCl, 2% SDS, 10% glycerol, and 4 M urea). Protein samples (50 Δg each) were diluted into a 20 Δl solution of lysis buffer and 5% 2-mercaptoethanol (Bio-Rad Laboratories, Hercules, Calif.) and heatedin a water bath at 95° C. for 5 min. Then, protein extracts were separated by 10% SDS-polyacrylamide gel electrophoresis (SDS-PAGE) in a vertical-slab gel electrophoresis cell (Bio-Rad). Next, the separated proteins were transferred from the gel to a nitrocellulose membrane (Hybond-ECL; Amersham Pharmacia Biotech, Buckinghamshire, England) and then blocked in a blocking solution (5% dry milk and 0.3% Tween 20 in PBS) for 1 h. Then, the membranes were incubated with the primary antibodies for MMP-2, MMP-9, and p85 P13K (Santa Cruz Biotechnology), phosphorylated FAK (Pharmingen, San Diego, Calif.), MDA-7 (Introgen Therapeutics, Inc., Houston, Tex.), and β-actin (Sigma Chemicals). The membranes were then incubated with horseradish peroxidase-labeled secondary antibodies (Amersham, England). Finally, the proteins were visualized on enhanced chemiluminescence film (Hyperfilm; Amersham, England) using Amersham's Enhanced Chemiluminescence Western blotting detection system. The relative change in the protein expression levels after various treatments were quantified using ImageQuant software (Amersham Pharmaci Biotech, Piscataway, N.J.) and expressed as a ratio with 1 being the value for PBS treated cells.

7. Experimental Lung Metastasis Model

To determine whether MDA-7/IL-24 production can inhibit metastasis, animal experiments were performed using an experimental lung metastasis model (Ramesh et al., 2001). Briefly, A549 tumor cells (5×10⁶) seeded in 150-mm tissue culture plates were treated with PBS, Ad-luc, or Ad-mda7 (2500 vp/cell). At 6 h after infection, cells were harvested, washed, and resuspended to a final volume of 1 ml in sterile PBS (1×10⁶ cells/100 μl). Cells were injected into female nude mice intravenously via the tail vein. There were five animals in teach treatment group. Three weeks after the injection of the cells, the animals were euthanized by CO2 inhalation. Lungs from eachof the mice from the three groups were injected intratracheally with India ink and fixed in Fekete's solution, as described previously (Ramesh et al., 2001). The effect of MDA-7/IL-24 on tumor metastasis was determined by counting the number of metastatuc tumors in each lung under a dissecting microscope. Experiments were performed two times, and the results were recorded as the mean of the two experiments.

8. Statistical Analysis

The statistical significance of the experimental results was calculated using ANOVA and the Mann-Whitney rank-sum test. The differences among groups were interpreted as statistically significant if the P value was less than 0.05.

Results

1. MDA-7/IL-24 Inhibits Tumor Cell Migration

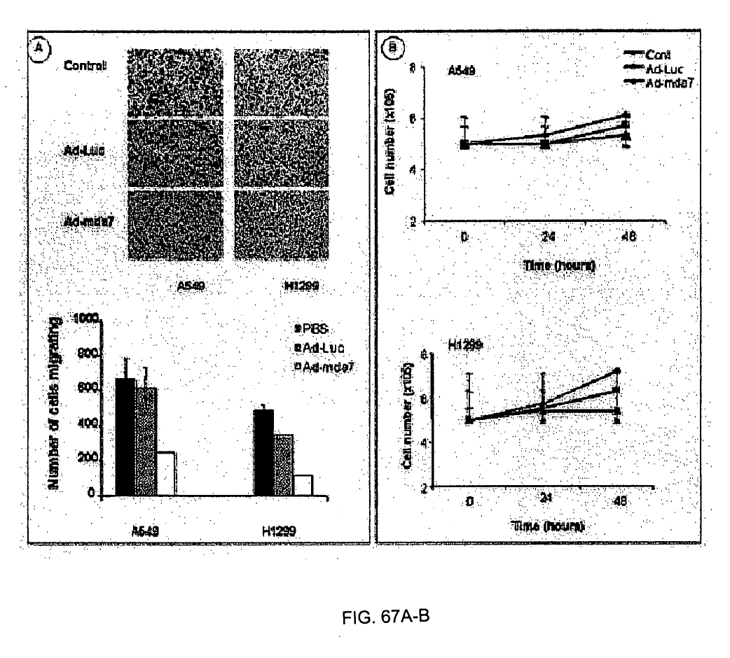

Tumor cells treated with Ad-mda7 were significantly (P=0.002) less able to migrate than were cells treated with Ad-luc or PBS (FIG. 67). The number of cells (A549 and H1299) migrating was significantly less after treatment with Ad-mda7 (<250 cells) than after treatment with PBS (>500) or Ad-luc (>350). The inhibitory effect was observed to be more effective at 48 h than at 24 h. To show that the inhibition of cell migration was not due to MDA-7/IL-24-mediated cell death, in a separate but parallel set of experiments, cells treated with PBS, Ad-luc, and Ad-mda7 were subjected to a cell viability assay at 24 h and 48 h after infection. No significant difference in cell viability was observed at this time-points indicating that the inhibition of migration by MDA-7/IL-24 was not due to cell death (FIG. 67B). Note that at 24 post-transduction, all three experimental groups are superimposable indicating no significant cell death. By 48 hr, some cell death is occurring, however, with this vector dose, significant Ad-mda 7 mediated death is only observed at 72 and 96 hr post transduction. These results show that MDA-7/IL-24 did inhibit cell migration.

2. MDA-7 Inhibits Tumor Cell Invasion

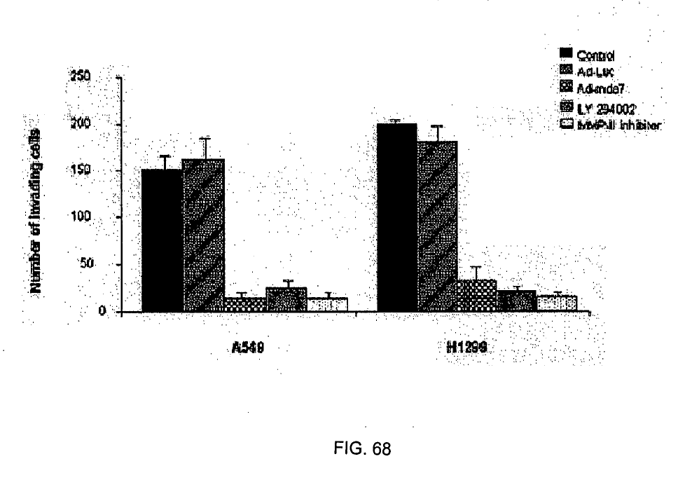

Tumor cells treated with Ad-mda7 were much less invasive, as indicated by the small number of cells on the outer membrane of the Matrigel invasion assay filter, than cells treated with PBS or Ad-luc (FIG. 68). In both A549 cells and H1299 cells, the number of invading cells was significantly less after treatment with Ad-mda7 (<50 cells; P=0.001) than with PBS (>140 cells) or Ad-luc (>150 cells). The inhibitory effect exerted by MDA-7 was similar to the inhibitory effect observed in cells treated with LY 94002, a PI3K inhibitor, or with MMP-II inhibitor. A cell viability assay showed that the inhibition was not a result of MDA-7/IL-24-mediated cell death. These results show that MDA-7/IL-24 did inhibit cell invasion.

3. MDA-7 Down-Regulates the Production of Proteins Associated with Cell Migration and Invasion

The regulation of proteins that are associated with cell migration and invasion signaling pathways was examined by western blot analysis. Cell lines containing wild type p53 (A549) and null for p53 (H1299) were used. MDA-7IL-24 production was not observed in PBS or Ad-luc treated tumor cells, but high levels of MDA-7WL-24 production were found in Ad-mda7 treated cells. Overproduction of the MDA-7/IL-24 protein in both H1299 and A549 tumor cell lines resulted in decreased production of p85 P13K and pFAK and increased production of pJNK, p38 MAPK, and p44/42 MAPK. In contrast, no significant change in the production of these proteins was observed in cells that were treated with PBS or Ad-luc. The inhibition of p85 P13K and pFAK was also observed in cells treated with the P13K inhibitor LY 294002, although the inhibition differed between the two cell lines. The decrease in pFAK expression in cell lines overproducing MDA-7 was greater than that observed with LY294004. Furthermore, LY 294002 treatment resulted in increased production of p38 MAPK and p44/42 MAPK in H1299 cells. In A549 cells, LY 294004 increased the production of pJNK, and p44/42 MAPK. These results indicate that MDA-7 like LY 294002 selectively inhibits P13K with no significant effect on other signaling molecules investigated in the present study.

4. Inhibition of Matrix Metalloproteinase Production in Tumor Cells by MDA-7/IL-24

Tumor cells overproducing MDA-7 were next examined for MMP regulation by zymography and western blot analysis. Zymography and Western blot analysis showed that production of the MMP-2 and -9 proteins was decreased in A549 tumor cells treated with Ad-mda7, compared with cells that were treated with PBS or Ad-luc. In H1299 cells treated with Ad-mda7, a decrease in MMP-2 but not in MMP-9 was observed compared with cells treated with PBS or Ad-luc. Results of zymography analysis correlated with the results of the Western blot analysis. Thus Ad-mda7 can modulate MMP expression and activity in both p53 wild type and p53 null NSCLC lines.

5. MDA-7/IL-24 Inhibits Experimental Lung Metastases

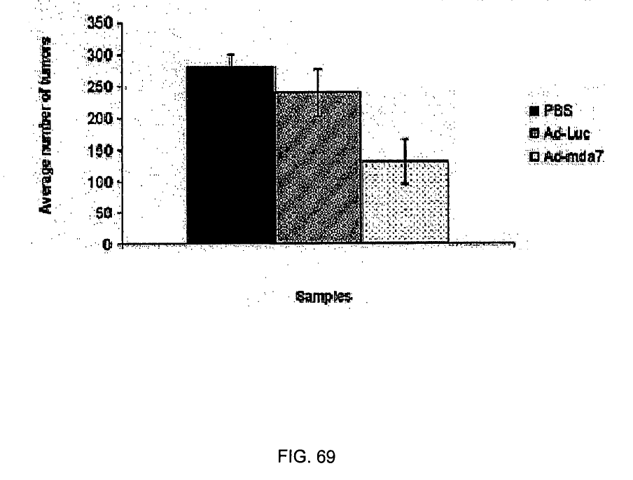

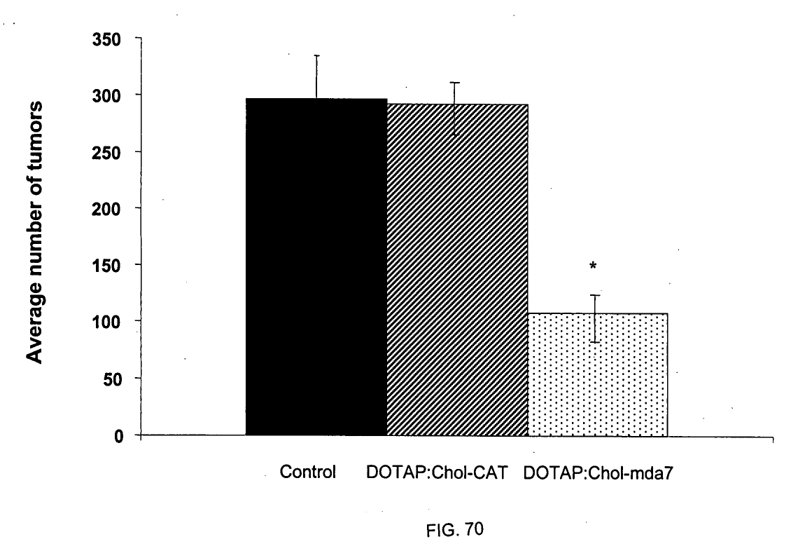

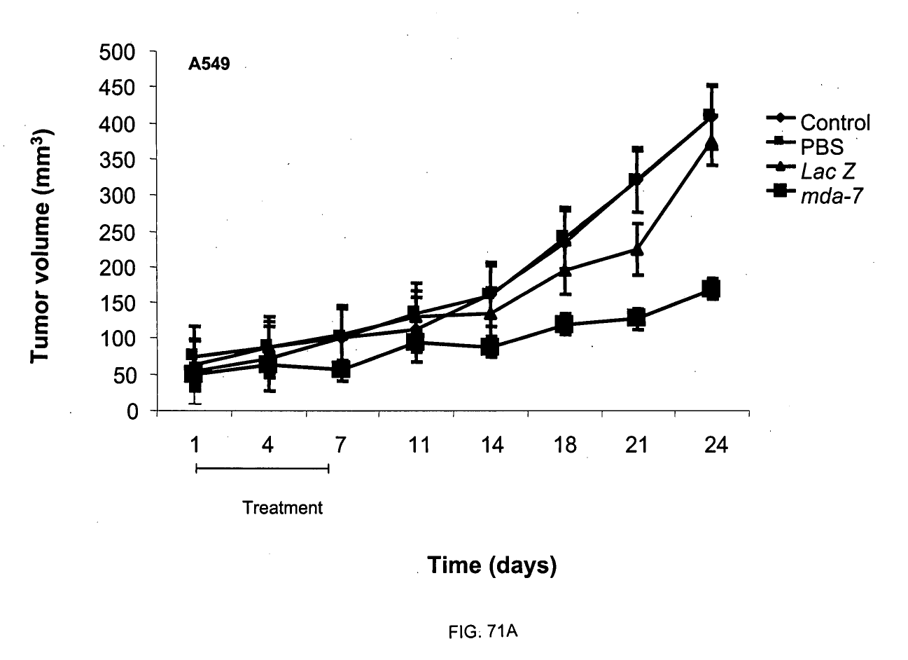

In an experimental lung metastasis model using A549 human lung cancer cells in nude mice, significantly (P=0.01) fewer lung tumor nodules formed in mice injected with tumor cells treated with Ad-mda7 than in mice injected with tumor cells treated with PBS or Ad-luc (FIG. 69). Inhibition of experimental metastasis correlated with histological tissue staining that demonstrated fewer numbers of tumors.

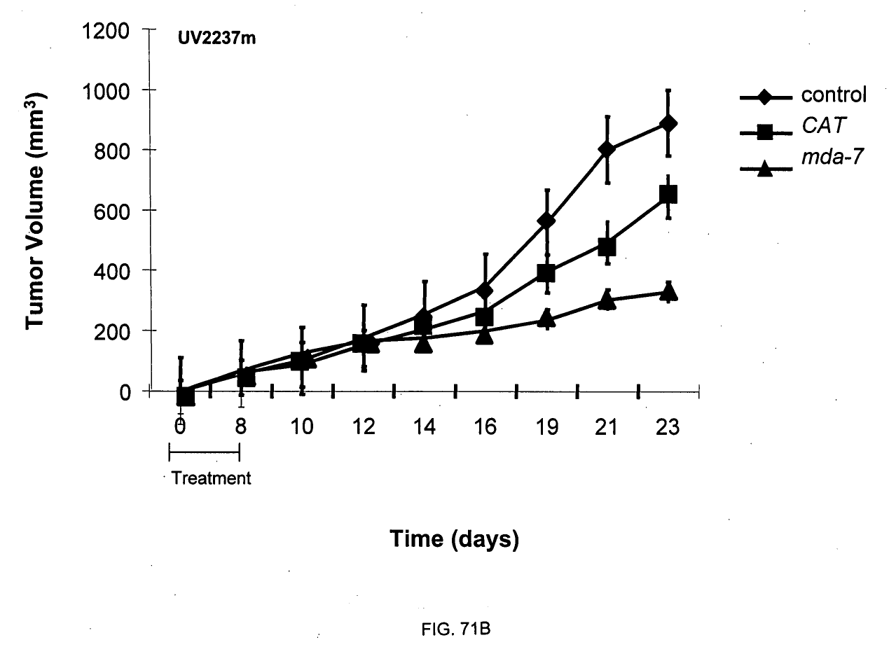

To further confirm that inhibition of experimental metastasis was not solely due to cell death, additional in vivo experiments were carried out. Treatment of lung tumor bearing animals with DOTAP:Chol-mda7 resulted in significant inhibition (P=0.001) of experimental metastasis compared to mice that were untreated or treated with DOTAP:Chol-CAT complex (FIG. 70). The ability to inhibit experimental metastasis is considered as an indirect correlate of inhibition of migration and invasion of tumor cells. These results demonstrate that MDA-7 inhibits tumor cell migration and invasion in vivo and is in agreement with observed in vitro results.

### Example 33

### Local and Systemic Inhibition of Lung Tumor Growth after Lipsome Mediated MDA-7/IL-24 Gene Delivery

Materials and Methods

1. Materials

All lipids (DOTAP, cholesterol) were purchased from Avanti Polar Lipids (Albaster, Ala.). Ham's/F12 medium and fetal bovine serum (FBS) were purchased from GIBCO-BRL-Life Technologies (New York, N.Y.). Polyclonal rabbit anti-human MDA-7 antibody was obtained from Introgen Therapeutics, Inc. (Houston, Tex.) and antimouse CD3 1 from Santa Cruz Biotechnology, Inc. (Palo Alto, Calif.).

2. Cell lines and Animals

Human non-small cell lung carcinoma cell line A549 was obtained from American Type Culture Collection and maintained in Ham's-F12 medium supplemented with 10% FBS, 1% glutamate, and antibiotics. Murine UV2237M cells were obtained from Dr. Isaiah J. Fidler (M. D. Anderson Cancer Center) and maintained as described elsewhere (Ramesh et al., 2001). Cells were regularly passaged and tested for presence of mycoplasma. Four- to six-week-old female BALB/c nude (nu/nu) mice (Harlan-Sprague Dawley Inc., Indianapolis, Ind.) and C3H/Ncr mice (National Cancer Institute, Fredericksburg, Md.) used in the study were maintained in a pathogen-free environment and handled according to institutional guidelines established for animal care and use.

3. Purification of plasmids

The plasmids used in the study were cloned in pVax plasmid vector ( Invitrogen, Carlsbad, Calif.) and purified as described elsewhere (Templeton et al., 1997; Gaensler et al., 1999). Briefly, plasmids carrying the bacterial β-galactosidase (Lac-Z), chloramphenicol acetyl transferase (CAT), or human mda-7 cDNA, under the control of cytomegalovirus (CMV) promoter, were grown under kanamycin selection in the Escherichia coli host strain DH5α. Endotoxin levels of purified plasmids were determined by using the chromogenic limulus amebocyte lysate kinetic assay kit (Kinetic-QCL; Biowhittaker, Walkersville, Md.). The concentration and purity of the purified plasmid DNA's were determined by OD 260/280 ratios.

4. Synthesis of DOTAP:Chol Liposomes and Preparation of DOTAP:Chol-DNA Mixtures

DOTAP:Chol liposomes were synthesized and extruded through Whatman filters (Kent, UK) of decreasing size (1.0, 0.45, 0.2, and 0.1 μm) as described elsewhere (Chada et al., 2003; Templeton et al., 1997). DOTAP:Chol-DNA complexes were prepared fresh two to three hours prior to injection in mice.

5. Particle Size Analysis.

Freshly prepared DOTAP:Chol-DNA complexes were analyzed for mean particle size by using the N4 particle size analyzer (Coulter, Miami, Fla.). The mean particle size of the liposome-DNA complexes ranged between 300 nm and 325 nm.

6. Effect of DOTAP:Chol-mda7 Complex on Subcutaneous Tumor Xenografts

In all the experiments, 5×10⁶ tumor cells (A549) suspended in 100 μl sterile phospate-buffered saline (PBS) were injected into the right dorsal flank. When the tumors reached a size of 4-5 mm², the animals were randomized into groups and treatment was initiated. Tumor-bearing animals were divided into four groups of six animals. Group 1 received no treatment, group 2 received PBS, group 3 received DOTAP:Chol-LacZ complex (50 μg/dose), and group 4 received DOTAP:Chol-mda-7 complex (50 μg/dose); all treatments were administered intratumorally and were given daily for a total of six doses. Animals were anesthetized with methoxyflurane (Schering-Plough, Kenilworth, N.J.) for intratumoral injections per institutional guidelines. Tumor measurements were recorded every other day by observers without knowledge of the treatment groups, and tumor volumes were calculated by using the formula V (mm³)=a×b²/2, where “a” is the largest dimension and “b” is the perpendicular diameter (Saeki et al., 2002; Ramesh et al., 2001). Antitumor efficacy data are presented as cumulative tumor volumes for all animals in each group to account for both size and number of tumors. In all experiments, the statistical significance of changes in tumor size was determined by ANOVA.

To test the effect of mda-7 on mouse tumor cells, a syngeneic tumor model was utilized. For this purpose, C3H mice were injected subcutaneously with murine UV2237m fibrosarcoma cells (1×10⁶) and divided into three groups (n=8/group). When the tumor size reached 4-5 mm²⁻, animals received intratumoral treatment as follows: no treatment (control), DOTAP:Chol-CAT complex, or DOTAP:Chol-mda-7 complex. Treatment schedule and analyses of the therapeutic effects were the same as already described for the A549 tumor model. Experiments were repeated two times for statistical analysis and significance.

7. Measurement of MDA-7, Apoptosis, and CD31

Subcutaneous A549 or UV2237m tumors established in nu/nu or C3H mice respectively were harvested and fixed in 4% buffered formalin, embedded in paraffin, and cut in 4-μm sections. Tissue sections were immunostained for MDA-7 transgene expression as described elsewhere (Saeki et al., 2002; Ramesh et al., 2003). The tumor cells staining positive for MDA-7 were analyzed under bright-field microscopy and quantitated by observers without knowledge of the treatment groups. At least five fields per specimen were analyzed. To determine the fate of tumor cells following treatment, sections of tumors were stained for apoptotic cell death with terminal deoxynucleotide transferase (Tdt) kit (Boehringer Mannheim, Indianapolis, Ind.) and counterstained with methylene blue or methyl green as described previously (Saeki et al., 2002; Ramesh et al., 2001). In all staining procedures, appropriate negative controls were included. For CD-31 staining, tissues were stained with anti-CD31 antibody as described (Saeki et al., 2002; Ramesh et al., 2003).

8. Tumor Characteristics after Treatment

To determine the therapeutic effects of the mda-7 gene, tumors were harvested from mice after the last treatment and subjected to hispathologic evaluation. Analysis was done by a pathologist without knowledge of the treatment groups.

9. Effect of DOTAP:Chol-mda7 Complex on Experimental Lung Metastasis

To test the effect of DOTAP:Chol-mda-7 complex on lung metastases, female nude mice were injected via tail vein with 10⁶ A549 tumor cells suspended in 100 μl of sterile PBS. Six days later, the mice were divided into three groups and treated as follows: no treatment (group 1), DOTAP:Chol-CAT complex (group 2), and DOTAP:Chol-mda-7 complex (group 3). There were eight mice in each group. All treatments comprised 50 μg liposome-DNA complex and were administered daily via tail vein using a 27-gauge needle for a total of six doses. Three weeks following the last dose, animals were euthanized by CO₂ inhalation. The lungs of each mouse were injected intratracheally with India ink and fixed in Feketes solution (Ramesh et al., 2003). The therapeutic effects of systemic mda-7 gene treatment were determined by counting the number of metastatic tumors in each lung under a dissecting microscope, by an observer without knowledge of the treatment groups. The data were analyzed, and differences among groups were interpreted as statistically significant if the P value was<0.05 by the Mann-Whitney rank-sum test.

As a syngeneic lung tumor model, C3H mice were injected with murine UV2237m fibrosarcoma cells (1×10⁶ ) and divided into three groups (n=7/group). Six days after injection, animals were treated as follows: no treatment, DOTAP:Chol-CAT complex, or DOTAP:Chol-mda-7complex. Treatment schedule and analyses of therapeutic effect were the same as already described for the A549 models. Experiments were performed two times for statistical significance.

Results

1. In Vitro Transfection of Tumor Cells with DOTAP:Chol-mda-7 Complex

The ability of DOTAP:Chol liposomes to deliver plasmid DNA into human (A549) and mouse (UV2237m) tumor cells by using expression plasmids encoding the human MDA-7/IL-24 protein was investigated. Transfection with DOTAP:Chol liposomes complexed with mda-7 plasmid DNA resulted in expression of exogenous MDA-7 protein in both A549 and UV2237m tumor cells at 24 and 48 h. MDA-7 expression was not observed in PBS treated control cells. Analysis of tissue culture supernatant from DOTAP:Chol-mda-7 transfected A549 and UV2237m cells showed secreted MDA-7 protein at 48 h but not at 24 h. Detection of secreted MDA-7 protein at 48 h is unlike that observed in Ad-mda7 treated cells where secreted MDA-7 protein is detectable at 24 h (Mhashilkar et al., 2001). This suggests that the transgenic MDA-7 expression achieved using DOTAP:Chol. liposome is less than that obtained with Ad-mda7. Secreted MDA-7 protein was not observed in PBS treated cells. Thus DOTAP:Chol liposomes could effectively deliver mda-7 DNA to tumor cells resulting in intracellular and secreted transgenic MDA-7 production albeit less than Ad-mda7.

2. MDA-7 Inhibits Subcutaneous Tumor Growth

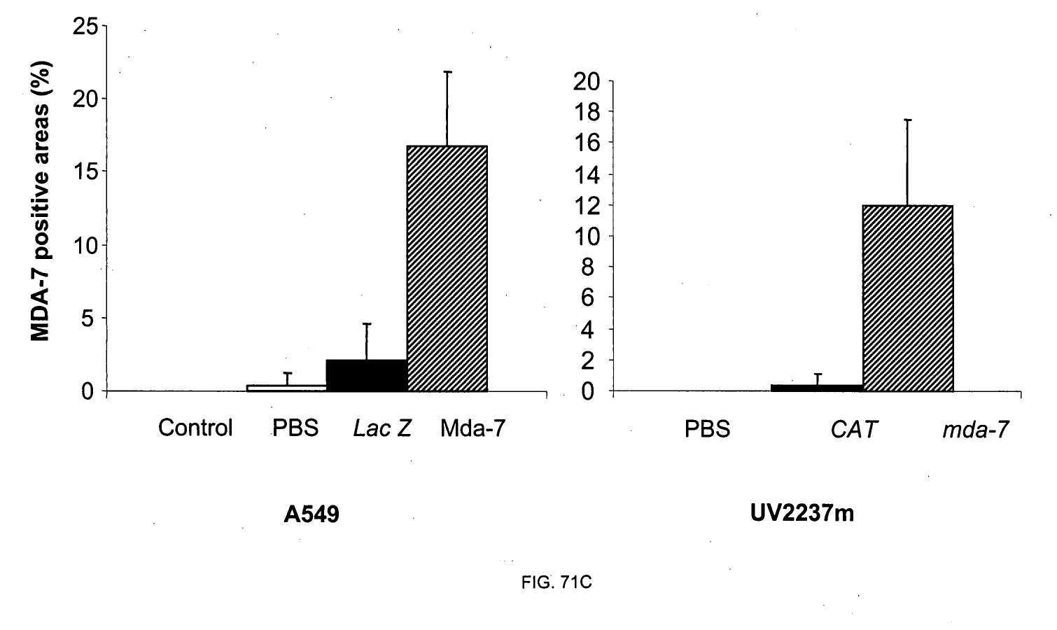

The ability of the DOTAP:Chol-mda-7 complex to suppress the growth of A549 human lung subcutaneous tumors was evaluated in nu/nu mice. Treatment of tumor-bearing mice with the DOTAP:Chol-mda-7 complex via the intratumoral route significantly inhibited tumor growth (P=0.001) as compared with tumor growth in animals that were untreated, treated with PBS, or treated with DOTAP:Chol-LacZ complex (FIG. 71A). Histopathological analysis of the tumors revealed no significant changes in the tumor infiltrating cells among the various treatment groups.

The therapeutic effects of the mda-7 gene on subcutaneous murine tumors in C3H mice were next evaluated. Mice bearing UV223M tumors were divided into three groups: one receiving no treatment, a second receiving treatment with DOTAP:Chol-CAT complex, and a third receiving treatment with the DOTAP:Chol-mda-7 complex. Growth of UV2237m tumors was significantly inhibited (P=0.01; FIG. 71B) in mice treated with intratumoral administration of the DOTAP:Chol-mda-7 complex when compared with tumor growth in the two control groups.

To demonstrate that the observed tumor-suppressive effects was due to mda-7 gene expression, subcutaneous A549 and UV2237m tumors obtained at 48 hours after injection were subjected to immunohistochemical analysis for MDA-7 protein expression. MDA-7 protein expression was seen in 18% and 13% of A549 and UV223m tumors respectively that were treated with the DOTAP:Chol-mda-7 complex (P=0.001; FIG. 71C), a significantly higher number than in the animals that were not treated, treated with PBS, treated with DOTAP:Chol LacZ or treated with DOTAP:Chol-CAT complex. Some level of non-specific staining was observed in A549 tumors that were treated with DOTAP:Chol-CAT complex. Analysis of the pattern of MDA-7 expression revealed intense intracellular staining in addition to a more diffuse staining pattern that appeared to be extracellular. This pattern of staining was observed in both human tumor xenografts and murine syngeneic tumors.

3. Apoptotic Cell Death in Lung Tumors treated with DOTAP:Chol-mda-7 Complex

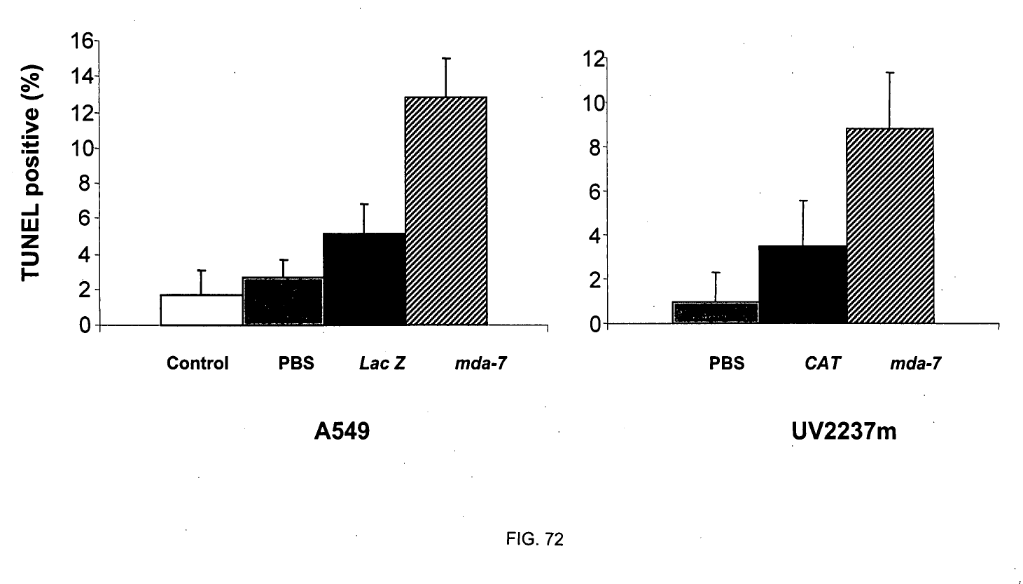

To determine the fate of tumor cells after treatment with the DOTAP:Chol-mda-7 complex, subcutaneous tumors (A549, UV2237m) from nu/nu mice and C3H mice were analyzed for apoptotic cell death as previously described Saeki et al., 2002). A significant (P=0.001) level of TUNEL positive cells (13% A549, and 9% UV2237m) indicative of apoptotic cell death was observed in tumors treated with DOTAP:Chol-mda-7 compared to tumors from control animals that were untreated, treated with PBS, treated with DOTAP:Chol-CAT or treated with DOTAP:Chol-LacZ (FIG. 72).

4. Reduced CD31-Positive Staining in Lung Tumors Treated with DOTAP:Chol-mda-7 Complex

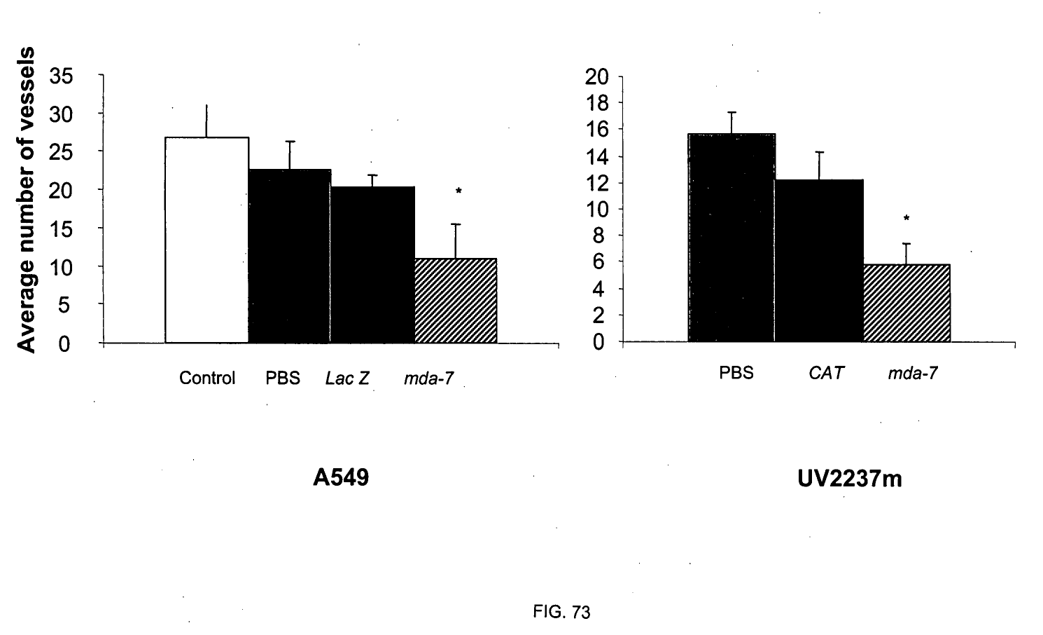

To determine the effect of mda-7 treatment on tumor vascularization, tumor tissues were subjected to CD31 staining as previously described (Saeki et al., 2002; Ramesh et al., 2003). Levels of CD31-positive staining was significantly (P=0.01) reduced in DOTAP:Chol-mda7 treated A549 (10%) and UV2237m (5.8%) tumor tissues compared to tumor tissues obtained from untreated, PBS-treated, DOTAP:Chol-LacZ complex treated, and DOTAP:Chol-CAT-treated mice (FIG. 73). Reduced CD31 staining is indicative of reduced vascularization.

5. MDA-7 Inhibits Experimental Lung Metastases

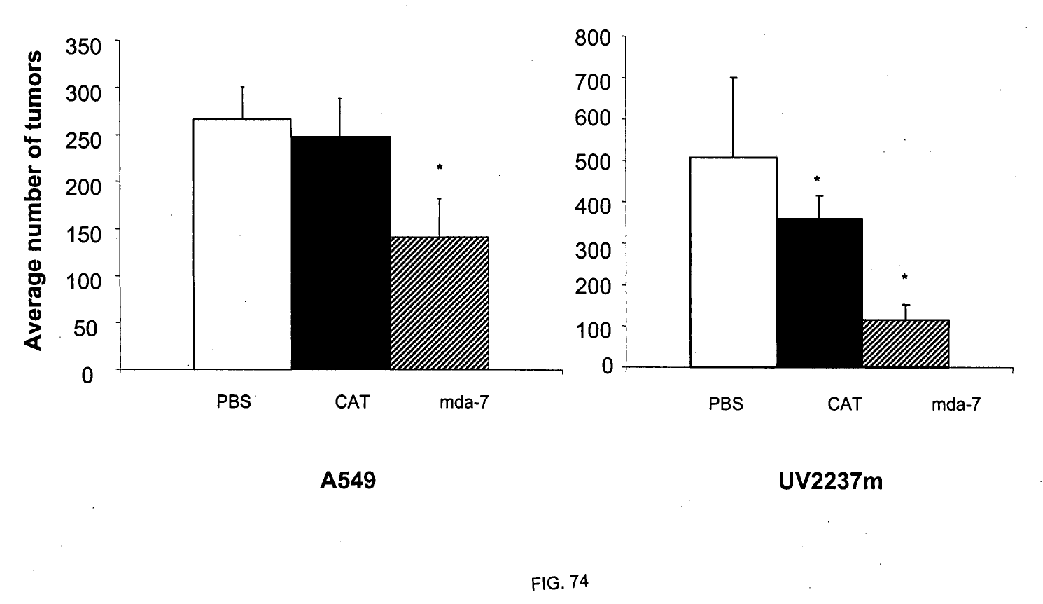

The activity of DOTAP:Chol-mda-7 complex was next investigated in an experimental lung metastases model using human A549 lung cancer cells or mouse UV227m cells. Intravenous delivery of tumor cells results in rapid tumor seeding of lungs, and animals succumb to overwhelming lung tumor burden after 30 days. Systemic treatment of A549 and UV2237m lung tumor-bearing nude or C3H mice with DOTAP:Chol-mda-7 complex resulted in a significantly (P<0.05) lower number of lung metastases than treatment with PBS or DOTAP:Chol-CAT complex (FIG. 74). In UV2237m mice, treatment with DOTAP:Chol-CAT complex resulted in a significant reduction in the number of tumor nodules when compared to those treated with PBS suggesting some non-specific antitumor activity (FIG. 74). Furthermore, the treatment was well tolerated with no treatment related toxicity observed as evidenced by lack of morbidity and mortality.

### Example 34

### Combination Therapy of AD-MDA7 and Trastuzumab Increases Cell Death in HER-2/NEU-Overexpressing Breast Cancer Cells

Materials and Methods

1. Cell Lines

SKBr3 and MCF-7 breast cancer cells were obtained from American Type Culture Collection. MCF-7-Her-18 cells were a gift from Dr. Mien-Chie Hung. The cells were maintained in high glucose DMEM media supplemented with 10% fetal bovine serum, 10-mmol/L glutamine, 100-U/ml penicillin, and 100 μg/ml streptomycin (GEBCO Invitrogen Corporation, Grand island, N.Y.) at 37° C. and 5% CO₂.

2. Adenovirus Transduction

The recombinant adenovirus vectors carrying the mda-7 gene (Ad-mda7) and the luciferase reporter gene (Ad-Luc) were obtained from Introgen Therapeutics (Introgen Therapeutics, Houston, Tex.). 6×10⁵ tumor cells were transduced with Ad-mda7 and Ad-Luc at an MOI of 2500 viral particles (vp) per cell. The dose of vector was selected to ensure >70% transduction efficiency. Herceptin was introduced into the cultures at a dose of 5 μg/ml.

3. Western Blot

Cells were lysed and protein concentration determined using the BioRad Assay (Bio Rad laboratories, Hercules, Calif.). Lysates were analyzed by western blot analysis using a 10% SDS gel. Lanes were loaded with 30-50 μg of protein and electrophoresed for 2 hrs at 90 V. Gels were transferred to a nitrocellulose membrane that was blocked with 1% dry milk and incubated with primary antibodies (β-catenin, Akt and p-Akt; Santa Cruz Biotechnology, Inc., Santa Cruz, Calif.) overnight at 4° C. Membranes were washed and incubated with secondary antibody for 1 hr at room temperature. Membranes were developed and protein signals detected using enhanced chemiluminescence (ECL)—western blotting detection reagents (Amersham Biosciences, Buckinghamshire, England). Membranes were incubated with antibody against β-actin (Santa Cruz) to assess equal protein loading. Results were subjected to densitometry.

4. Animals Studies

The nude mice were obtained from Charles River Laboratories (Wilmington, Mass.). Estrogen pellets were injected in the posterior cervical region at a dose of 0.5 mg. Two days later, MCF-7-Her-18 cells were injected into the thoracic mammary fat pad at a density of 5×10⁶ cells per mouse. When the tumors reached 100 mm³ in size, the mice were divided into six treatment groups: Phosphate buffered saline (PBS), Ad-Luc alone, Ad-mda7 alone, Herceptin alone, Ad-Luc+Herceptin, and Ad-mda7+Herceptin. The viral vectors were injected directly into the tumor at a dose of 2×10¹⁰ viral particles once per week for three weeks. Herceptin was delivered by intraperitoneal injection at a dose of 110 μg per animal, twice weekly for three weeks. The tumor size was measured and recorded twice a week until sacrifice.

5. Statistical Analysis

The data reported in the figures represent the means of three independent experiments with standard deviations. Differences in size of the tumors were analyzed for significance by the student t test. The densitometry of the Westerns was also analyzed for significance by the student t test.

Results

1. Ad-mda7 Treatment Inhibits Growth of Breast Cancer Cells

Breast cancer cell lines SKBr3 (Her2+) and MCF-7 (Her2-) were treated with Ad-mda7 or Ad-luc and cell viability was assessed. As shown in FIG. 1, Ad-mda7 inhibits growth of both Her2+ and Her2− breast cell lines, indicating that MDA-7 mediated killing is independent of Her2 status. This result was confirmed in additional Her2+ and Her2− breast tumor lines. To evaluate the effect of combining Herceptin with Ad-mda7, the same cell lines were treated with Herceptin alone, or in combination with Ad-mda7 or Ad-luc. Herceptin induced killing of Her2+ SKBr3 breast tumor cells, but not Her2− MCF-7 cells. When combined with low doses of Herceptin, the cytolytic activity of Ad-mda7 was greatly enhanced only in Her2+ cells, and results in supra-additive killing. In contrast, in Her2− cells, there was no significant difference in cytotoxicity provided by Herceptin. When Ad-luc was combined with Herceptin, the effects were comparable to Herceptin alone. Further studies on these and additional breast cell lines indicated that the enhanced cytolytic activity observed in FIG. 1 is due to increased apoptosis induction in Her2+ cells treated with the combination of Ad-mda7+Herceptin.

The mechanism of Herceptin synergy with Ad-mda7 was evaluated and also the combinatorial synergy in a xenograft model. Unfortunately, the Her2+ cell line discussed in the previous paragraph does not form tumors in nude mice and thus we modified model systems to take advantage of the MCF-7-Her-18 cell line. This cell line was derived from Her2− MCF-7 cells into which a Her-2/neu expression vector was stably transfected.

2. Ad-mda7 Decreases Levels of β-catenin, Akt and p-Akt

Ad-mda7 treatment of breast and lung cancer cell lines decreases expression of Akt and p-Akt². It has also been shown that β-catenin is negatively regulated by Ad-mda7. To investigate whether Herceptin in addition to Ad-mda7 will cause a greater decrease in the levels of β-catenin, Akt, and p-Akt, MCF-7-Her-18 cells were transduced with Ad-mda7 or Ad-luc individually or in combination with Herceptin. Decreased levels of Akt, p-Akt, and β-catenin were noted after being transfected with 2500 vp/cell Ad-mda7. There is a greater decrease in the steady-state levels of all three proteins when the cells are treated with the combination of Ad-mda7+Herceptin. Western blot analysis showed decreased expression of Akt in MCF-7-Her 18 cells after being transfected with Ad-mda7 and Herceptin. Decreased protein expression was observed with Ad-mda7 and Herceptin, in contrast to PBS. However, the combination of Herceptin+Ad-mda7 showed a more significant decrease in contrast to either Ad-mda-7 or Herceptin alone (p<0.05). The control adenoviral luciferase vector showed little or no decrease in protein levels of Akt. Likewise, the Western blot analysis of p-Akt showed similar results. The p-Akt protein level after treatment with Ad-mda-7 and Herceptin was significantly less than both PBS and Ad-luc. Compared to the Ad-mda7+Herceptin mediated inhibition of Akt, the combination of Herceptin+Ad-mda-7 showed an even greater reduction in p-Akt. Another Western blot analysis of β-catenin revealed that the combination therapy of Herceptin+Ad-mda-7 led to a greater decrease in expression of β-catenin in contrast to Ad-mda7 or Herceptin alone.

3. Ad-mda7+Herceptin Inhibits Tumor Growth in Nude Mice

Nude mice bearing subcutaneous tumors produced from MCF-7-Her-18 cells were injected intratumorally with Ad-mda7 or Ad-Luc when the tumors reached 100 mm³ and Herceptin was delivered systemically by intraperitoneal injection. The tumors that were treated with PBS or Ad-Luc continued to increase in size, while the tumors injected with Ad-mda7 alone or Herceptin+Ad-mda7 showed significant inhibition of growth compared to the controls. Note that treatment of all the groups of animals started when tumors reached approximately 100 mm³. The growth rate of PBS and Ad-luc treated tumors was comparable. However, it is clear from the kinetic analysis shown in FIG. 5 that the growth rate of tumors treated with Ad-mda7 or Herceptin slowed considerably. Tumors treated with Herceptin and Ad-Luc+Herceptin showed very similar growth patterns, confirming the lack of anti-tumor effect of Ad-Luc. Tumors treated with Ad-mda7 monotherapy exhibited somewhat greater growth inhibition than that attained with Herceptin monotherapy. However, the combination of Ad-mda7+Herceptin demonstrated enhanced activity and near-complete growth suppression for at least 15 days, after which tumors appear to grow slowly. A commonly used surrogate for growth rate is time to tumor doubling. For PBS and Ad-Luc controls, this would occur at day 5 and 11 respectively. Herceptin and Ad-Luc+Herceptin treated tumors doubled by day 16 and 14, respectively. The tumors treated with Ad-mda7 doubled in size by approximately day 21, whereas the combination of Ad-mda7+Herceptin resulted in tumor doubling times of greater than 28 days.

4. Increased Apoptosis in HER2/NEU Overexpressing Breast Cancer Cells by Combination Therapy of Ad-mda7 and Herceptin

The MCF-7 breast cancer cell line overexpressing HER-2/neu (MCF/HER2-18) was transduced with Ad-mda7, herceptin, control adenoviral luciferase vectors (Ad-Luc) and the combination of herceptin with Ad-mda7 and Ad-Luc, respectively. Cell viability was evaluated with a colorimetric (MTT) assay, and apoptosis with fluorescence-activated cell sorting analysis (FACS). Bcl-2 and PARP expression were evaluated by western blot.

The combination of Ad-mda7 and herceptin in MCF/HER2-18 cells resulted in a sharp decrease in growth when compared to herceptin or Ad-mda7 alone demonstrated by the MTT assay (P<0.01 ANOVA). In addition, FACS analysis showed significant apoptotic cell death with this combination therapy. Apoptosis was confirmed by PARP cleavage and decreased Bcl-2 expression on western blot analysis.

### Example 35

### MDA-7 Physically Associates with the Double Stranded RNA Activated Protein Kinase PKR

Materials and Methods

1. Cell lines and Reagents

A549 (wt p53) and H1299 (p53 null) human lung cancer cell lines were obtained from the American Type Culture Collection have been previously described (Pataer et al., 2002). PKR+/+ and PKR−/− cells obtained from Dr. Glen N Barber (University of Miami School of Medicine). PKR+/+ and PKR−/− cells were maintained in Dulbecco's modified Eagle's medium (DMEM) containing 10% fetal bovine serum, 10 mM glutamine, 100 units/ml penicillin, 100 μg/ml streptomycin (Life Technologies, Inc., Grand Island, N.Y.) in a 5% CO₂ atmosphere at 37° C. Cycloheximide (CHX) was obtained from Sigrna Chemical Co. (St. Louis, Mo.). Recombinant MDA-7 protein was obtained from Introgen Therapeutics (Houston, Tex.).

2. Adenovirus Production

Constructions of the Ad-mda7, Ad-bak, Ad-lacZ and Ad-Luc vectors have been previously reported (Pataer et al., 2002). The transduction efficiencies of adenoviral vectors in various cancer cell lines were determined by infecting cells with Ad-LacZ and then determining the titers needed to transduce at least 70% of the cells.

3. Flow Cytometry Analysis

The number of apoptotic cells was measured by propidium iodide staining and FACS analysis. Cells were harvested, pelleted by centrifugation and resuspended in phosphate-buffered saline containing 50 μg/ml propidium iodide, 0.1% Triton X-100, and 0.1% sodium citrate. Samples were stored at 4° C. for 1-3 hours and vortexed prior to FACS analysis (Becton-Dickenson FACScan, Mountain View, Calif.; FL-3 channel).

4. Real-Time PCR

RT-PCR was carried out using the Thermoscript RT-PCR system kit according to the instructions provided by the manufacturer (Life Technologies, Gaithersburg, Md., USA). Total RNA was isolated according to the protocol for TRIzol extraction (Life Technologies), and 1 μg of each sample was reverse-transcribed to complementary DNA (cDNA) by a reaction containing 2 mM deoxynucleotide mix, 100 mM DTT, 40 units of RNase inhibitor, 50 ng of random primer, and 15 units of Thermoscript reverse transcriptase. Real-time PCR was performed using the protocol reportered previously (Vorburger et al., 2002).

5. Western Blot Analysis

48 h after transfection, cell extracts were prepared and immunoblot assays performed as described (Pataer et al., 2002) using the antibodies indicated. The following antibodies were used: monoclonal antibody to p53 was purchased from PharMingen (San Diego, Calif.). Anti-PKR (K-17), anti-p-actin, anti-stat3 (F-2) and phospho-specific anti-stat3 (B-7) antibodies were from Santa Cruz Biotechnology (Santa Cruz, Calif.). The polyclonal or monoclonal antibody to MDA-7 was obtained from Introgen Therapeutics Inc (Houston, Tex.).

6. Co-Immunoprecipitation Analysis

Cells were treated with Ad-mda7 or Ad-Luc for 48 hrs, and then lysed in RIPA buffer (1× PBS, 1% Nonidet P-40, 0.5% Sodium deoxycholate, 0.1% SDS). 500 μl cell lysate were incubated with 20 μl protein A/G Agarose at 4° C. for 30 minutes. Primary antibody was added to pre-cleared cell extracts and incubated with gentle rocking overnight at 4° C. Protein A/G agarose was added to the mix and incubated for 4 hrs. Pellet beads by centrifugation at 2500 rpm for 5 min at 4° C. Pellet washed 4 times with 1 ml of RIPA buffer. After the last wash, 50 μl of 1× SDS-PAGE sample buffer added to beads. The mixture was vortexed, then boiled for 5 min. Mixture was centrifuged at 2500 rpm for 1 min before the supernatant was loaded on gel.

7. Cellular Localization Studies

A549 and H1299 cells (5×10⁴ cells/well) were grown on chamber slides until 70% confluence and then transfected with Ad-luc, Ad-mda7, or PBS. Forty-eight hours later, cells were washed with PBS and fixed with freshly made 4% paraformaldehyde/PBS for 15 minutes. Cells were then permeabilized for 20 min at 4° C. with 0.2% Triton X-100 and blocked one hour with 1% normal goat serum. Rabbit polyclonal anti-PKR (K-17) and mouse monoclonal anti-MDA-7 were incubated overnight at 4° C. and developed with rhodamine or fluorescein-5-isothiocyanate secondary antibodies for 30 min at 37° C. Cells were then visualized under the fluorescence microscope and further evaluated by confocal microscopy.

8. Statistical Analysis

The data reported represent the mean of three or independent experiments and the bars show the standard deviation (SD).

Results

1. Ad-mda7 Induces Apoptosis and PKR Induction on Human Lung Cancer Cells

Flow cytometric analysis of apoptosis was performed on the A549 (wt p53) and H1299 (null p53) human lung cancer cells at 48 hrs following infection with Ad-mda7, Ad-luc and PBS. Compared with Ad-luc and PBS, Ad-mda7 resulted in high levels of apoptosis in both A549 and H1299 human lung cancer cells. To determine if Ad-mda7 could induce PKR induction in vitro, Ad-mda7 treated A549 cells were evaluated by Western blot analysis. Ad-mda7 treatment resulted in a dose dependent induction of PKR on A549 cells. Even low levels of Ad-mda7 (500 vp/cell; 25 pfu/cell) caused PKR induction, however PKR levels continued to increase with Ad-mda7 dose. Similar PKR induction was seen in H1299 cells.

2. Intracellular MDA-7 Protein Plays an Important Role in Ad-mda7-Induced Cell Death

Recent studies have shown that Ad-mda7 transduced cells secrete a soluble form of MDA-7 protein that may trigger a bystander killing effect on neighboring non-transduced cancer cells (Mhashilkar et al., 2001). Ad-mda7 transduced cell lysates show an immunoreactive doublet of MDA-7 bands at approximately 30 and 23 kD, whereas the supernatant sample showed a novel protein band at approximately 40 kD (Mhashilkar et al., 2001). Therefore the anti-tumor effects of secreted MDA-7 on A549 (wt p53) and H1299 (null p53) human lung cancer cells was evaluated. MDA-7 secreted protein did not induce cell growth inhibition or cell death on A549 or H1299 lung cancer cell lines. In contrast, Ad-mda7 treatment resulted in cell growth inhibition and concomitant apoptosis on both lung cancer cell lines. To determine if MDA-7 secreted protein could induce PKR induction in vitro, MDA-7 protein treated cells were evaluated by Western blot analysis. MDA-7 secreted protein did not induce PKR in A549 cells. In contrast, Ad-mda7 treated A549 cells induced robust expression PKR and phosphorylation of Stat3.

3. MDA-7 Physically Interacts with the dsRNA-Dependent Protein Kinase PKR

Immunofluorescence staining and confocal laser-scanning microscopy were used to examine the subcellular localization of both MDA-7 and PKR proteins in A549 and H1299 cells. Previous studies have shown that PKR is localized in the cytosol and nucleus (Taylor et al., 1999). Immunofluorescence studies showed MDA-7 protein distribution is predominantly cytoplasmic. MDA-7 induced endogenous PKR protein, and also colocalized with PKR in both NSCLC cell lines.

Several dsRNA-binding proteins, including cellular proteins such as NF90 (Nuclear factor 90) and PACT (protein activator of PKR), and viral proteins TRBP (TAR RNA binding protein), and vaccinia virus E3L protein, interact with PKR (Yin et al., 2003). Whether MDA-7 protein can physically interact with PKR was tested in A549 and H1299 cells. PKR proteins were immunoprecipitated from PBS, Ad-luc and Ad-mda7 treated lysates using anti-PKR antibody and the samples were analyzed by immunoblotting with the antibodies against MDA-7. Anti-PKR antibodies did not immunoprecipitate MDA-7 protein in the PBS or Ad-luc treated cells. In contrast, in Ad-mda7 transduced cells, MDA-7 could be immunoprecipitated with antibodies against PKR. One major MDA-7-immunoreactive band was recognized by MDA-7 antibody. To confirm the protein-protein interaction, the reciprocal experiment was performed When PBS, Ad-luc and Ad-mda7 treated cell lysate was immunoprecipitated with an antibody against MDA-7, PKR could be detected only in Ad-mda7 treated cell lystates. Coimmunoprecipitation studies showed that MDA-7, like TRBP, PACT, NF90 and E3L, can physically interact with the dsRNA-dependent protein kinase PKR.

4. Role of the PKR Protein in the Ad-mda7-Induced Cell Death

To evaluate the contribution of PKR induction to Ad-mda7-induced cell death, isogenic PKR deficient cell lines (PKR+/+ and PKR−/−) were utilized. Despite adequate transduction and expression of MDA-7 protein in both PKR null (−/−) and wild-type PKR cell lines, only the wild-type PKR (+/+) cell line underwent cell death following Ad-mda7 treatment suggesting that Ad-mda7 induced cell killing was dependent on PKR. As expected, Ad-mda7 induced PKR induction on PKR+/+cell lines. Unlike Ad-mda7, Ad-Bak mediated cell killing was not dependent on PKR genomic status with cell death occurring in both PKR null and wild type PKR cell lines. Immunoprecipitation assays were then performed to detect phosphorylated forms of MDA-7 and PKR by using PKR+/+ and PKR−/− cell lines. The phosphorylated form of MDA-7 and PKR could only be detected in PKR+/+ cells, but not on PKR−/− cells. Both PKR and MDA-7 proteins were phosphorylated at threonine and serine sites in Ad-mda7 transduced cells. No tyrosine phosphorylation of MDA-7 or PKR proteins could be detected in Ad-mda7 transduced cells.

### Example 36

### Recombinant Human MDA-7/IL-24 Protein Suppresses the Expression of Proteins Involved in DNA Repair and Inhibition of Apoptosis in Endothelial Cells

Recombinant MDA-7/IL-24 protein suppresses the growth of and radiosensitizes human endothelial cells in vitro.

To assess the molecular effects of MDA-7/IL-24 protein that might mediate a restoration of apoptosis and radiosensitization, human umbilical vein endothelial cells (HUVECs) were treated with 10 ng/ml of MDA-7/IL-24 protein for 12 hrs and the cells analyzed by Western blot. Activation of pSTAT3 and pATF but a reduction in pAKT and p-p38 were observed. Protein levels of Bcl-xL and survivin, proteins involved in apoptosis, were also suppressed. Also observed were downregulation of Ku70 and XRCC4, proteins involved in repair of radiation-induced DNA damage, and suppression of IL-8, a protein involved in angiogenesis, following treatment with MDA-7/IL-24. The levels of many other proteins, i.e,. Bax, p21, p27, and p53, remained constant in these treatments.

To test whether the changes in protein expression following MDA-7/IL-24 treatment in HUVECs are due to the suppression of transcription of the genes that code for these proteins, HUVECs treated with MDA-7/IL-24 were analyzed by RPA analysis. The results suggested that MDA-7/IL-24 treatment of HUVECs affected the transcription of several genes whose protein products are critically involved in repair of radiation-induced DNA damage. Therefore, MDA-7/IL-24 protein may initiate a signaling cascade that leads to changes in gene transcription. These in turn lead to a suppressed expression of proteins involved in DNA repair. Even though the individual changes in these proteins are relatively small, several critical repair proteins are suppressed and together they may negatively affect DNA repair capacity leading to radiosensitization.

### Example 37

### Involvement of Perk in Human MDA-7/IL-24 Mediated Inhibition of Murine Tumor Cell Growth

The mechanism of MDA-7 mediated tumor-suppressive activity varies among tumor cell types, with PKR, p38MAPK, and pJNK identified as the major pathways for inducing apoptosis.

In the present study it was investigated whether MDA-7/IL-24 could exert a similar inhibitory activity on murine tumor cells. Treatment of murine fibrosarcoma (UV2237m, and MCA-16) with an adenoviral vector carrying the mda-7/IL-24 gene (Ad-mda7; 10,000 vp/cell) resulted in exogenous expression of MDA-7/IL-24 protein and in a significant (P=0.001) growth inhibition at G2/M phase when compared to cells that were treated with PBS or treated with Ad-luc (vector control). In contrast, no growth inhibitory effects were observed in normal murine fibroblast (10T1/2) cells when treated with Ad-mda7. Studies investigating the mechanisms of growth inhibition and apoptosis revealed that in murine tumor cells unlike in human cells activation of PKR, p38MAPK, and pJNK was not observed. In contrast, activation of PERK and its downstream targets, elF2-α, and caspase-12, was observed that subsequently resulted in caspase-9, caspase-3 activation, and PARP cleavage.

Additionally, treatment of UV2237m tumors established subcutaneously in sygeneic C3H/Ncr mice with the mda-7/IL-24 gene encapsulated in a DOTAP:cholesterol (DOTAP:Chol) liposomal vector resulted in significant growth inhibition compared to tumors that were treated with PBS or treated with a control plasmid encapsulated in DOTAP:Chol. liposome. Animals were treated by intratumoral injection three-times a week (50 μg DNA/dose) for 3-weeks. Additionally, complete regression was observed in 40% of the animals treated with mda-7/IL-24. These results show that human MDA-7/IL-24 can inhibit growth of murine tumors both in vitro and in vivo and via the PERK pathway.

### Example 38

### Adenoviral-Mediated MDA-7 Expression Suppresses DNA Repari Capacity and Radiosensitizes Non-Small Cell Lung Cancer Cells

Materials and methods

1. Cell Culture

The human non-small cell lung cancer (NSCLC) cell line, A549, normal human lung fibroblast cell line (NHLF), CCD-16, and human glioma cell lines, MO59J and MO59K, were obtained from the American Type Culture Collection (ATCC). All cell lines were maintained as specified by ATCC.

2. Adenoviral Vectors and Gene Delivery

Ad-mda7, Ad-Luc, and Ad-β-gal were all obtained from Introgen Therapeutics, Inc. (Houston, Tex., USA). Ad-Luc was used as a control vector, and Ad-β-gal was used as a reporter vector. The vectors were tested for the presence and determined to be free of replication-competent adenovirus and mycoplasma. Forty-eight hours after plating, cells were incubated for 1 h with a purified vector in 1 ml of medium without serum. After incubation, fresh medium supplemented with 10% fetal bovine serum was added to each flask. Serum-free medium was used in a protocol that was identical except for the inclusion of a vector for mock transfection. Cell numbers for multiplicity of infection calculations were determined by counting the cells in an untreated flask.

3. Nuclear Protein Extract

Cells were rinsed twice with ice cold phosphate-buffered saline, harvested with a cell scraper, and centrifuged at 500×g for 10 min at 4° C. The cell pellet was then resuspended in 400 μl of cold lysis buffer (10.0 mM HEPES, pH 7.9, 10.0 mM KCl, 0.1 mM ethylenediaminetetraacetic acid, 0.1 mM egtazic acid, 1.0 mM dithiothreitol, 2.0 μg/ml leupeptin, 2.0 μg/ml aprotinin, 0.5 mM phenylmethylsulfonyl fluoride) and incubated on ice for 10 min; afterwards, 12.5 μl of 10% NP-40 was added, and the mixture was vortexed for 5 s. Next, the lysate was centrifuged at 500 x g for 5 min at 4° C. and the supernatant was removed and stored as a cytosolic extract. The pellet was resuspended in 30 μl of extraction buffer (20.0 mM HEPES, pH 7.9, 400.0 mM NaCl, 1.0 mM ethylenediaminetetraacetic, acid, 1.0 mM egtazic acid, 1.0 mM dithiothreitol, 2.0 μg/ml leupeptin, 2.0 μg/ml aprotinin, 0.5 mM phenylmethylsulfonyl fluoride), mixed thoroughly, and incubated on ice for 30 min. The pellet was then vortexed every 10 min. After 30 min, the extract was centrifuged for 10 min at maximum speed in a microcentrifuge. The supernatant was designated as a nuclear extract, aliquoted, and stored at −70° C. The amount of nuclear protein obtained was determined using the Bio-Rad Protein Assay (Bio-Rad Laboratories, Richmond, Calif., USA) in accordance with the manufacture's instructions and using bovine serum albumin as a protein standard.

4. Antibodies

Rabbit polyclonal antibodies against DNA-PKcs and XRCC4 were purchased from GeneTex (San Antonio, Tex., USA). Goat polyclonal antibodies against Ku70 and β-actin were purchased from Santa Cruz Biotechnology (Santa Cruz, Calif., USA). A mouse monoclonal antibody against Ku86, clone Ku15, was purchased from Sigma-Aldrich (St. Louis, Mo., USA). A rabbit polyclonal antibody against DNA ligase IV was purchased from Serotec (Raleigh, N.C., USA).

5. Western Blot Analysis

Equal amounts of nuclear protein were separated by via sodium dodecyl sulfate-polyacrylamide gel electrophoresis (8%-12% polyacrylamide with a 4% stacking gel) and then transferred to Ihmobilon-P membranes (Millipore, Bedford, Mass., USA). Nonfat dry milk, 5%, in TBS-T (TBS with 0.05% Tween-20) was used as a blocking solution, while TBS-T alone was used as a washing buffer. The membranes were treated with primary antibodies in a blocking solution at 4° C. overnight and with secondary antibodies in a blocking solution at room temperature for 1 h. The results were visualized with enhanced chemiluminescence (Amersham, Arlington Heights, Ill., USA) according to the manufacturer's protocol.

6. RNase Protection Assay

Cells were transfected with either Ad-mda7 or Ad-Luc. After 48 h of incubation, RNA was extracted using TRizol Reagent (Invitrogen, Carlsbad, Calif., USA). Probes were labeled with ³²P-UTP and hybridized with the isolated RNA. The probe set used was hDSBR-2 (Pharmingen, San Diego, Calif., USA). The housekeeping genes L32 and GAPDH, were used for sample normalization. After RNase digestion, the protected fragments were separated via polyacrylamide gel electrophoresis. All of the steps were performed according to the manufacturer's recommendations.

7. DNA DSB Repair Assay

Assay of DNA DSB repair activity following exposure to ionizing radiation was performed as described previously (Kurimasa et al., 1999; Story et al., 1994). Briefly, 48 h after vector treatment, cells were irradiated on ice using a ¹¹⁷Cs unit (3.5 Gy/min), receiving a cumulative dose of 40 Gy. Immediately following irradiation, the cold medium was replaced with medium that had been warmed to 37° C., and the cells were placed in a 37° C. tissue culture incubator for the appropriate time (0 min, 15 min, 30 min, 1 h, 2 h, 4 h or 24 h) to allow the DSBs to be repaired. Cells were then trypsinized on ice, washed, embedded in agarose plugs, lysed, and digested with proteinase K. The DNA was separated using contour-clamped homogeneous electric field PFGE (CHEF-DR III system, Bio-Rad Laboratories) at 1.5 V/cm for 20 h at 25° C. in 0.5× TBE buffer. The gel was transferred to a nylon membrane for 3 days at room temperature. The membrane was then hybridized with a ³²P-labeled human Alu⁺ probe for 18 h at 45° C. The fraction of activity released from the plug into the lane was determined using a storage phosphorimaging system with the ImageQuant software program (Molecular Dynamics, Sunnyvale, Calif., USA).

8. HCR

A modified version of the HCR assay was used to assess DNA repair capacity (Eady et al., 1992; McDonald et al., 1996; Rainbow, 1974; Rainbow and Mak, 1972). Briefly, cells were seeded in a six-well plate and pretreated with Ad-mda7, Ad-Luc, or mock as described above. Cells were then transfected with non-irradiated or irradiated Ad-β-gal 48 h after pretreatment. Ad-β-gal was irradiated using a high-dose-rate ¹³⁷Cs unit (34.3 Gy/min) at room temperature at a dilution of 1/100 in medium supplemented with 1% fetal bovine serum. Twenty-four hours after transfection with Ad-β-gal, cells were fixed with 2.00% formaldehyde and 0.05% glutaraldehyde in phosphate-buffered saline, and stained with X-Gal (5-bromo-4-chloro-3-indolyl-β-D-galactopyranoside). The capacity of the cellular repair systems was assayed by counting the positively stained cells under a microscope. The results using irradiated Ad-β-gal were compared with those in cells treated with non-irradiated Ad-β-gal. The experiments were done in triplicate.

Results

1. Ad-mda7 Suppresses the Expression of Proteins Involved in DNA Repair in NSCLC Cells but not in Normal Fibroblasts

Ad-mda7 radiosensitize NSCLC cells but not normal fibroblasts and this correlates with the ability of this vector to induce activation of the c-Jun transcription factor in the NSCLC cells but not in fibroblasts. Thus, it was of interest to test for changes in the expression of genes that are important in governing radiosensitivity such as those in the pathways of DNA repair.

In mammalian cells, non-homologous end joining (NHEJ) is the predominant pathway for repairing DSBs induced by exposure to ionizing radiation. In order to determine whether Ad-mda7 affects the expression level of the proteins involved in the NHEJ pathway, western blot analysis was performed on nuclear protein extracts. The expression levels of Ku70, XRCC4 and DNA ligase IV protein were reduced by Ad-mda7 treatment in A549 cells. On the other hand, no reduction in the expression of any of these proteins was observed in CCD16 cells when treated in an identical manner. The effects in the NSCLC cells appeared to be specific to Ad-mda7 as a control vector, Ad-Luc, did not affect the expression of those proteins in either cell line.

2. Ad-mda7 Reduces the Expression of DNA Repair Gene mRNA in A549 Cells

To determine whether the suppressed protein levels reflected a reduced transcription of the genes that code for these proteins, RNAse protection assays were conducted for the genes of interest. The results showed that mRNA levels for several DNA repair genes were lower in A549 cells treated with Ad-mda7 compared to untreated controls. For some genes, the control vector, Ad-Luc, also affected transcription. However, transcription for several of the genes of interest—KU70, Lig4, XRCC4, and DNA-PK—was affected to a greater degree by Ad-mda7 than by Ad-Luc.

3. DNA DSB Repair after Irradiation is Suppressed by Ad-mda7 Transfection

Although the suppressed expression of genes whose protein products are crucial for repairing radiation-induced DSBs as illustrated above is consistent with the radiosensitizing effects of Ad-mda7, it was necessary to confirm that repair of these lesions was actually compromised following this treatment. Thus, the total DSB induction and rejoining following irradiation was examined using pulsed field gel electrophoresis (PFGE) to determine whether Ad-mda7 transfection affected DSB rejoining kinetics in A549 cells. Resaults from this analysis showed that a substantial amount of fragmented DNA migrated out of the plugs and into the lanes following delivery of a dose of 40 Gy when examined at time point 0. This effect was similar for Ad-mda7-treated cells and controls, indicating that Ad-mda7 treatment does not enhance the induction of DSBs by radiation. However, as a function of time, the proportion of DNA in the lane was reduced as the DSBs were rejoined; this appeared to be a slower process in the Ad-mda7-treated cells than in the controls. A number of gels were quantified, and the average values were plotted to evaluate the kinetics of DSB repair following the different treatments. Although the overall kinetics of repair were very similar in the different groups, it appeared that pretreatment with Ad-mda7 resulted in a higher level of residual unrejoined lesions at 24 h compared with untreated controls or cells pretreated with Ad-Luc.

4. Host Cell Rreactivation (HCR) is Suppressed by Ad-mda7 in A549 Cells but not in CCD16 Cells

Strictly speaking, the PFGE assay for DSB repair, when used as described above, actually measures the total DSB rejoining, which includes the inappropriate rejoining of broken DNA fragments such as those that lead to chromosomal translocations. Furthermore, since it does not detect misrepaired lesions that result in deletions, it overestimates the amount of repair that contributes to cell survival. Thus, to investigate the fidelity and total capacity for DNA repair, the HCR assay was used as a further test of Ad-mda7's ability to suppress the DNA repair pathways that govern radiosensitivity. Gamma-irradiated adenoviral-mediated β-galactosidase (Ad-β-gal) was used as the reporter vector, with β-galactosidase activity used as the readout. The extent of repair was assessed by measuring the reporter gene expression in A549 cells and CCD16 cells that were pretreated with Ad-mda7 or Ad-Luc or by mock transfection. The results indicated that HCR was suppressed in A549 pretreated with Ad-mda7 when compared with control cells pretreated either with Ad-Luc or via mock transfection. In contrast, significant suppression of HCR was not observed in CCD16 cells following pretreatment with Ad-mda7. To confirm that the HCR assay was sufficiently sensitive to detect known defects in DNA repair, the assay was tested on the human glioblastoma cell lines MO59K and MO59J, which are proficient and deficient in DNA-dependent protein kinase (DNA-PK), respectively. HCR was significantly reduced in MO59J cells when compared with that in MO59K cells by factors approaching 50%. DNA-PK, which comprises the Ku70/Ku80 heterodimer and the catalytic subunit DNA-PKcs, is required for the NHEJ pathway of DSB repair. Cells lacking DNA-PK activity because of defects in DNA-PK components, such as M059J cells, have shown hypersensitivity to ionizing radiation and compromised DSB rejoining kinetics (Allalunis-Tumer et al., 1993; Lees-Miller et al., 1995).

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Pat. No. 4,196,265
U.S. Pat. No. 4,367,110
U.S. Pat. No. 4,452,901
U.S. Pat. No. 4,554,101
U.S. Pat. No. 4,797,368
U.S. Pat. No. 4,870,287
U.S. Pat. No. 5,028,592
U.S. Pat. No. 5,139,941
U.S. Pat. No. 5,187,260
U.S. Pat. No. 5,221,605
U.S. Pat. No. 5,238,808
U.S. Pat. No. 5,310,687
U.S. Pat. No. 5,399,363
U.S. Pat. No. 5,466,468
U.S. Pat. No. 5,543,158
U.S. Pat. No. 5,552,293
U.S. Pat. No. 5,633,016
U.S. Pat. No. 5,641,515
U.S. Pat. No. 5,739,169
U.S. Pat. No. 5,760,395
U.S. Pat. No. 5,798,339
U.S. Pat. No. 5,801,005
U.S. Pat. No. 5,824,311
U.S. Pat. No. 5,824,348
U.S. Pat. No. 5,830,880
U.S. Pat. No. 5,846,225
U.S. Pat. No. 5,846,233
U.S. Pat. No. 5,846,945
U.S. Pat. No. 6,132,980
U.S. Pat. No. 6,177,074
U.S. Pat. No. 6,204,022
U.S. Pat. No. 6,207,145
U.S. Pat. No. 6,250,469
U.S. Pat. No. 6,326,466
U.S. Pat. No. 6,331,525
U.S. Pat. No. 6,350,589
U.S. Pat. No. 6,372,218
U.S. Pat. No. 6,379,701
U.S. patent application Ser. No. 09/615,154
U.S. patent application Ser. No. 10/391,068
U.S. patent application Ser. No. 60/404,932
U.S. patent application Ser. No. 60/370,335
U.S. patent application Ser. No. 60/361,755
U.S. patent application Ser. No. 10/017,472
Adams et al., Biochemistry, 37:12927-12932, 1998.
Ahnen et al., Eur. J. Surg. Suppl., 582:111-114, 1998.
Aksentijevich et al., Hum Gene Ther 7(9):1111-22, 1996.
Albert et al., Nature, 392(6671):86-89, 1998.
Allalunis-Tumer et al, Radiat. Res., 134, 349-354, 1993.
Allen et al., Nature 287:408-411, 1980.
Angel et al., Cell 49(6):729-39, 1987.
Angel et al., Mol Cell Biol 7(6):2256-66, 1987.
Angiolillo et al., Ann. NY Acad. Sci., 795:158-167, 1996.
Angiolillo et al., J. Exp. Med., 182(1):155-162, 1995.
Aringer et al., Life Sci 64:2173-86, 1999.
Ashkenazi et al., Science, 281:1305-1308, 1998.
Atchinson et al., Cell 46(2):253-62, 1986.
Ausubel et al., In:Current Protocols in Molecular Biology, Green Pub. Assoc., Inc., and John Wiley and Sons, Inc., NY, (I):2.10.3, 1989.
Baichwal and Sugden, In: Gene Transfer, Kucherlapati (ed.), New York, Plenum Press, 117-148, 1986.
Baier et al., Nucleic Acids Res. 21:4830-4835, 1993.
Bakhshi et al., Cell, 41(3):899-906, 1985.
Balachandran et al., Embo J., 17:6888-6902, 1998.
Baneiji et al., Cell 27(2):299-308, 1981.
Baneiji et al., Cell 33(3):729-40, 1983.
Barany and Merrifield, In:The Peptides, Gross and Meienhofer (Eds.), Academic Press, NY, 1-284, 1979.
Barber, Cell Death Differ. 2001 Feb;8(2):113-26.
Baud et al., Trends Cell Biol, 11, 372-377, 2001.
Began et al., Proc. Am. Assoc. Cancer Res. 44:2382, 2003.
Berens et al., Clin. Exp. Metastasis 12:405-415, 1994.
Beretta etal., Oncogene, 4:12(7):1593-15936, 1996.
Berkhout et al., J. Virol., 63(12):5501-5504, 1989.
Berrie, Expert Opin. Investig. Drugs, 10(6):1085-1098, 2001.
Bianco et al., Clin Cancer Res, 8:3250-3258, 2002.
Blanar et al., Embo. J., 8(4):1139-1144, 1989.
Blumberg et al., Cell, 104:9-19, 2001.
Bodine et al., Embo. J., 6(10):2997-3004, 1987.
Boshart et al., Cell, 41(2):521-530, 1985.
Bosze et al., Embo. J., 5(7):1615-1623, 1986.
Bowman et al., Oncogene, 19, 2474-2488, 2000.
Braddock et al., Cell, 58(2):269-279, 1989.
Brizel, Semin. Radiat. Oncol., 8(4):237-246, 1998.
Bryans et al., Mutat. Res., 433, 53-58, 1999.
Bukowski et al., Clin. Cancer Res., 4(10)-2337-2347, 1998.
Bulla et al., J. Virol., 62(4):1437-1441, 1988.
Bunn et al., Semin. Oncol. 29:87-94, 2002.
Caley et al., J. Virol., 71(4):3031-3038, 1997.
Campbell et al., Mol. Cell Biol., 8(5):1993-2004, 1988.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Camper et al., Biotechnology, 16:81-7, 1991.
Camphausen et al., Cancer Res, 61:2207-2211, 2001.
Campo et al., Nature, 303(5912):77-80, 1983.
Cao et al., Mol Med., 12:9-76, 2002.
Cao et al., Mol. Med. 8:869-976, 2002.
Caudell et al., J Immunol., 168:6041-6046, 2002.
Cavallaro et al., Cancer Lett., 176(2):123-8, 2002.
Celander et al., J. Virol., 61(2):269-75, 1987.
Celander et al., J. Virol., 62(4):1314-22, 1988.
Chada et al., Cancer Gene Ther. 8:S3, 2001, 2001.
Chada et al., Int. Immunopharmcol. (in press), 2004.
Chada et al., Mol. Ther. 7:S446, 2003.
Chan et al., Clin. Cancer Res. 8:904-912,2002.
Chang et al., Mol. Cell Biol., 9(5):2153-62, 1989.
Chang et al., Nature, 410:37-40, 2001.
Chang, Hepatology, 14:134A, 1991.
Chattergoon et al., Nature Biotechnology, 18:974-979, 2000.
Chattedjee et al., Proc. Natl. Acad. Sci. USA, 86(23):9114-8, 1989.
Chen and Tan, Oncogene, 17:173-178, 1998.
Chen et al., J. Biol. Chem., 271:31929-31936, 1996b.
Chen et al., J. Biol. Chem., 271:631-634, 1996a.
Chen et al., Mol. Ther. 8:207-219, 2003.
Chen et al., Nat. Biotechnol., 19:537-542, 2001.
Choi et al., Apoptosis, 8:301-305, 2003.
Chol et al., Eur J Biochem 239(3):579-87, 1996.
Chomczynski and Sacchi, Anal. Biochem., 162:156-159, 1987.
Christodoulides et al., Microbiology, 144(Pt 11):3027-3037, 1998.
Chu et al., Mol Cell Biol. 1998 Jan;18(1):58-68, 1998.
Ciardiello et al., Clin Cancer Res, 7:1459-1465, 2001.
Clake et al., Nucleic Acids Res., 19:243-248 (1991).
Clark et al., Hum. Gene Ther., 6(10):1329-41, 1995.
Cleary et al., Cell, 47(1):19-28, 1986.
Cleary et al., Proc. Natl. Acad. Sci. USA, 82(21):7439-43, 1985.
Cobb et al., Prog. Biophys. Mol. Biol., 71:479-500, 1999.
Coffin, In:Fields Virology, Fields (Ed.), NY, Raven Press, 1437-1500, 1990.
Cohen et al., J. Cell Physiol. Suppl., 5:75-81, 1987.
Costa et al., Mol. Cell Biol., 8(1):81-90, 1988.
Couch, Am. Rev. Resp. Dis., 88:394-403,1963.
Coupar et al., Gene, 68(1):1-10, 1988.
Coux et al., Annu. Rev. Biochem., 65:801-847, 1996.
Cripe et al., Embo. J., 6(12):3745-53, 1987.
Critchlow et al., Trends Biochem. Sci., 23, 394-398, 1998.
Cross et al., Science, 267:1353-6, 1995.
Cryns et al., Genes Dev., 12, 1551-1570, 1998.
Cuddihy et al., Mol. Cell. Biol., 19:2475-2484, 1999.
Culotta et al., Mol. Cell. Biol., 9(3):1376-1380, 1989.
D'Amico et al., J. Biol. Chem., 275(42):32649-32657, 2000.
Dagon et al., Oncogene, 20:8045-8056, 2001.
Dandolo et al., J. Virol., 47(1):55-64, 1983.
Dantuma et al., FEBS Lett., 529:22-26, 2002.
Davidson et al., J. Immunother., 21(5):389-398, 1998.
Davis et al., J. Virol., 70(6):3781-3787, 1996.
Davis, Biochem. Soc. Symp., 64:1-12, 1999.
De Jager et al., Semin. Nucl. Med., 23(2):165-179, 1993.
Deb et al., J. Immunol., 166:6170-6180, 2001.
Dell'Albani et al., J. Neurosci. Res., 65(5):417-424, 2001.
Der et al., Proc. Natl. Acad. Sci., 94:3279-3283, 1997.
Deregibus et al., J. Biol. Chem. 278:18008-18014, 2003.
Deschamps et al., Science, 230(4730):1174-1177, 1985.
Dimmeler et al., Circ. Res., 81:970-976, 1997.
Doolittle and Ben-Zeev, Methods Mol. Biol., 109:215-237, 1999.
Dumoutier et al., J Immunol, 167:3545-3549, 2001.
Eady et al., Br. J. Cancer, 66, 113-118, 1992.
Easwaran et al., J. Biol. Chem., 274(23):16641-16645, 1999.
Edbrooke et al., Mol. Cell Biol., 9(5):1908-1916, 1989.
Edery et al., Cell, 56:303-312, 1989.
Edlund et al., Science, 230(4728):912-916, 1985.
Ekmekcioglu et al., Intl. J. Cancer, 94(1):54-59, 2001.
Ekmekcioglu et al., Melanoma Res., 9(3):261-272, 1999.
el-Kareh et al., Crit. Rev. Biomed. Eng., 25(6):503-571, 1997.
Ellerbroek et al., Cancer Res. 61:1855-1861, 2001.
Ellerhorst et al., J Clin Oncol, 20:1069-1074, 2002.
Ellerhorst et al., J Clin. Oncol. 15:1069-1074, 2001.
Erlandsson, Cancer Genet. Cytogenet., 104(1): 1-18, 1998.
Fan et al., Nat. Med. 9:315-321, 2003.
Fathallah-Shaykh et al., J. Immunol., 164(1):217-222, 2000.
Felgner et al., Proc. Natl. Acad. Sci. USA, 84(21):7413-7417, 1987.
Feng et al., Nature, 334(6178):165-167, 1988.
Feng et al., PNAS USA, 89(12):5447-5451, 1992.
Fickenscher et al., Trends Immunol., 23:89-96, 2002.
Firak et al., Mol. Cell Biol., 6(11):3667-3676.
Firulli et al., In vitro Cell. Dev. Biol. Animal, 34:217-226, 1998.
Fisher et al., Cancer Biol. Ther., 2:S23-S37, 2003.
Flotte et al., Am. J. Respir. Cell Mol. Biol., 7(3):349-356, 1992.
Flotte et al., Proc. Natl. Acad. Sci. USA, 90(22):10613-10617, 1993.
Flotte, et al., Gene Ther., 2(1):29-37, 1995.
Foecking et al., Gene, 45(1):101-105, 1986.
Folkman, N. Engl. J. Med., 320(18):1211-1212, 1989.
Folkman, Nat Med, 1:27-31, 1995.
Fraley et al., Proc, Natl, Acad, Sci, USA, 76(7):3348-3352, 1979:
Freifelder, In:Physical Biochemistry Applications to Biochemistry and Molecular Biology, 2nd ed. Wm. Freeman and Co., NY, 1982.
Friedmann, Science, 244(4910):1275-1281, 1989.
Fry, Breast Cancer Res., 3(5):304-312, 2001.
Fujita et al., Cell, 49(3):357-367, 1987.
Gabizon et al., Cancer Res., 50(19):6371-6378, 1990.
Gaensler et al., Nat. Biotechnol. 17:1188-1192, 1999.
Geng et al., Cancer Res, 61:2413-2419, 2001.
Ghadge et al., J. Virol., 68:4137-4151, 1994.
Ghosh et al., Targeted Diagn Ther., 4:87-103, 1991.
Ghosh-Choudhury et al., Embo. J., 6(6):1733-1739, 1987.
Giaccone, Oncogene, 21:6970-6981, 2002.
Giese et al., Neuro-Oncology 1:3-13, 1999.
Gil et al., Apoptosis. 2000 Apr;5(2):107-14, 2000.
Gil et al., Mol. Cell. Biol., 19:4653-4663, 1999.
Gillies et al., Cell, 33(3):717-728, 1983.
Gloss et al., Embo. J., 6(12):3735-3743, 1987.
Godbout et al., Mol. Cell. Biol., 8:1169, 1988.
Goding, In:Monoclonal Antibodies:Principles and Practice, 2d ed., Orlando, Fla., Academic Press, 60-61, 65-66, 71-74, 1986.
Goh et al., Embo. J., 19:4292-4297, 2000.
Goldfarb et al., Semin. Thromb. Hemostasis, 12:294-307, 1986.
Gomez-Foix et al., J. Biol. Chem., 267(35):25129-25134, 1992.
Goncharova et al., Am. J. Physiol. Lung Cell. Mol. Physiol. 283:L354-L363, 2002.
Goodboum and Maniatis, Cell, 41(2):509-520, 1985.
Goodboum et al., Cell, 45(4):601-610, 1986.
Gopalan et al., Proc. Am. Assoc. Cancer Res. 44:331, 2003.
Gorski et al., Cancer Res, 58:5686-5689, 1998.
Gorski et al., Cancer Res, 59:3374-3378, 1999.
Grawunder et al., Nature, 388, 492-495, 1997.
Greene et al., Adv. Exp. Med. Biol., 254:55-60, 1989.
Grosschedl et al., Cell, 41(3):885-897, 1985.
Grunhaus et al., Seminars in Virology, 3:237-252, 1992.
Gu et al., Proc. Natl. Acad. Sci. U S.A., 94, 8076-8081, 1997a.
Gulbis and Galand, Hum. Pathol., 24(12):1271-1285, 1993.
Gunnery et al., Proc. Natl. Acad. Sci. USA, 87:8687-8691, 1990.
Gupta et al., Nat Med. 6:974-975, 2000.
Haber et al., Trends Genet., 16, 259-264, 2000.
Haines et al., Virchows Arch. B. Cell Pathol., 62:151-158, 1992.
Hanahan et al., Cell, 86:353-364, 1996.
Hanibuchi et al., Int. J Cancer, 78(4):480-485, 1998.
Hanna et al., Cancer J, 6:287-293, 2000.
Harlow and Lane, Antibodies:A Laboratory Manual. Cold Spring Harbor Laboratory Press, Cold Spring harbor, N.Y., 553-612, 1988.
Haslinger et al., Proc. Natl. Acad. Sci. USA, 82(24):8572-8576, 1985.
Hauber et al., J. Virol., 62(3):673-679, 1988.
He et al., Oncogene. 2003.
Hellstrand et al., Acta Oncol., 37(4):347-353, 1998.
Hen et al., Nature, 321(6067):249-251, 1986.
Hensel et al., Lymphokine Res., 8(3):347-351, 1989.
Herbst et al., Clin. Cancer Res. 6:790-797, 2000.
Hernonat et al., Proc. Natl. Acad. Sci. USA, 81(20):6466-6470, 1984.
Herr et al., Cell, 45(3):461-470, 1986.
Herz et al., Proc. Natl. Acad. Sci. USA, 90(7):2812-2816, 1993.
Hesdorffer et al., DNA Cell Biol., 9(10):717-723, 1990.
Hess et al., Br J Cancer, 85:2010-2016, 2001.
Hirochika et al., J. Virol., 61(8):2599-2606, 1987.
Hirsch et al., Mol. Cell Biol., 10(5):1959-1968, 1990.
Ho et al., Cancer, 83(9):1894-1907, 1998.
Holbrook et al., Virology, 159(1):178-182, 1987.
Hopfner et al., Curr. Opin. Struct. Biol., 12, 115-122, 2002.
Horlick et al., Mol. Cell Biol., 9(6):2396-2413, 1989.
Horwich et al., J. Virol., 64(2):642-650, 1990.
Hovanessian, J. Interferon Res., 9:641-647, 1989.
Huang and Kontos, Arterioscler Tromb. Vasc. Biol., 22:745-751, 2002.
Huang et al., Cell, 27(2 Pt 1):245-255, 1981.
Huang et al., Mol. Pharmacol., 62:1515-1521, 2002.
Huang et al., Oncogene, 20:7051-7063, 2001.
Hug et al., Mol. Cell Biol., 8(8):3065-3079, 1988.
Hui et al., Infect. Immun., 66(11):5329-5336, 1998.
Hwang et al., Mol. Cell Biol., 10(2):585-592, 1990.
Icely et al., J. Biolog. Chem., 266(24):16073-16077, 1991.
Imagawa et al., Cell, 51(2):251-260, 1987.
Imbra, et al., Nature, 323(6088):555-558, 1986.
Imler et al., Mol. Cell Biol., 7(7):2558-2567, 1987.
Imperiale et al., Mol. Cell Biol., 4(5):875-882, 1984.
Invitrogen Catalog Nos. V890-20, V891-20, V892-20, and V893-20, “pShooter Vector Manual I (pEF/myc vectors),” on the internet at invitrogen.com/content/sfs/manuals/pshooter_pef_man.pdf
Ito et al., Mol. Ther. (in press).
Ito et al., Mol. Ther. 7: 409-418, 2003.
Ivanov et al., Biochem. (Mosc), 66:1, 2001.
Jagus et al., Int. J. Biochem., 31:123-138, 1999.
Jakobovits et al., Mol. Cell Biol., 8(6):2555-2561, 1988.
Jameel et al., Mol. Cell Biol., 6(2):710-715, 1986.
Jaynes et al., Mol. Cell Biol., 8(1):62-70, 1988.
Jeggo et al., Radiat. Res., 150, S80-91, 1998.
Jiang et al., Oncogene 11:2477-2486, 1995.
Jiang et al., Oncogene, 11:1179-1189, 1995a.
Jiang et al., Proc Natl Acad Sci USA, 93:9160-9165, 1996.
Jimenez et al., Nat. Med., 6:41-48, 2001.
Johnson et al., Mol. Cell Biol., 9(8):3393-3399, 1989.
Joki et al., Nat. Biotech., 19:29-34, 2001.
Jones et al., Cell, 13(1):181-188, 1978.
Judware et al., J. Interferon Res., 13(2):153-160, 1993.
Judware et al., Mol. Cell Biol., 1 1(6):3259-3267, 1991.
Junop et al., EMBO J., 19, 5962-5970, 2000.
Kabat et al., Sequences of Proteins of Immunological Interest, U.S. Department of Health and Human Services, Washington, D.C., 1987.
Kadesch et al., Mol. Cell Biol., 6(7):2593-2601, 1986.
Kadri et al., Cell Growth Differ. 11:573-580, 2000.
Kaneda et al., Science, 243(4889):375-378, 1989.
Kaplitt et al., Nat. Genet., 8(2):148-154, 1994.
Karin et al., Mol. Cell Biol., 7(2):606-613, 1987.
Karin, Ann. N.Y. Acd. Sci., 851:139-146, 1998.
Karlsson et al., Embo. J., 5(9):2377-2385, 1986.
Katinka et al., Cell, 20(2):393-399, 1980.
Kato et al., J. Biol. Chem., 266(6):3361-3364, 1991.
Katso et al., Annu. Rev. Call. Dev. Biol., 17:615-675, 2001.
Kaufmann et al., Trends Cell Biol. 11, 526-534, 2001.
Kawabe et al., Int J Radiat Biol, 77:185-194, 2001.
Kawabe et al., Mol Ther, 6:637-644, 2002.
Kawamoto et al., Mol. Cell Biol., 8(1):267-272, 1988.
Kerr et al., Br. J. Cancer, 26(4):239-257, 1972.
Khanna et al., Nat. Genet., 27, 247-254, 2001.
Kibler et al., J Virol., 71(3):1992-2003, 1997.
Kiledjian et al., Mol. Cell Biol., 8(1):145-152, 1988.
Kim et al., J. Clin. Invest., 100:1006-1014, 1997.
Kitajewski et al., Cell, 45:195-200, 1986.
Klamut et al., Mol. Cell Biol., 10(1): 193-205, 1990.
Koch, et al., Mol. Cell Biol., 9(1):303-311, 1989.
Koch, et al., Rev. Physiol. Biochem. Pharmacol. 146:55-94, 2003.
Kolmel et al., J. Neurooncol., 38(2-3):121-125, 1998.
Koromilas et al., Science, 257(5077):1685-1689, 1992
Kotenko et al., Cytokine Growth Factor Rev., 13:223-240, 2002.
Kotenko et al., Oncogene, 19:2557-2565, 2000.
Kotenko et al., Proc. Natl. Acad. Sci. USA, 97(4):1695-1700, 2000.
Kotin et al., Proc. Natl. Acad. Sci. USA, 87(6):2211-2215, 1990.
Kozin et al., Cancer Res, 61:39-44, 2001.
Kriegleret al., Blood, 63(6):1348-1352, 1984.
Kriegler et al., Cell, 38(2):483-491, 1984.
Kriegler et al., Mol. Cell Biol., 3(3):325-339, 1983.
Kuhen et al., Gene, 178:191-193, 1996.
Kuhl et al., Cell, 50(7):1057-1069, 1987.
Kumar et al., Proc. Natl. Acad. Sci. USA, 91(14):6288-6292, 1994.
Kunz et al., Nucleic Acids Res., 17(3):1121-1138, 1989.
Kurimasa et al., Mol. Cell Biol., 19, 3877-3884, 1999.
Kyte and Doolittle, J. Mol. Biol., 157(1):105-132, 1982.
Lakka et al., Clin. Exp. Metastasis. 18:245-252, 2000.
Lancaster et al., Dig. Dis., 12:170-1766, 1994.
Larsen et al., J. Biol. Chem., 261(31):14373-14376, 1986.
Laspia et al., Cell, 59(2):283-292, 1989.
Latimer et al., Mol. Cell Biol., 10(2):760-769, 1990.
Laughlin et al., J. Virol., 60(2):515-524. 1986.
Le Gal La Salle et al., Science, 259(5097):988-990, 1993.
Lebedeva et al., Oncogene, 21(5):708-18, 2002.
Lebkowski et al., Mol. Cell Biol., 8(10):3988-3996, 1988.
Lee and Esteban, Virology, 199(2):491-496, 1994.
Lee et al., Mol. Cell Biol., 16(6):3023-3034, 1996.
Lee et al., Nature, 294(5838):228-232, 1981.
Lee et al., Nucleic Acids Res., 12(10):4191-4206, 1984.
Lees-Miller et al., Science, 267, 1183-1185, 1995.
Levine et al., Nature 351:453-456, 1991.
Levrero et al., Gene, 101(2):195-202, 1991.
Li et al., Cancer Res., 63, 3268-3274, 2003.
Li et al., Cell, 94:491-501, 1998.
Li et al., J. Virology, 75:5405-5409, 2001.
Lieber et al., Genes Cells, 4, 77-85, 1999.
Lim et al., Biochem Pharmacol. 58:1097-1107,1999.
Lin et al., J Gene Med. 5:868-875, 2003.
Lin et al., Mol. Cell Biol., 10(2):850-853, 1990.
Liotta et al., Cancer Res. 46:1-7, 1986.
Liu et al., J. Biol. Chem., 270(42):24864-2486470, 1995.
Liu et al., Nucleic Acids Res. 25;23(10):1758-65, 1995.
Locksley et al., Cell, 104:487-501, 2001.
Lund et al., Clin Cancer Res, 6:971-978, 2000.
Luria et al., EMBO. J., 6(11):3307-3312, 1987.
Lusky et al., Mol. Cell Biol., 3(6): 1108-1122, 1983.
Lusky et al., Proc. Natl. Acad. Sci. USA, 83(11):3609-3613, 1986.
Macejak and Samow, Nature, 353:90-94, 1991.
Madireddi et al., Adv Exp Med Biol, 465:239-261, 2000.
Madireddi et al., In Cancer Gene Therapy:Past Achievements and Future Challenges, (N. Habib, Ed.), pp. 239-261, Kluwer Academic/Plenum Publishers, New York, 2000.
Madireddi et al., J Cell Physiol, 185:36-46,2000.
Magi-Galluzzi et al., Anal. Quant. Cytol. Histol., 20(5):343-350, 1998.
Maheshwari et al., J. Cell Physiol., 146:164-169, 1991.
Majors et al., Proc. Natl. Acad. Sci. USA, 80(19):5866-5870, 1983.
Majumder et al., J. Neurosci. Res., 54(2):169-180, 1998.
Mangray and King, Front Biosci., 3:D1 148-1160, 1998.
Mangray et al., Front Biosci., 3:D1148-11460, 1998.
Mann et al., Cell, 33(1):153-159, 1983.
Markowitz et al., J. Virol., 62(4): 1120-1124, 1988.
Matrisian et al., Bioessays 14:455-463, 1992.
Mauceri et al., Nature, 394:287-291, 1998.
Mayer et al., Cancer Metastasis Rev., 17(2):211-218, 1998.
McCarty et al., J. Virol., 65(6):2936-2945, 1991.
McCormkick, Trends in Cell Biol., 12:53-86, 1999.
McDonald et al., Cancer Res., 56, 2250-2255, 1996.
McEntee et al., Carcinogenesis. 20:635-640,1999.
McLaughlin et al., J. Virol., 62(6):1963-1973, 1988.
McNeall et al., Gene, 76(1):81-88, 1989.
Merrifield, Science, 232:341-347, 1986.
Meurs et al., Proc. Natl. Acad. Sci. USA, 90(1):232-236, 1993.
Mhashilkar et al., Mol Med 2001; 7:271-282, 2001.
Mhashilkar et al., Mol. Ther. 8:220-229, 2003.
Mhashilkar et al., Mol. Ther. 8:207-219, 2003.
Mhashilkar, A., et al. MDA-7 regulates tumor suppressor gene activation and oncogene inactivation via β-catenin and P13K pathways in breast and lung tumor cells. Mol. Ther. (in press), 2003.
Michaelson et al., Oncogene, 20(37):5093-5099, 2001.
Miksicek et al., Cell, 46(2):283-290, 1986.
Millauer et al., Nature, 367(6463):576-579, 1994.
Molina et al., Cancer Res. 59 :4356-4362, 1999.
Mordacq et al., Genes Dev., 3(6):760-769, 1989.
Moreau et al., Nucleic Acids Res., 9(22):6047-6068, 1981.
Mougin et al., Ann. Biol. Clin., 56(1):21-28, 1998.
Muesing et al., Cell, 48(4):691-701, 1987.
Mumby et al., Cell Regul., 2(8):589-598, 1991.
Muzio et al., Cell, 85:817-827, 1996.
Muzyczka, Curr. Top Microbiol. Immunol., 158:97-129, 1992.
Nakamura et al., In:Handbook of Experimental Immunology (₄th Ed.), Weir et al., (eds). 1:27, Blackwell Scientific Publ., Oxford, 1987.
Nakopoulou et al., Breast Cancer Res. Treat. 77:145-155, 2003.
Natoli et al., Biochem. Pharmacol., 56(8):915-920, 1998.
Natoli et al., Science, 275:200-203, 1997.
Neuberger et al., Nucleic Acids Res., 16(14B):6713-6724, 1988.
Neudauer et al., Exp. Cell Res. 286:128-137, 2003.
Ng et al., Nucleic Acids Res., 17(2):601-615, 1989.
Nicholson et al., Nature, 376:37-43, 1995.
Nicolas and Rubenstein, In: Vectors:A survey of molecular cloning vectors and their uses, Rodriguez and Denhardt (eds.), Stoneham:Butterworth, pp. 493-513, 1988.
Ning et al., Radiat Res, 157:45-51, 2002.
Nishikawa et al., Proc. Am. Assoc. Cancer Res. 44:331, 2003.
O'Reilly et al., J. Biol. Chem., 274(41):29568-29571, 1994.
Oberg et al., Anticancer Res. 20:1085-1091, 2000.
Oehm et al., J Biol Chem 267:10709-10715.
Ohara, Gan. To Kagaku Ryoho, 25(6):823-828, 1998.
Ohbayashi et al., Curr. Protein Pept. Sci. 3:409-421, 2002.
Ohi et al., Gene, 89(2):279-282, 1990.
Olive, Radiat. Res., 150, S42-51, 1998.
Omori et al., DNA Repair (Amst), 1, 299-310, 2002.
Ondek et al., EMBO. J., 6(4):1017-1025, 1987.
Ono et al., Cell. Signal. 12, 1-13,2 000.
O'Reilly et al., Cell, 79:315-328, 1994.
O'Reilly et al., Cell, 88:277-285, 1997.
Ornitz et al., Mol. Cell Biol., 7(10):3466-3472, 1987.
Palmiter et al., Cell, 29(2):701-710, 1982.
Parker et al., Br. J. Cancer, 85(12):1958-1963, 2001.
Parker et al., CA Cancer J. Clin., 47, 5-27, 1997.
Parker et al., Cancer statistics, 1997. CA Cancer J Clin, 47:5-27, 1997.
Paskind et al., Virology, 67(1):242-248, 1975.
Pataer et al., Cancer Res., 62:2239-2243, 2002.
Pataer et al., J Thorac Cardiovasc Surg. 125(6):1328-35, 2003.
PCT Appl. WO 00/05356
PCT Appl. WO 00/26368
PCT Appl. WO 0005356
PCT Appl. WO 0026368
PCT Appl. WO 98/07408
PCT Appl. WO 98/28425
PCT Appl. WO 9807408
PCT Appl. WO 9828425
Pech et al., Mol. Cell Biol., 9(2):396-405, 1989.
Peifer et al., Science, 14:1837-1851, 2000.
Pelletier et al., Nature, 334(6180):320-325, 1988.
Peng et al., Science, 277:1501-1505, 1997.
Perez-Stable et al., Mol. Cell Biol., 10(3): 1116-1125, 1990.
Petryshyn et al., J. Biol. Chem., 259(23):14736-14742, 1984.
Petryshyn et al., Proc. Natl. Acad. Sci. USA, 85(5):1427-1431, 1988
Philip et al., J. Biol. Chem., 268(22):16087-16090, 1993.
Piazza et al., Cancer Res., 55:3110-3116, 1995.
Piazza et al., Cancer Res., 57:2909-2915, 1997.
Picard et al., EMBO. J., 4(11):2831-2838, 1985.
Pietras et al., Oncogene, 17(17):2235-2249, 1998.
Pinkert et al., Genes Dev., 1(3):268-276, 1987.
Ponta et al., Proc. Natl. Acad. Sci. USA, 82(4):1020-1024, 1985.
Pritchard et al., Oncol. Rep. 8:421-424, 2001.
Qin et al., Proc. Natl. Acad. Sci. USA, 95(24):14411-14416, 1998.
Queen and Baltimore, Cell, 35:741, 1983.
Quinn et al., Mol. Cell Biol., 9(11):4713-4721, 1989.
Rainbow et al., Radiat. Res., 50, 319-333, 1972.
Rainbow, Radiat. Res., 60, 155-164, 1974.
Ramesh et al., Cancer Res., 63(16):5105-5113, 2003.
Ramesh et al., Mol. Ther. (in press).
Ramesh et al., Mol. Ther., 3(3):337-350, 2001.
Ramesh, R., et al. The melanoma differentiation associated-7 gene (mda-7)/interleukin-24 (1L-24) is a novel ligand that regulates angiogenesis via the WL-22 receptor. Cancer Res. (in press), 2003.
Redondo et al., Science, 247(4947):1225-1229, 1990.
Reisman et al., Oncogene, 4(8):945-953, 1989.
Remington's Pharmaceutical Sciences, 15th ed., pages 1035-1038 and 1570-1580, Mack Publishing Company, Easton, Pa., 1980.
Renan, Radiother. Oncol., 19(3):197-218, 1990.
Resendez et al., Mol. Cell Biol., 8(10):4579-4584, 1988.
Restifo, J. Immunother., 24(3):193-194, 2001.
Riballo et al., Curr. Biol., 9, 699-702, 1999.
Rippe et al., Mol. Cell Biol., 9(5):2224-2227, 1989.
Rittling et al., Nucleic Acids Res., 17(4): 1619-1633, 1989.
Roth et al., Semin. Oncol., 28:50-56, 2001.
Rothman et al., Circulaton, 86:1977-1986, 1992.
Rothman et al., J. Biol. Chem., 269:6399-6404, 1994.
Rye et al., J Biol Chem. 278:24003-24010, 2003.
Sado et al., J. Biol. Chem., 276, 9742-9748, 2001.
Saeki et al., Gene Ther. 7:2051-2057, 2000.
Saeki et al., Oncogene 21:4558-4566, 2002.
Saelens et al., J. Biol. Chem., 276:41620-41628, 2001.
Saito et al., Proc. Am. Assoc. Cancer Res. 44:247, 2003, 2003.
Sambrook et al., In: Molecular cloning, Cold Spring Harbor Laboratory Press, Cold Spring Harbor, N.Y., 2001.
Samulski et al., J. Virol., 63(9):3822-38228, 1989.
Sanchez-Alcazar et al., Lung Cancer. 40:33-44, 2003.
Sarkar et al., Biotechniques. 8:30-39, 2002.
Sarkar et al., Proc Natl Acad Sci USA, 99:10054-10059, 2002.
Sasaki et al., Nat. Biotechnol., 19(6):543-547, 2001.
Saura et al., J. Mol. Biol., 289(3):459-471, 1999.
Schaefer et al., J Immunol. 15:5859-5863, 2001.
Schaefer et al., J. Immunol., 166:5859, 2001.
Schaffner et al., J. Mol. Biol., 201(1):81-90, 1988.
Searle et al., Mol. Cell Biol., 5(6):1480-1489, 1985.
Shapiro et al., Curr. Opin. Cell Biol. 10:602-608, 1998.
Sharp et al., Cell, 59(2):229-230, 1989.
Shaul et al., EMBO. J., 6(7):1913-1920, 1987.
Shelling et al., Gene Ther., 1(3):165-169, 1994.
Sherman et al., Proc. Natl. Acad. Sci. USA, 86(17):6739-6743, 1989.
Shi et al., J Interferon Cytokine Res, 21:553-566, 2001.
Sibanda et al., Nat. Struct. Biol., 8, 1015-1019, 2001.
Sieger et al., Mol. Ther. (in press), 2004.
Sieger et al., Mol. Ther. (in press).
Sienel et al., Int. J. Cancer 103:647-651, 2003.
Sleigh and Lockett, EMBO. J., 4:3831, 1985.
Smith et al., Genes Dev., 13, 916-934, 1999.
Solodin et al., Biochemistry, 34(41):13537-13544, 1995.
Solyanik et al., Cell Prolif., 28(5):263-278, 1995.
Soo et al., J. Cell. Biochem., 74(1):1-10, 1999.
Soriano et al., Cancer Res., 59:6178-6184, 1999.
Spalholz et al., Cell, 42:183, 1985.
Spandau et al., J. Virol., 62(2):427-434, 1988.
Spandidos et al., EMBO. J., 2(7):1193-1199, 1983.
Spitz et al., Clin Cancer Res, 2:1665-1671, 1996.
Stephens et al., Biochem. J., 248(1):1-11, 1987.
Stewart and Young, In:Solid Phase Peptide Synthesis, 2d. ed., Pierce Chemical Co., 1984.
Stewart et al., Mol. Med., 8:451-461, 2002.
Stokke et al., Cell Prolif., 30(5):197-218, 1997.
Story et al., Int. J. Radiat. Biol., 65, 523-528. 1004.
Stuart et al., Nature, 317(6040):828-831, 1985.
Su et al., Oncogene, 22:1164-1180, 2003.
Su et al., Proc Natl Acad Sci U S A, 95:14400-14405, 1998.
Su et al., Proc Natl Acad Sci USA, 98:10332-10337, 2001.
Sudhakar et al., Biochem., 39:12929-12938, 2000.
Sullivan et al., Mol. Cell Biol., 7(9):3315-3319, 1987.
Sundararajan et al., J. Biol. Chem., 276:45120-45127, 2001.
Swartzendruber et al., J. Cell Physiol., 85(2 Pt 1):179-187, 1975.
Swisher et al., Clin Cancer Res, 9:93-101, 2003.
Swisher et al., Curr Oncol Rep. 4(4):334-40, 2002.
Swisher et al., J Natl Cancer Inst 91:763-771, 1999.
Takebe et al., Mol. Cell Biol., 8(1):466-472, 1988.
Tam et al., J. Am. Chem. Soc., 105:6442, 1983.
Tang et al., Mol. Cell, 8:1005-1016, 2001.
Tartaglia et al., Immunol. Today, 13:151-153, 1992.
Tavernier et al., Nature, 301(5901):634-636, 1983.
Taylor et al., Mol. Cell Biol, 10(1): 176-183, 1990.
Taylor et al., Science. 1999 Jul 2;285(5424):107-10, 1999.
Tegeder et al., FASEB J. 15:2057-2072, 2001
Temin, In:Gene Transfer, Kucherlapati (ed.), NY, Plenum Press, 149-188, 1986.
Templeton et al., Nat. Biotechnol. 15:647-652, 1997.
Thierry et al., Proc. Natl. Acad. Sci. USA, 92(21):9742-9746, 1995.
Thiesen et al., J. Virol., 62(2):614-618, 1988.
Thompson et al., Cancer Res. 57:267-271, 1997.
Thompson et al., Cancer Res. 60:3338-3342., 2000.
Top et al., J. Infect. Dis., 124(2):155-60, 1971.
Toyoshima et al., Cell, 78:67-74, 1994.
Tratschin et al., Mol. Cell Biol., 4(10):2072-2081, 1984.
Tratschin et al., Mol. Cell Biol., 5(11):3251-3260, 1985.
Treisman et al., Cell, 42(3):889-902, 1985.
Tronche et al., Mol. Biol. Med., 7(2):173-185, 1990.
Tronche et al., Mol. Cell Biol., 9(11):4759-4766, 1989.
Trudel et al., Genes Dev., 1(9):954-961, 1987.
Tsuiki et al., Oncogene, 20:420-429, 2001.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Turnier et al., Science (Wash. D.C.), 288:870-874, 2000.
Tyndall et al., Nucleic Acids Res., 9(23):6231-6250, 1981.
Vane et al., Annu Rev Pharmacol Toxicol. 38:97-120, 1998.
Vamavski et al., J Virol. 76:5711-5719, 2002.
Vasseur et al., Proc. Natl. Acad. Sci. USA, 77(2):1068-1072, 1980.
Vattem et al., Eur. J. Biochem., 268:3674-3684, 2001.
Vlachaki et al., Mol Ther. 6:342-348, 2002.
Vorburger et al., Oncogene. 2002 Sep 12;21(41):6278-88, 2002.
Walker et al., Nature, 412, 607-614, 2001.
Walsh et al., J. Clin. Invest., 94(4):1440-1448, 1994.
Walter and Johnson, Annu. Rev. Cell Biol. 10:87-119, 1994.
Wang et al., Cell, 47(2):241-7, 1986.
Wang et al., Exp. Cell Res. 247:17-28, 1999.
Wang et al., J Biol Chem, 277:7341-7347, 2002.
Wang et al., J. Biol. Chem., 281:1680-1683, 1998.
Wang et al., Science, 281:1680-1683, 1998.
Weber et al., Cell, 36(4):983-992, 1984.
Wei et al., Gene Ther., 1(4):261-268, 1994.
Weidner et al., N Engl J Med, 324:1-8, 1991.
Williams et al., Oncogene. 18 :7908-7916, 1999.
Williams et al., Sci STKE. 3;2001(89):RE2, 2001.
Winoto et al., EMBO. J. 8(3):729-733, 1989.
Wolf et al., J. Biol. Chem. 274, 20049-20052, 1999.
Wong et al., Gene, 10(2):87-94, 1980.
Woodfield et al., Biochem. J., 360(Pt 2):335-344, 2001.
Woolson, In: Statistical Methods for the Analysis of Biomedical Data, John Wiley & Sons, 206, NY, 1987.
Yacoub et al., Cancer Biol. Ther., 2003.
Yacoub et al., Clin Cancer Res. 9:3272-3281, 2003.
Yacoub et al., Mol Cancer Ther. 2:623-632, 2003.
Yang et al., Carcinogenesis. 24:605-611, 2003.
Yang et al., Int. J. Oncol., 18(3):541-548, 2001.
Yang et al., J. Virol., 68(8):4847-4856, 1994.
Yang et al., Proc. Natl. Acad. Sci. USA, 87(24):9568-9572, 1990.
Yeung et al., Proc. Natl. Acad. Sci. USA, 91(24):11413-11416, 1994.
Yin et al., J Biol Chem. 20;278(25):22838-45. Epub Apr 04, 2003.
Yoder et al., Blood, 82(Suppl.):347A, 1994.
Yonehara et al., J Exp Med 169:1747-1756, 1989.
Yutzey et al., Mol. Cell Biol., 9(4):1397-1405, 1989.
Zamanian-Daryoush et al., Oncogene, 18:315-326, 1999.
Zhang et al., Biochem. Biophys. Res. Commun., 290:1123-1127, 2002.
Zhang et al., J. Biol. Chem., 275:24436-24443, 2000.
Zhang et al., J. Biol. Chem., 276:26946-24958, 2001.
Zhang et al., Science, 290:989-992, 2000.
Zhou et al., Exp. Hematol., 21(7):928-933, 1993.
Zhou et al., J. Exp. Med., 179(6):1867-1875, 1994.
Zhu et al., Science, 261(5118):209-211, 1993.

What is claimed is:

1. A method for purifying MDA-7 protein from a cell comprising subjecting a cell extract or supernatant comprising MDA-7 protein to affinity chromatography.

2. The method of claim 1, wherein the MDA-7 protein is purified to at least 20% homogeneity and is active.

3. The method of claim 1, wherein the MDA-7 protein is glycosylated and is the secreted form of the protein.

4. The method of claim 1, further comprising adding a protein carrier to the extract or supernatant before, during, or after subjection to affinity chromatography.

5. The method of claim 1, further comprising subjecting the affinity-purified MDA-7 protein to anion exchange chromatography, wherein the resulting MDA-7 protein is purified to at least 30% homogeneity and is active.

6. The method of claim 5, wherein the anion exchange chromatography involves a step gradient of salt up to a concentration of 1.0 M.

7. The method of claim 6, wherein the MDA-7 protein is eluted in a solution with a salt concentration of about 0.9 M to 1.0 M.

8. The method of claim 5, wherein the resulting protein is purified to 50%-70% homogeneity.

9. The method of claim 8, wherein the resulting protein is purified to 70%-90% homogeneity.

10. The method of claim 9, wherein the resulting protein is purified to at least 90% homogeneity.

11. The method of claim 10, wherein the resulting protein is purified to at least 95% homogeneity.

12. The method of claim 1, wherein the affinity chromatography involves an affinity resin comprising at least one anti-MDA-7 polyclonal antibody.

13. The method of claim 1, wherein the affinity chromatography involves an affinity resin comprising at least one anti-MDA-7 monoclonal antibody.

14. The method of claim 1, wherein the affinity chromatography comprises contacting the cell extract or supernatant with an affinity resin, washing the resin, and eluting MDA-7 protein from the resin.

15. The method of claim 14, wherein the MDA-7 protein is eluted with a solution comprising 1 M salt and a pH below 5.0.

16. The method of claim 14, further comprising neutralizing the eluted MDA-7 protein with a buffer.

17. The method of claim 14, further comprising incubating the eluted MDA-7 protein with Protein A.

18. The method of claim 17, wherein the Protein A is coupled or attached to a nonreacting material.

19. The method of claim 1, further comprising subjecting the affinity chromatography-purified MDA-7 protein to size resolution purification.

20. The method of claim 19, wherein size resolution purification occurs before and/or after anion exchange chromatography.

21. The method of claim 19, wherein size resolution purification involves a protein gel or a size exclusion column.

22. A method for purifying active, glycosylated, secreted MDA-7 protein from a cell comprising:
a) subjecting a cell extract or supernatant comprising secreted MDA-7 protein to affinity chromatography involving an anti-MDA antibody;
b) subjecting the affinity chromatography-purified MDA-7 protein to size resolution purification; and,
c) subjecting the size resolution-purified MDA-7 to anion exchange chromatography.

23. The method of claim 22, wherein the MDA-7 protein is purified to at least 80% homogeneity.

24. Purified MDA-7 protein, wherein the protein is active.

25. The purified MDA-7 protein of claim 24, wherein the MDA-7 protein is purified to at least about 25% homogeneity.

26. The MDA-7 protein of claim 25, wherein the MDA-7 protein is purified to at least about 40% homogeneity.

27. The MDA-7 protein of claim 26, wherein the MDA-7 protein is purified to at least about 50% homogeneity.

28. The MDA-7 protein of claim 27, wherein the MDA-7 protein is purified to at least about 60% homogeneity.

29. The MDA-7 protein of claim 28, wherein the MDA-7 protein is purified to at least about 70% homogeneity.

30. The MDA-7 protein of claim 29, wherein the MDA-7 protein is purified to at least about 80% homogeneity.

31. The MDA-7 protein of claim 30, wherein the MDA-7 protein is purified to at least about 90% homogeneity.

32. The MDA-7 protein of claim 31, wherein the MDA-7 protein is purified to at least about 95% homogeneity.

33. A method for treating a cancer patient comprising administering to the patient an effective amount of a pharmaceutically acceptable composition comprising purified secreted MDA-7 protein of claim 24.

34. The method of claim 33, wherein the MDA-7 protein is active and at least about 80% homogeneous with respect to proteins in the composition.

35. The method of claim 33, further comprising subjecting the patient to radiotherapy or chemotherapy.

36. The method of claim 35, wherein the cancer patient has an epithelial cell cancer.

37. The method of claim 33, wherein the cancer patient has a melanoma or pancreatic cancer.

38. A method for radiosensitizing a cancer cell comprising administering to the cell an effective amount of an adenovirus vector comprising a nucleic acid encoding MDA-7, wherein the nucleic acid is under the control of a promoter operable in the cell.

39. The method of claim 38, wherein the cancer cell is an epithelial cell.

40. The method of claim 38, further comprising subjecting the cancer cell to radiation within 72 hours after administration of the adenoviral vector.

41. A method of treating cancer in a patient comprising administering to the patient an NF-κB inhibitor and a composition comprising either MDA-7 protein or an adenovirus vector comprising a nucleic acid encoding MDA-7 under the control of a promoter.

42. The method of claim 41, wherein the NF-κB inhibitor is Sulindac.

43. A protein comprising amino acids 100 to 206 of SEQ ID NO:2 and an endoplasmic reticulum targeting sequence.

44. A method for inhibiting or preventing local invasiveness and/or metastasis of cancer in a patient, comprising administering to the patient an effective amount of a pharmaceutically acceptable composition comprising MDA-7 protein, wherein the MDA-7 inhibits or prevents the local invasiveness and/or metastasis of the cancer.

45. The method of claim 44, wherein the cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder.

46. The method of claim 45, wherein the cancer is lung cancer.

47. A method for inhibiting or preventing local invasiveness and/or metastasis of cancer in a patient, comprising administering to the patient an effective amount of a pharmaceutically acceptable composition comprising a polynucleotide encoding an MDA-7 protein, wherein the MDA-7 protein inhibits or prevents the local invasiveness and/or metastasis of the cancer.

48. The method of claim 47, wherein the polynucleotide encoding an MDA-7 protein comprises an expression construct.

49. The method of claim 48, wherein the expression construct comprises an adenovirus vector comprising a nucleic acid, under the control of a promoter, encoding the MDA-7 protein.


SEQUENCE LISTING (3 sequences)

SEQ ID NO: 1 - DNA, 718 bp, Homo sapiens
acaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - DNA, 21 bp, Artificial Sequence
  Description of Artificial Sequence  Synthetic Primer
cgcttgatgagtcagccggaa